(19) United States
(12) Patent Application Publication
(10) Pub. No.: US 2015/0336960 A1
(43) Pub. Date: Nov. 26, 2015

(54) ARYL-SUBSTITUTED FUSED BICYCLIC PYRIDAZINE COMPOUNDS

(71) Applicants: Matthew BURGER, Emeryville, CA (US); Gisele NISHIGUCHI, Emeryville, CA (US); Alice RICO, Emeryville, CA (US); Benjamin TAFT, Emeryville, CA (US); NOVARTIS AG, Basel (CH)

(72) Inventors: Matthew BURGER, Belmont, MA (US); Gisele NISHIGUCHI, Arlington, MA (US); Alice RICO, Castrol Valley, CA (US); Benjamin TAFT, Oakland, CA (US)

(73) Assignee: NOVARTIS AG, Basel (CH)

(21) Appl. No.: 14/651,996

(22) PCT Filed: Dec. 7, 2013
(86) PCT No.: PCT/US2013/075613

§ 371 Date: Jun. 12, 2015

Related U.S. Application Data
(60) Provisional application No. 61/739,522, filed on Dec. 19, 2012.

(51) Int. Cl. C07D 487/04; A61K 45/06; A61K 31/5025

(52) U.S. Cl. CPC C07D 487/04; A61K 31/5025; A61K 45/06

(57) ABSTRACT

The present invention provides a compound of formula (I) as described herein, and pharmaceutically acceptable salts, enantiomers, rotamers, tautomers, or racemates thereof. Also provided are methods of treating a disease or condition mediated by PIM kinase using the compounds of Formula (I), and pharmaceutical compositions comprising such compounds.

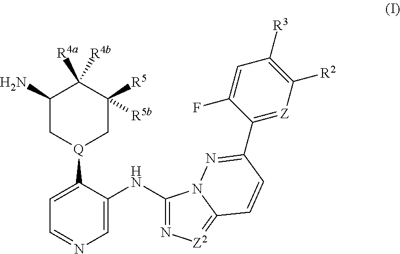

### FIELD OF THE INVENTION

The present invention relates to new compounds and compositions of the new compounds together with pharmaceutically acceptable carriers, and uses of the new compounds, either alone or in combination with at least one additional therapeutic agent, in the prophylaxis or treatment of cancer and other cellular proliferation disorders.

### BACKGROUND

Infection with the Moloney retrovirus and genome integration in the host cell genome results in development of lymphomas in mice. Provirus Integration of Moloney Kinase (PIM-Kinase) was identified as one of the frequent proto-oncogenes capable of being transcriptionally activated by this retrovirus integration event (Cuypers H T et al., “Murine leukemia virus-induced T-cell lymphomagenesis: integration of proviruses in a distinct chromosomal region,” Cell 37(1): 141-50 (1984); Selten G, et al., “Proviral activation of the putative oncogene Pim-1 in MuLV induced T-cell lymphomas” EMBO J 4(7): 1793-8 (1985)), thus establishing a correlation between over-expression of this kinase and its oncogenic potential. Sequence homology analysis demonstrated that there are three highly homologous Pim-Kinases (Pim1, 2 & 3), Pim1 being the proto-oncogene originally identified by retrovirus integration. Furthermore, transgenic mice over-expressing Pim1 or Pim2 show increased incidence of T-cell lymphomas (Breuer M et al., “Very high frequency of lymphoma induction by a chemical carcinogen in pim-1 transgenic mice” Nature 340(6228):61-3 (1989)), while over-expression in conjunction with c-myc is associated with incidence of B-cell lymphomas (Verbeek S et al., “Mice bearing the E mu-myc and E mu-pim-1 transgenes develop pre-B-cell leukemia prenatally” Mol Cell Biol 11(2): 1176-9 (1991)). Thus, these animal models establish a strong correlation between Pim over-expression and oncogenesis in hematopoietic malignancies. In addition to these animal models, Pim over-expression has been reported in many human malignancies, particularly in hematopoietic and in prostate cancer. Furthermore, mutational activation of several well known oncogenes in hematopoietic malignancies is thought to exert its effects at least in part through Pim(s). For example, targeted down-regulation of Pim expression impairs survival of hematopoietic cells transformed by Flt3 and BCR/ABL (Adam et al. 2006).

Pim1, 2 & 3 are Serine/Threonine kinases that normally function in survival and proliferation of hematopoietic cells in response to growth factors and cytokines Substrates for Pim kinases include regulators of apoptosis such as the Bcl-2 family member BAD. The effects of Pim(s) in these regulators are consistent with a role in protection from apoptosis and promotion of cell proliferation and growth. Thus, over-expression of Pim(s) in cancer is thought to play a role in promoting survival and proliferation of cancer cells and, therefore, their inhibitions should be an effective way of treating cancers in which they are over-expressed. In fact several reports indicate that knocking down expression of Pim(s) with siRNA results in inhibition of proliferation and cell death (Dai J M, et al., “Antisense oligodeoxynucleotides targeting the serine/threonine kinase Pim-2 inhibited proliferation of DU-145 cells,” Acta Pharmacol Sin 26(3):364-8 (2005); Fujii et al. 2005; Li et al. 2006). Thus, inhibitors to Pim1, 2 and 3 would be useful in the treatment of these malignancies.

In addition to a potential role in cancer treatment and myeloproliferative diseases, such inhibitors could be useful to control expansion of immune cells in other pathologic condition such as autoimmune diseases, allergic reactions and in organ transplantation rejection syndromes. Recent reports demonstrate that Pim kinase inhibitors show activity in animal models of inflammation and autoimmune diseases. See JE Robinson “Targeting the Pim Kinase Pathway for Treatment of Autoimmune and Inflammatory Diseases,” for the Second Annual Conference on Anti-Inflammatories: Small Molecule Approaches,” San Diego, Calif. (Conf. April 2011; Abstract published earlier on-line).

A continuing need exists for compounds that inhibit the proliferation of capillaries, inhibit the growth of tumors, treat cancer, modulate cell cycle arrest, and/or inhibit molecules such as Pim1, Pim2 and Pim3, and pharmaceutical formulations and medicaments that contain such compounds. A need also exists for methods of administering such compounds, pharmaceutical formulations, and medicaments to patients or subjects in need thereof. The present invention addresses such needs.

Earlier patent applications have described compounds that inhibit Pims and function as anticancer therapeutics, see, e.g., WO2012/004217, WO2010/026124, WO 2008/106692 and WO2011/124580, and as treatment for inflammatory conditions such as Crohn's disease, inflammatory bowel disease, rheumatoid arthritis, and chronic inflammatory diseases, see e.g., WO 2008/022164. The present invention provides novel compounds that inhibit activity of one or more Pims, preferably two or more Pims, more preferably Pim1, Pim2 and Pim3, at nanomolar levels (e.g., IC-50 under 50 nM) and exhibit distinctive characteristics that may provide improved therapeutic effects and pharmacokinetic properties, such as reduced drug-drug interactions associated with inhibition of cytochrome oxidases, relative to compounds previously disclosed. Compounds of the invention contain novel substitution combinations on one or more rings that provide these distinctive properties and are suitable for treating Pim-related conditions such as those described herein.

### SUMMARY OF THE INVENTION

The invention provides unsaturated compounds of Formula (I) that inhibit one or more Pim kinases:

The invention provides bicyclic pyridazine compounds of Formula (I):

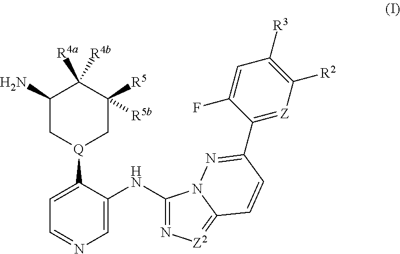

wherein:
Z is CH, CF or N, typically Z is CF or N;
Z² is CH or N, in many embodiments Z² is CH;
Q is CH or N; when Q is CH, the compound has the relative stereochemistry as shown;
R² is H or —C(O)NR*₂; preferably R² is H or —C(O)NHR*, and typically R³ is H when R² is —C(O)NHR*;
R^{4a} and R^{4b} are each selected from H, CN, halo, azido, amino, R⁶, —OR⁶, C₁₋₄ alkyl, C₁₋₄ haloalkyl, —O(CH₂)₁₋₃—OR⁶, —NRC(O)R⁶, —NRCOOR⁶, NRSO₂R⁶, —SO₂R⁶, N-pyridonyl, or 1-triazolyl (e.g., N-1,2,3-triazolyl); preferably R^{4a} and R^{4b} are selected from H, halo, Me, CF₃, OH, OR⁶, SO₂R⁶, NRC(═O)R⁶, or NRC(═O)OR⁶;
   where each R is H or C₁₋₄ alkyl;
   provided that when R^{4a} is H and R^{4b} is H or OH, R² and R³ cannot both be H;
R⁵ is H or C₁₋₄ alkyl;
R^{5b} is H, or R^{4b} and R^{5b} taken together form a double bond between the carbon atoms to which they are attached;
R⁶ is C₁₋₄ alkyl optionally substituted with up to three groups selected from halo, CN, C₁₋₄ alkylsulfonyl, hydroxy, and C₁₋₄ alkoxy;
each R³ is independently selected from CN, hydroxy, C₁₋₄ haloalkyl, —S(O)ₚ—R*, C₁₋₄ haloalkoxy, —(CH₂)₀₋₃—OR*, —O—(CH₂)₁₋₃—OR*, —CONR*₂, —(CR′₂)₁₋₃—OR′, —O—(CR′₂)₁₋₃—OR′, and an optionally substituted member selected from the group consisting of -L-C₁₋₆ alkyl, -L-C₁₋₆ alkylsulfonyl, -L-C₃₋₇ cycloalkyl, and -L-C₄₋₇ heterocycloalkyl, wherein each L is selected from a bond, —O—, —CH₂—, —CH₂—O— and —O—CH₂—, and each C₁₋₆ alkyl, C₁₋₆ alkylsulfonyl, C₃₋₇ cycloalkyl, and C₄₋₇ heterocycloalkyl contains one or two heteroatoms selected from N, O and S, and is optionally substituted with up to two groups selected from halo, CN, hydroxy, C₁₋₄ alkoxy, and R*;
or R³ can be H when R² is —C(O)NHR*;
   where each R′ is independently H or Me or Et,
   and each R* is independently H or a 4-7 membered cyclic ether, 3-6 membered cycloalkyl, pyrrolidine, or C₁₋₆ alkyl, each of which is optionally substituted with up to three groups selected from halo, oxo, C₁₋₄ alkyl, C₁₋₄ alkoxy, OH, OMe, OEt, and CN; and
p is 0, 1 or 2;
   and pharmaceutically acceptable salts thereof

Embodiments of these compounds and pharmaceutical compositions and uses for these compounds and compositions are described below.

These compounds are inhibitors of Pim kinases as further discussed herein. These compounds and their pharmaceutically acceptable salts, and pharmaceutical compositions containing these compounds and salts, are useful for therapeutic methods such as treatment of cancers and autoimmune disorders that are caused by or exacerbated by excessive levels of Pim kinase activity.

### DETAILED DESCRIPTION OF EMBODIMENTS OF THE INVENTION

“PIM inhibitor” is used herein to refer to a compound that exhibits an IC₅₀ with respect to PIM Kinase activity of no more than about 100 μM and more typically not more than about 5 μM, as measured in the PIM assays described herein for at least one of Pim1, Pim2 and Pim3. Preferred compounds have on IC₅₀ below about 1 micromolar on at least one Pim, and generally have an IC₅₀ below 100 nM on each of Pim1, Pim2 and Pim3.

The phrase “alkyl” refers to hydrocarbon groups that do not contain heteroatoms, i.e., they consist of carbon atoms and hydrogen atoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —

CH(CH₃)₂, —CH(CH₃)(CH₂CH₃), —CH(CH₂CH₃)₃, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)—
CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂—
CH(CH₃)₂, —CH(CH₃)CH(CH₃)CH(CH₃)₂, —CH(CH₂CH₃)CH(CH₃)CH(CH₃)(CH₂CH₃), and others. Thus the term ‘alkyl’ includes primary alkyl groups, secondary alkyl groups, and tertiary alkyl groups. Alkyl groups are described herein according to the number of carbon atoms they contain, e.g., an alkyl group containing up to six carbon atoms is described as a C1-6 or C₁₋₆, or C1-C6 alkyl. Typical alkyl groups include straight and branched chain alkyl groups having 1 to 12 carbon atoms, preferably 1-6 carbon atoms. The term ‘lower alkyl’ or “loweralkyl” and similar terms refer to alkyl groups containing up to 6 carbon atoms.

The term “alkenyl” refers to alkyl groups as defined above, wherein there is at least one carbon-carbon double bond, i.e., wherein two adjacent carbon atoms are attached by a double bond. The term “alkynyl” refers to alkyl groups wherein two adjacent carbon atoms are attached by a triple bond. Typical alkenyl and alkynyl groups contain 2-12 carbon atoms, preferably 2-6 carbon atoms. Lower alkenyl or lower alkynyl refers to groups having up to 6 carbon atoms. An alkenyl or alkynyl group may contain more than one unsaturated bond, and may include both double and triple bonds, but of course their bonding is consistent with well-known valence limitations.

The term ‘alkoxy” refers to —OR, wherein R is alkyl.

As used herein, the term “halogen” or “halo” refers to chloro, bromo, fluoro and iodo groups. Typical halo substituents are F and/or Cl. “Haloalkyl” refers to an alkyl radical substituted with one or more halogen atoms, typically 1-3 halogen atoms. The term “haloalkyl” thus includes monohalo alkyl, dihalo alkyl, trihalo alkyl, perhaloalkyl, and the like.

“Amino” refers herein to the group —NH₂. The term “alkylamino” refers herein to the group —NRR′ where R and R′ are each independently selected from hydrogen or a lower alkyl, provided —NRR′ is not —NH₂. The term “arylamino” refers herein to the group —NRR′ where R is aryl and R′ is hydrogen, a lower alkyl, or an aryl. The term “aralkylamino” refers herein to the group —NRR′ where R is a lower aralkyl and R′ is hydrogen, a loweralkyl, an aryl, or a loweraralkyl.

The term “alkoxyalkyl” refers to the group -alk₁-O-alk₂ where alk₁ is an alkyl linking group, and alk₂ is alkyl, e.g., a group such as —O—(CH₂)₂—O—CH₃. The term “loweralkoxyalkyl” refers to an alkoxyalkyl where alk₁ is loweralkyl and alk₂ is loweralkyl. The term “aryloxyalkyl” refers to the group -alkyl-O-aryl, where -alkyl- is a C₁₋₁₂ straight or branched chain alkyl linking group, preferably C₁₋₆. The term “aralkoxyalkyl” refers to the group -alkyl-O-aralkyl, where aralkyl is preferably a loweraralkyl.

The term “aminocarbonyl” refers herein to the group —C(O)—NH₂. “Substituted aminocarbonyl” refers herein to the group —C(O)—NRR′ where R is loweralkyl and R′ is hydrogen or a loweralkyl. In some embodiments, R and R′, together with the N atom attached to them may be taken together to form a “heterocycloalkylcarbonyl” group. The term “arylaminocarbonyl” refers herein to the group —C(O)—NRR′ where R is an aryl and R′ is hydrogen, loweralkyl or aryl. “aralkylaminocarbonyl” refers herein to the group —C(O)—NRR′ where R is loweraralkyl and R′ is hydrogen, loweralkyl, aryl, or loweraralkyl.

“Aminosulfonyl” refers herein to the group —S(O)₂—NH₂. “Substituted aminosulfonyl” refers herein to the group —S(O)₂—NRR′ where R is loweralkyl and R′ is hydrogen or a loweralkyl. The term “aralkylaminosulfonlyaryl” refers herein to the group -aryl-S(O)₂—NH-aralkyl, where the aralkyl is loweraralkyl.

“Carbonyl” refers to the divalent group —C(O)—. “Carboxy” refers to —C(═O)—OH. “Alkoxycarbonyl” refers to ester —C(═O)—OR wherein R is optionally substituted lower alkyl. “Loweralkoxycarbonyl” refers to ester —C(═O)—OR wherein R is optionally substituted lower loweralkyl. “Cycloalkyloxycarbonyl” refers to —C(═O)—OR wherein R is optionally substituted C3-C8 cycloalkyl.

“Cycloalkyl” refers to a mono- di- or poly-cyclic, carbocyclic alkyl substituent in which all ring atoms are carbon. Typical cycloalkyl groups have from 3 to 8 backbone (i.e., ring) atoms. When used in connection with cycloalkyl substituents, the term “polycyclic” refers herein to fused and non-fused alkyl cyclic structures, including spirocyclic ring systems. The term “partially unsaturated cycloalkyl”, “partially saturated cycloalkyl”, and “cycloalkenyl” all refer to a cycloalkyl group wherein there is at least one unsaturated carbon-carbon bond in the ring, i.e., wherein two adjacent ring atoms are connected by a double bond or a triple bond. Such rings typically contain 1 or 2 double bonds for 5-6 membered rings, and 1-2 double bonds or one triple bond for 7-8 membered rings. Illustrative examples include cyclohexenyl, cyclooctynyl, cyclopropenyl, cyclobutenyl, cyclohexadienyl, and the like.

The term “heterocycloalkyl” refers herein to cycloalkyl substituents that have from 1 to 5, and more typically from 1 to 3 heteroatoms as ring members in place of carbon atoms. Preferably, heterocycloalkyl or “heterocyclyl” groups contain one or two heteroatoms as ring members, typically only one heteroatom for 3-5 membered rings and 1-2 heteroatoms for 6-8 membered rings. Suitable heteroatoms employed in heterocyclic groups of the present invention are nitrogen, oxygen, and sulfur. Representative heterocycloalkyl moieties include, for example, pyrrolidinyl, tetrahydrofuranyl, oxirane, oxetane, oxepane, thiirane, thietane, azetidine, morpholino, piperazinyl, piperidinyl and the like.

The terms “substituted heterocycle”, “heterocyclic group” or “heterocycle” as used herein refers to any 3- or 4-membered ring containing a heteroatom selected from nitrogen, oxygen, and sulfur or a 5- or 6-membered ring containing from one to three heteroatoms, preferably 1-2 heteroatoms, selected from the group consisting of nitrogen, oxygen, or sulfur; wherein the 5-membered ring has 0-2 double bonds and the 6-membered ring has 0-3 double bonds; wherein the nitrogen and sulfur atom may be optionally oxidized; wherein the nitrogen and sulfur heteroatoms may be optionally quaternized; and including any bicyclic group in which any of the above heterocyclic rings is fused to a benzene ring or another 5- or 6-membered heterocyclic ring or heteroaryl as described herein. Preferred heterocycles include, for example: diazapinyl, pyrrolinyl, pyrrolidinyl, pyrazolinyl, pyrazolidinyl, imidazolinyl, imidazolidinyl, piperidinyl, piperazinyl, N-methyl piperazinyl, azetidinyl, N-methylazetidinyl, oxazolidinyl, isoxazolidinyl, morpholinyl, thiazolidinyl, isothiazolidinyl, and oxiranyl. The heterocyclic groups may be attached at various positions as will be apparent to those having skill in the organic and medicinal chemistry arts in conjunction with the disclosure herein.

Heterocyclic moieties can be unsubstituted or they can be substituted with one or more substituents independently selected from hydroxy, halo, oxo (C═O), alkylimino (RN═, wherein R is a loweralkyl or loweralkoxy group), amino, alkylamino, dialkylamino, acylaminoalkyl, alkoxy, thioalkoxy, lower alkoxyalkoxy, loweralkyl, cycloalkyl or haloalkyl. Typically, substituted heterocyclic groups will have up to four substituent groups.

The term “cyclic ether” as used herein refers to a 3-7 membered ring containing one oxygen atom (O) as a ring member. Where the cyclic ether is “optionally substituted” it can be substituted at any carbon atom with a group suitable as a substituent for a heterocyclic group, typically up to three substituents selected from lower alkyl, lower alkoxy, halo, hydroxy, amino, —C(O)-lower alkyl, and —C(O)-lower alkoxy. In preferred embodiments, halo, hydroxy and lower alkoxy are not attached to the carbon atoms of the ring that are bonded directly to the oxygen atom in the cyclic ether ring. Specific examples include oxirane, oxetane (e.g., 3-oxetane), tetrahydrofuran (including 2-tetrahydrofuranyl and 3-tetrahydrofuranyl), tetrahydropyran (e.g., 4-tetrahydropyranyl), and oxepane.

“Aryl” refers to monocyclic and polycyclic aromatic groups having from 5 to 14 backbone carbon atoms, and includes both carbocyclic aryl groups. When used in connection with aryl substituents, the term “polycyclic aryl” refers herein to fused and non-fused cyclic structures in which at least one cyclic structure is aromatic, such as, for example, benzodioxozolo (which has a heterocyclic structure fused to a phenyl group, naphthyl, and the like.

The term “heteroaryl” refers herein to aryl groups having from 1 to 4 heteroatoms as ring atoms in an aromatic ring with the remainder of the ring atoms being carbon atoms, in a 5-14 atom aromatic ring system that can be monocyclic or polycyclic. Monocyclic heteroaryl rings are typically 5-6 atoms in size. Exemplary heteroaryl moieties employed as substituents in compounds of the present invention include pyridyl, pyrimidinyl, thiazolyl, indolyl, imidazolyl, oxadiazolyl, tetrazolyl, pyrazinyl, triazolyl, thiophenyl, furanyl, quinolinyl, purinyl, benzothiazolyl, benzopyridyl, and benzimidazolyl, and the like.

“Aralkyl” or “arylalkyl” refers to an aryl group connected to a structure through an alkylene linking group, e.g., a structure such as —(CH₂)₁₋₄—Ar, where Ar represents an aryl group. “Lower aralkyl” or similar terms indicate that the alkyl linking group has up to 6 carbon atoms.

“Optionally substituted” or “substituted” refers to the replacement of one or more hydrogen atoms with a non-hydrogen group. Alkyl, alkenyl, alkynyl, cycloalkyl, heterocycloalkyl, aryl, and heteroaryl groups described herein may be substituted or unsubstituted. Suitable substitution groups include, for example, hydroxy, nitro, amino, imino, cyano, halo, thio, sulfonyl, thioamido, amidino, imidino, oxo, oxamidino, methoxamidino, imidino, guanidino, sulfonamido, carboxyl, formyl, loweralkyl, haloloweralkyl, loweralkylamino, haloloweralkylamino, lower alkoxy, lower haloalkoxy, lower alkoxyalkyl, alkylcarbonyl, aminocarbonyl, arylcarbonyl, aralkylcarbonyl, heteroarylcarbonyl, heteroaralkylcarbonyl, alkylthio, aminoalkyl, cyanoalkyl, aryl and the like, provided that oxo, imidino or other divalent substitution groups are not placed on aryl or heteroaryl rings due to the well known valence limitations of such rings. In preferred embodiments, unless otherwise specified, optional substituents for alkyl, alkenyl, alkynyl, cycloalkyl, and heterocycloalkyl groups are 1-3 groups selected from halo, hydroxy, amino, cyano, lower alkoxy, lower alkylsulfonyl, oxy, carboxy, and lower alkoxycarbonyl. In preferred embodiments, unless otherwise specified, optional substituents for aryl and heteroaryl groups are 1-3 groups selected from halo, hydroxy, amino, cyano, lower alkyl, lower alkoxy, lower alkylsulfonyl, carboxy, and lower alkoxycarbonyl.

The substitution group can itself be substituted where valence permits, i.e., where the substitution group contains at least one CH, NH or OH having a hydrogen atom that can be replaced. The group substituted onto the substitution group can be carboxyl, halo (on carbon only); nitro, amino, cyano, hydroxy, loweralkyl, loweralkoxy, C(O)R, —OC(O)R, —OC(O)OR, —NRCOR, —CONR₂, —NRCOOR, —C(S)NR₂, —NRC(S)R, —OC(O)NR₂, —SR, —SO₃H, —SO₂R or C3-8 cycloalkyl or 3-8 membered heterocycloalkyl, where each R is independently selected from hydrogen, lower haloalkyl, lower alkoxyalkyl, and loweralkyl, and where two R on the same atom or on directly connected atoms can be linked together to form a 5-6 membered heterocyclic ring. Unless indicated as optionally substituted, these substitution groups are typically unsubstituted.

When a substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

It is understood that the above definitions are not intended to include impermissible substitution patterns (e.g., methyl substituted with five fluoro groups or a halogen atom substituted with another halogen atom). Such impermissible substitution patterns are well known to the skilled artisan.

“Syn” as used herein has its ordinary meaning, and is used in connection with Formula I to indicate that the specified groups are attached to sp³ hybridized (tetrahedral) carbon centers and extend out from one face of the cyclohexyl or piperidinyl ring, i.e., those groups all project toward the ‘alpha’ face of the ring, or they all project toward the ‘beta’ face of the ring. This is thus used as a convenient way to define the relative orientations of two or more groups on a ring, without limiting the compounds to a specific absolute chiral configuration. This reflects the fact that the compounds of the invention have such groups in a specific relative orientation, but are not limited to either enantiomer of that specific relative orientation. Accordingly, unless described as optically active, such compounds may be racemic, but also include each of the two enantiomers having the specified relative stereochemistry. In some embodiments, the compounds of the invention are optically active form as further described herein, and in preferred embodiments of the invention, the compounds are obtained and used in optically active form. Preferably, the enantiomer having greater potency as an inhibitor of at least two of Pim1, Pim2 and Pim3 is selected.

It will also be apparent to those skilled in the art that the compounds of the invention, as well as the pharmaceutically acceptable salts, esters, metabolites and prodrugs of any of them, may be subject to tautomerization and may therefore exist in various tautomeric forms wherein a proton of one atom of a molecule shifts to another atom and the chemical bonds between the atoms of the molecules are consequently rearranged. See, e.g., March, Advanced Organic Chemistry: Reactions, Mechanisms and Structures, Fourth Edition, John Wiley & Sons, pages 69-74 (1992). As used herein, the term “tautomer” refers to the compounds produced by the proton shift, and it should be understood that all tautomeric forms, insofar as they may exist, are included within the invention.

The compounds of the invention comprise one or more asymmetrically substituted carbon atoms. Such asymmetrically substituted carbon atoms can result in the compounds of the invention existing in enantiomers, diastereomers, and other stereoisomeric forms that may be defined, in terms of absolute stereochemistry, such as in (R)- or (S)-forms. The compounds of the invention are sometimes depicted herein as single enantiomers, and are intended to encompass the specific configuration depicted and the enantiomer of that specific configuration (the mirror image isomer of the depicted configuration), unless otherwise specified—e.g., where a structure is labeled ‘chiral’, it represents the specified absolute stereochemistry as a single substantially pure (i.e., at least about 95% pure) enantiomer. The depicted structures herein describe the relative stereochemistry of the compounds where two or more chiral centers, but the invention is not limited to the depicted enantiomer's absolute stereochemistry unless otherwise stated. The invention includes both enantiomers, each of which will exhibit Pim inhibition, even though one enantiomer will be more potent than the other. In some instances, compounds of the invention have been synthesized in racemic form and separated into individual isomers by chiral chromatography or similar conventional methods, and the analytical data about the two enantiomers do not provide definitive information about absolute stereochemical configuration. In such cases, the absolute stereochemistry of the most active enantiomer has been identified based on correlation with similar compounds of known absolute stereochemistry, rather than by a definitive physical method such as X-ray crystallography. Therefore, in certain embodiments, the preferred enantiomer of a compound described herein is the specific isomer depicted or its opposite enantiomer, whichever has the lower IC-50 for Pim kinase inhibition using the assay methods described herein, i.e., the enantiomer that is more potent as a Pim inhibitor for at least two of Pim1, Pim2, and Pim3.

The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

As used herein, the term “pharmaceutically acceptable salts” refers to the nontoxic acid or base addition salts of the compounds of Formulas I, II, etc., wherein the compound acquires a positive or negative charge as a result of adding or removing a proton; the salt then includes a counterion of opposite charge from the compound itself, and the counterion is preferably one suitable for pharmaceutical administration under the conditions where the compound would be used. These salts can be prepared in situ during the final isolation and purification of the compounds of Formula I or II, or by separately reacting the base or acid functions with a suitable organic or inorganic acid or base, respectively. Representative salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalenesulfonate, oxalate, pamoate, pectinate, persulfate, 3-phenylproionate, picrate, pivalate, propionate, succinate, sulfate, tartrate, thiocyanate, p-toluenesulfonate and undecanoate.

Also, a basic nitrogen-containing group in compounds of the invention can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained. These quaternized ammonium salts when paired with a pharmaceutically acceptable anion can also serve as pharmaceutically acceptable salts.

Examples of acids which may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutically acceptable metal cation or with ammonia, or an organic primary, secondary or tertiary amine. Counterions for pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular pharmaceutically acceptable esters include formates, acetates, propionates, maleates, lactates, hydroxyacetates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, PRO-DRUGS AS NOVEL DELIVERY SYSTEMS, Vol. 14 of the A.C.S. Symposium Series, and in Edward B. Roche, ed., BIOREVERSIBLE CARRIERS IN DRUG DESIGN, American Pharmaceutical Association and Pergamon Press, 1987, both of which are incorporated herein by reference.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d⁶-acetone, d⁶-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

In one aspect, the invention provides compounds of Formula I:

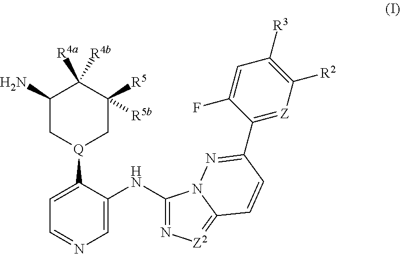

wherein:
Z is CH, CF or N, typically Z is CF or N;
Z² is CH or N, in many embodiments Z² is CH;
Q is CH or N; when Q is CH, the compound has the relative stereochemistry as shown;
R² is H or —C(O)NR*₂; preferably R² is H or —C(O)NHR*, and typically R³ is H when R² is —C(O)NHR*;
R^{4a} and R^{4b} are each selected from H, CN, halo, azido, amino, R⁶, —OR⁶, C₁₋₄ alkyl, C₁₋₄ haloalkyl, —O(CH₂)₁₋₃—OR⁶, —NRC(O)R⁶, —NRCOOR⁶, NRSO₂R⁶, —SO₂R⁶, N-pyridonyl, or 1-triazolyl (e.g., N-1,2,3-triazolyl); preferably R^{4a} and R^{4b} are selected from H, halo, Me, CF₃, OH, OR⁶, SO₂R⁶, NRC(═O)R⁶, or NRC(═O)OR⁶;
   where each R is H or C₁₋₄ alkyl;
   provided that when R^{4a} is H and R^{4b} is H or OH, R² and R³ cannot both be H;
R⁵ is H or C₁₋₄ alkyl;
R^{5b} is H, or R^{4b} and R^{5b} taken together form a double bond between the carbon atoms to which they are attached;
R⁶ is C₁₋₄ alkyl optionally substituted with up to three groups selected from halo, CN, C₁₋₄ alkylsulfonyl, hydroxy, and C₁₋₄ alkoxy;
each R³ is independently selected from CN, hydroxy, C₁₋₄ haloalkyl, —S(O)ₚ—R*, C₁₋₄ haloalkoxy, —(CH₂)₀₋₃—OR*, —O—(CH₂)₁₋₃—OR*, —CONR*₂, —(CR′₂)₁₋₃—OR′, —O—(CR′₂)₁₋₃—OR′, and an optionally substituted member selected from the group consisting of -L-C₁₋₆ alkyl, -L-C₁₋₆ alkylsulfonyl, -L-C₃₋₇ cycloalkyl, and -L-C₄₋₇ heterocycloalkyl, wherein each L is selected from a bond, —O—, —CH₂—, —CH₂—O— and —O—CH₂—, and each C₁₋₆ alkyl, C₁₋₆ alkylsulfonyl, C₃₋₇ cycloalkyl, and C₄₋₇ heterocycloalkyl contains one or two heteroatoms selected from N, O and S, and is optionally substituted with up to two groups selected from halo, CN, hydroxy, C₁₋₄ alkoxy, and R*;
or R³ can be H when R² is —C(O)NHR*;
   where each R′ is independently H or Me or Et,
   and each R* is independently H or a 4-7 membered cyclic ether, 3-6 membered cycloalkyl, pyrrolidine, or C₁₋₆ alkyl, each of which is optionally substituted with up to three groups selected from halo, oxo, C₁₋₄ alkyl, C₁₋₄ alkoxy, OH, OMe, OEt, and CN; and
p is 0, 1 or 2;

or a pharmaceutically acceptable salt thereof.

The following enumerated embodiments represent selected aspects of the invention:

1. A compound of Formula (I):

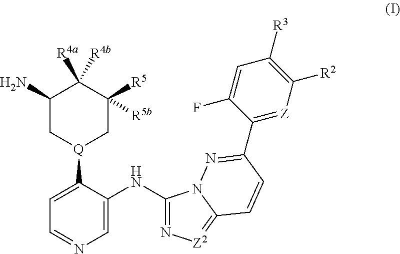

wherein:
Z is CF or N;
Z² is CH or N;
Q is CH or N;
R² is H or —C(O)NHR*;
R^{4a} is H, halo, Me, CF₃, OH, OR⁶, SO₂R⁶, NR′C(═O)R⁶ or NR′C(═O)OR⁶;
R^{4b} is H, halo, Me, CF₃, OH, OR⁶, SO₂R⁶, NR′C(═O)R⁶ or NR′C(═O)OR⁶,
or R^{4b} can be taken together with R^{5b} to form a double bond;
   provided that when R^{4a} is H and R^{4b} is H or OH, R² and R³ cannot both be H;
R⁵ is H or C₁₋₄ alkyl;
R^{5b} is H, or R^{4b} and R^{5b} taken together form a double bond between the carbon atoms to which they are attached;
R⁶ is C₁₋₄ alkyl optionally substituted with up to three groups selected from halo, CN, C₁₋₄ alkylsulfonyl, hydroxy, and C₁₋₄ alkoxy;
each R³ is independently selected from CN, hydroxy, C₁₋₄ haloalkyl, —S(O)ₚ—R*, C₁₋₄ haloalkoxy, —(CH₂)₀₋₃—OR*, —O—(CH₂)₁₋₃—OR*, —CONR*₂, —(CR′₂)₁₋₃—OR′ or —O—(CR′₂)₁₋₃—OR′, and an optionally substituted member selected from the group consisting of -L-C₁₋₆ alkyl, -L-C₁₋₆ alkylsulfonyl, -L-C₃₋₇ cycloalkyl, and -L-C₄₋₇ heterocycloalkyl,
   wherein each L is selected from a bond, —O—, —CH₂—, —CH₂—O— and —O—CH₂—,
   and each C₁₋₆ alkyl, C₁₋₆ alkylsulfonyl, C₃₋₇ cycloalkyl, and C₄₋₇ heterocycloalkyl is optionally substituted with up to two groups selected from halo, CN, hydroxy, C₁₋₄ alkoxy, and R*;
or R³ can be H when R² is —C(O)NHR*;
each R′ is independently H or Me or Et;
each R* is independently H or a 4-7 membered cyclic ether, 3-6 membered cycloalkyl, pyrrolidine, or C₁₋₆ alkyl, each of which is optionally substituted with up to three groups selected from halo, oxo, C₁₋₄ alkyl, C₁₋₄ alkoxy, OH, OMe, OEt, and CN; and
p is 0, 1 or 2;
or a pharmaceutically acceptable salt thereof.
   2. The compound of embodiment 1, wherein R³ is selected from OH, OMe, OEt, —SO₂Me, -L-CH₂A,

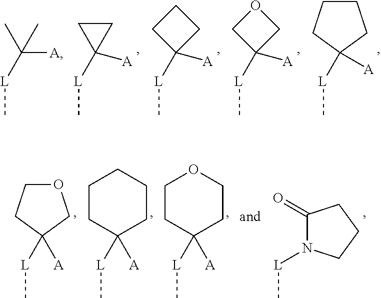

where each L is selected from a bond, —O—, —CH₂—, —OCH₂— and —CH₂O—,
each A is selected from H, OH, F, CN, —OMe and —OEt,
and the dashed bonds indicate where R³ is attached to the ring in Formula I. In some of these embodiments L is a bond; in other embodiments, L is CH₂ or O; in other embodiments, L is —CH₂O— or −O—CH₂—.
   3. The compound of embodiment 1 or 2, wherein R² is H.
   4. The compound of any of embodiments 1-3, wherein Z is CF.
   5. The compound of any of embodiments 1-3, wherein Z is N.
   6. The compound of any of embodiments 1-5, wherein Z² is CH; or the compound of any of embodiments 1-5 wherein Z² is N.
   7. The compound of any of embodiments 1-6, wherein R^{4b} is H.
   8. The compound of any of embodiments 1-7, wherein Q is CH.
   9. The compound of any one of embodiments 1-7, wherein Q is N.
   10. The compound of any of embodiments 1-9, wherein R^{4b} and R⁵ taken together form a double bond; or the compound of any of embodiments 1-9, wherein R^{5b} is H.
   11. The compound of any of embodiments 1-10, wherein R^{4a} is not H.
   12. The compound of any of embodiments 1-11, wherein R⁵ is Me.
   13. The compound of any one of embodiments 1-12, wherein R³ is of the formula:

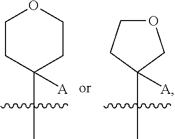

wherein A is H, CN, OH, OMe, or F.

14. The compound of any one of embodiments 1-12, wherein R³ is of the formula:

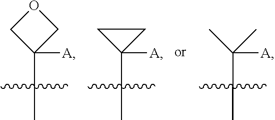

wherein A is H, CN, OH, OMe, or F.

15. The compound of any of embodiments 1-12, wherein the ring in Formula I that contains Q is selected from:

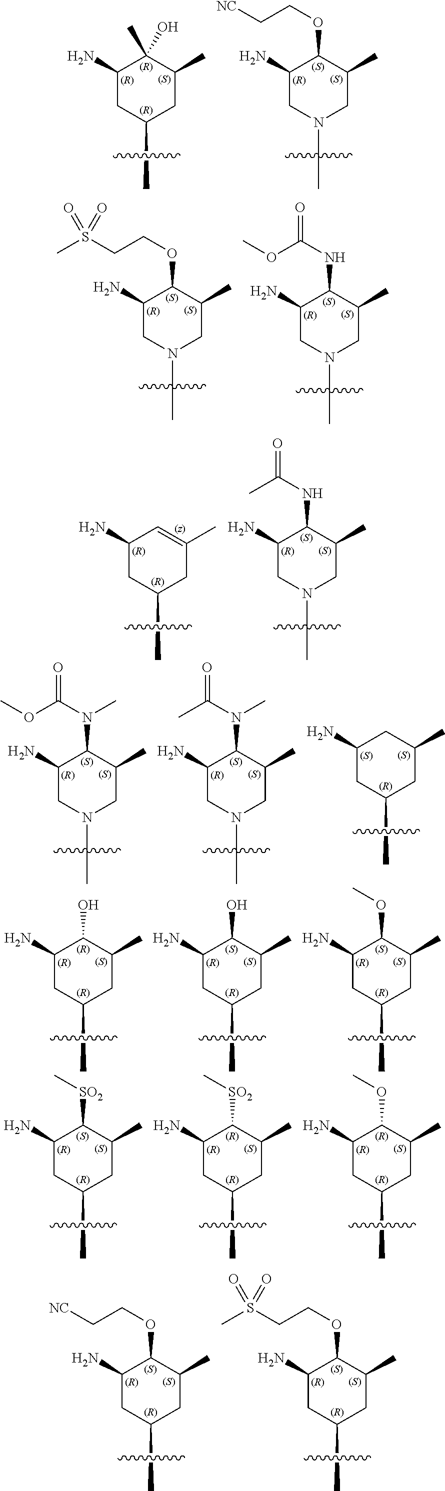

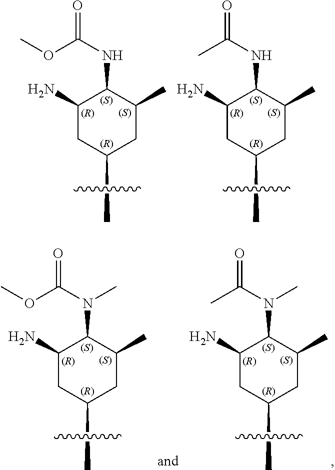

or, when R³ is not H, the ring containing Q can be

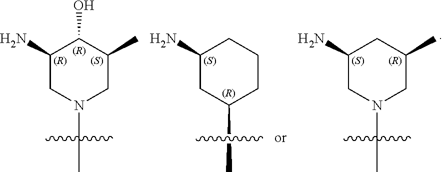

16. The compound of any of embodiments 1-15, wherein the ring in Formula I that contains Z is selected from:

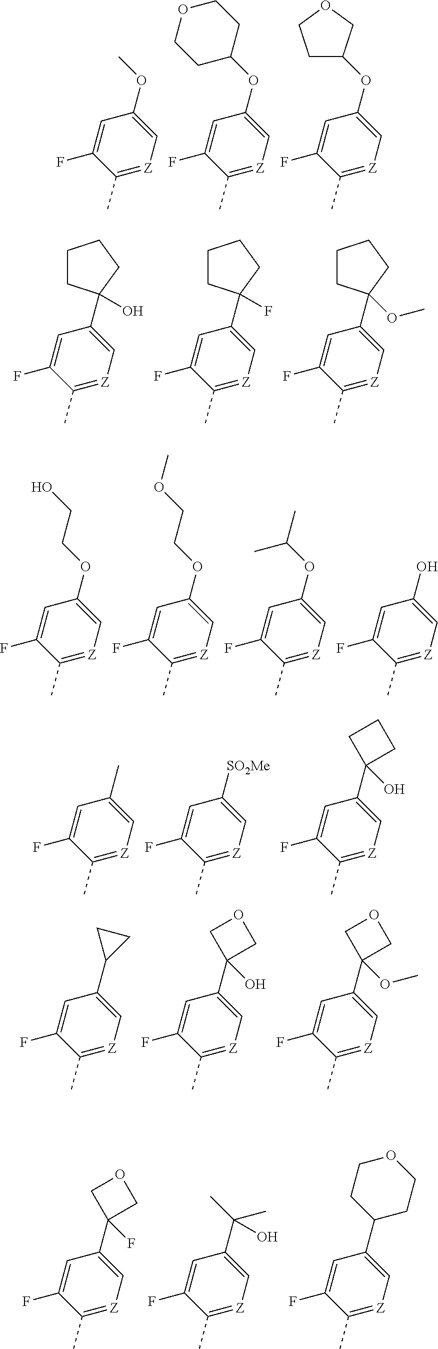

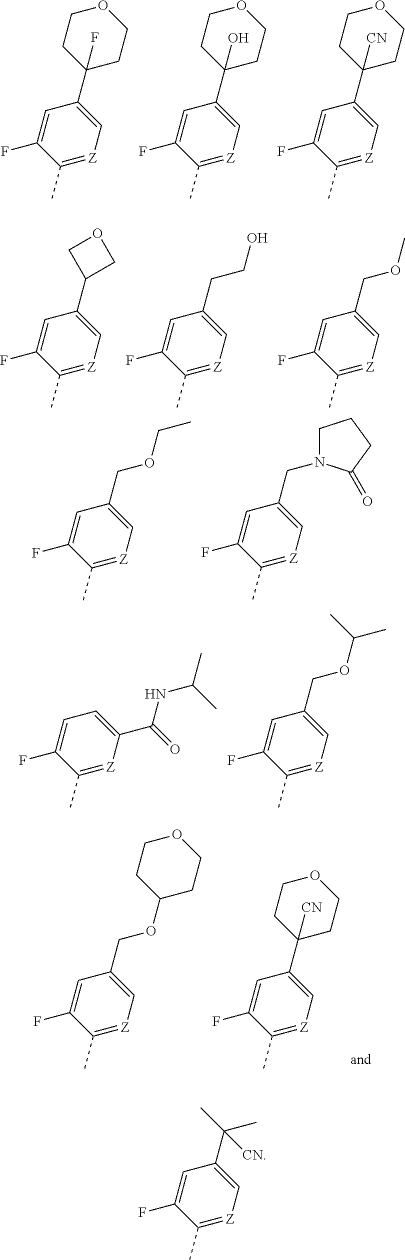

In these formulas, the dashed line represents the point of attachment of the ring to the pyridazine ring in Formula I.
17. The compound of embodiment 1, which is selected from the compounds in Table 1 and pharmaceutically acceptable salts of these compounds.
18. A pharmaceutical composition comprising a compound of any of the preceding embodiments and at least one pharmaceutically acceptable excipient. In some embodiments, the composition comprises at least two pharmaceutically acceptable excipients.
19. The pharmaceutical composition of embodiment 18, further comprising a co-therapeutic agent.
20. The pharmaceutical composition of embodiment 19, wherein the co-therapeutic agent is selected from MEK inhibitors, Velcade, dexamethasone, clofarabine, Mylotarg, lenalidomide, bortezomib, irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, thalidomide, bortezomib, and trastuzumab.
21. A method to treat a condition caused or exacerbated by excessive Pim kinase activity, which comprises administering to a subject in need thereof an effective amount of a compound of any one of embodiments 1-17. The invention also includes methods to use the compounds of any of embodiments 1-17 for the manufacture of a medicament, particularly for manufacture of a medicament to treat conditions named in embodiment 23.
22. The method of embodiment 21, wherein the condition is a cancer.
23. The method of embodiment 21, wherein the cancer is selected from carcinoma of the lungs, pancreas, thyroid, ovary, bladder, breast, prostate, or colon, melanoma, myeloid leukemia, multiple myeloma, erythroleukemia, villous colon adenoma, gastric cancers, and osteosarcoma; or the autoimmune disorder is selected from Crohn's disease, inflammatory bowel disease, rheumatoid arthritis, and chronic inflammatory diseases.
24. A compound according to any of embodiments 1-17 for use in therapy, particularly therapy for cancers including the conditions named in embodiment 23.
25. Use of a compound according to any of embodiments 1-17 for the preparation of a medicament.

In compounds of the invention (Formula I and the various embodiments described herein), Z can be N; in preferred embodiments, Z is CF.

In many embodiments of the compounds of Formula I, R^{5b} is H. In other embodiments, R^{5b} is taken together with R^{4b} to form a double bond between the carton atoms to which these groups are attached.

In many embodiments of the foregoing compounds, R′ when present is H or Me.

In certain embodiments of the foregoing compounds, R⁵ is Me.

In some embodiments of the foregoing compounds, R^{4b} is H. In other embodiments, R^{4a} is H or C₁₋₄ alkyl (e.g., Me) or CF₃, and R^{4b} is —OH. When R^{4b} or R^{4a} is —OH, the other of R^{4b} and R^{4a} is typically H, Me or CF₃. When R^{4b} or R^{4a} is attached through oxygen, nitrogen, or sulfur, the other of R^{4a} and R^{4b} is not —OH; preferably when R^{4a} is attached to Formula I through oxygen, nitrogen, or sulfur, R^{4b} is H.

In some embodiments, R^{4a} is selected from —OH, -OMe, and —O(CH₂CH₂)X where X is CN or —SO₂Me. In other embodiments, R^{4a} is selected from —NHCO(C₁₋₄ alkyl), —SO₂(C₁₋₄ alkyl), and —NHC(O)O(C₁₋₄ alkyl). In these embodiments, R^{4b} is H or Me, preferably H.

Some preferred embodiments of the foregoing compounds, the ring in Formula I that contains Q is selected from:

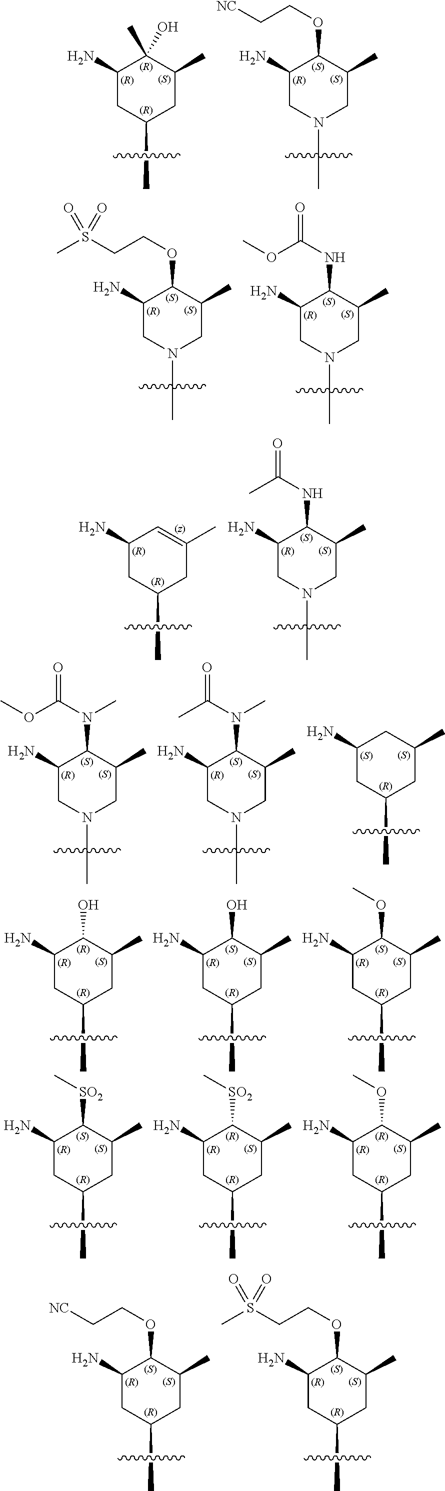

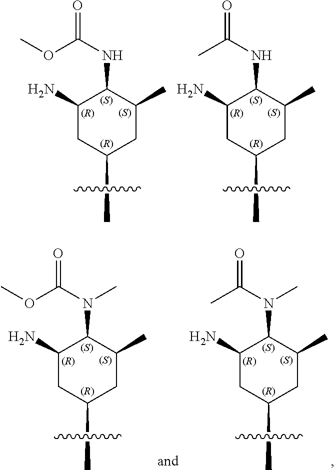

or, when R³ is not H, the ring containing Q can also be

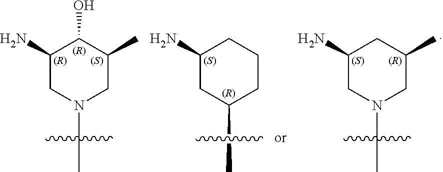

In some preferred embodiments of the foregoing compounds, the ring containing Z in Formula I is selected from:

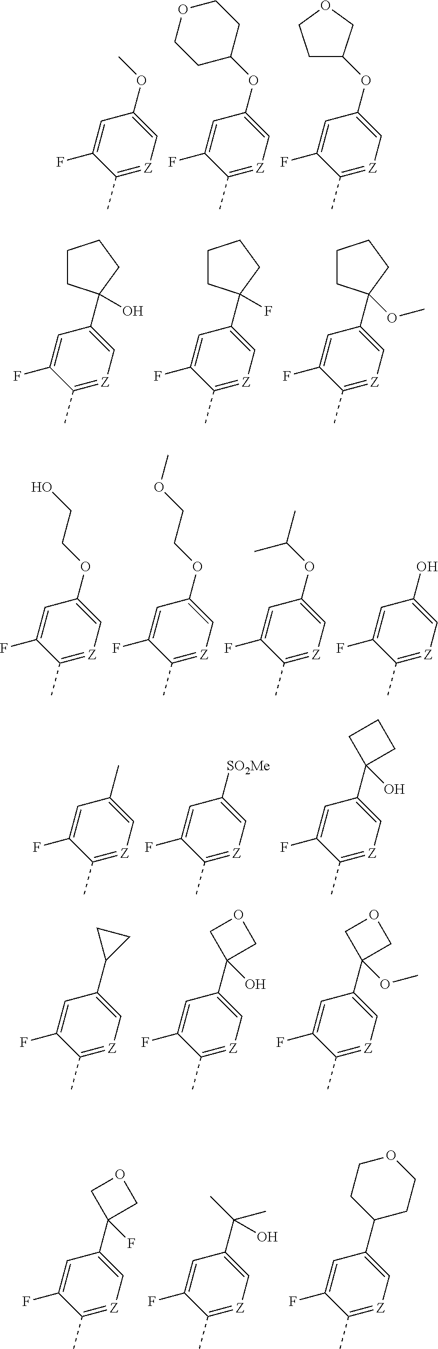

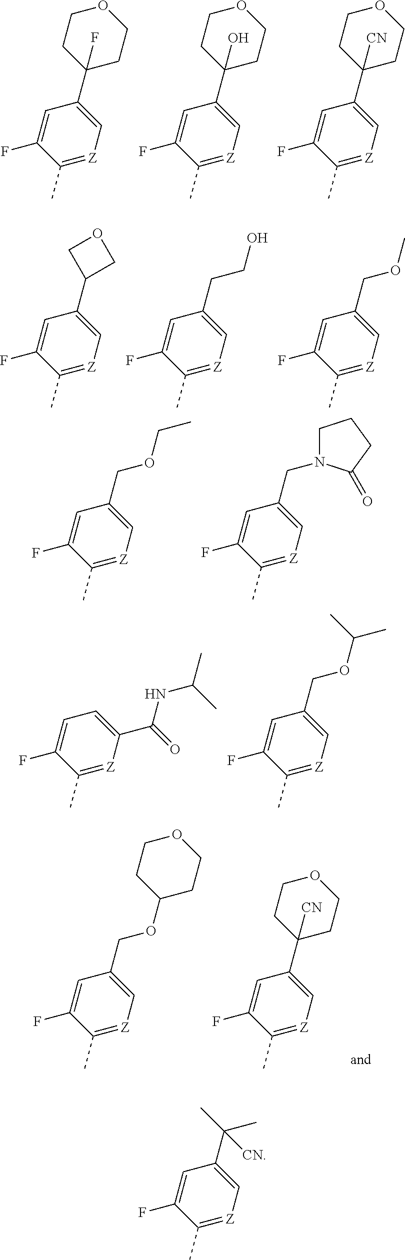

In these rings, Z is CF or N, preferably CF.

Each compound of Formula I having any combination of any of these preferred features (substituted rings containing Z, and substituted rings containing Q), wherein Z is CF or N, including compounds wherein Z² is either CH or N, is a specifically contemplated embodiment of the invention.

Each of the species in Table 1 is a preferred embodiment of the invention.

The compounds of the invention contain at least one chiral center; the depicted structures show the relative stereochemistry when two or more chiral centers are shown. The invention includes both enantiomers of the depicted structure as well as mixtures of the enantiomers, including racemic mixtures. In some embodiments, the compounds have the absolute stereochemistry shown in the depicted structures, and are enriched in this enantiomer to an enantiomeric excess of at least 90%, preferably at least 95%.

TABLE 1
Compounds of Formula I that can be prepared by methods disclosed herein
in combination with methods and starting materials known in the art.

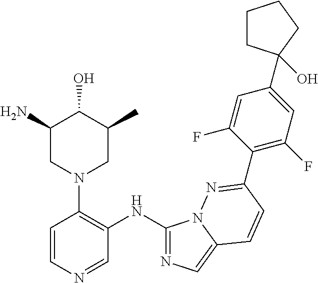 [SMILES: C[C@H]1CN(c2ccncc2Nc2ncc3ccc(-c4c(F)cc(C5(O)CCCC5)cc4F)nn23)C[C@@H](N)[C@@H]1O]

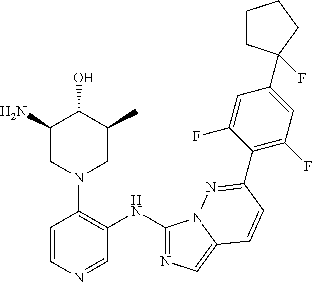 [SMILES: C[C@H]1CN(c2ccncc2Nc2ncc3ccc(-c4c(F)cc(C5(F)CCCC5)cc4F)nn23)C[C@@H](N)[C@@H]1O]

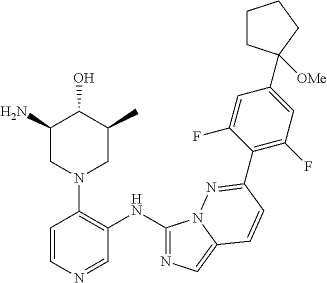 [SMILES: COC1(c2cc(F)c(-c3ccc4cnc(Nc5cnccc5N5C[C@@H](N)[C@H](O)[C@@H](C)C5)n4n3)c(F)c2)CCCC1]

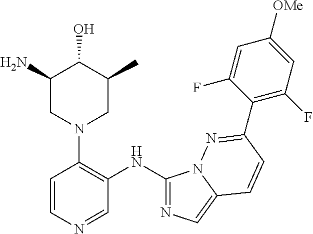 [SMILES: COc1cc(F)c(-c2ccc3cnc(Nc4cnccc4N4C[C@@H](N)[C@H](O)[C@@H](C)C4)n3n2)c(F)c1]

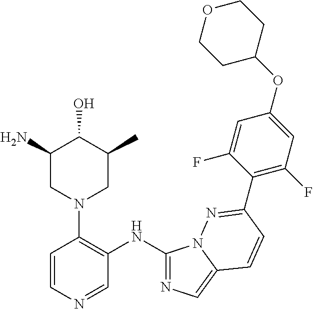 [SMILES: C[C@H]1CN(c2ccncc2Nc2ncc3ccc(-c4c(F)cc(OC5CCOCC5)cc4F)nn23)C[C@@H](N)[C@@H]1O]

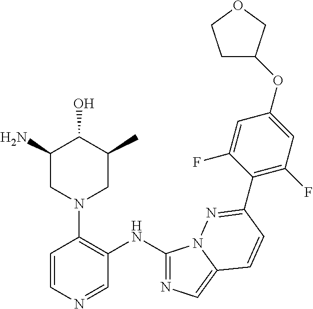 [SMILES: C[C@H]1CN(c2ccncc2Nc2ncc3ccc(-c4c(F)cc(OC5CCOC5)cc4F)nn23)C[C@@H](N)[C@@H]1O]

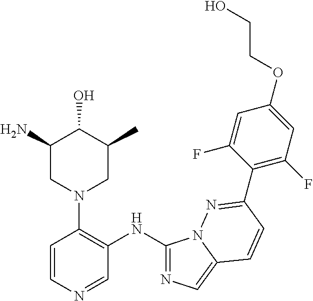 [SMILES: C[C@H]1CN(c2ccncc2Nc2ncc3ccc(-c4c(F)cc(OCCO)cc4F)nn23)C[C@@H](N)[C@@H]1O]

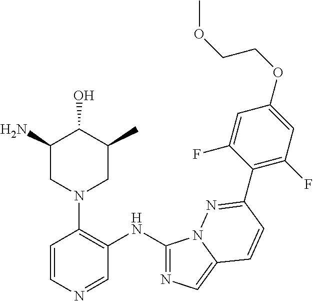 [SMILES: COCCOc1cc(F)c(-c2ccc3cnc(Nc4cnccc4N4C[C@@H](N)[C@H](O)[C@@H](C)C4)n3n2)c(F)c1]

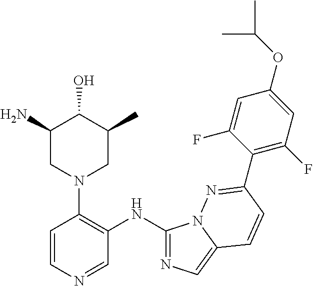 [SMILES: CC(C)Oc1cc(F)c(-c2ccc3cnc(Nc4cnccc4N4C[C@@H](N)[C@H](O)[C@@H](C)C4)n3n2)c(F)c1]

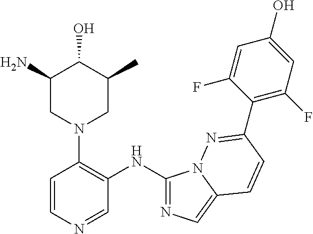 [SMILES: C[C@H]1CN(c2ccncc2Nc2ncc3ccc(-c4c(F)cc(O)cc4F)nn23)C[C@@H](N)[C@@H]1O]

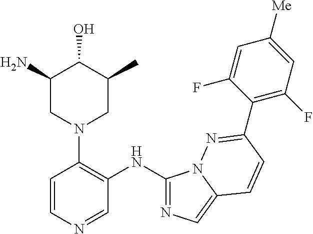 [SMILES: Cc1cc(F)c(-c2ccc3cnc(Nc4cnccc4N4C[C@@H](N)[C@H](O)[C@@H](C)C4)n3n2)c(F)c1]

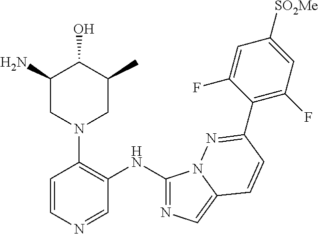 [SMILES: C[C@H]1CN(c2ccncc2Nc2ncc3ccc(-c4c(F)cc(S(C)(=O)=O)cc4F)nn23)C[C@@H](N)[C@@H]1O]

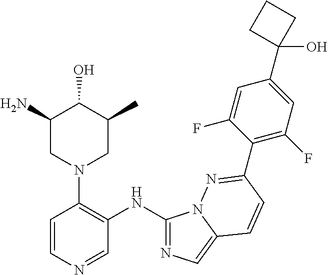 [SMILES: C[C@H]1CN(c2ccncc2Nc2ncc3ccc(-c4c(F)cc(C5(O)CCC5)cc4F)nn23)C[C@@H](N)[C@@H]1O]

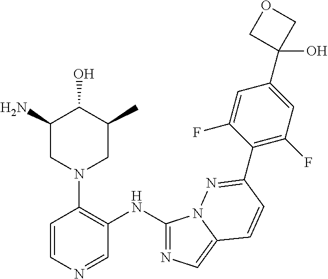 [SMILES: C[C@H]1CN(c2ccncc2Nc2ncc3ccc(-c4c(F)cc(C5(O)COC5)cc4F)nn23)C[C@@H](N)[C@@H]1O]

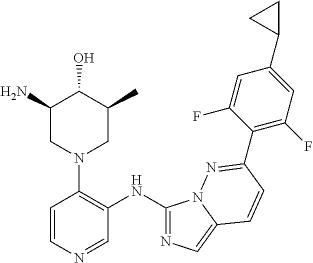 [SMILES: C[C@H]1CN(c2ccncc2Nc2ncc3ccc(-c4c(F)cc(C5CC5)cc4F)nn23)C[C@@H](N)[C@@H]1O]

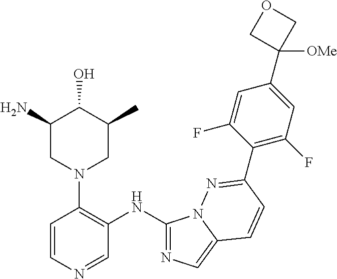 [SMILES: COC1(c2cc(F)c(-c3ccc4cnc(Nc5cnccc5N5C[C@@H](N)[C@H](O)[C@@H](C)C5)n4n3)c(F)c2)COC1]

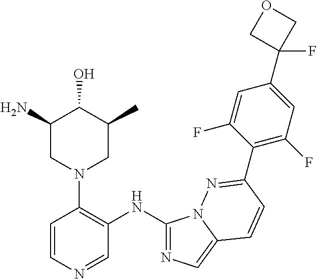 [SMILES: C[C@H]1CN(c2ccncc2Nc2ncc3ccc(-c4c(F)cc(C5(F)COC5)cc4F)nn23)C[C@@H](N)[C@@H]1O]

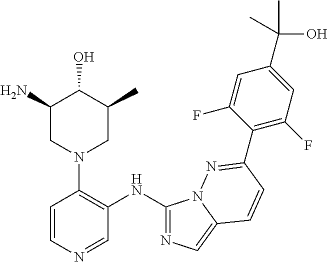 [SMILES: C[C@H]1CN(c2ccncc2Nc2ncc3ccc(-c4c(F)cc(C(C)(C)O)cc4F)nn23)C[C@@H](N)[C@@H]1O]

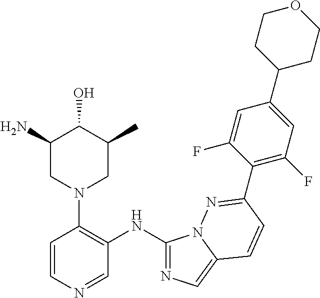 [SMILES: C[C@H]1CN(c2ccncc2Nc2ncc3ccc(-c4c(F)cc(C5CCOCC5)cc4F)nn23)C[C@@H](N)[C@@H]1O]

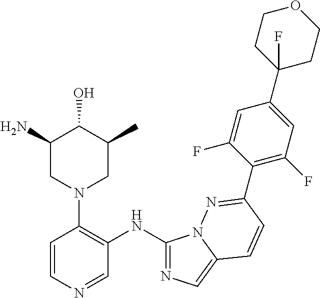

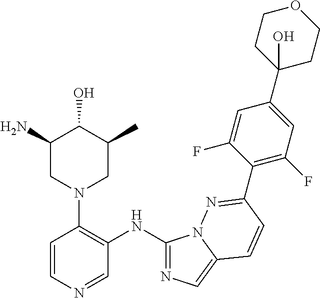

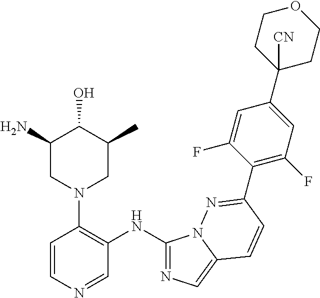

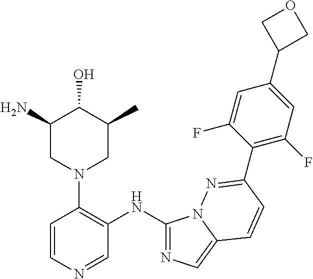 [SMILES: C[C@H]1CN(c2ccncc2Nc2ncc3ccc(-c4c(F)cc(C5COC5)cc4F)nn23)C[C@@H](N)[C@@H]1O]

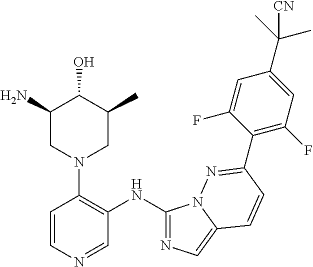 [SMILES: C[C@H]1CN(c2ccncc2Nc2ncc3ccc(-c4c(F)cc(C(C)(C)C#N)cc4F)nn23)C[C@@H](N)[C@@H]1O]

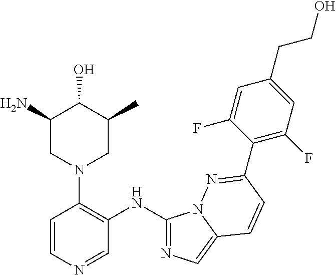 [SMILES: C[C@H]1CN(c2ccncc2Nc2ncc3ccc(-c4c(F)cc(CCO)cc4F)nn23)C[C@@H](N)[C@@H]1O]

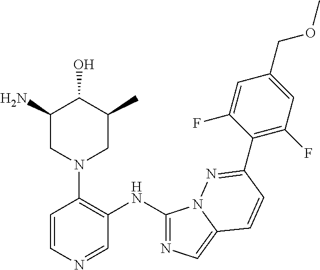 [SMILES: COCc1cc(F)c(-c2ccc3cnc(Nc4cnccc4N4C[C@@H](N)[C@H](O)[C@@H](C)C4)n3n2)c(F)c1]

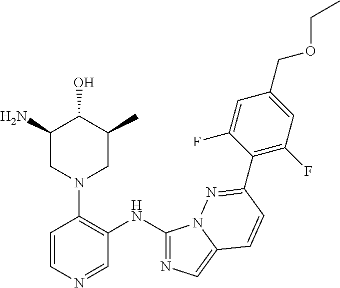 [SMILES: CCOCc1cc(F)c(-c2ccc3cnc(Nc4cnccc4N4C[C@@H](N)[C@H](O)[C@@H](C)C4)n3n2)c(F)c1]

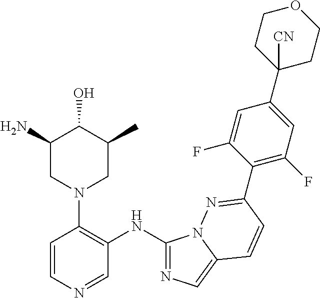

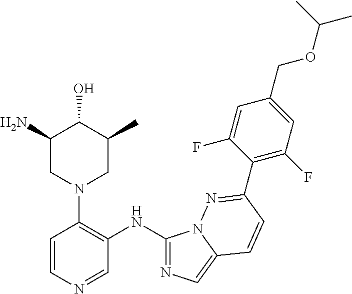 [SMILES: CC(C)OCc1cc(F)c(-c2ccc3cnc(Nc4cnccc4N4C[C@@H](N)[C@H](O)[C@@H](C)C4)n3n2)c(F)c1]

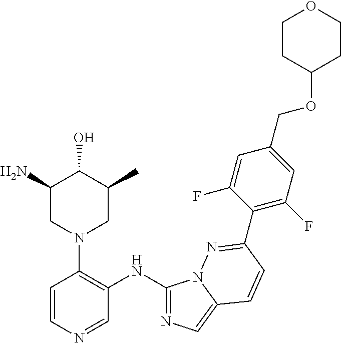 [SMILES: C[C@H]1CN(c2ccncc2Nc2ncc3ccc(-c4c(F)cc(COC5CCOCC5)cc4F)nn23)C[C@@H](N)[C@@H]1O]

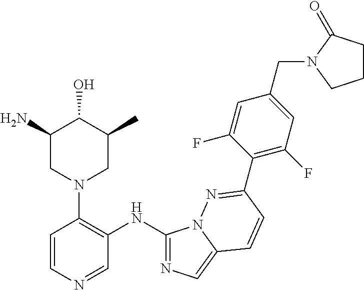 [SMILES: C[C@H]1CN(c2ccncc2Nc2ncc3ccc(-c4c(F)cc(CN5CCCC5=O)cc4F)nn23)C[C@@H](N)[C@@H]1O]

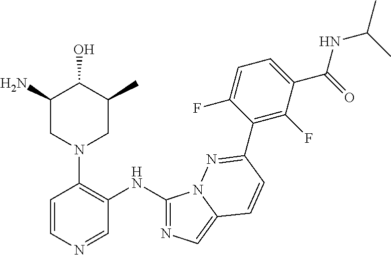 [SMILES: CC(C)NC(=O)c1ccc(F)c(-c2ccc3cnc(Nc4cnccc4N4C[C@@H](N)[C@H](O)[C@@H](C)C4)n3n2)c1F]

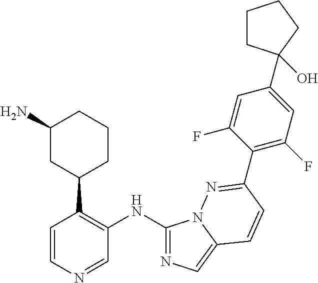 [SMILES: N[C@H]1CCC[C@@H](c2ccncc2Nc2ncc3ccc(-c4c(F)cc(C5(O)CCCC5)cc4F)nn23)C1]

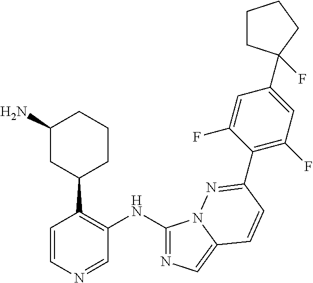 [SMILES: N[C@H]1CCC[C@@H](c2ccncc2Nc2ncc3ccc(-c4c(F)cc(C5(F)CCCC5)cc4F)nn23)C1]

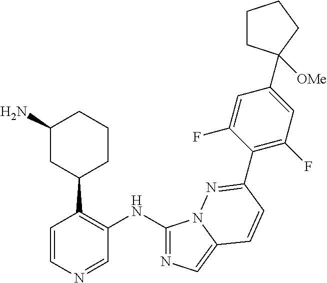 [SMILES: COC1(c2cc(F)c(-c3ccc4cnc(Nc5cnccc5[C@@H]5CCC[C@H](N)C5)n4n3)c(F)c2)CCCC1]

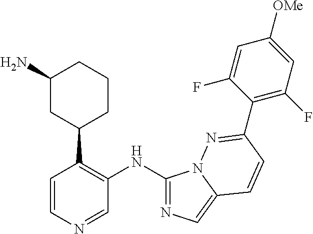 [SMILES: COc1cc(F)c(-c2ccc3cnc(Nc4cnccc4[C@@H]4CCC[C@H](N)C4)n3n2)c(F)c1]

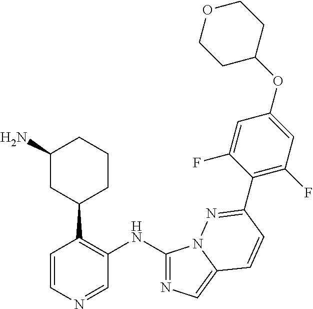 [SMILES: N[C@H]1CCC[C@@H](c2ccncc2Nc2ncc3ccc(-c4c(F)cc(OC5CCOCC5)cc4F)nn23)C1]

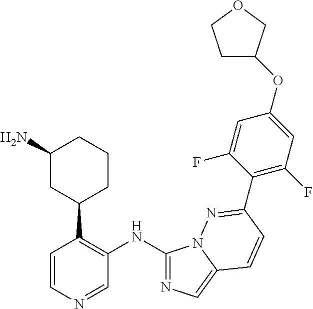 [SMILES: N[C@H]1CCC[C@@H](c2ccncc2Nc2ncc3ccc(-c4c(F)cc(OC5CCOC5)cc4F)nn23)C1]

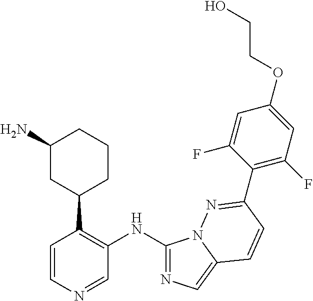 [SMILES: N[C@H]1CCC[C@@H](c2ccncc2Nc2ncc3ccc(-c4c(F)cc(OCCO)cc4F)nn23)C1]

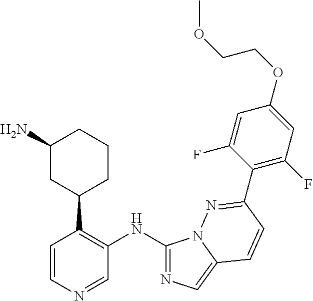 [SMILES: COCCOc1cc(F)c(-c2ccc3cnc(Nc4cnccc4[C@@H]4CCC[C@H](N)C4)n3n2)c(F)c1]

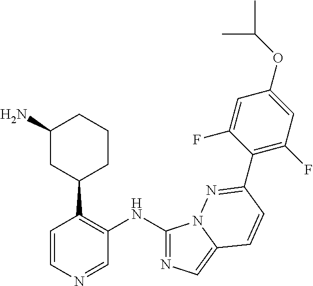 [SMILES: CC(C)Oc1cc(F)c(-c2ccc3cnc(Nc4cnccc4[C@@H]4CCC[C@H](N)C4)n3n2)c(F)c1]

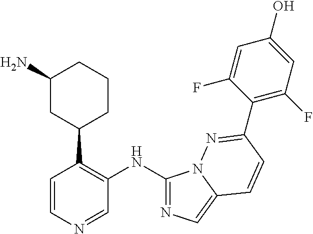 [SMILES: N[C@H]1CCC[C@@H](c2ccncc2Nc2ncc3ccc(-c4c(F)cc(O)cc4F)nn23)C1]

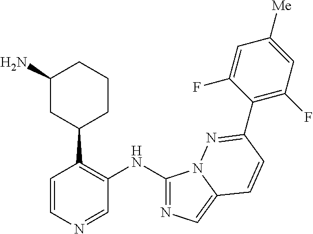 [SMILES: Cc1cc(F)c(-c2ccc3cnc(Nc4cnccc4[C@@H]4CCC[C@H](N)C4)n3n2)c(F)c1]

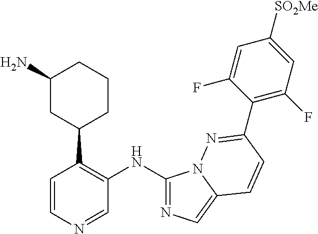 [SMILES: CS(=O)(=O)c1cc(F)c(-c2ccc3cnc(Nc4cnccc4[C@@H]4CCC[C@H](N)C4)n3n2)c(F)c1]

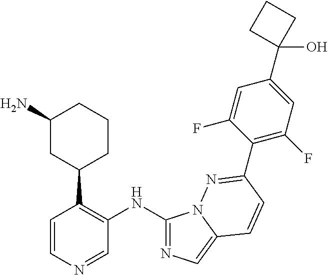 [SMILES: N[C@H]1CCC[C@@H](c2ccncc2Nc2ncc3ccc(-c4c(F)cc(C5(O)CCC5)cc4F)nn23)C1]

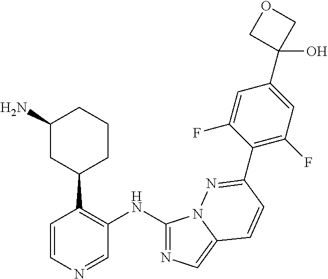 [SMILES: N[C@H]1CCC[C@@H](c2ccncc2Nc2ncc3ccc(-c4c(F)cc(C5(O)COC5)cc4F)nn23)C1]

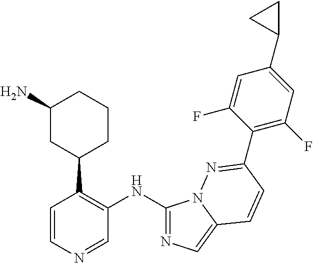 [SMILES: N[C@H]1CCC[C@@H](c2ccncc2Nc2ncc3ccc(-c4c(F)cc(C5CC5)cc4F)nn23)C1]

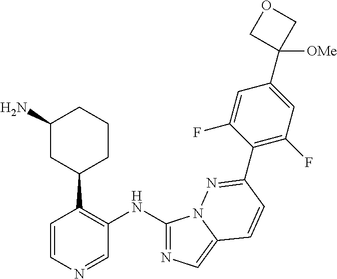 [SMILES: COC1(c2cc(F)c(-c3ccc4cnc(Nc5cnccc5[C@@H]5CCC[C@H](N)C5)n4n3)c(F)c2)COC1]

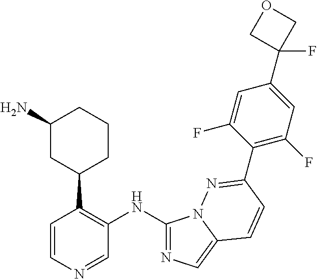 [SMILES: N[C@H]1CCC[C@@H](c2ccncc2Nc2ncc3ccc(-c4c(F)cc(C5(F)COC5)cc4F)nn23)C1]

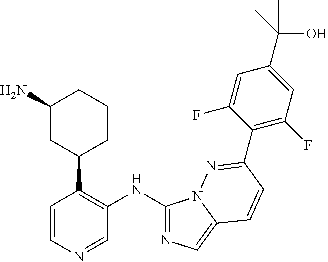 [SMILES: CC(C)(O)c1cc(F)c(-c2ccc3cnc(Nc4cnccc4[C@@H]4CCC[C@H](N)C4)n3n2)c(F)c1]

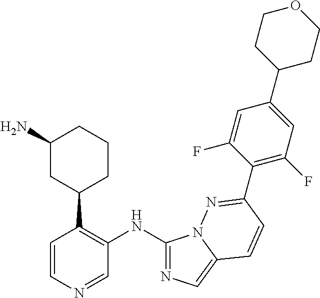 [SMILES: N[C@H]1CCC[C@@H](c2ccncc2Nc2ncc3ccc(-c4c(F)cc(C5CCOCC5)cc4F)nn23)C1]

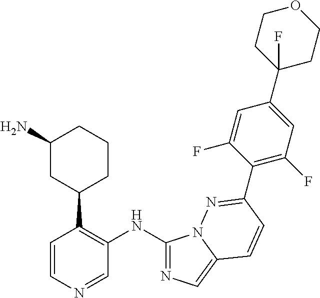

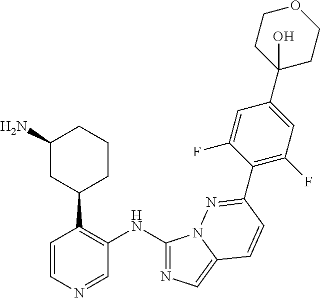

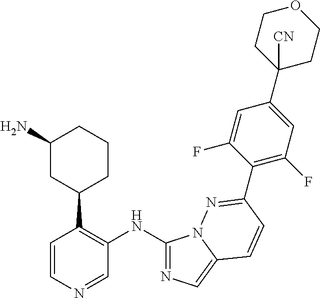

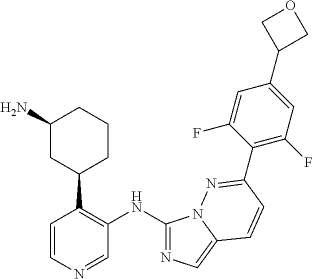 [SMILES: N[C@H]1CCC[C@@H](c2ccncc2Nc2ncc3ccc(-c4c(F)cc(C5COC5)cc4F)nn23)C1]

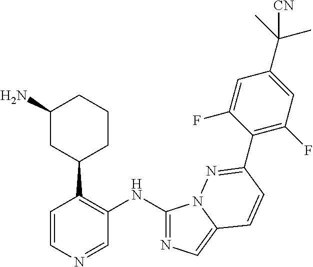 [SMILES: CC(C)(C#N)c1cc(F)c(-c2ccc3cnc(Nc4cnccc4[C@@H]4CCC[C@H](N)C4)n3n2)c(F)c1]

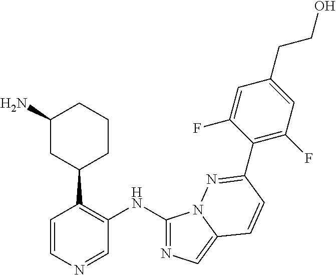 [SMILES: N[C@H]1CCC[C@@H](c2ccncc2Nc2ncc3ccc(-c4c(F)cc(CCO)cc4F)nn23)C1]

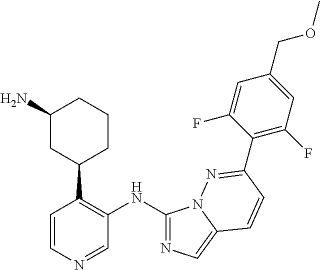 [SMILES: COCc1cc(F)c(-c2ccc3cnc(Nc4cnccc4[C@@H]4CCC[C@H](N)C4)n3n2)c(F)c1]

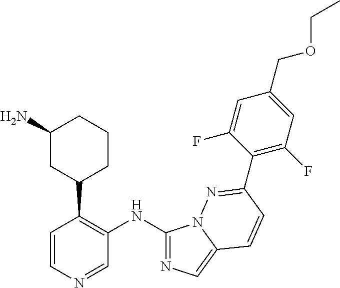

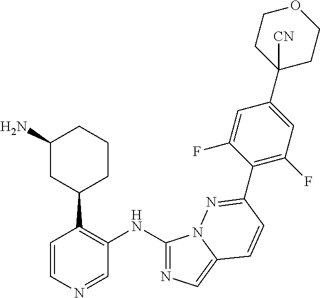

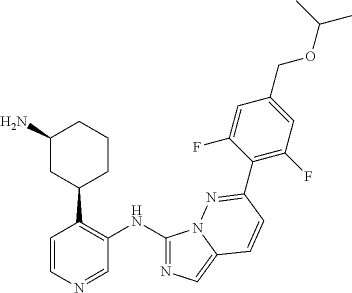 [SMILES: CC(C)OCc1cc(F)c(-c2ccc3cnc(Nc4cnccc4[C@@H]4CCC[C@H](N)C4)n3n2)c(F)c1]

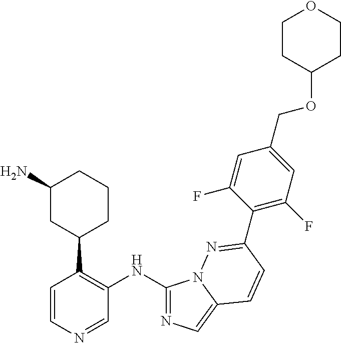 [SMILES: N[C@H]1CCC[C@@H](c2ccncc2Nc2ncc3ccc(-c4c(F)cc(COC5CCOCC5)cc4F)nn23)C1]

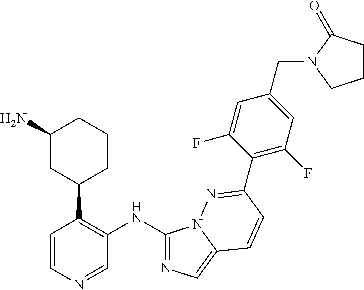 [SMILES: N[C@H]1CCC[C@@H](c2ccncc2Nc2ncc3ccc(-c4c(F)cc(CN5CCCC5=O)cc4F)nn23)C1]

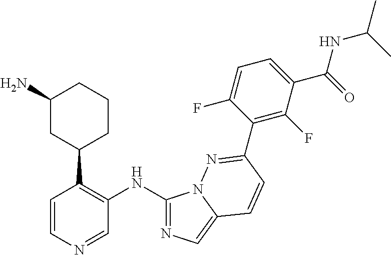 [SMILES: CC(C)NC(=O)c1ccc(F)c(-c2ccc3cnc(Nc4cnccc4[C@@H]4CCC[C@H](N)C4)n3n2)c1F]

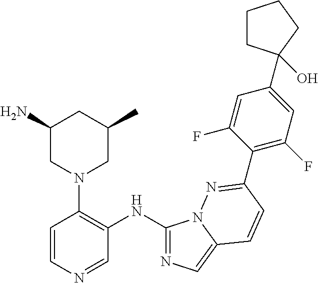 [SMILES: C[C@@H]1C[C@H](N)CN(c2ccncc2Nc2ncc3ccc(-c4c(F)cc(C5(O)CCCC5)cc4F)nn23)C1]

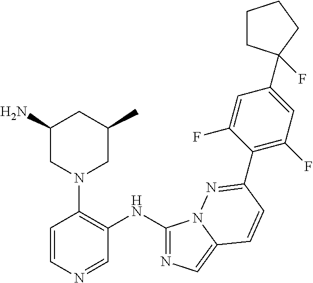 [SMILES: C[C@@H]1C[C@H](N)CN(c2ccncc2Nc2ncc3ccc(-c4c(F)cc(C5(F)CCCC5)cc4F)nn23)C1]

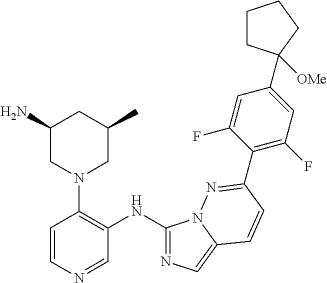 [SMILES: COC1(c2cc(F)c(-c3ccc4cnc(Nc5cnccc5N5C[C@H](C)C[C@H](N)C5)n4n3)c(F)c2)CCCC1]

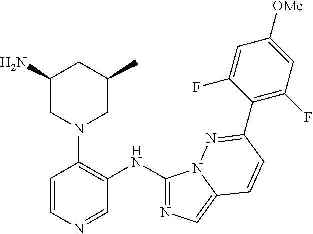 [SMILES: COc1cc(F)c(-c2ccc3cnc(Nc4cnccc4N4C[C@H](C)C[C@H](N)C4)n3n2)c(F)c1]

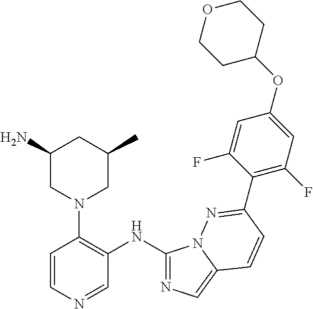 [SMILES: C[C@@H]1C[C@H](N)CN(c2ccncc2Nc2ncc3ccc(-c4c(F)cc(OC5CCOCC5)cc4F)nn23)C1]

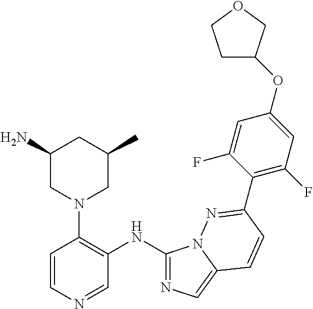 [SMILES: C[C@@H]1C[C@H](N)CN(c2ccncc2Nc2ncc3ccc(-c4c(F)cc(OC5CCOC5)cc4F)nn23)C1]

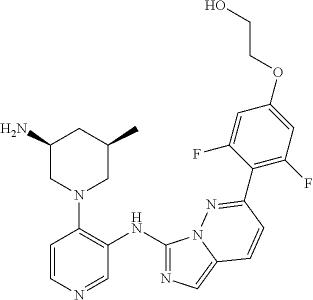 [SMILES: C[C@@H]1C[C@H](N)CN(c2ccncc2Nc2ncc3ccc(-c4c(F)cc(OCCO)cc4F)nn23)C1]

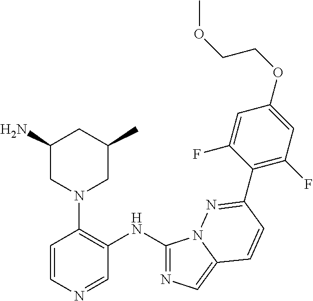 [SMILES: COCCOc1cc(F)c(-c2ccc3cnc(Nc4cnccc4N4C[C@H](C)C[C@H](N)C4)n3n2)c(F)c1]

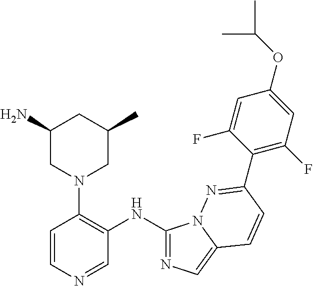 [SMILES: CC(C)Oc1cc(F)c(-c2ccc3cnc(Nc4cnccc4N4C[C@H](C)C[C@H](N)C4)n3n2)c(F)c1]

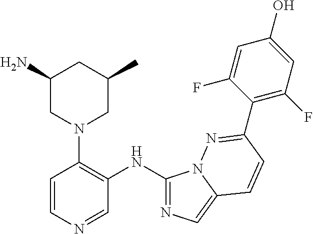 [SMILES: C[C@@H]1C[C@H](N)CN(c2ccncc2Nc2ncc3ccc(-c4c(F)cc(O)cc4F)nn23)C1]

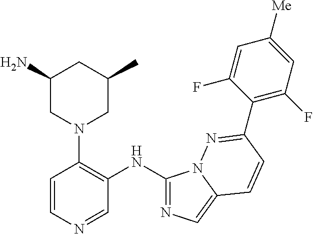 [SMILES: Cc1cc(F)c(-c2ccc3cnc(Nc4cnccc4N4C[C@H](C)C[C@H](N)C4)n3n2)c(F)c1]

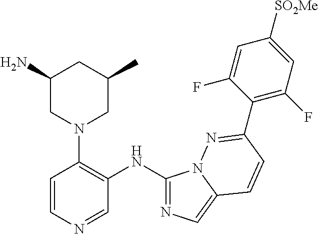 [SMILES: C[C@@H]1C[C@H](N)CN(c2ccncc2Nc2ncc3ccc(-c4c(F)cc(S(C)(=O)=O)cc4F)nn23)C1]

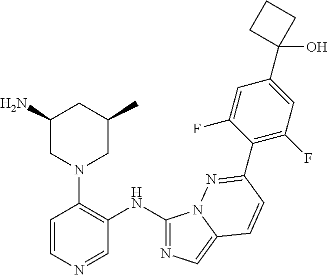 [SMILES: C[C@@H]1C[C@H](N)CN(c2ccncc2Nc2ncc3ccc(-c4c(F)cc(C5(O)CCC5)cc4F)nn23)C1]

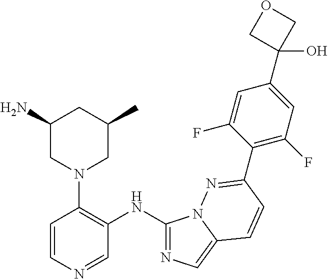 [SMILES: C[C@@H]1C[C@H](N)CN(c2ccncc2Nc2ncc3ccc(-c4c(F)cc(C5(O)COC5)cc4F)nn23)C1]

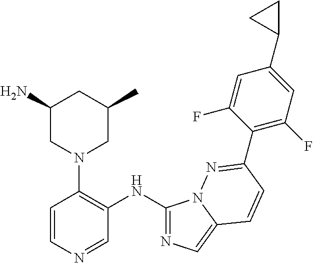 [SMILES: C[C@@H]1C[C@H](N)CN(c2ccncc2Nc2ncc3ccc(-c4c(F)cc(C5CC5)cc4F)nn23)C1]

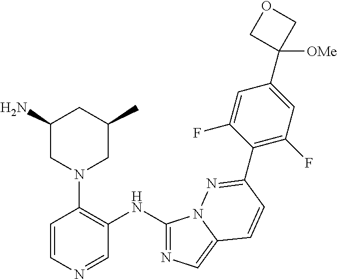 [SMILES: COC1(c2cc(F)c(-c3ccc4cnc(Nc5cnccc5N5C[C@H](C)C[C@H](N)C5)n4n3)c(F)c2)COC1]

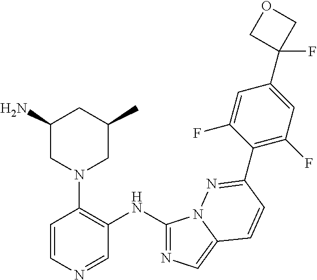 [SMILES: C[C@@H]1C[C@H](N)CN(c2ccncc2Nc2ncc3ccc(-c4c(F)cc(C5(F)COC5)cc4F)nn23)C1]

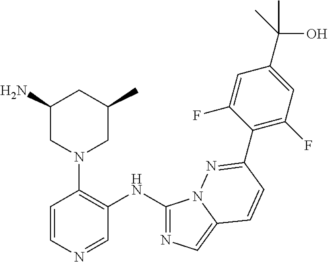 [SMILES: C[C@@H]1C[C@H](N)CN(c2ccncc2Nc2ncc3ccc(-c4c(F)cc(C(C)(C)O)cc4F)nn23)C1]

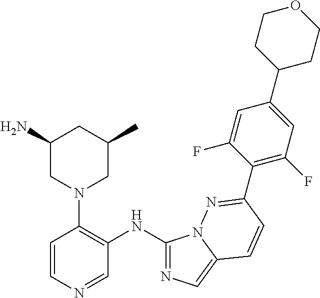 [SMILES: C[C@@H]1C[C@H](N)CN(c2ccncc2Nc2ncc3ccc(-c4c(F)cc(C5CCOCC5)cc4F)nn23)C1]

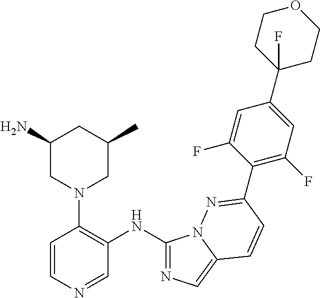

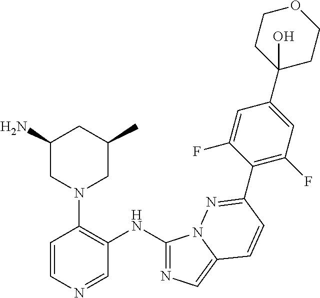

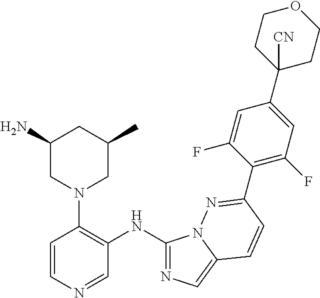

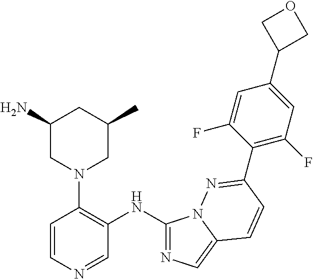 [SMILES: C[C@@H]1C[C@H](N)CN(c2ccncc2Nc2ncc3ccc(-c4c(F)cc(C5COC5)cc4F)nn23)C1]

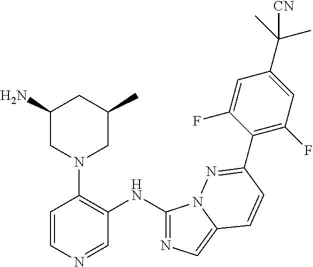 [SMILES: C[C@@H]1C[C@H](N)CN(c2ccncc2Nc2ncc3ccc(-c4c(F)cc(C(C)(C)C#N)cc4F)nn23)C1]

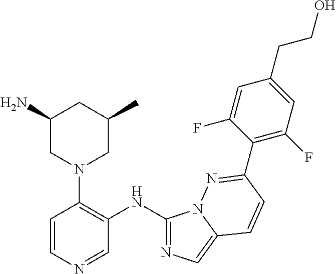 [SMILES: C[C@@H]1C[C@H](N)CN(c2ccncc2Nc2ncc3ccc(-c4c(F)cc(CCO)cc4F)nn23)C1]

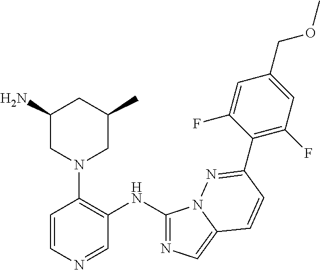 [SMILES: COCc1cc(F)c(-c2ccc3cnc(Nc4cnccc4N4C[C@H](C)C[C@H](N)C4)n3n2)c(F)c1]

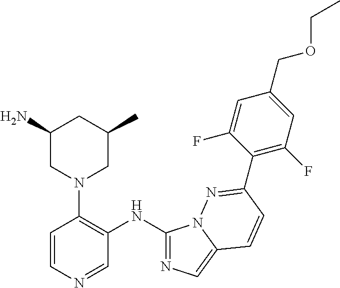 [SMILES: CCOCc1cc(F)c(-c2ccc3cnc(Nc4cnccc4N4C[C@H](C)C[C@H](N)C4)n3n2)c(F)c1]

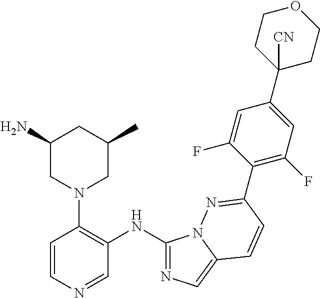

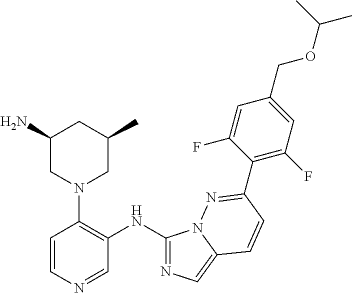 [SMILES: CC(C)OCc1cc(F)c(-c2ccc3cnc(Nc4cnccc4N4C[C@H](C)C[C@H](N)C4)n3n2)c(F)c1]

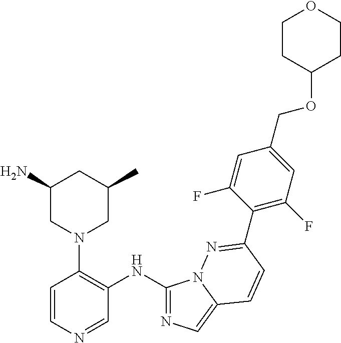 [SMILES: C[C@@H]1C[C@H](N)CN(c2ccncc2Nc2ncc3ccc(-c4c(F)cc(COC5CCOCC5)cc4F)nn23)C1]

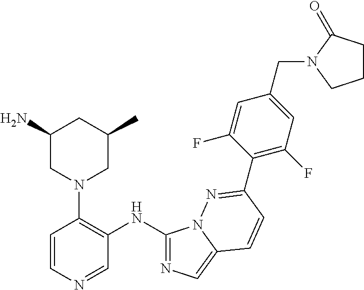 [SMILES: C[C@@H]1C[C@H](N)CN(c2ccncc2Nc2ncc3ccc(-c4c(F)cc(CN5CCCC5=O)cc4F)nn23)C1]

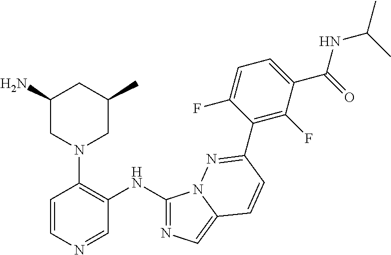 [SMILES: CC(C)NC(=O)c1ccc(F)c(-c2ccc3cnc(Nc4cnccc4N4C[C@H](C)C[C@H](N)C4)n3n2)c1F]

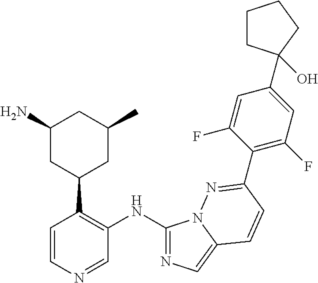 [SMILES: C[C@@H]1C[C@H](N)C[C@H](c2ccncc2Nc2ncc3ccc(-c4c(F)cc(C5(O)CCCC5)cc4F)nn23)C1]

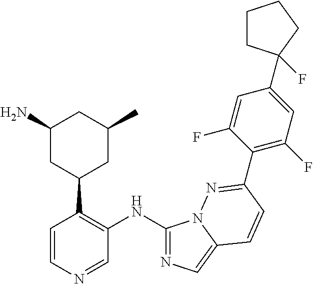 [SMILES: C[C@@H]1C[C@H](N)C[C@H](c2ccncc2Nc2ncc3ccc(-c4c(F)cc(C5(F)CCCC5)cc4F)nn23)C1]

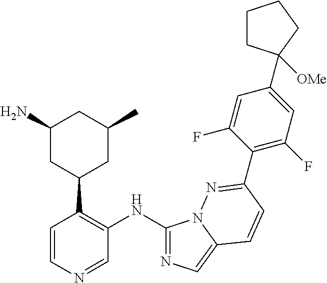 [SMILES: COC1(c2cc(F)c(-c3ccc4cnc(Nc5cnccc5[C@@H]5C[C@H](C)C[C@H](N)C5)n4n3)c(F)c2)CCCC1]

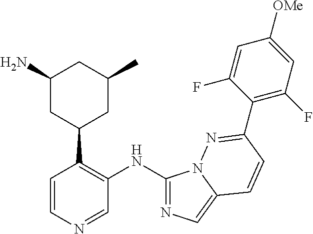 [SMILES: COc1cc(F)c(-c2ccc3cnc(Nc4cnccc4[C@@H]4C[C@H](C)C[C@H](N)C4)n3n2)c(F)c1]

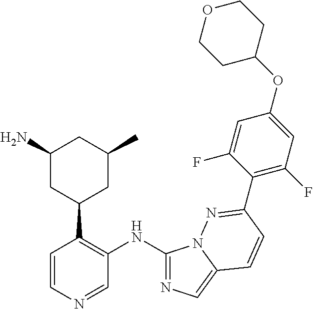 [SMILES: C[C@@H]1C[C@H](N)C[C@H](c2ccncc2Nc2ncc3ccc(-c4c(F)cc(OC5CCOCC5)cc4F)nn23)C1]

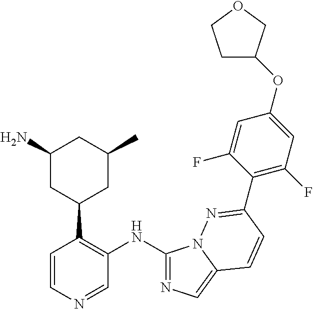 [SMILES: C[C@@H]1C[C@H](N)C[C@H](c2ccncc2Nc2ncc3ccc(-c4c(F)cc(OC5CCOC5)cc4F)nn23)C1]

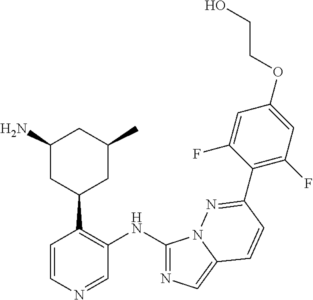 [SMILES: C[C@@H]1C[C@H](N)C[C@H](c2ccncc2Nc2ncc3ccc(-c4c(F)cc(OCCO)cc4F)nn23)C1]

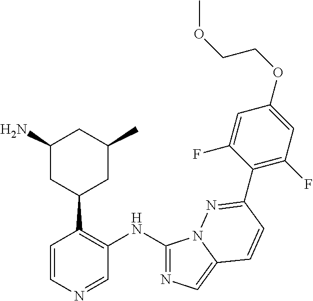 [SMILES: COCCOc1cc(F)c(-c2ccc3cnc(Nc4cnccc4[C@@H]4C[C@H](C)C[C@H](N)C4)n3n2)c(F)c1]

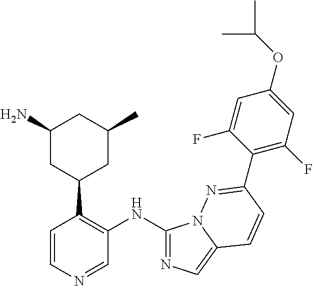 [SMILES: CC(C)Oc1cc(F)c(-c2ccc3cnc(Nc4cnccc4[C@@H]4C[C@H](C)C[C@H](N)C4)n3n2)c(F)c1]

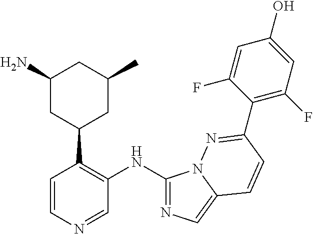 [SMILES: C[C@@H]1C[C@H](N)C[C@H](c2ccncc2Nc2ncc3ccc(-c4c(F)cc(O)cc4F)nn23)C1]

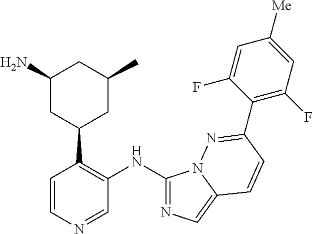 [SMILES: Cc1cc(F)c(-c2ccc3cnc(Nc4cnccc4[C@@H]4C[C@H](C)C[C@H](N)C4)n3n2)c(F)c1]

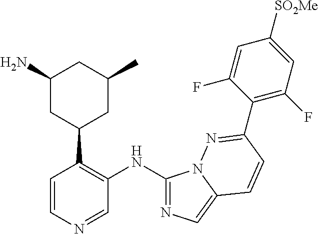 [SMILES: C[C@@H]1C[C@H](N)C[C@H](c2ccncc2Nc2ncc3ccc(-c4c(F)cc(S(C)(=O)=O)cc4F)nn23)C1]

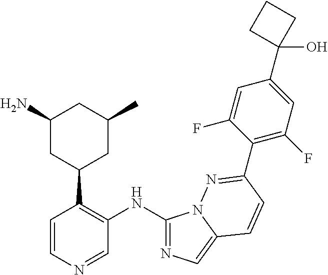 [SMILES: C[C@@H]1C[C@H](N)C[C@H](c2ccncc2Nc2ncc3ccc(-c4c(F)cc(C5(O)CCC5)cc4F)nn23)C1]

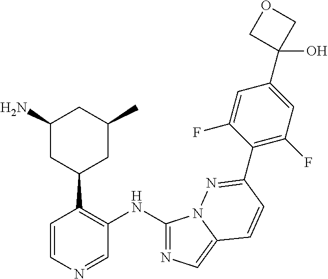 [SMILES: C[C@@H]1C[C@H](N)C[C@H](c2ccncc2Nc2ncc3ccc(-c4c(F)cc(C5(O)COC5)cc4F)nn23)C1]

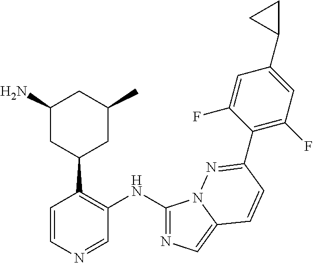 [SMILES: C[C@@H]1C[C@H](N)C[C@H](c2ccncc2Nc2ncc3ccc(-c4c(F)cc(C5CC5)cc4F)nn23)C1]

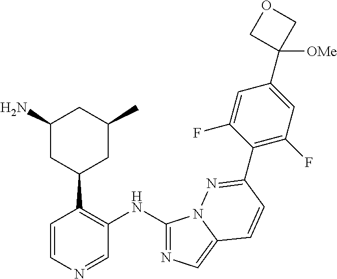 [SMILES: COC1(c2cc(F)c(-c3ccc4cnc(Nc5cnccc5[C@@H]5C[C@H](C)C[C@H](N)C5)n4n3)c(F)c2)COC1]

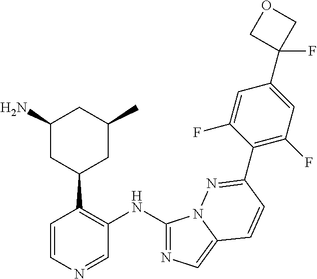 [SMILES: C[C@@H]1C[C@H](N)C[C@H](c2ccncc2Nc2ncc3ccc(-c4c(F)cc(C5(F)COC5)cc4F)nn23)C1]

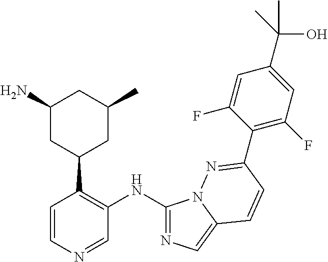 [SMILES: C[C@@H]1C[C@H](N)C[C@H](c2ccncc2Nc2ncc3ccc(-c4c(F)cc(C(C)(C)O)cc4F)nn23)C1]

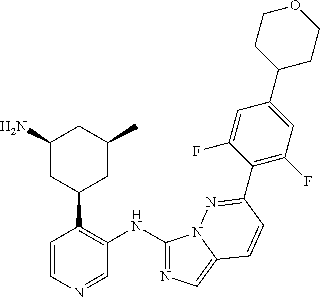 [SMILES: C[C@@H]1C[C@H](N)C[C@H](c2ccncc2Nc2ncc3ccc(-c4c(F)cc(C5CCOCC5)cc4F)nn23)C1]

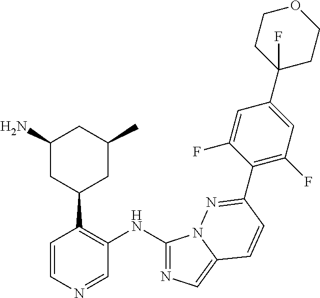

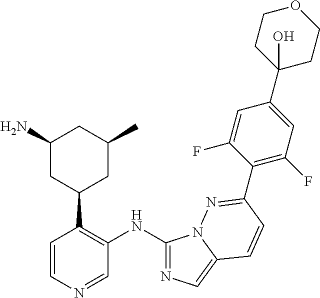

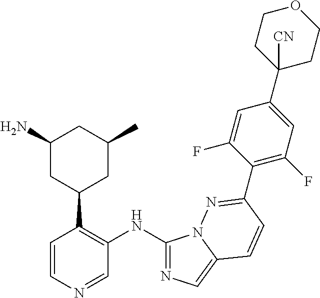

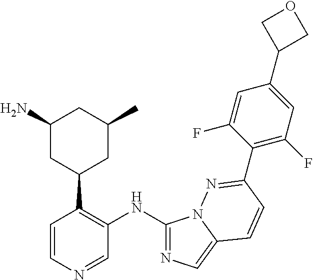 [SMILES: C[C@@H]1C[C@H](N)C[C@H](c2ccncc2Nc2ncc3ccc(-c4c(F)cc(C5COC5)cc4F)nn23)C1]

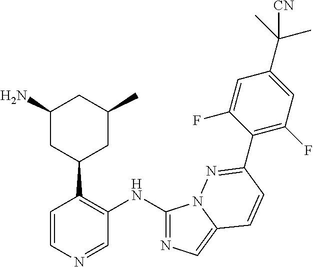 [SMILES: C[C@@H]1C[C@H](N)C[C@H](c2ccncc2Nc2ncc3ccc(-c4c(F)cc(C(C)(C)C#N)cc4F)nn23)C1]

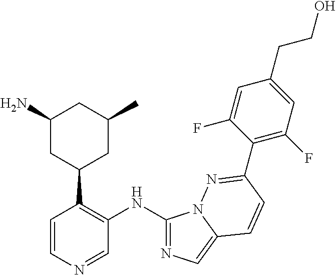 [SMILES: C[C@@H]1C[C@H](N)C[C@H](c2ccncc2Nc2ncc3ccc(-c4c(F)cc(CCO)cc4F)nn23)C1]

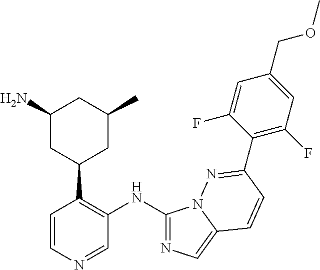 [SMILES: COCc1cc(F)c(-c2ccc3cnc(Nc4cnccc4[C@@H]4C[C@H](C)C[C@H](N)C4)n3n2)c(F)c1]

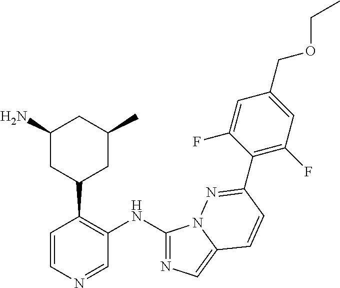

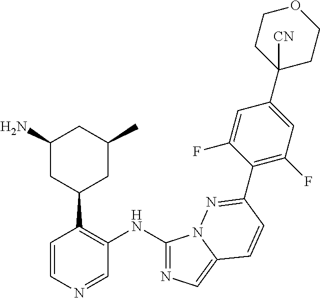

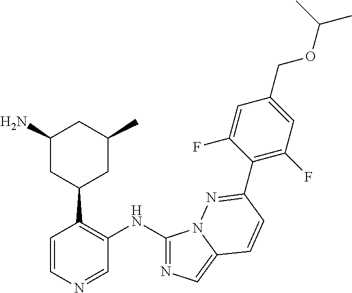 [SMILES: CC(C)OCc1cc(F)c(-c2ccc3cnc(Nc4cnccc4[C@@H]4C[C@H](C)C[C@H](N)C4)n3n2)c(F)c1]

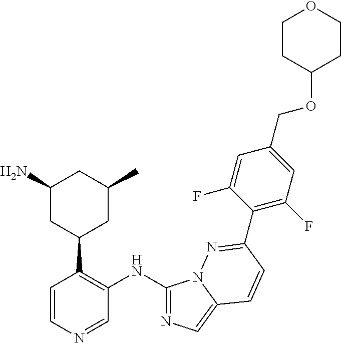 [SMILES: C[C@@H]1C[C@H](N)C[C@H](c2ccncc2Nc2ncc3ccc(-c4c(F)cc(COC5CCOCC5)cc4F)nn23)C1]

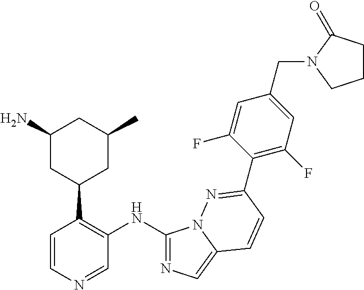 [SMILES: C[C@@H]1C[C@H](N)C[C@H](c2ccncc2Nc2ncc3ccc(-c4c(F)cc(CN5CCCC5=O)cc4F)nn23)C1]

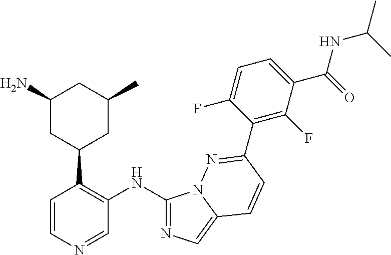 [SMILES: CC(C)NC(=O)c1ccc(F)c(-c2ccc3cnc(Nc4cnccc4[C@@H]4C[C@H](C)C[C@H](N)C4)n3n2)c1F]

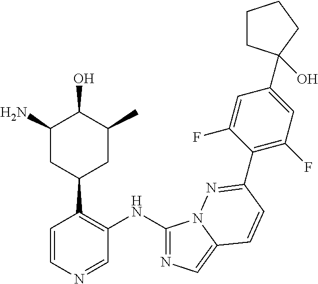 [SMILES: C[C@H]1C[C@@H](c2ccncc2Nc2ncc3ccc(-c4c(F)cc(C5(O)CCCC5)cc4F)nn23)C[C@@H](N)[C@H]1O]

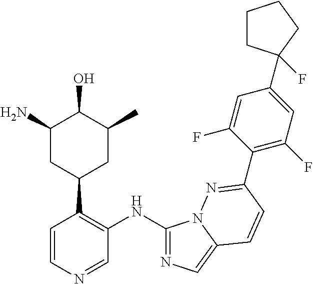 [SMILES: C[C@H]1C[C@@H](c2ccncc2Nc2ncc3ccc(-c4c(F)cc(C5(F)CCCC5)cc4F)nn23)C[C@@H](N)[C@H]1O]

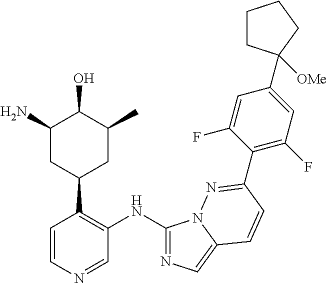 [SMILES: COC1(c2cc(F)c(-c3ccc4cnc(Nc5cnccc5[C@H]5C[C@@H](N)[C@@H](O)[C@@H](C)C5)n4n3)c(F)c2)CCCC1]

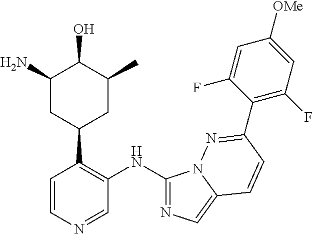 [SMILES: COc1cc(F)c(-c2ccc3cnc(Nc4cnccc4[C@H]4C[C@@H](N)[C@@H](O)[C@@H](C)C4)n3n2)c(F)c1]

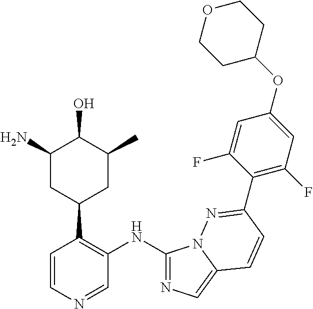 [SMILES: C[C@H]1C[C@@H](c2ccncc2Nc2ncc3ccc(-c4c(F)cc(OC5CCOCC5)cc4F)nn23)C[C@@H](N)[C@H]1O]

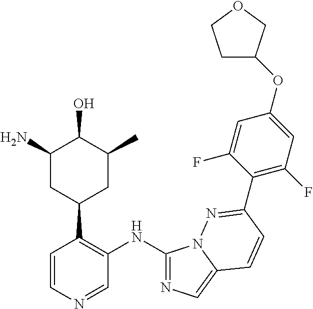 [SMILES: C[C@H]1C[C@@H](c2ccncc2Nc2ncc3ccc(-c4c(F)cc(OC5CCOC5)cc4F)nn23)C[C@@H](N)[C@H]1O]

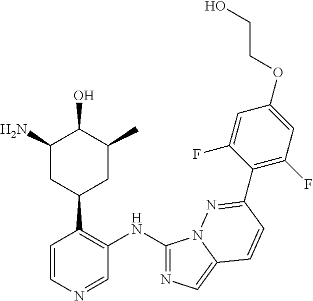 [SMILES: C[C@H]1C[C@@H](c2ccncc2Nc2ncc3ccc(-c4c(F)cc(OCCO)cc4F)nn23)C[C@@H](N)[C@H]1O]

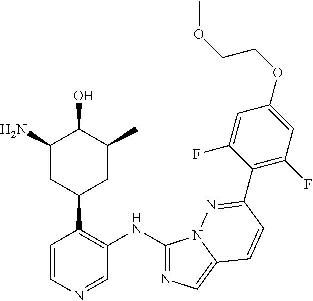 [SMILES: COCCOc1cc(F)c(-c2ccc3cnc(Nc4cnccc4[C@H]4C[C@@H](N)[C@@H](O)[C@@H](C)C4)n3n2)c(F)c1]

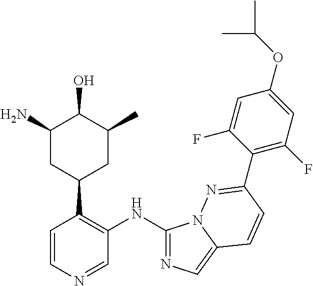 [SMILES: CC(C)Oc1cc(F)c(-c2ccc3cnc(Nc4cnccc4[C@H]4C[C@@H](N)[C@@H](O)[C@@H](C)C4)n3n2)c(F)c1]

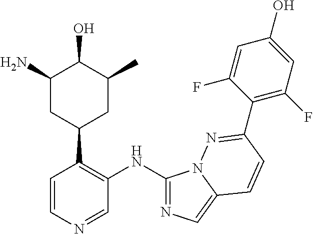 [SMILES: C[C@H]1C[C@@H](c2ccncc2Nc2ncc3ccc(-c4c(F)cc(O)cc4F)nn23)C[C@@H](N)[C@H]1O]

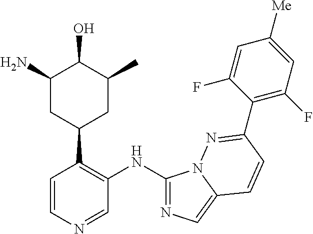 [SMILES: Cc1cc(F)c(-c2ccc3cnc(Nc4cnccc4[C@H]4C[C@@H](N)[C@@H](O)[C@@H](C)C4)n3n2)c(F)c1]

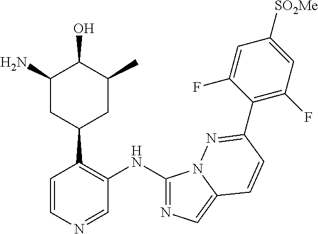 [SMILES: C[C@H]1C[C@@H](c2ccncc2Nc2ncc3ccc(-c4c(F)cc(S(C)(=O)=O)cc4F)nn23)C[C@@H](N)[C@H]1O]

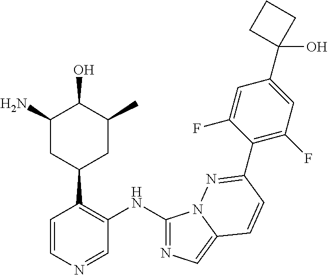 [SMILES: C[C@H]1C[C@@H](c2ccncc2Nc2ncc3ccc(-c4c(F)cc(C5(O)CCC5)cc4F)nn23)C[C@@H](N)[C@H]1O]

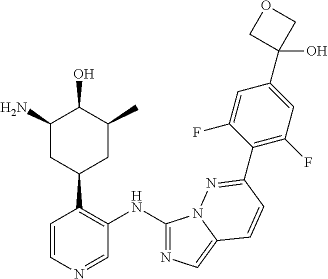 [SMILES: C[C@H]1C[C@@H](c2ccncc2Nc2ncc3ccc(-c4c(F)cc(C5(O)COC5)cc4F)nn23)C[C@@H](N)[C@H]1O]

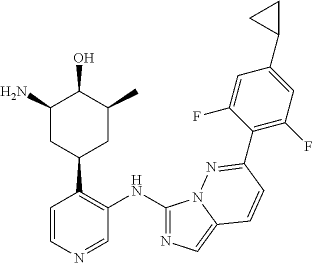 [SMILES: C[C@H]1C[C@@H](c2ccncc2Nc2ncc3ccc(-c4c(F)cc(C5CC5)cc4F)nn23)C[C@@H](N)[C@H]1O]

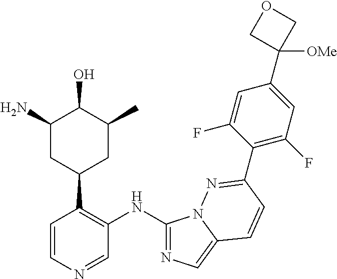 [SMILES: COC1(c2cc(F)c(-c3ccc4cnc(Nc5cnccc5[C@H]5C[C@@H](N)[C@@H](O)[C@@H](C)C5)n4n3)c(F)c2)COC1]

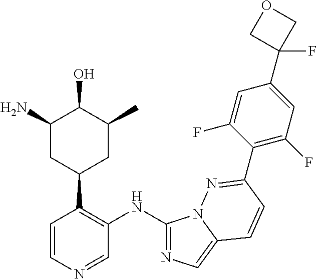 [SMILES: C[C@H]1C[C@@H](c2ccncc2Nc2ncc3ccc(-c4c(F)cc(C5(F)COC5)cc4F)nn23)C[C@@H](N)[C@H]1O]

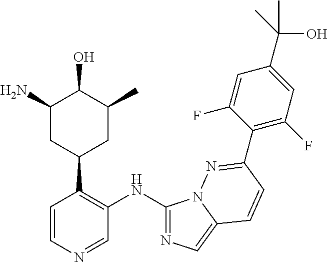 [SMILES: C[C@H]1C[C@@H](c2ccncc2Nc2ncc3ccc(-c4c(F)cc(C(C)(C)O)cc4F)nn23)C[C@@H](N)[C@H]1O]

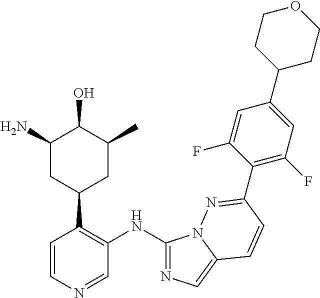 [SMILES: C[C@H]1C[C@@H](c2ccncc2Nc2ncc3ccc(-c4c(F)cc(C5CCOCC5)cc4F)nn23)C[C@@H](N)[C@H]1O]

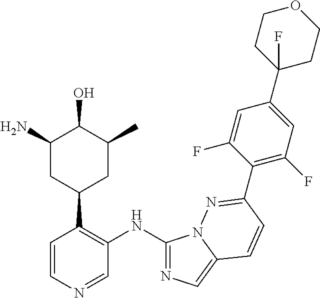

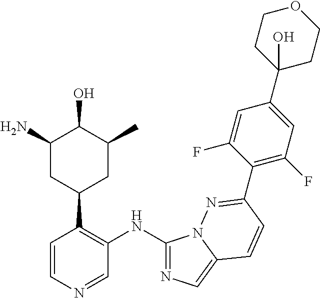

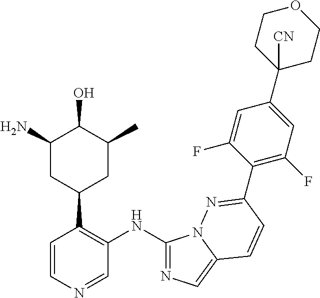

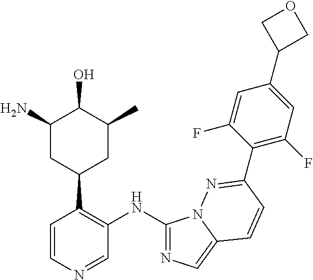 [SMILES: C[C@H]1C[C@@H](c2ccncc2Nc2ncc3ccc(-c4c(F)cc(C5COC5)cc4F)nn23)C[C@@H](N)[C@H]1O]

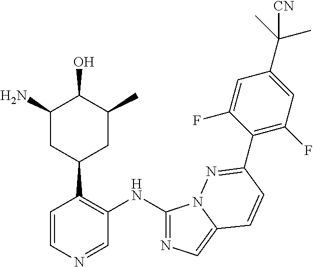 [SMILES: C[C@H]1C[C@@H](c2ccncc2Nc2ncc3ccc(-c4c(F)cc(C(C)(C)C#N)cc4F)nn23)C[C@@H](N)[C@H]1O]

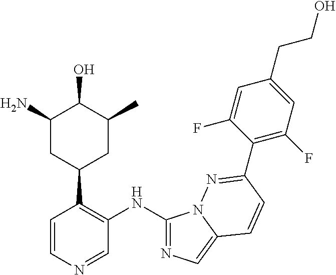 [SMILES: C[C@H]1C[C@@H](c2ccncc2Nc2ncc3ccc(-c4c(F)cc(CCO)cc4F)nn23)C[C@@H](N)[C@H]1O]

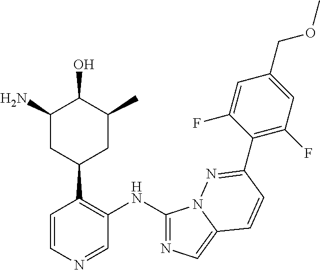 [SMILES: COCc1cc(F)c(-c2ccc3cnc(Nc4cnccc4[C@H]4C[C@@H](N)[C@@H](O)[C@@H](C)C4)n3n2)c(F)c1]

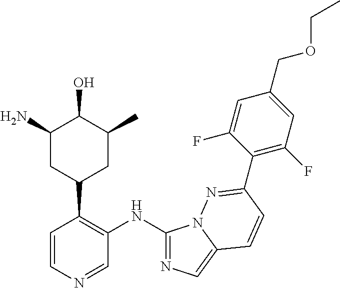

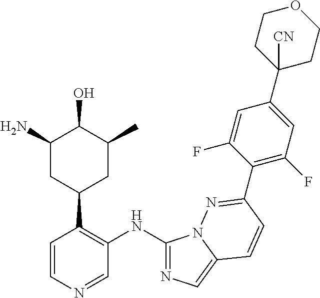

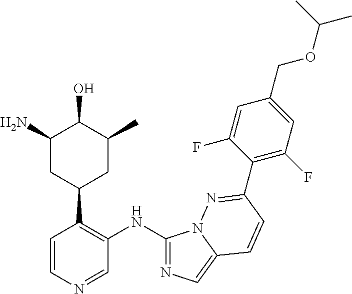 [SMILES: CC(C)OCc1cc(F)c(-c2ccc3cnc(Nc4cnccc4[C@H]4C[C@@H](N)[C@@H](O)[C@@H](C)C4)n3n2)c(F)c1]

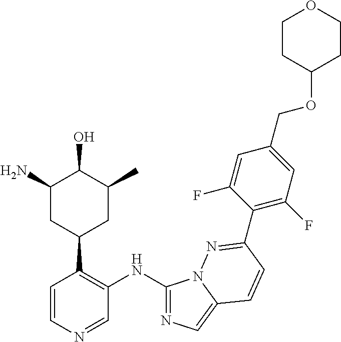 [SMILES: C[C@H]1C[C@@H](c2ccncc2Nc2ncc3ccc(-c4c(F)cc(COC5CCOCC5)cc4F)nn23)C[C@@H](N)[C@H]1O]

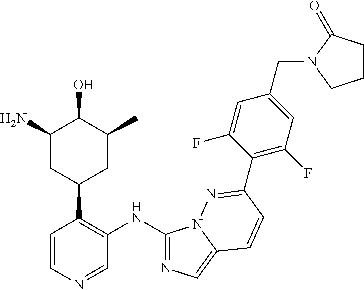 [SMILES: C[C@H]1C[C@@H](c2ccncc2Nc2ncc3ccc(-c4c(F)cc(CN5CCCC5=O)cc4F)nn23)C[C@@H](N)[C@H]1O]

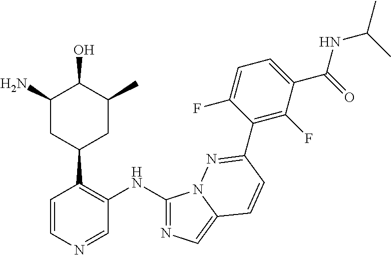 [SMILES: CC(C)NC(=O)c1ccc(F)c(-c2ccc3cnc(Nc4cnccc4[C@H]4C[C@@H](N)[C@@H](O)[C@@H](C)C4)n3n2)c1F]

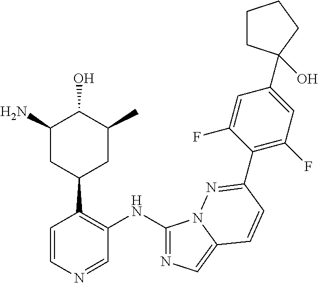 [SMILES: C[C@H]1C[C@@H](c2ccncc2Nc2ncc3ccc(-c4c(F)cc(C5(O)CCCC5)cc4F)nn23)C[C@@H](N)[C@@H]1O]

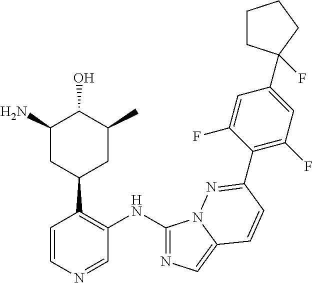 [SMILES: C[C@H]1C[C@@H](c2ccncc2Nc2ncc3ccc(-c4c(F)cc(C5(F)CCCC5)cc4F)nn23)C[C@@H](N)[C@@H]1O]

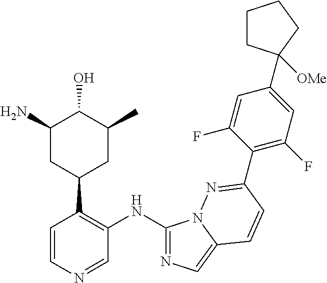 [SMILES: COC1(c2cc(F)c(-c3ccc4cnc(Nc5cnccc5[C@H]5C[C@@H](N)[C@H](O)[C@@H](C)C5)n4n3)c(F)c2)CCCC1]

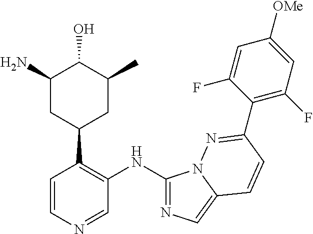 [SMILES: COc1cc(F)c(-c2ccc3cnc(Nc4cnccc4[C@H]4C[C@@H](N)[C@H](O)[C@@H](C)C4)n3n2)c(F)c1]

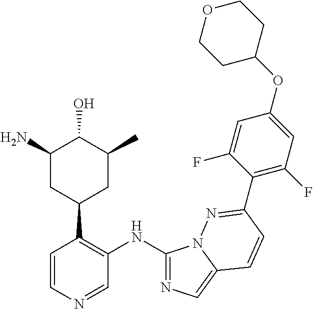 [SMILES: C[C@H]1C[C@@H](c2ccncc2Nc2ncc3ccc(-c4c(F)cc(OC5CCOCC5)cc4F)nn23)C[C@@H](N)[C@@H]1O]

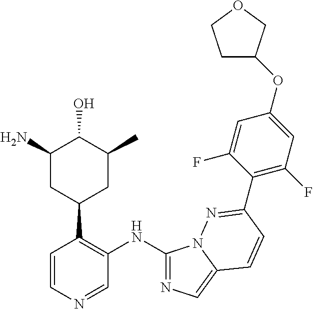 [SMILES: C[C@H]1C[C@@H](c2ccncc2Nc2ncc3ccc(-c4c(F)cc(OC5CCOC5)cc4F)nn23)C[C@@H](N)[C@@H]1O]

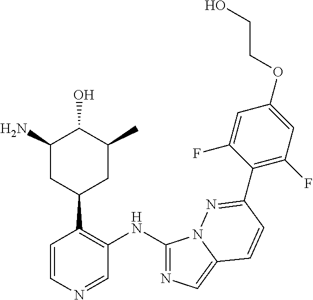 [SMILES: C[C@H]1C[C@@H](c2ccncc2Nc2ncc3ccc(-c4c(F)cc(OCCO)cc4F)nn23)C[C@@H](N)[C@@H]1O]

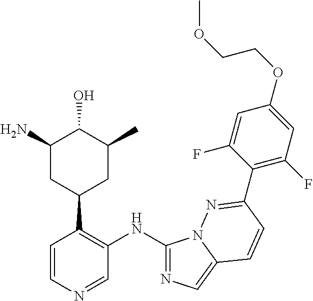 [SMILES: COCCOc1cc(F)c(-c2ccc3cnc(Nc4cnccc4[C@H]4C[C@@H](N)[C@H](O)[C@@H](C)C4)n3n2)c(F)c1]

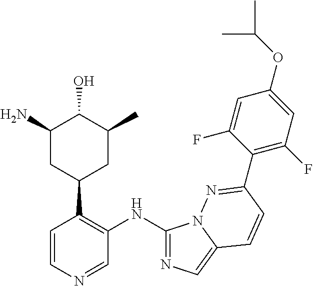 [SMILES: CC(C)Oc1cc(F)c(-c2ccc3cnc(Nc4cnccc4[C@H]4C[C@@H](N)[C@H](O)[C@@H](C)C4)n3n2)c(F)c1]

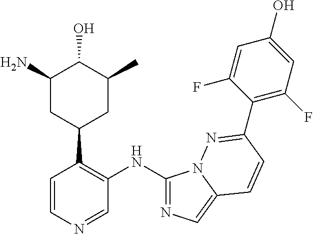 [SMILES: C[C@H]1C[C@@H](c2ccncc2Nc2ncc3ccc(-c4c(F)cc(O)cc4F)nn23)C[C@@H](N)[C@@H]1O]

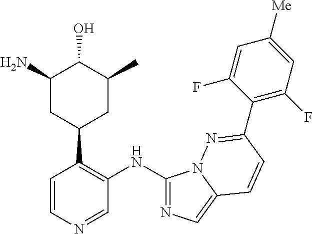 [SMILES: Cc1cc(F)c(-c2ccc3cnc(Nc4cnccc4[C@H]4C[C@@H](N)[C@H](O)[C@@H](C)C4)n3n2)c(F)c1]

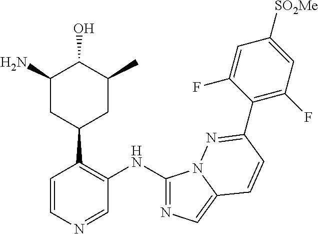 [SMILES: C[C@H]1C[C@@H](c2ccncc2Nc2ncc3ccc(-c4c(F)cc(S(C)(=O)=O)cc4F)nn23)C[C@@H](N)[C@@H]1O]

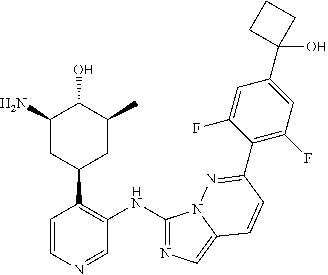 [SMILES: C[C@H]1C[C@@H](c2ccncc2Nc2ncc3ccc(-c4c(F)cc(C5(O)CCC5)cc4F)nn23)C[C@@H](N)[C@@H]1O]

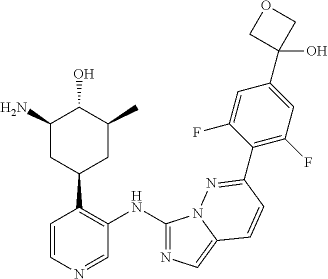 [SMILES: C[C@H]1C[C@@H](c2ccncc2Nc2ncc3ccc(-c4c(F)cc(C5(O)COC5)cc4F)nn23)C[C@@H](N)[C@@H]1O]

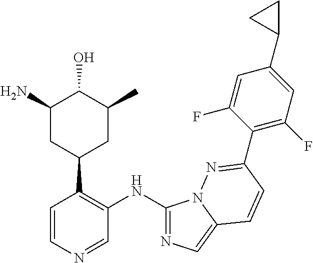 [SMILES: C[C@H]1C[C@@H](c2ccncc2Nc2ncc3ccc(-c4c(F)cc(C5CC5)cc4F)nn23)C[C@@H](N)[C@@H]1O]

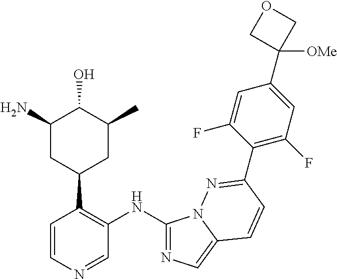 [SMILES: COC1(c2cc(F)c(-c3ccc4cnc(Nc5cnccc5[C@H]5C[C@@H](N)[C@H](O)[C@@H](C)C5)n4n3)c(F)c2)COC1]

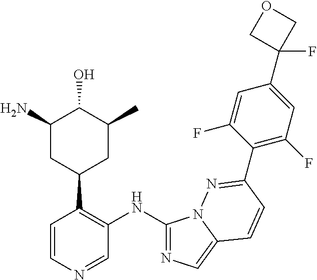 [SMILES: C[C@H]1C[C@@H](c2ccncc2Nc2ncc3ccc(-c4c(F)cc(C5(F)COC5)cc4F)nn23)C[C@@H](N)[C@@H]1O]

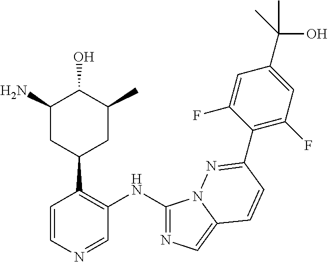 [SMILES: C[C@H]1C[C@@H](c2ccncc2Nc2ncc3ccc(-c4c(F)cc(C(C)(C)O)cc4F)nn23)C[C@@H](N)[C@@H]1O]

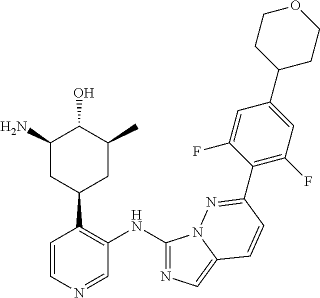 [SMILES: C[C@H]1C[C@@H](c2ccncc2Nc2ncc3ccc(-c4c(F)cc(C5CCOCC5)cc4F)nn23)C[C@@H](N)[C@@H]1O]

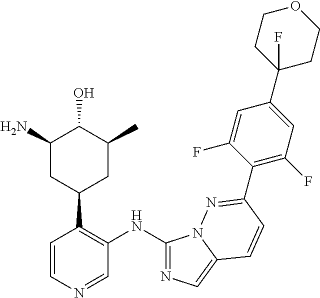

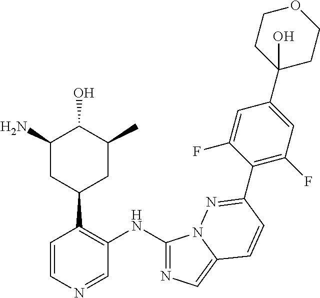

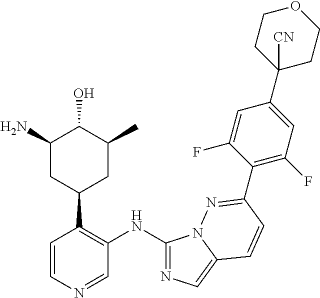

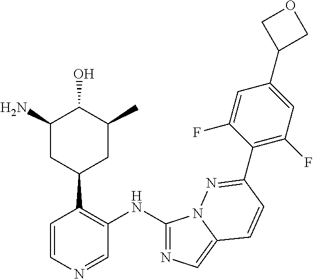 [SMILES: C[C@H]1C[C@@H](c2ccncc2Nc2ncc3ccc(-c4c(F)cc(C5COC5)cc4F)nn23)C[C@@H](N)[C@@H]1O]

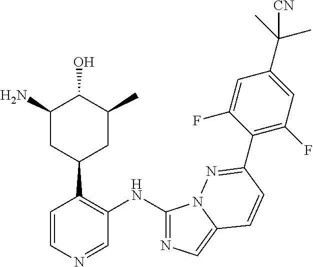 [SMILES: C[C@H]1C[C@@H](c2ccncc2Nc2ncc3ccc(-c4c(F)cc(C(C)(C)C#N)cc4F)nn23)C[C@@H](N)[C@@H]1O]

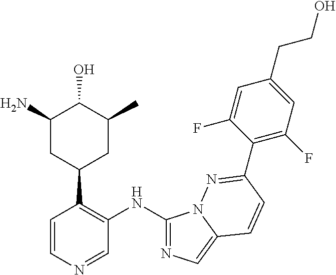 [SMILES: C[C@H]1C[C@@H](c2ccncc2Nc2ncc3ccc(-c4c(F)cc(CCO)cc4F)nn23)C[C@@H](N)[C@@H]1O]

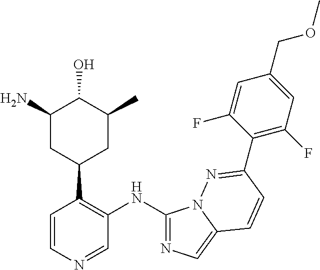 [SMILES: COCc1cc(F)c(-c2ccc3cnc(Nc4cnccc4[C@H]4C[C@@H](N)[C@H](O)[C@@H](C)C4)n3n2)c(F)c1]

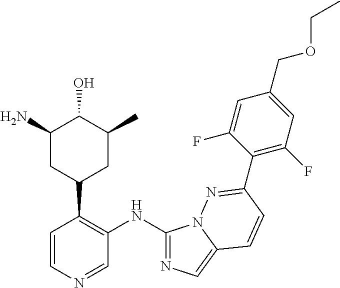

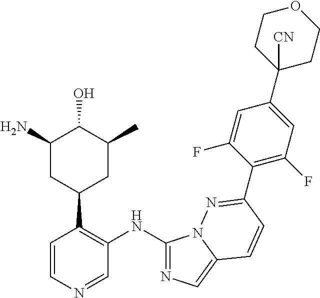

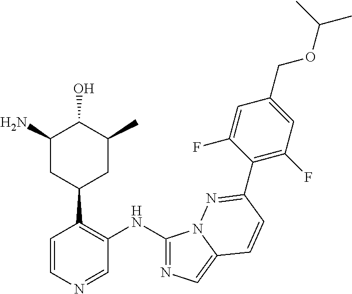 [SMILES: CC(C)OCc1cc(F)c(-c2ccc3cnc(Nc4cnccc4[C@H]4C[C@@H](N)[C@H](O)[C@@H](C)C4)n3n2)c(F)c1]

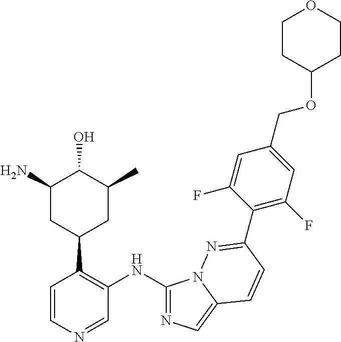 [SMILES: C[C@H]1C[C@@H](c2ccncc2Nc2ncc3ccc(-c4c(F)cc(COC5CCOCC5)cc4F)nn23)C[C@@H](N)[C@@H]1O]

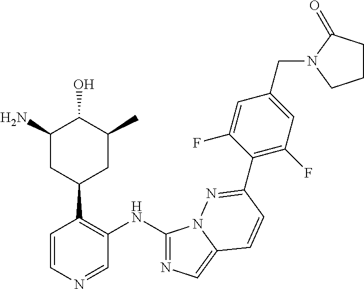 [SMILES: C[C@H]1C[C@@H](c2ccncc2Nc2ncc3ccc(-c4c(F)cc(CN5CCCC5=O)cc4F)nn23)C[C@@H](N)[C@@H]1O]

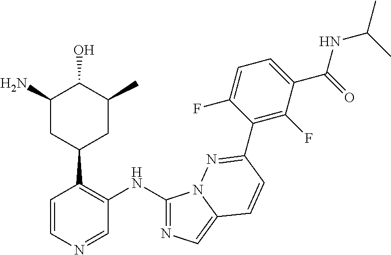 [SMILES: CC(C)NC(=O)c1ccc(F)c(-c2ccc3cnc(Nc4cnccc4[C@H]4C[C@@H](N)[C@H](O)[C@@H](C)C4)n3n2)c1F]

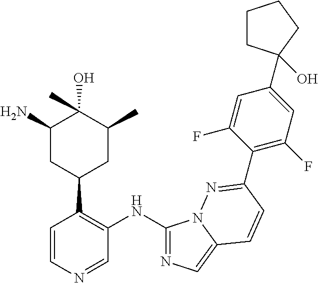 [SMILES: C[C@H]1C[C@@H](c2ccncc2Nc2ncc3ccc(-c4c(F)cc(C5(O)CCCC5)cc4F)nn23)C[C@@H](N)[C@]1(C)O]

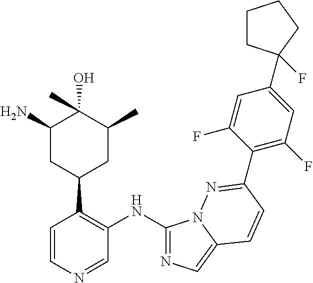 [SMILES: C[C@H]1C[C@@H](c2ccncc2Nc2ncc3ccc(-c4c(F)cc(C5(F)CCCC5)cc4F)nn23)C[C@@H](N)[C@]1(C)O]

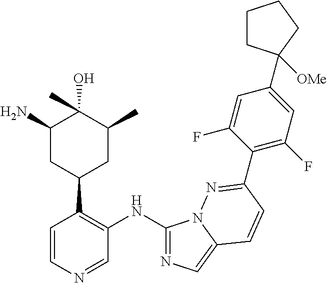 [SMILES: COC1(c2cc(F)c(-c3ccc4cnc(Nc5cnccc5[C@H]5C[C@@H](N)[C@](C)(O)[C@@H](C)C5)n4n3)c(F)c2)CCCC1]

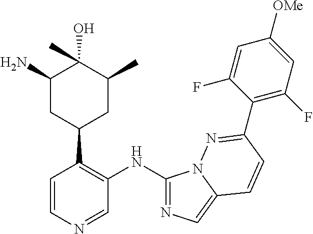 [SMILES: COc1cc(F)c(-c2ccc3cnc(Nc4cnccc4[C@H]4C[C@@H](N)[C@](C)(O)[C@@H](C)C4)n3n2)c(F)c1]

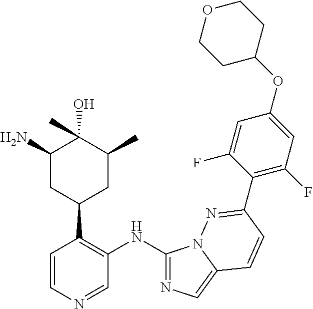 [SMILES: C[C@H]1C[C@@H](c2ccncc2Nc2ncc3ccc(-c4c(F)cc(OC5CCOCC5)cc4F)nn23)C[C@@H](N)[C@]1(C)O]

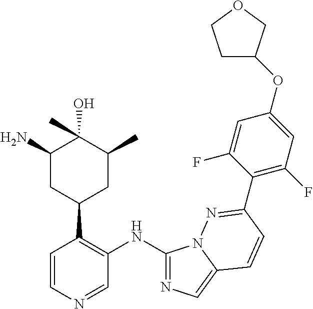 [SMILES: C[C@H]1C[C@@H](c2ccncc2Nc2ncc3ccc(-c4c(F)cc(OC5CCOC5)cc4F)nn23)C[C@@H](N)[C@]1(C)O]

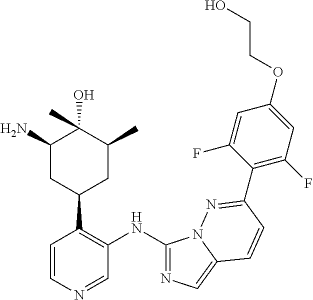 [SMILES: C[C@H]1C[C@@H](c2ccncc2Nc2ncc3ccc(-c4c(F)cc(OCCO)cc4F)nn23)C[C@@H](N)[C@]1(C)O]

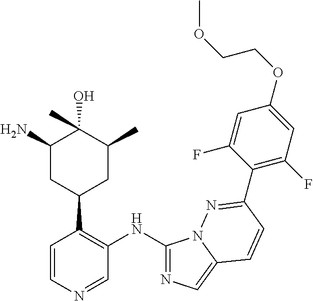 [SMILES: COCCOc1cc(F)c(-c2ccc3cnc(Nc4cnccc4[C@H]4C[C@@H](N)[C@](C)(O)[C@@H](C)C4)n3n2)c(F)c1]

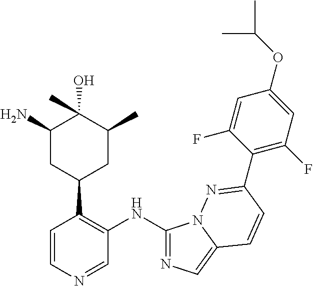 [SMILES: CC(C)Oc1cc(F)c(-c2ccc3cnc(Nc4cnccc4[C@H]4C[C@@H](N)[C@](C)(O)[C@@H](C)C4)n3n2)c(F)c1]

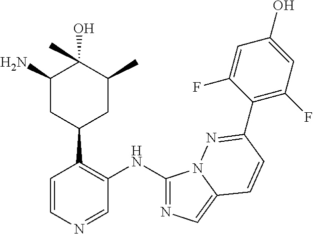 [SMILES: C[C@H]1C[C@@H](c2ccncc2Nc2ncc3ccc(-c4c(F)cc(O)cc4F)nn23)C[C@@H](N)[C@]1(C)O]

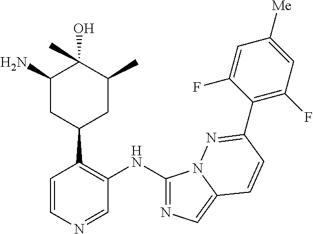 [SMILES: Cc1cc(F)c(-c2ccc3cnc(Nc4cnccc4[C@H]4C[C@@H](N)[C@](C)(O)[C@@H](C)C4)n3n2)c(F)c1]

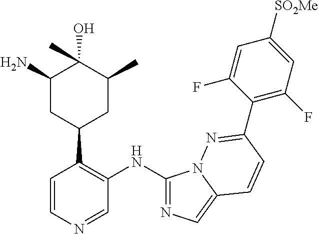 [SMILES: C[C@H]1C[C@@H](c2ccncc2Nc2ncc3ccc(-c4c(F)cc(S(C)(=O)=O)cc4F)nn23)C[C@@H](N)[C@]1(C)O]

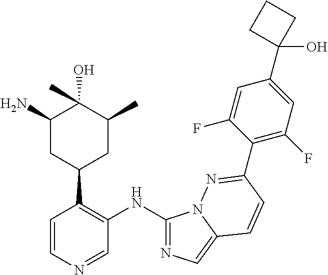 [SMILES: C[C@H]1C[C@@H](c2ccncc2Nc2ncc3ccc(-c4c(F)cc(C5(O)CCC5)cc4F)nn23)C[C@@H](N)[C@]1(C)O]

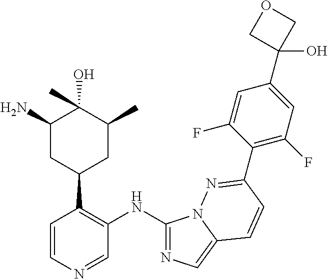 [SMILES: C[C@H]1C[C@@H](c2ccncc2Nc2ncc3ccc(-c4c(F)cc(C5(O)COC5)cc4F)nn23)C[C@@H](N)[C@]1(C)O]

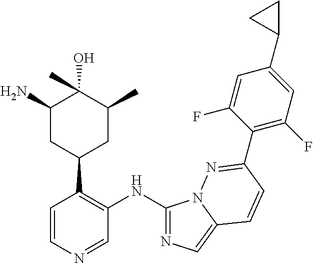 [SMILES: C[C@H]1C[C@@H](c2ccncc2Nc2ncc3ccc(-c4c(F)cc(C5CC5)cc4F)nn23)C[C@@H](N)[C@]1(C)O]

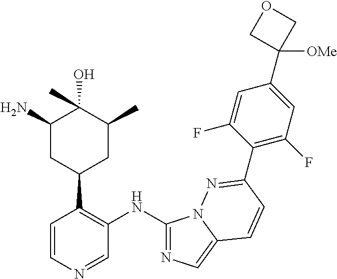 [SMILES: COC1(c2cc(F)c(-c3ccc4cnc(Nc5cnccc5[C@H]5C[C@@H](N)[C@](C)(O)[C@@H](C)C5)n4n3)c(F)c2)COC1]

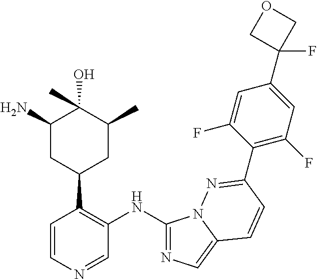 [SMILES: C[C@H]1C[C@@H](c2ccncc2Nc2ncc3ccc(-c4c(F)cc(C5(F)COC5)cc4F)nn23)C[C@@H](N)[C@]1(C)O]

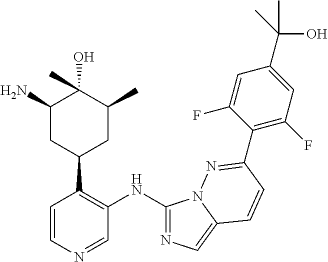 [SMILES: C[C@H]1C[C@@H](c2ccncc2Nc2ncc3ccc(-c4c(F)cc(C(C)(C)O)cc4F)nn23)C[C@@H](N)[C@]1(C)O]

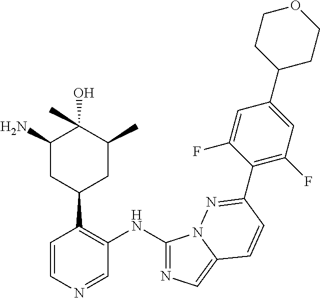 [SMILES: C[C@H]1C[C@@H](c2ccncc2Nc2ncc3ccc(-c4c(F)cc(C5CCOCC5)cc4F)nn23)C[C@@H](N)[C@]1(C)O]

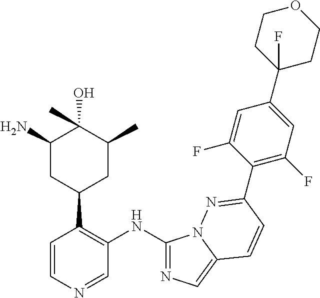

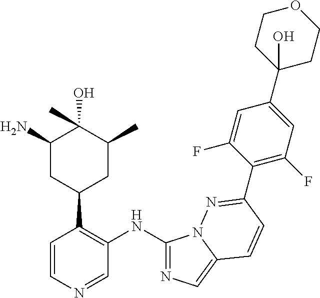

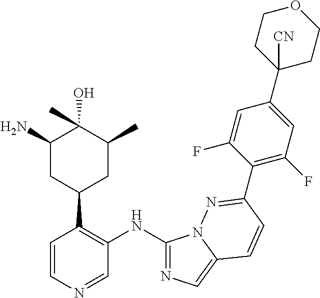

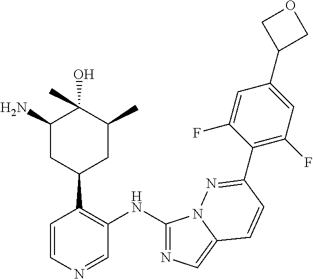 [SMILES: C[C@H]1C[C@@H](c2ccncc2Nc2ncc3ccc(-c4c(F)cc(C5COC5)cc4F)nn23)C[C@@H](N)[C@]1(C)O]

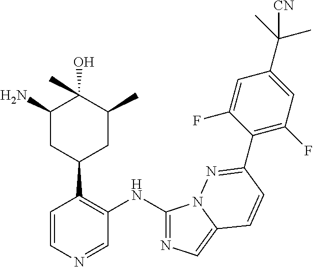 [SMILES: C[C@H]1C[C@@H](c2ccncc2Nc2ncc3ccc(-c4c(F)cc(C(C)(C)C#N)cc4F)nn23)C[C@@H](N)[C@]1(C)O]

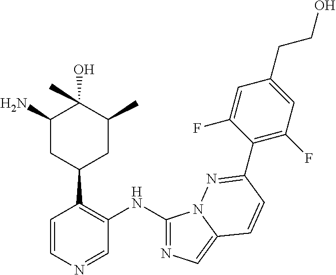 [SMILES: C[C@H]1C[C@@H](c2ccncc2Nc2ncc3ccc(-c4c(F)cc(CCO)cc4F)nn23)C[C@@H](N)[C@]1(C)O]

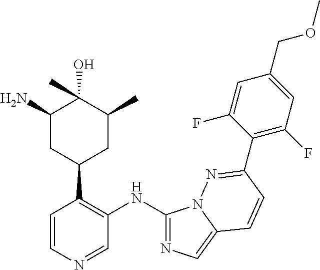 [SMILES: COCc1cc(F)c(-c2ccc3cnc(Nc4cnccc4[C@H]4C[C@@H](N)[C@](C)(O)[C@@H](C)C4)n3n2)c(F)c1]

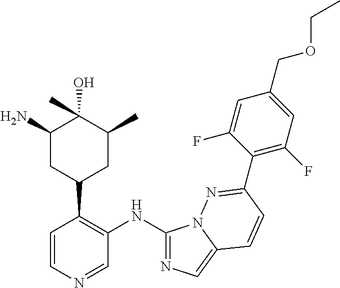

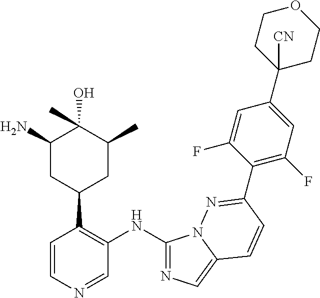

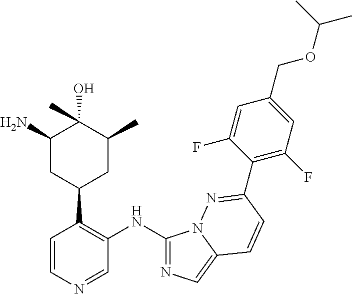 [SMILES: CC(C)OCc1cc(F)c(-c2ccc3cnc(Nc4cnccc4[C@H]4C[C@@H](N)[C@](C)(O)[C@@H](C)C4)n3n2)c(F)c1]

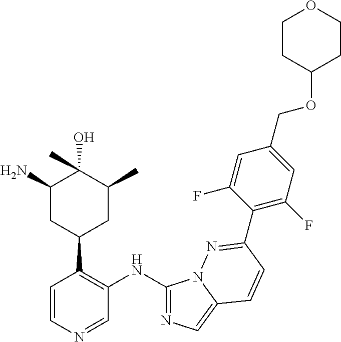 [SMILES: C[C@H]1C[C@@H](c2ccncc2Nc2ncc3ccc(-c4c(F)cc(COC5CCOCC5)cc4F)nn23)C[C@@H](N)[C@]1(C)O]

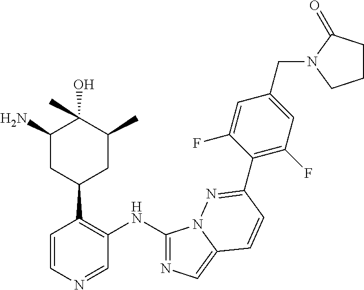 [SMILES: C[C@H]1C[C@@H](c2ccncc2Nc2ncc3ccc(-c4c(F)cc(CN5CCCC5=O)cc4F)nn23)C[C@@H](N)[C@]1(C)O]

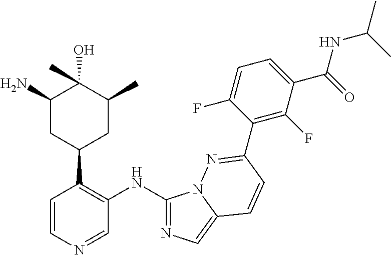 [SMILES: CC(C)NC(=O)c1ccc(F)c(-c2ccc3cnc(Nc4cnccc4[C@H]4C[C@@H](N)[C@](C)(O)[C@@H](C)C4)n3n2)c1F]

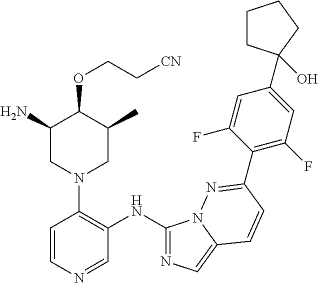 [SMILES: C[C@H]1CN(c2ccncc2Nc2ncc3ccc(-c4c(F)cc(C5(O)CCCC5)cc4F)nn23)C[C@@H](N)[C@H]1OCCC#N]

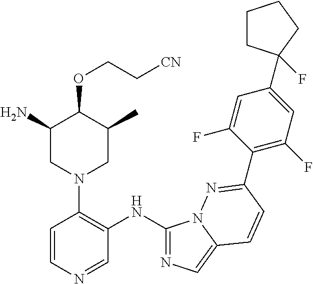 [SMILES: C[C@H]1CN(c2ccncc2Nc2ncc3ccc(-c4c(F)cc(C5(F)CCCC5)cc4F)nn23)C[C@@H](N)[C@H]1OCCC#N]

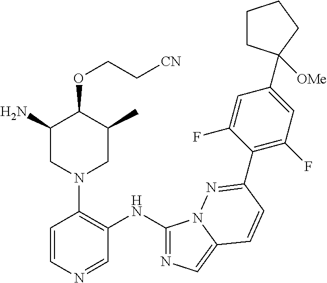 [SMILES: COC1(c2cc(F)c(-c3ccc4cnc(Nc5cnccc5N5C[C@@H](N)[C@@H](OCCC#N)[C@@H](C)C5)n4n3)c(F)c2)CCCC1]

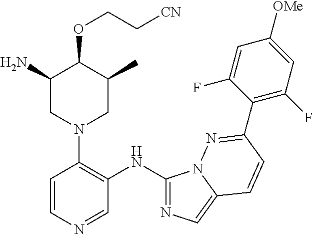 [SMILES: COc1cc(F)c(-c2ccc3cnc(Nc4cnccc4N4C[C@@H](N)[C@@H](OCCC#N)[C@@H](C)C4)n3n2)c(F)c1]

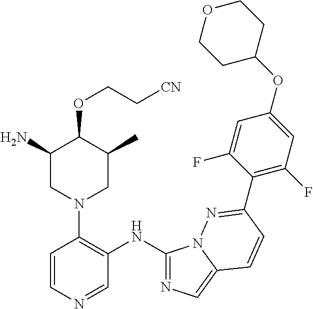 [SMILES: C[C@H]1CN(c2ccncc2Nc2ncc3ccc(-c4c(F)cc(OC5CCOCC5)cc4F)nn23)C[C@@H](N)[C@H]1OCCC#N]

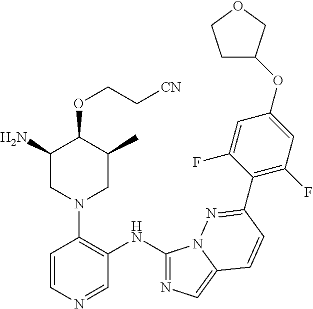 [SMILES: C[C@H]1CN(c2ccncc2Nc2ncc3ccc(-c4c(F)cc(OC5CCOC5)cc4F)nn23)C[C@@H](N)[C@H]1OCCC#N]

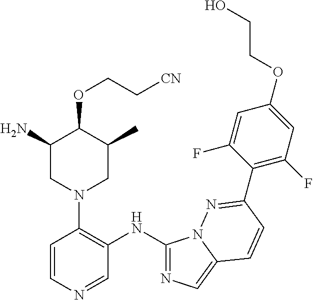 [SMILES: C[C@H]1CN(c2ccncc2Nc2ncc3ccc(-c4c(F)cc(OCCO)cc4F)nn23)C[C@@H](N)[C@H]1OCCC#N]

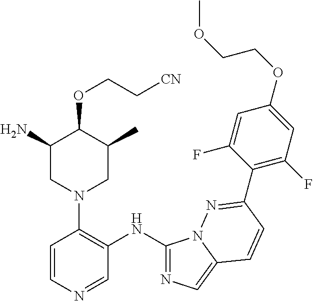 [SMILES: COCCOc1cc(F)c(-c2ccc3cnc(Nc4cnccc4N4C[C@@H](N)[C@@H](OCCC#N)[C@@H](C)C4)n3n2)c(F)c1]

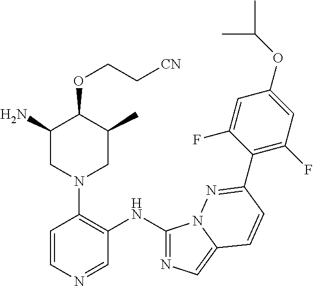 [SMILES: CC(C)Oc1cc(F)c(-c2ccc3cnc(Nc4cnccc4N4C[C@@H](N)[C@@H](OCCC#N)[C@@H](C)C4)n3n2)c(F)c1]

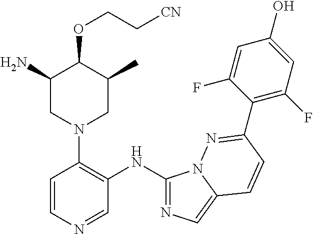 [SMILES: C[C@H]1CN(c2ccncc2Nc2ncc3ccc(-c4c(F)cc(O)cc4F)nn23)C[C@@H](N)[C@H]1OCCC#N]

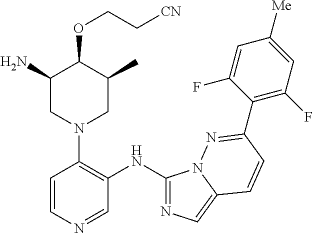 [SMILES: Cc1cc(F)c(-c2ccc3cnc(Nc4cnccc4N4C[C@@H](N)[C@@H](OCCC#N)[C@@H](C)C4)n3n2)c(F)c1]

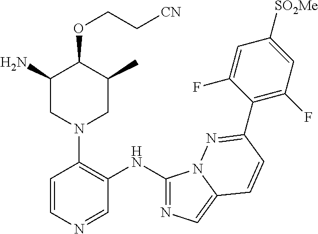 [SMILES: C[C@H]1CN(c2ccncc2Nc2ncc3ccc(-c4c(F)cc(S(C)(=O)=O)cc4F)nn23)C[C@@H](N)[C@H]1OCCC#N]

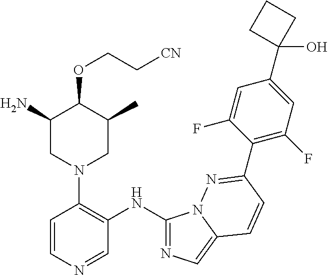 [SMILES: C[C@H]1CN(c2ccncc2Nc2ncc3ccc(-c4c(F)cc(C5(O)CCC5)cc4F)nn23)C[C@@H](N)[C@H]1OCCC#N]

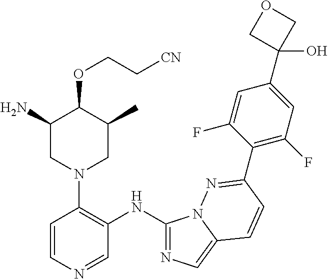 [SMILES: C[C@H]1CN(c2ccncc2Nc2ncc3ccc(-c4c(F)cc(C5(O)COC5)cc4F)nn23)C[C@@H](N)[C@H]1OCCC#N]

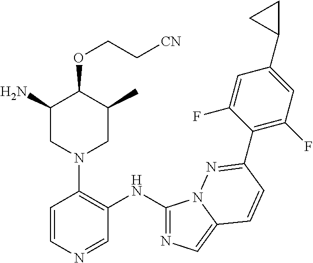 [SMILES: C[C@H]1CN(c2ccncc2Nc2ncc3ccc(-c4c(F)cc(C5CC5)cc4F)nn23)C[C@@H](N)[C@H]1OCCC#N]

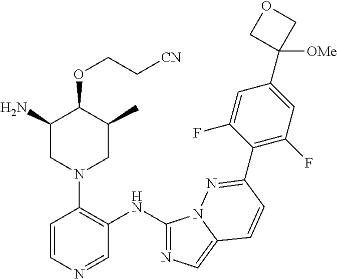 [SMILES: COC1(c2cc(F)c(-c3ccc4cnc(Nc5cnccc5N5C[C@@H](N)[C@@H](OCCC#N)[C@@H](C)C5)n4n3)c(F)c2)COC1]

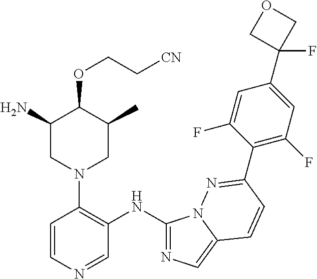 [SMILES: C[C@H]1CN(c2ccncc2Nc2ncc3ccc(-c4c(F)cc(C5(F)COC5)cc4F)nn23)C[C@@H](N)[C@H]1OCCC#N]

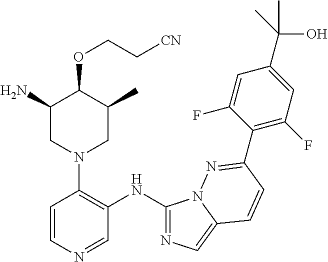 [SMILES: C[C@H]1CN(c2ccncc2Nc2ncc3ccc(-c4c(F)cc(C(C)(C)O)cc4F)nn23)C[C@@H](N)[C@H]1OCCC#N]

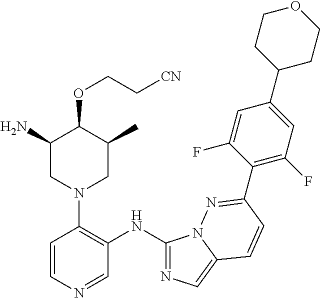 [SMILES: C[C@H]1CN(c2ccncc2Nc2ncc3ccc(-c4c(F)cc(C5CCOCC5)cc4F)nn23)C[C@@H](N)[C@H]1OCCC#N]

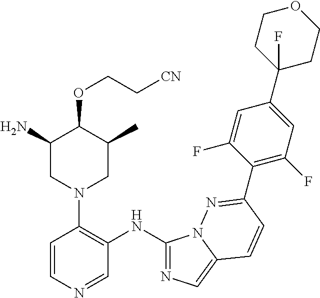

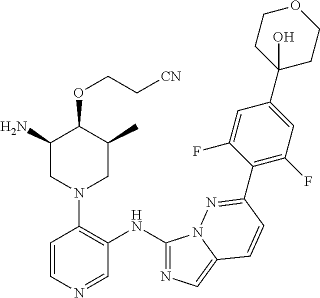

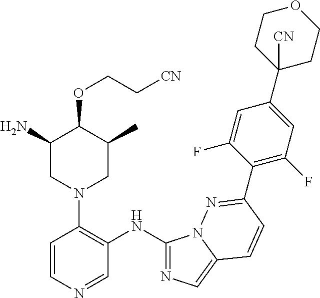

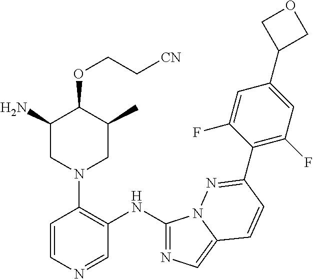 [SMILES: C[C@H]1CN(c2ccncc2Nc2ncc3ccc(-c4c(F)cc(C5COC5)cc4F)nn23)C[C@@H](N)[C@H]1OCCC#N]

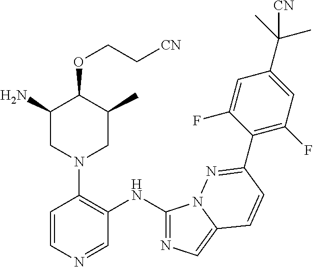 [SMILES: C[C@H]1CN(c2ccncc2Nc2ncc3ccc(-c4c(F)cc(C(C)(C)C#N)cc4F)nn23)C[C@@H](N)[C@H]1OCCC#N]

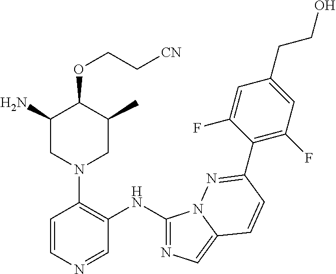 [SMILES: C[C@H]1CN(c2ccncc2Nc2ncc3ccc(-c4c(F)cc(CCO)cc4F)nn23)C[C@@H](N)[C@H]1OCCC#N]

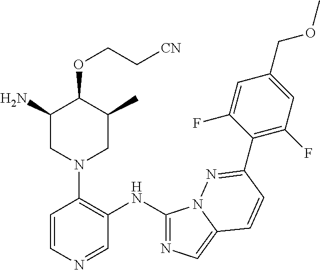 [SMILES: COCc1cc(F)c(-c2ccc3cnc(Nc4cnccc4N4C[C@@H](N)[C@@H](OCCC#N)[C@@H](C)C4)n3n2)c(F)c1]

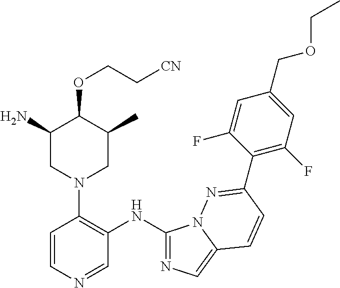 [SMILES: CCOCc1cc(F)c(-c2ccc3cnc(Nc4cnccc4N4C[C@@H](N)[C@@H](OCCC#N)[C@@H](C)C4)n3n2)c(F)c1]

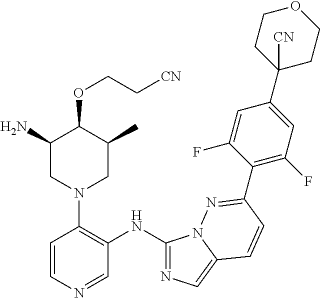

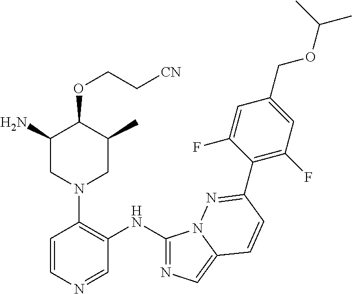 [SMILES: CC(C)OCc1cc(F)c(-c2ccc3cnc(Nc4cnccc4N4C[C@@H](N)[C@@H](OCCC#N)[C@@H](C)C4)n3n2)c(F)c1]

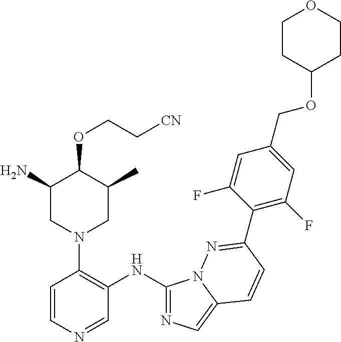 [SMILES: C[C@H]1CN(c2ccncc2Nc2ncc3ccc(-c4c(F)cc(COC5CCOCC5)cc4F)nn23)C[C@@H](N)[C@H]1OCCC#N]

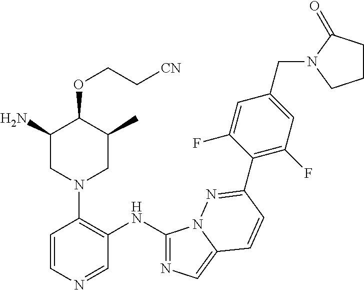 [SMILES: C[C@H]1CN(c2ccncc2Nc2ncc3ccc(-c4c(F)cc(CN5CCCC5=O)cc4F)nn23)C[C@@H](N)[C@H]1OCCC#N]

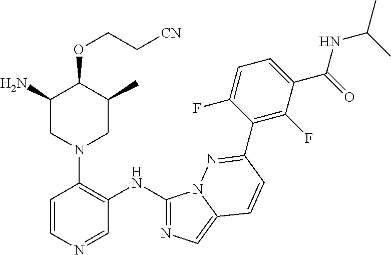 [SMILES: CC(C)NC(=O)c1ccc(F)c(-c2ccc3cnc(Nc4cnccc4N4C[C@@H](N)[C@@H](OCCC#N)[C@@H](C)C4)n3n2)c1F]

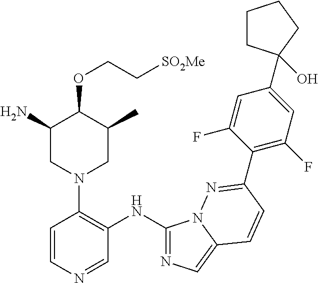 [SMILES: C[C@H]1CN(c2ccncc2Nc2ncc3ccc(-c4c(F)cc(C5(O)CCCC5)cc4F)nn23)C[C@@H](N)[C@H]1OCCS(C)(=O)=O]

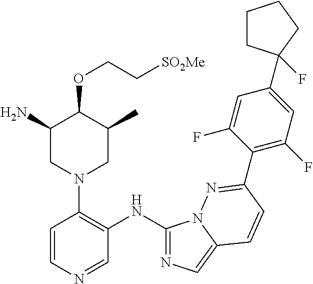 [SMILES: C[C@H]1CN(c2ccncc2Nc2ncc3ccc(-c4c(F)cc(C5(F)CCCC5)cc4F)nn23)C[C@@H](N)[C@H]1OCCS(C)(=O)=O]

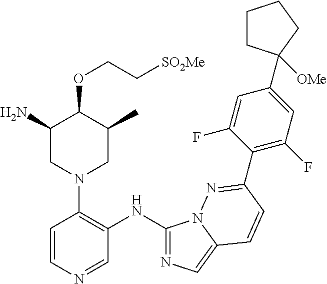 [SMILES: COC1(c2cc(F)c(-c3ccc4cnc(Nc5cnccc5N5C[C@@H](N)[C@@H](OCCS(C)(=O)=O)[C@@H](C)C5)n4n3)c(F)c2)CCCC1]

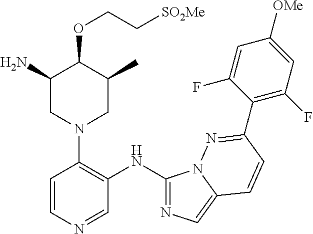 [SMILES: COc1cc(F)c(-c2ccc3cnc(Nc4cnccc4N4C[C@@H](N)[C@@H](OCCS(C)(=O)=O)[C@@H](C)C4)n3n2)c(F)c1]

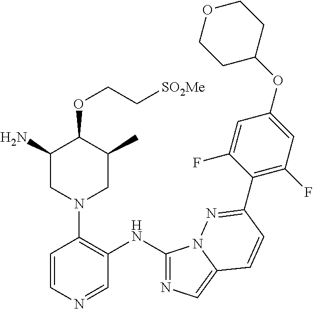 [SMILES: C[C@H]1CN(c2ccncc2Nc2ncc3ccc(-c4c(F)cc(OC5CCOCC5)cc4F)nn23)C[C@@H](N)[C@H]1OCCS(C)(=O)=O]

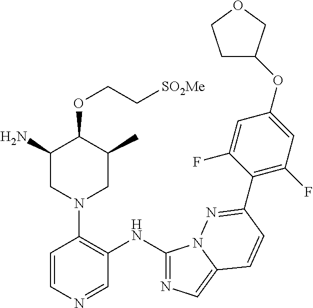 [SMILES: C[C@H]1CN(c2ccncc2Nc2ncc3ccc(-c4c(F)cc(OC5CCOC5)cc4F)nn23)C[C@@H](N)[C@H]1OCCS(C)(=O)=O]

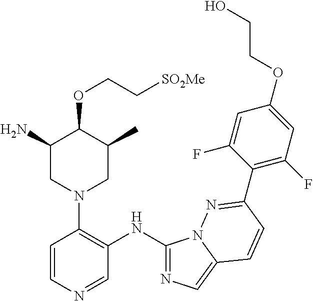 [SMILES: C[C@H]1CN(c2ccncc2Nc2ncc3ccc(-c4c(F)cc(OCCO)cc4F)nn23)C[C@@H](N)[C@H]1OCCS(C)(=O)=O]

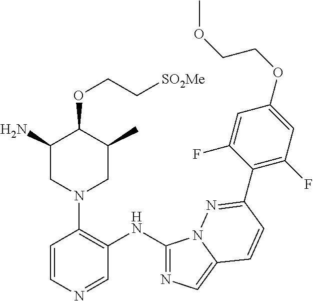 [SMILES: COCCOc1cc(F)c(-c2ccc3cnc(Nc4cnccc4N4C[C@@H](N)[C@@H](OCCS(C)(=O)=O)[C@@H](C)C4)n3n2)c(F)c1]

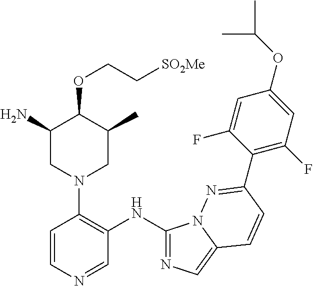 [SMILES: CC(C)Oc1cc(F)c(-c2ccc3cnc(Nc4cnccc4N4C[C@@H](N)[C@@H](OCCS(C)(=O)=O)[C@@H](C)C4)n3n2)c(F)c1]

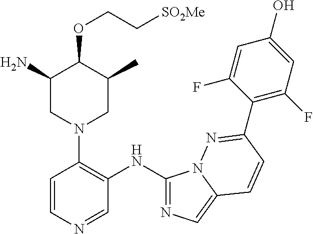 [SMILES: C[C@H]1CN(c2ccncc2Nc2ncc3ccc(-c4c(F)cc(O)cc4F)nn23)C[C@@H](N)[C@H]1OCCS(C)(=O)=O]

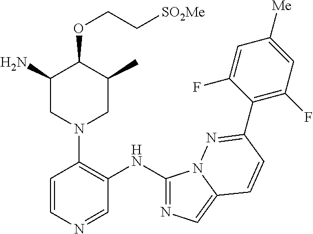 [SMILES: Cc1cc(F)c(-c2ccc3cnc(Nc4cnccc4N4C[C@@H](N)[C@@H](OCCS(C)(=O)=O)[C@@H](C)C4)n3n2)c(F)c1]

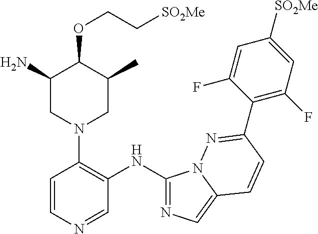 [SMILES: C[C@H]1CN(c2ccncc2Nc2ncc3ccc(-c4c(F)cc(S(C)(=O)=O)cc4F)nn23)C[C@@H](N)[C@H]1OCCS(C)(=O)=O]

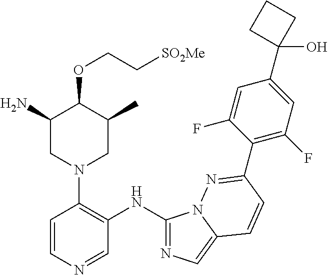 [SMILES: C[C@H]1CN(c2ccncc2Nc2ncc3ccc(-c4c(F)cc(C5(O)CCC5)cc4F)nn23)C[C@@H](N)[C@H]1OCCS(C)(=O)=O]

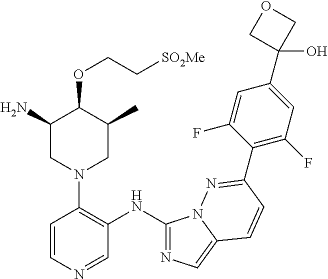 [SMILES: C[C@H]1CN(c2ccncc2Nc2ncc3ccc(-c4c(F)cc(C5(O)COC5)cc4F)nn23)C[C@@H](N)[C@H]1OCCS(C)(=O)=O]

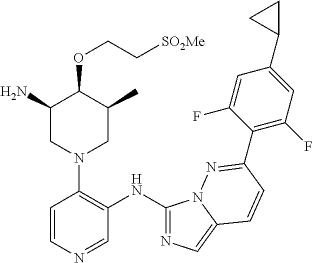 [SMILES: C[C@H]1CN(c2ccncc2Nc2ncc3ccc(-c4c(F)cc(C5CC5)cc4F)nn23)C[C@@H](N)[C@H]1OCCS(C)(=O)=O]

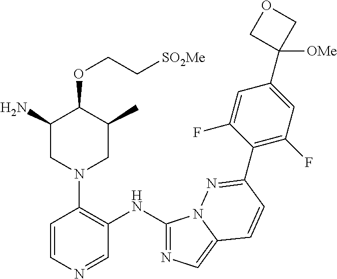 [SMILES: COC1(c2cc(F)c(-c3ccc4cnc(Nc5cnccc5N5C[C@@H](N)[C@@H](OCCS(C)(=O)=O)[C@@H](C)C5)n4n3)c(F)c2)COC1]

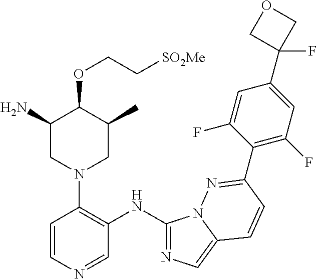 [SMILES: C[C@H]1CN(c2ccncc2Nc2ncc3ccc(-c4c(F)cc(C5(F)COC5)cc4F)nn23)C[C@@H](N)[C@H]1OCCS(C)(=O)=O]

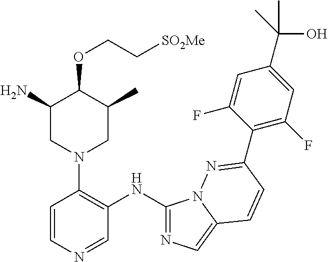 [SMILES: C[C@H]1CN(c2ccncc2Nc2ncc3ccc(-c4c(F)cc(C(C)(C)O)cc4F)nn23)C[C@@H](N)[C@H]1OCCS(C)(=O)=O]

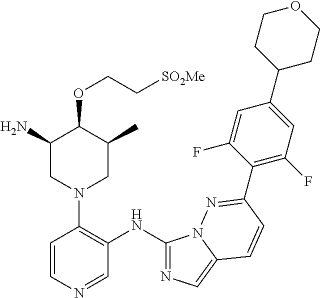 [SMILES: C[C@H]1CN(c2ccncc2Nc2ncc3ccc(-c4c(F)cc(C5CCOCC5)cc4F)nn23)C[C@@H](N)[C@H]1OCCS(C)(=O)=O]

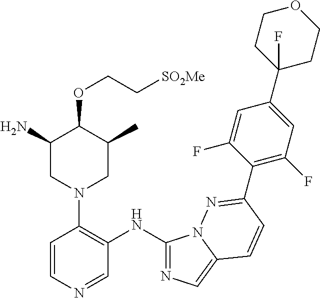

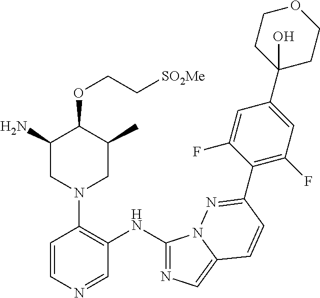

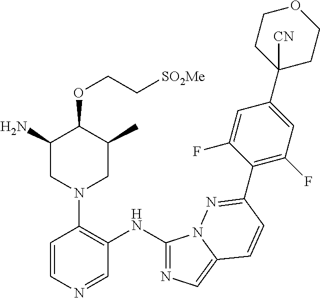

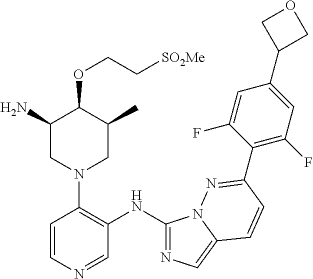 [SMILES: C[C@H]1CN(c2ccncc2Nc2ncc3ccc(-c4c(F)cc(C5COC5)cc4F)nn23)C[C@@H](N)[C@H]1OCCS(C)(=O)=O]

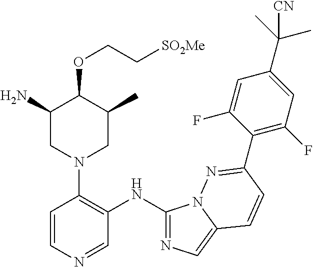 [SMILES: C[C@H]1CN(c2ccncc2Nc2ncc3ccc(-c4c(F)cc(C(C)(C)C#N)cc4F)nn23)C[C@@H](N)[C@H]1OCCS(C)(=O)=O]

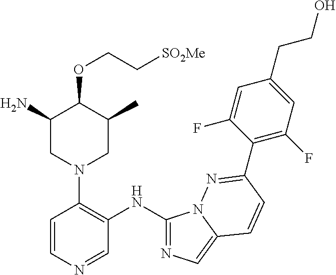 [SMILES: C[C@H]1CN(c2ccncc2Nc2ncc3ccc(-c4c(F)cc(CCO)cc4F)nn23)C[C@@H](N)[C@H]1OCCS(C)(=O)=O]

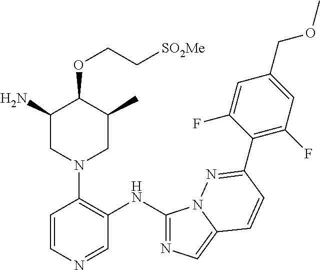 [SMILES: COCc1cc(F)c(-c2ccc3cnc(Nc4cnccc4N4C[C@@H](N)[C@@H](OCCS(C)(=O)=O)[C@@H](C)C4)n3n2)c(F)c1]

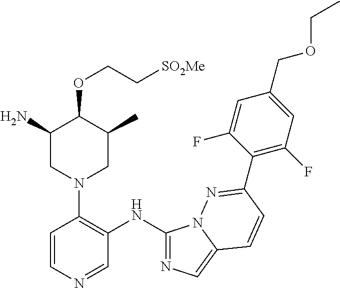 [SMILES: CCOCc1cc(F)c(-c2ccc3cnc(Nc4cnccc4N4C[C@@H](N)[C@@H](OCCS(C)(=O)=O)[C@@H](C)C4)n3n2)c(F)c1]

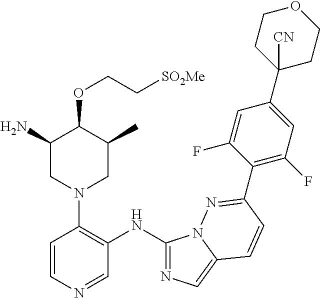

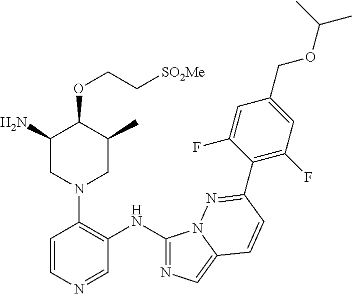 [SMILES: CC(C)OCc1cc(F)c(-c2ccc3cnc(Nc4cnccc4N4C[C@@H](N)[C@@H](OCCS(C)(=O)=O)[C@@H](C)C4)n3n2)c(F)c1]

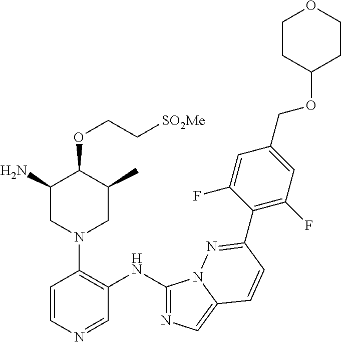 [SMILES: C[C@H]1CN(c2ccncc2Nc2ncc3ccc(-c4c(F)cc(COC5CCOCC5)cc4F)nn23)C[C@@H](N)[C@H]1OCCS(C)(=O)=O]

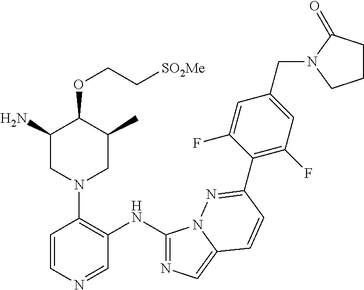 [SMILES: C[C@H]1CN(c2ccncc2Nc2ncc3ccc(-c4c(F)cc(CN5CCCC5=O)cc4F)nn23)C[C@@H](N)[C@H]1OCCS(C)(=O)=O]

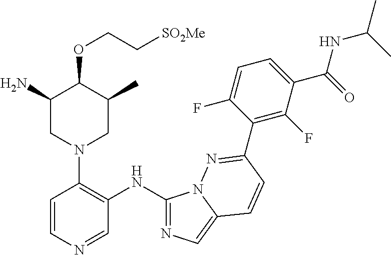 [SMILES: CC(C)NC(=O)c1ccc(F)c(-c2ccc3cnc(Nc4cnccc4N4C[C@@H](N)[C@@H](OCCS(C)(=O)=O)[C@@H](C)C4)n3n2)c1F]

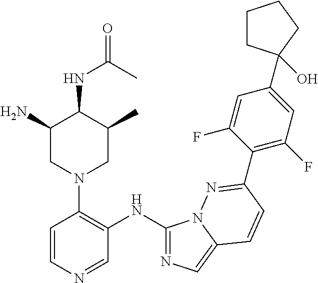 [SMILES: CC(=O)N[C@@H]1[C@H](N)CN(c2ccncc2Nc2ncc3ccc(-c4c(F)cc(C5(O)CCCC5)cc4F)nn23)C[C@@H]1C]

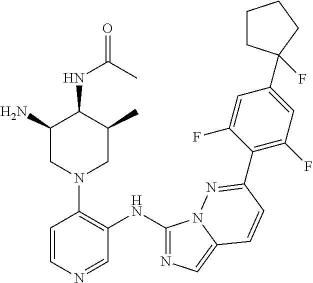 [SMILES: CC(=O)N[C@@H]1[C@H](N)CN(c2ccncc2Nc2ncc3ccc(-c4c(F)cc(C5(F)CCCC5)cc4F)nn23)C[C@@H]1C]

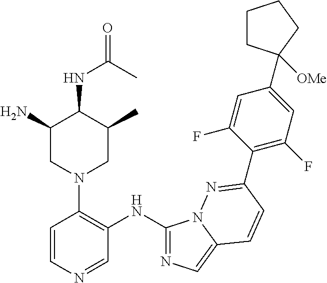 [SMILES: COC1(c2cc(F)c(-c3ccc4cnc(Nc5cnccc5N5C[C@@H](N)[C@@H](NC(C)=O)[C@@H](C)C5)n4n3)c(F)c2)CCCC1]

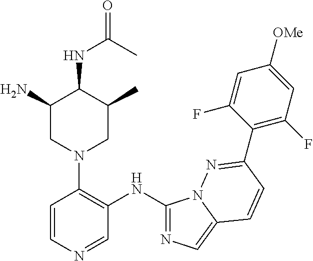 [SMILES: COc1cc(F)c(-c2ccc3cnc(Nc4cnccc4N4C[C@@H](N)[C@@H](NC(C)=O)[C@@H](C)C4)n3n2)c(F)c1]

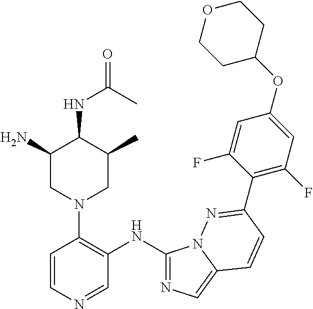 [SMILES: CC(=O)N[C@@H]1[C@H](N)CN(c2ccncc2Nc2ncc3ccc(-c4c(F)cc(OC5CCOCC5)cc4F)nn23)C[C@@H]1C]

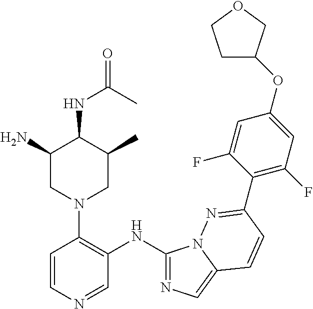 [SMILES: CC(=O)N[C@@H]1[C@H](N)CN(c2ccncc2Nc2ncc3ccc(-c4c(F)cc(OC5CCOC5)cc4F)nn23)C[C@@H]1C]

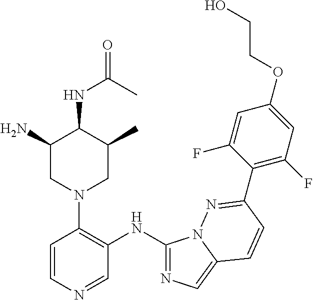 [SMILES: CC(=O)N[C@@H]1[C@H](N)CN(c2ccncc2Nc2ncc3ccc(-c4c(F)cc(OCCO)cc4F)nn23)C[C@@H]1C]

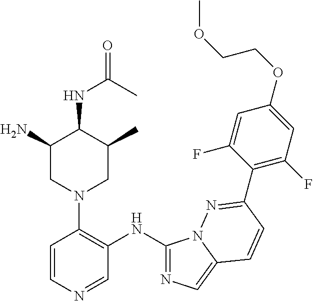 [SMILES: COCCOc1cc(F)c(-c2ccc3cnc(Nc4cnccc4N4C[C@@H](N)[C@@H](NC(C)=O)[C@@H](C)C4)n3n2)c(F)c1]

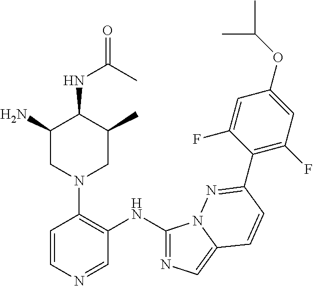 [SMILES: CC(=O)N[C@@H]1[C@H](N)CN(c2ccncc2Nc2ncc3ccc(-c4c(F)cc(OC(C)C)cc4F)nn23)C[C@@H]1C]

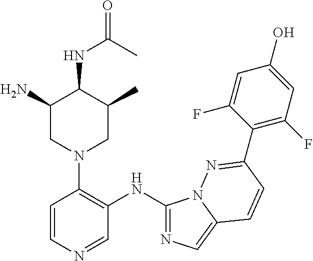 [SMILES: CC(=O)N[C@@H]1[C@H](N)CN(c2ccncc2Nc2ncc3ccc(-c4c(F)cc(O)cc4F)nn23)C[C@@H]1C]

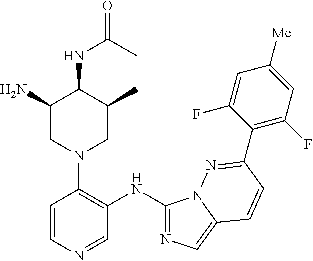 [SMILES: CC(=O)N[C@@H]1[C@H](N)CN(c2ccncc2Nc2ncc3ccc(-c4c(F)cc(C)cc4F)nn23)C[C@@H]1C]

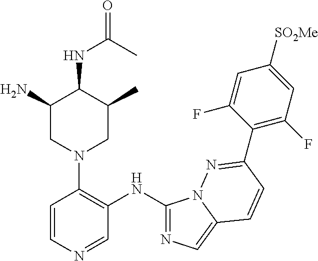 [SMILES: CC(=O)N[C@@H]1[C@H](N)CN(c2ccncc2Nc2ncc3ccc(-c4c(F)cc(S(C)(=O)=O)cc4F)nn23)C[C@@H]1C]

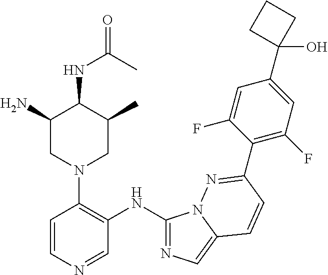 [SMILES: CC(=O)N[C@@H]1[C@H](N)CN(c2ccncc2Nc2ncc3ccc(-c4c(F)cc(C5(O)CCC5)cc4F)nn23)C[C@@H]1C]

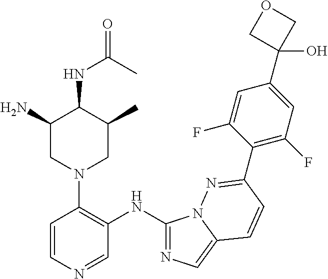 [SMILES: CC(=O)N[C@@H]1[C@H](N)CN(c2ccncc2Nc2ncc3ccc(-c4c(F)cc(C5(O)COC5)cc4F)nn23)C[C@@H]1C]

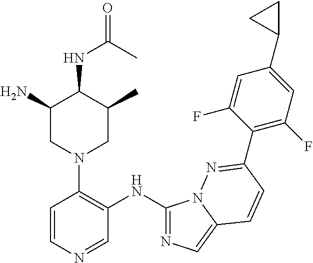 [SMILES: CC(=O)N[C@@H]1[C@H](N)CN(c2ccncc2Nc2ncc3ccc(-c4c(F)cc(C5CC5)cc4F)nn23)C[C@@H]1C]

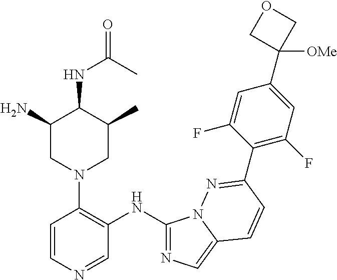 [SMILES: COC1(c2cc(F)c(-c3ccc4cnc(Nc5cnccc5N5C[C@@H](N)[C@@H](NC(C)=O)[C@@H](C)C5)n4n3)c(F)c2)COC1]

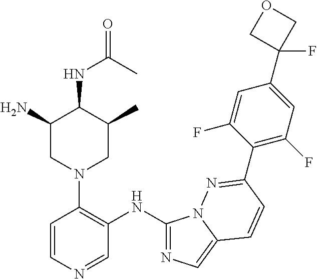 [SMILES: CC(=O)N[C@@H]1[C@H](N)CN(c2ccncc2Nc2ncc3ccc(-c4c(F)cc(C5(F)COC5)cc4F)nn23)C[C@@H]1C]

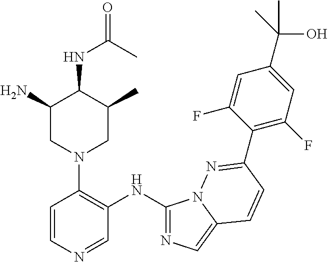 [SMILES: CC(=O)N[C@@H]1[C@H](N)CN(c2ccncc2Nc2ncc3ccc(-c4c(F)cc(C(C)(C)O)cc4F)nn23)C[C@@H]1C]

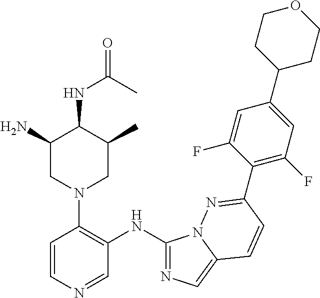 [SMILES: CC(=O)N[C@@H]1[C@H](N)CN(c2ccncc2Nc2ncc3ccc(-c4c(F)cc(C5CCOCC5)cc4F)nn23)C[C@@H]1C]

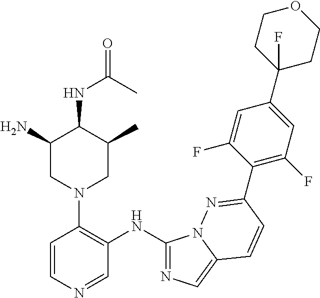

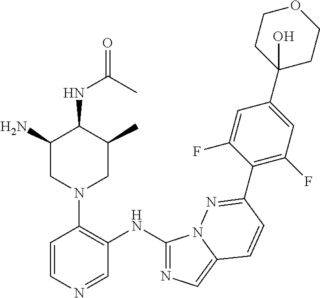

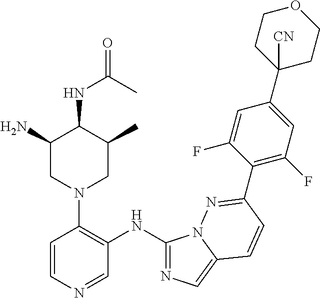

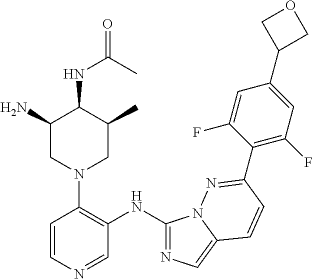 [SMILES: CC(=O)N[C@@H]1[C@H](N)CN(c2ccncc2Nc2ncc3ccc(-c4c(F)cc(C5COC5)cc4F)nn23)C[C@@H]1C]

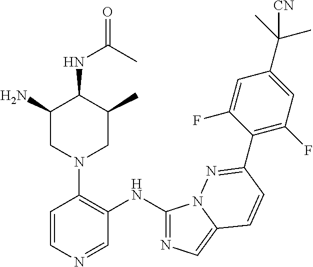 [SMILES: CC(=O)N[C@@H]1[C@H](N)CN(c2ccncc2Nc2ncc3ccc(-c4c(F)cc(C(C)(C)C#N)cc4F)nn23)C[C@@H]1C]

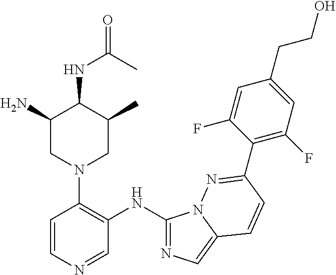 [SMILES: CC(=O)N[C@@H]1[C@H](N)CN(c2ccncc2Nc2ncc3ccc(-c4c(F)cc(CCO)cc4F)nn23)C[C@@H]1C]

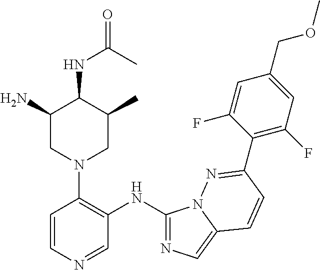 [SMILES: COCc1cc(F)c(-c2ccc3cnc(Nc4cnccc4N4C[C@@H](N)[C@@H](NC(C)=O)[C@@H](C)C4)n3n2)c(F)c1]

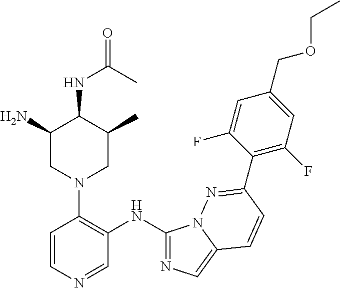 [SMILES: CCOCc1cc(F)c(-c2ccc3cnc(Nc4cnccc4N4C[C@@H](N)[C@@H](NC(C)=O)[C@@H](C)C4)n3n2)c(F)c1]

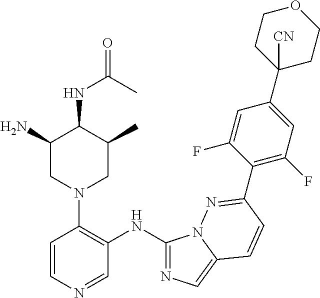

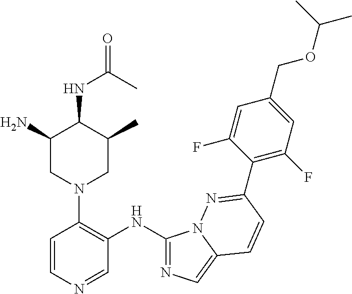 [SMILES: CC(=O)N[C@@H]1[C@H](N)CN(c2ccncc2Nc2ncc3ccc(-c4c(F)cc(COC(C)C)cc4F)nn23)C[C@@H]1C]

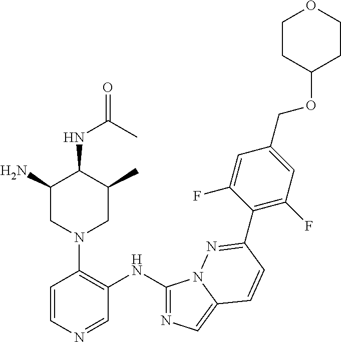 [SMILES: CC(=O)N[C@@H]1[C@H](N)CN(c2ccncc2Nc2ncc3ccc(-c4c(F)cc(COC5CCOCC5)cc4F)nn23)C[C@@H]1C]

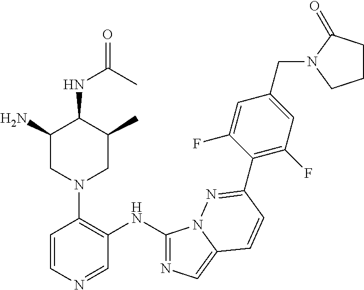 [SMILES: CC(=O)N[C@@H]1[C@H](N)CN(c2ccncc2Nc2ncc3ccc(-c4c(F)cc(CN5CCCC5=O)cc4F)nn23)C[C@@H]1C]

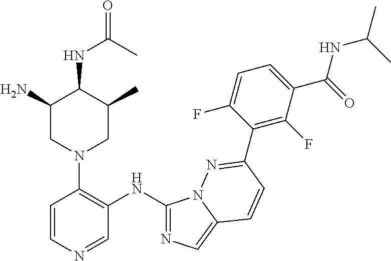 [SMILES: CC(=O)N[C@@H]1[C@H](N)CN(c2ccncc2Nc2ncc3ccc(-c4c(F)ccc(C(=O)NC(C)C)c4F)nn23)C[C@@H]1C]

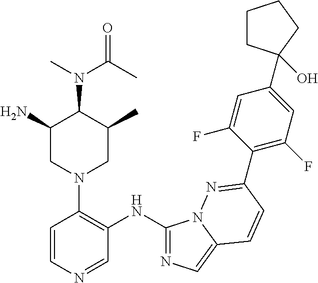 [SMILES: CC(=O)N(C)[C@@H]1[C@H](N)CN(c2ccncc2Nc2ncc3ccc(-c4c(F)cc(C5(O)CCCC5)cc4F)nn23)C[C@@H]1C]

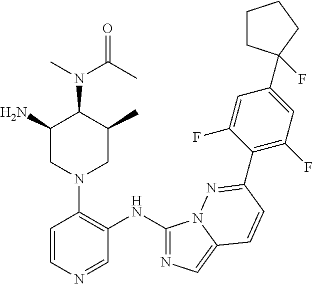 [SMILES: CC(=O)N(C)[C@@H]1[C@H](N)CN(c2ccncc2Nc2ncc3ccc(-c4c(F)cc(C5(F)CCCC5)cc4F)nn23)C[C@@H]1C]

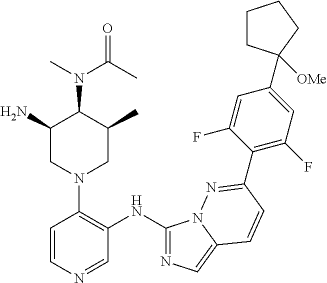 [SMILES: COC1(c2cc(F)c(-c3ccc4cnc(Nc5cnccc5N5C[C@@H](N)[C@@H](N(C)C(C)=O)[C@@H](C)C5)n4n3)c(F)c2)CCCC1]

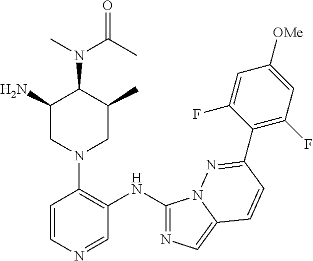 [SMILES: COc1cc(F)c(-c2ccc3cnc(Nc4cnccc4N4C[C@@H](N)[C@@H](N(C)C(C)=O)[C@@H](C)C4)n3n2)c(F)c1]

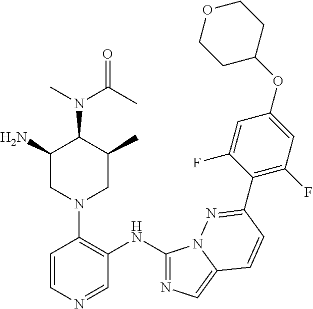 [SMILES: CC(=O)N(C)[C@@H]1[C@H](N)CN(c2ccncc2Nc2ncc3ccc(-c4c(F)cc(OC5CCOCC5)cc4F)nn23)C[C@@H]1C]

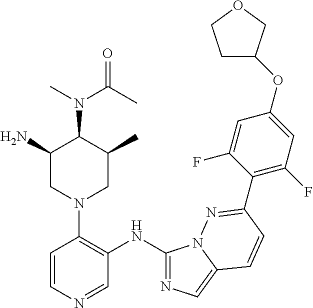 [SMILES: CC(=O)N(C)[C@@H]1[C@H](N)CN(c2ccncc2Nc2ncc3ccc(-c4c(F)cc(OC5CCOC5)cc4F)nn23)C[C@@H]1C]

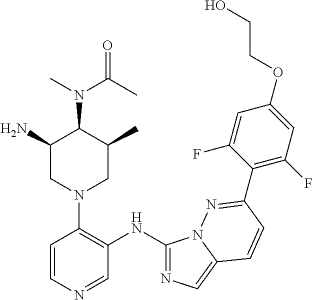 [SMILES: CC(=O)N(C)[C@@H]1[C@H](N)CN(c2ccncc2Nc2ncc3ccc(-c4c(F)cc(OCCO)cc4F)nn23)C[C@@H]1C]

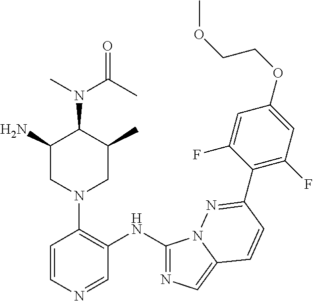 [SMILES: COCCOc1cc(F)c(-c2ccc3cnc(Nc4cnccc4N4C[C@@H](N)[C@@H](N(C)C(C)=O)[C@@H](C)C4)n3n2)c(F)c1]

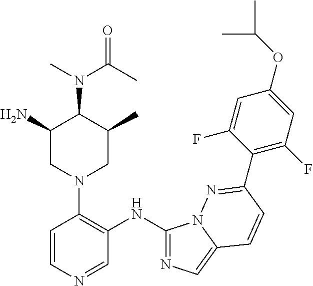 [SMILES: CC(=O)N(C)[C@@H]1[C@H](N)CN(c2ccncc2Nc2ncc3ccc(-c4c(F)cc(OC(C)C)cc4F)nn23)C[C@@H]1C]

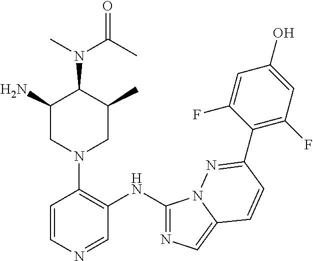 [SMILES: CC(=O)N(C)[C@@H]1[C@H](N)CN(c2ccncc2Nc2ncc3ccc(-c4c(F)cc(O)cc4F)nn23)C[C@@H]1C]

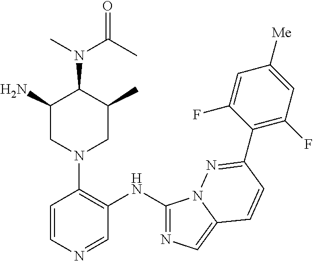 [SMILES: CC(=O)N(C)[C@@H]1[C@H](N)CN(c2ccncc2Nc2ncc3ccc(-c4c(F)cc(C)cc4F)nn23)C[C@@H]1C]

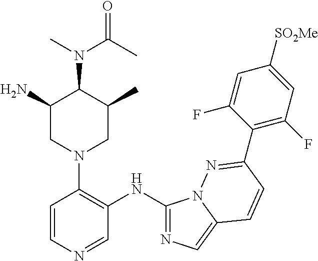 [SMILES: CC(=O)N(C)[C@@H]1[C@H](N)CN(c2ccncc2Nc2ncc3ccc(-c4c(F)cc(S(C)(=O)=O)cc4F)nn23)C[C@@H]1C]

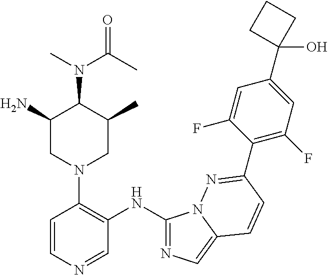 [SMILES: CC(=O)N(C)[C@@H]1[C@H](N)CN(c2ccncc2Nc2ncc3ccc(-c4c(F)cc(C5(O)CCC5)cc4F)nn23)C[C@@H]1C]

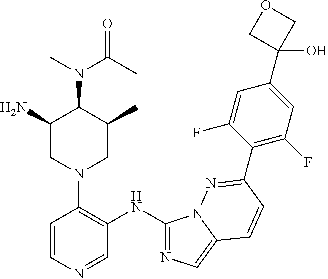 [SMILES: CC(=O)N(C)[C@@H]1[C@H](N)CN(c2ccncc2Nc2ncc3ccc(-c4c(F)cc(C5(O)COC5)cc4F)nn23)C[C@@H]1C]

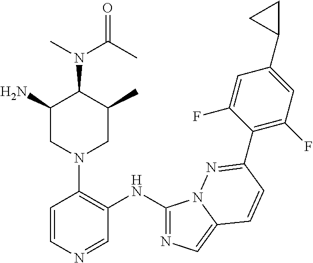 [SMILES: CC(=O)N(C)[C@@H]1[C@H](N)CN(c2ccncc2Nc2ncc3ccc(-c4c(F)cc(C5CC5)cc4F)nn23)C[C@@H]1C]

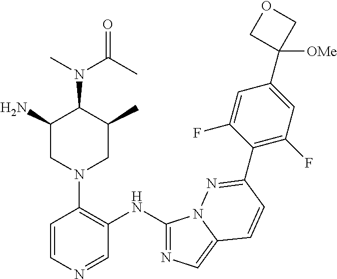 [SMILES: COC1(c2cc(F)c(-c3ccc4cnc(Nc5cnccc5N5C[C@@H](N)[C@@H](N(C)C(C)=O)[C@@H](C)C5)n4n3)c(F)c2)COC1]

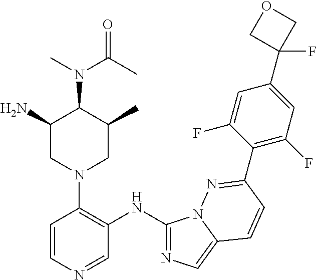 [SMILES: CC(=O)N(C)[C@@H]1[C@H](N)CN(c2ccncc2Nc2ncc3ccc(-c4c(F)cc(C5(F)COC5)cc4F)nn23)C[C@@H]1C]

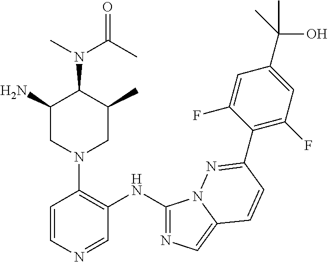 [SMILES: CC(=O)N(C)[C@@H]1[C@H](N)CN(c2ccncc2Nc2ncc3ccc(-c4c(F)cc(C(C)(C)O)cc4F)nn23)C[C@@H]1C]

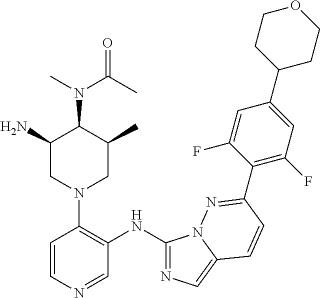 [SMILES: CC(=O)N(C)[C@@H]1[C@H](N)CN(c2ccncc2Nc2ncc3ccc(-c4c(F)cc(C5CCOCC5)cc4F)nn23)C[C@@H]1C]

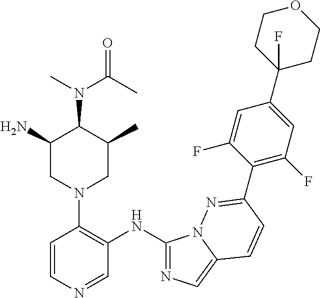

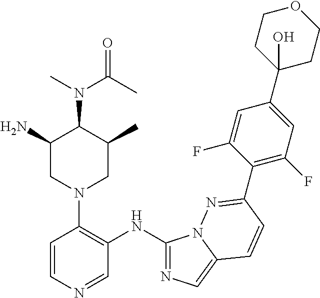

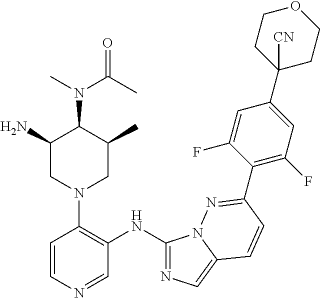

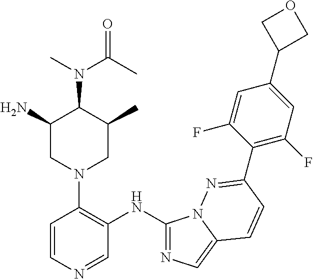 [SMILES: CC(=O)N(C)[C@@H]1[C@H](N)CN(c2ccncc2Nc2ncc3ccc(-c4c(F)cc(C5COC5)cc4F)nn23)C[C@@H]1C]

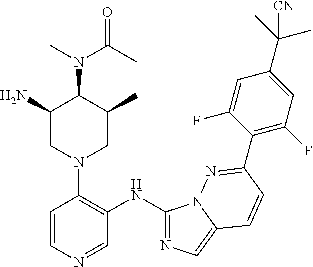 [SMILES: CC(=O)N(C)[C@@H]1[C@H](N)CN(c2ccncc2Nc2ncc3ccc(-c4c(F)cc(C(C)(C)C#N)cc4F)nn23)C[C@@H]1C]

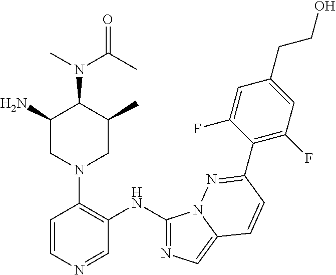 [SMILES: CC(=O)N(C)[C@@H]1[C@H](N)CN(c2ccncc2Nc2ncc3ccc(-c4c(F)cc(CCO)cc4F)nn23)C[C@@H]1C]

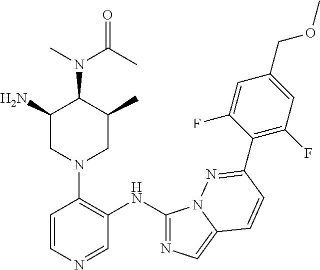 [SMILES: COCc1cc(F)c(-c2ccc3cnc(Nc4cnccc4N4C[C@@H](N)[C@@H](N(C)C(C)=O)[C@@H](C)C4)n3n2)c(F)c1]

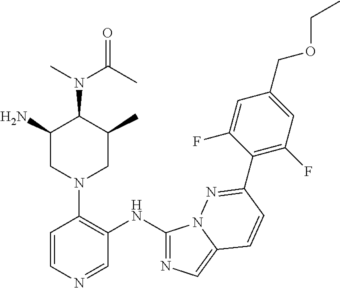 [SMILES: CCOCc1cc(F)c(-c2ccc3cnc(Nc4cnccc4N4C[C@@H](N)[C@@H](N(C)C(C)=O)[C@@H](C)C4)n3n2)c(F)c1]

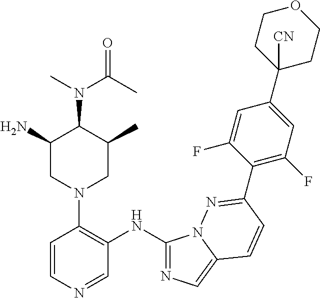

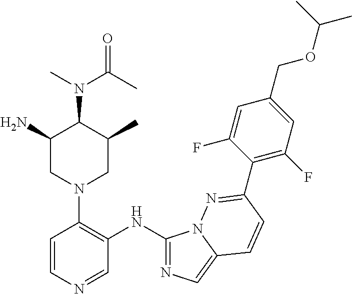 [SMILES: CC(=O)N(C)[C@@H]1[C@H](N)CN(c2ccncc2Nc2ncc3ccc(-c4c(F)cc(COC(C)C)cc4F)nn23)C[C@@H]1C]

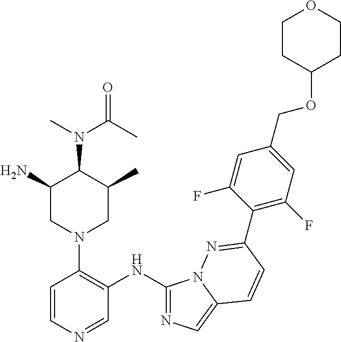 [SMILES: CC(=O)N(C)[C@@H]1[C@H](N)CN(c2ccncc2Nc2ncc3ccc(-c4c(F)cc(COC5CCOCC5)cc4F)nn23)C[C@@H]1C]

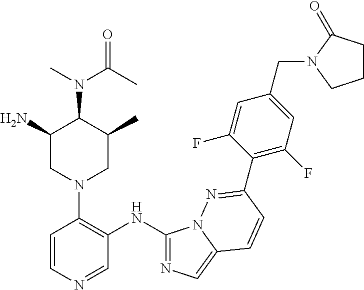 [SMILES: CC(=O)N(C)[C@@H]1[C@H](N)CN(c2ccncc2Nc2ncc3ccc(-c4c(F)cc(CN5CCCC5=O)cc4F)nn23)C[C@@H]1C]

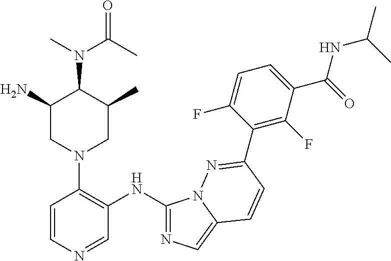 [SMILES: CC(=O)N(C)[C@@H]1[C@H](N)CN(c2ccncc2Nc2ncc3ccc(-c4c(F)ccc(C(=O)NC(C)C)c4F)nn23)C[C@@H]1C]

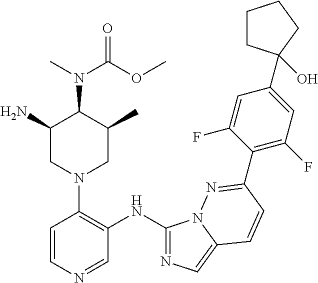 [SMILES: COC(=O)N(C)[C@@H]1[C@H](N)CN(c2ccncc2Nc2ncc3ccc(-c4c(F)cc(C5(O)CCCC5)cc4F)nn23)C[C@@H]1C]

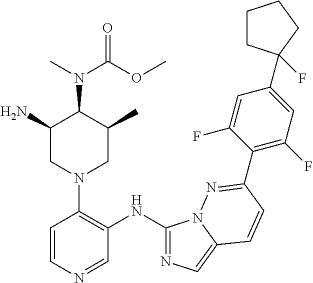 [SMILES: COC(=O)N(C)[C@@H]1[C@H](N)CN(c2ccncc2Nc2ncc3ccc(-c4c(F)cc(C5(F)CCCC5)cc4F)nn23)C[C@@H]1C]

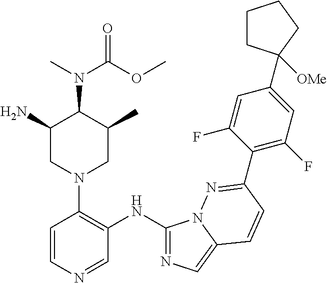 [SMILES: COC(=O)N(C)[C@@H]1[C@H](N)CN(c2ccncc2Nc2ncc3ccc(-c4c(F)cc(C5(OC)CCCC5)cc4F)nn23)C[C@@H]1C]

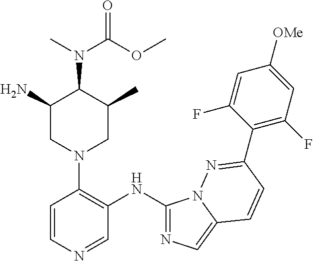 [SMILES: COC(=O)N(C)[C@@H]1[C@H](N)CN(c2ccncc2Nc2ncc3ccc(-c4c(F)cc(OC)cc4F)nn23)C[C@@H]1C]

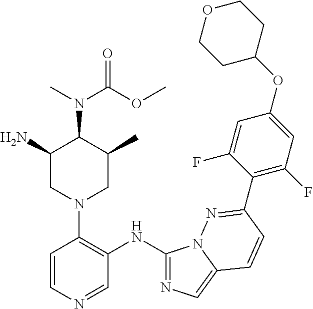 [SMILES: COC(=O)N(C)[C@@H]1[C@H](N)CN(c2ccncc2Nc2ncc3ccc(-c4c(F)cc(OC5CCOCC5)cc4F)nn23)C[C@@H]1C]

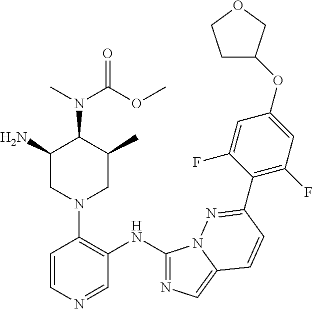 [SMILES: COC(=O)N(C)[C@@H]1[C@H](N)CN(c2ccncc2Nc2ncc3ccc(-c4c(F)cc(OC5CCOC5)cc4F)nn23)C[C@@H]1C]

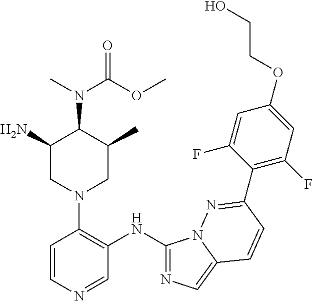 [SMILES: COC(=O)N(C)[C@@H]1[C@H](N)CN(c2ccncc2Nc2ncc3ccc(-c4c(F)cc(OCCO)cc4F)nn23)C[C@@H]1C]

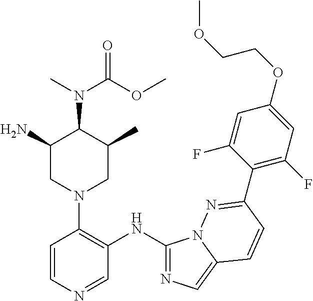 [SMILES: COCCOc1cc(F)c(-c2ccc3cnc(Nc4cnccc4N4C[C@@H](N)[C@@H](N(C)C(=O)OC)[C@@H](C)C4)n3n2)c(F)c1]

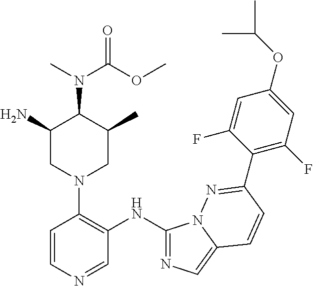 [SMILES: COC(=O)N(C)[C@@H]1[C@H](N)CN(c2ccncc2Nc2ncc3ccc(-c4c(F)cc(OC(C)C)cc4F)nn23)C[C@@H]1C]

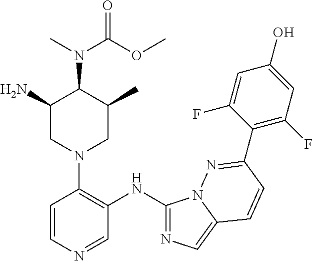 [SMILES: COC(=O)N(C)[C@@H]1[C@H](N)CN(c2ccncc2Nc2ncc3ccc(-c4c(F)cc(O)cc4F)nn23)C[C@@H]1C]

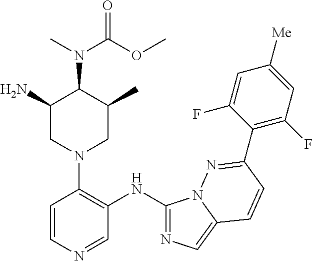 [SMILES: COC(=O)N(C)[C@@H]1[C@H](N)CN(c2ccncc2Nc2ncc3ccc(-c4c(F)cc(C)cc4F)nn23)C[C@@H]1C]

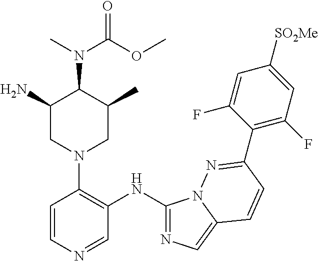 [SMILES: COC(=O)N(C)[C@@H]1[C@H](N)CN(c2ccncc2Nc2ncc3ccc(-c4c(F)cc(S(C)(=O)=O)cc4F)nn23)C[C@@H]1C]

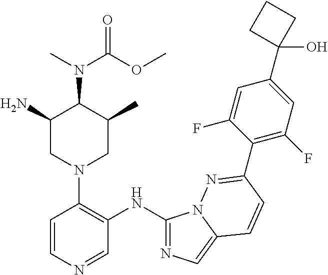 [SMILES: COC(=O)N(C)[C@@H]1[C@H](N)CN(c2ccncc2Nc2ncc3ccc(-c4c(F)cc(C5(O)CCC5)cc4F)nn23)C[C@@H]1C]

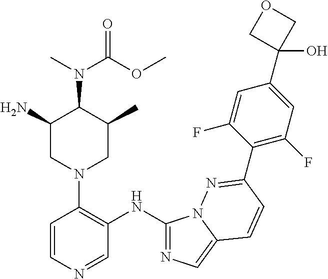 [SMILES: COC(=O)N(C)[C@@H]1[C@H](N)CN(c2ccncc2Nc2ncc3ccc(-c4c(F)cc(C5(O)COC5)cc4F)nn23)C[C@@H]1C]

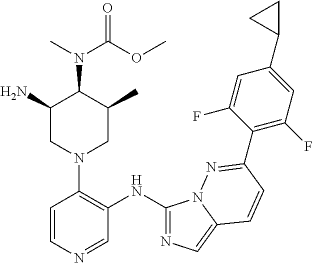 [SMILES: COC(=O)N(C)[C@@H]1[C@H](N)CN(c2ccncc2Nc2ncc3ccc(-c4c(F)cc(C5CC5)cc4F)nn23)C[C@@H]1C]

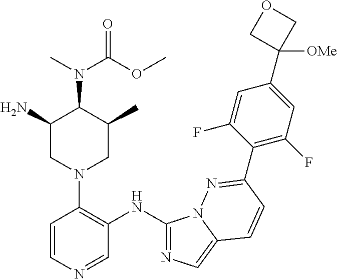 [SMILES: COC(=O)N(C)[C@@H]1[C@H](N)CN(c2ccncc2Nc2ncc3ccc(-c4c(F)cc(C5(OC)COC5)cc4F)nn23)C[C@@H]1C]

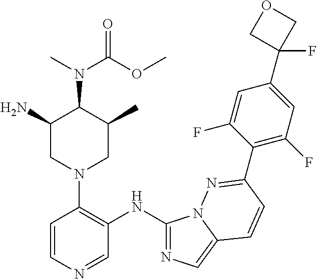 [SMILES: COC(=O)N(C)[C@@H]1[C@H](N)CN(c2ccncc2Nc2ncc3ccc(-c4c(F)cc(C5(F)COC5)cc4F)nn23)C[C@@H]1C]

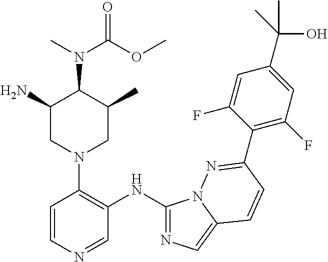 [SMILES: COC(=O)N(C)[C@@H]1[C@H](N)CN(c2ccncc2Nc2ncc3ccc(-c4c(F)cc(C(C)(C)O)cc4F)nn23)C[C@@H]1C]

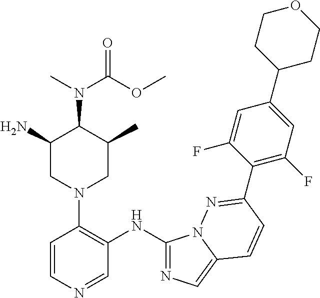 [SMILES: COC(=O)N(C)[C@@H]1[C@H](N)CN(c2ccncc2Nc2ncc3ccc(-c4c(F)cc(C5CCOCC5)cc4F)nn23)C[C@@H]1C]

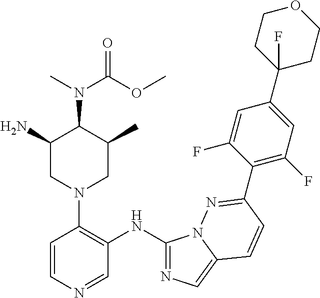

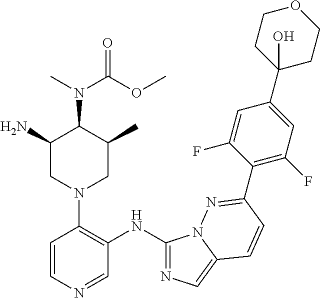

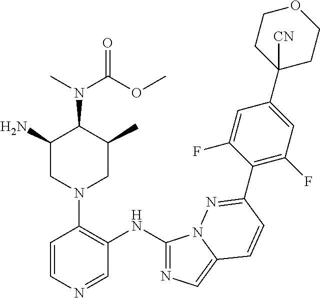

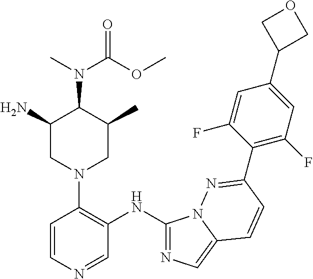 [SMILES: COC(=O)N(C)[C@@H]1[C@H](N)CN(c2ccncc2Nc2ncc3ccc(-c4c(F)cc(C5COC5)cc4F)nn23)C[C@@H]1C]

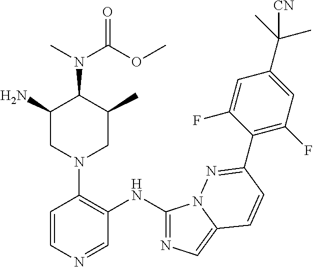 [SMILES: COC(=O)N(C)[C@@H]1[C@H](N)CN(c2ccncc2Nc2ncc3ccc(-c4c(F)cc(C(C)(C)C#N)cc4F)nn23)C[C@@H]1C]

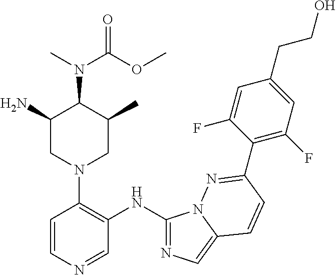 [SMILES: COC(=O)N(C)[C@@H]1[C@H](N)CN(c2ccncc2Nc2ncc3ccc(-c4c(F)cc(CCO)cc4F)nn23)C[C@@H]1C]

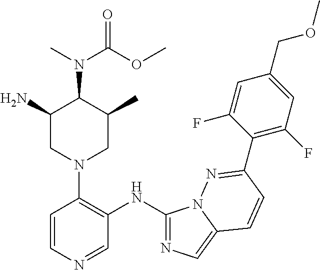 [SMILES: COCc1cc(F)c(-c2ccc3cnc(Nc4cnccc4N4C[C@@H](N)[C@@H](N(C)C(=O)OC)[C@@H](C)C4)n3n2)c(F)c1]

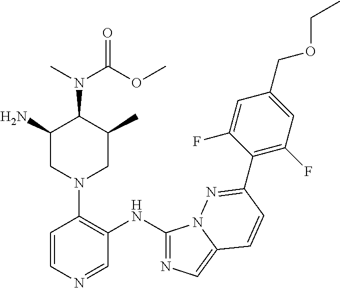 [SMILES: CCOCc1cc(F)c(-c2ccc3cnc(Nc4cnccc4N4C[C@@H](N)[C@@H](N(C)C(=O)OC)[C@@H](C)C4)n3n2)c(F)c1]

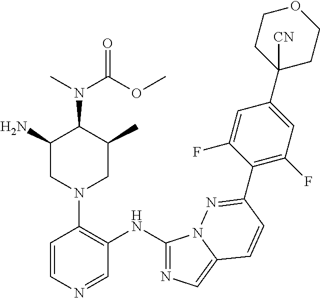

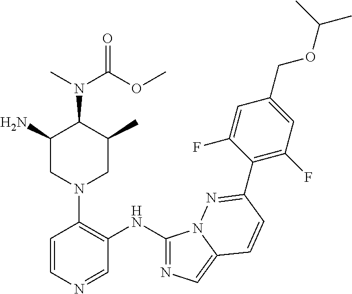 [SMILES: COC(=O)N(C)[C@@H]1[C@H](N)CN(c2ccncc2Nc2ncc3ccc(-c4c(F)cc(COC(C)C)cc4F)nn23)C[C@@H]1C]

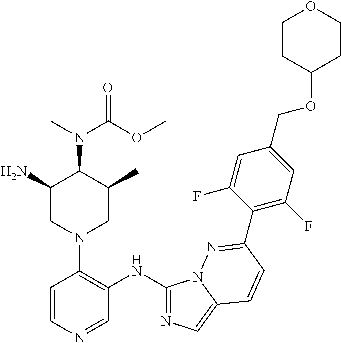 [SMILES: COC(=O)N(C)[C@@H]1[C@H](N)CN(c2ccncc2Nc2ncc3ccc(-c4c(F)cc(COC5CCOCC5)cc4F)nn23)C[C@@H]1C]

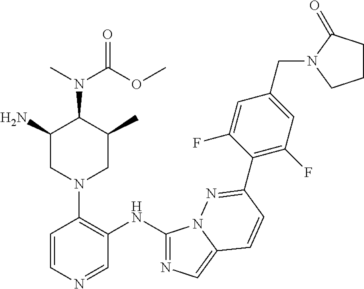 [SMILES: COC(=O)N(C)[C@@H]1[C@H](N)CN(c2ccncc2Nc2ncc3ccc(-c4c(F)cc(CN5CCCC5=O)cc4F)nn23)C[C@@H]1C]

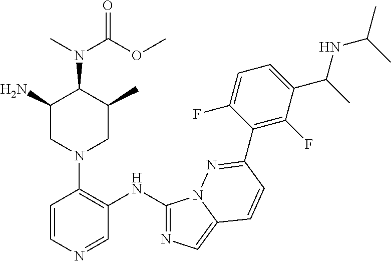 [SMILES: COC(=O)N(C)[C@@H]1[C@H](N)CN(c2ccncc2Nc2ncc3ccc(-c4c(F)ccc(C(C)NC(C)C)c4F)nn23)C[C@@H]1C]

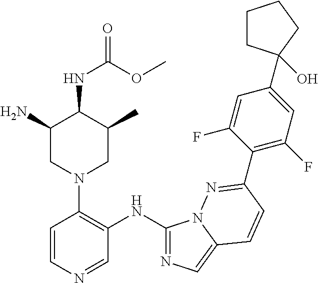 [SMILES: COC(=O)N[C@@H]1[C@H](N)CN(c2ccncc2Nc2ncc3ccc(-c4c(F)cc(C5(O)CCCC5)cc4F)nn23)C[C@@H]1C]

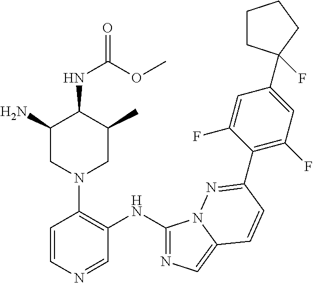 [SMILES: COC(=O)N[C@@H]1[C@H](N)CN(c2ccncc2Nc2ncc3ccc(-c4c(F)cc(C5(F)CCCC5)cc4F)nn23)C[C@@H]1C]

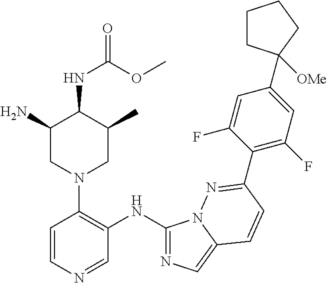 [SMILES: COC(=O)N[C@@H]1[C@H](N)CN(c2ccncc2Nc2ncc3ccc(-c4c(F)cc(C5(OC)CCCC5)cc4F)nn23)C[C@@H]1C]

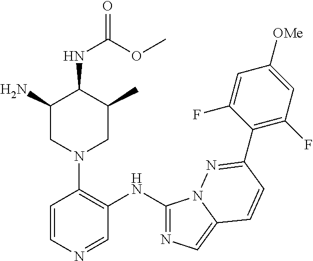 [SMILES: COC(=O)N[C@@H]1[C@H](N)CN(c2ccncc2Nc2ncc3ccc(-c4c(F)cc(OC)cc4F)nn23)C[C@@H]1C]

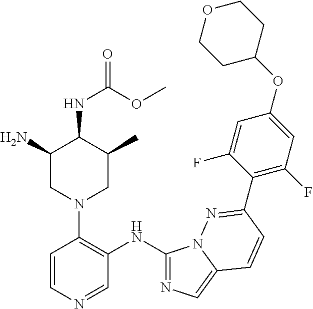 [SMILES: COC(=O)N[C@@H]1[C@H](N)CN(c2ccncc2Nc2ncc3ccc(-c4c(F)cc(OC5CCOCC5)cc4F)nn23)C[C@@H]1C]

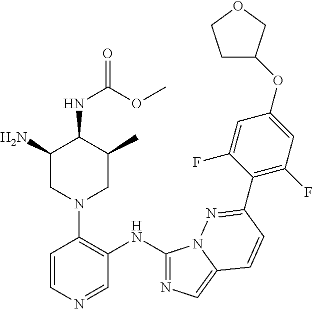 [SMILES: COC(=O)N[C@@H]1[C@H](N)CN(c2ccncc2Nc2ncc3ccc(-c4c(F)cc(OC5CCOC5)cc4F)nn23)C[C@@H]1C]

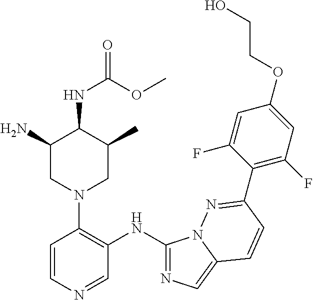 [SMILES: COC(=O)N[C@@H]1[C@H](N)CN(c2ccncc2Nc2ncc3ccc(-c4c(F)cc(OCCO)cc4F)nn23)C[C@@H]1C]

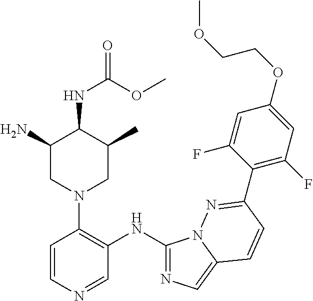 [SMILES: COCCOc1cc(F)c(-c2ccc3cnc(Nc4cnccc4N4C[C@@H](N)[C@@H](NC(=O)OC)[C@@H](C)C4)n3n2)c(F)c1]

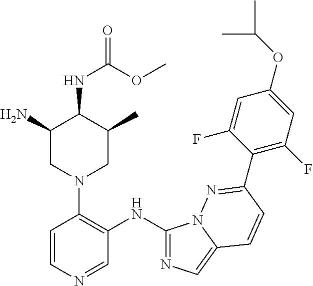 [SMILES: COC(=O)N[C@@H]1[C@H](N)CN(c2ccncc2Nc2ncc3ccc(-c4c(F)cc(OC(C)C)cc4F)nn23)C[C@@H]1C]

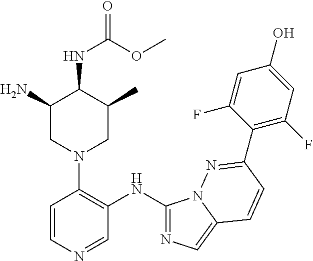 [SMILES: COC(=O)N[C@@H]1[C@H](N)CN(c2ccncc2Nc2ncc3ccc(-c4c(F)cc(O)cc4F)nn23)C[C@@H]1C]

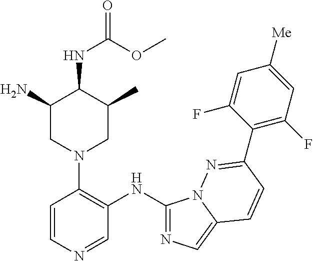 [SMILES: COC(=O)N[C@@H]1[C@H](N)CN(c2ccncc2Nc2ncc3ccc(-c4c(F)cc(C)cc4F)nn23)C[C@@H]1C]

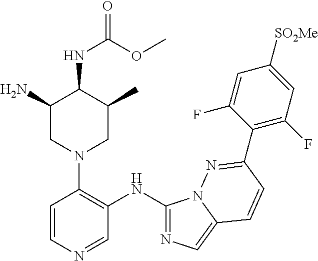 [SMILES: COC(=O)N[C@@H]1[C@H](N)CN(c2ccncc2Nc2ncc3ccc(-c4c(F)cc(S(C)(=O)=O)cc4F)nn23)C[C@@H]1C]

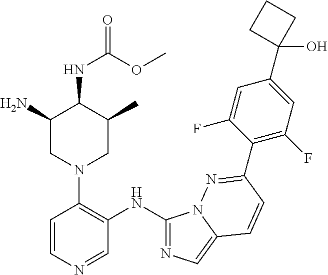 [SMILES: COC(=O)N[C@@H]1[C@H](N)CN(c2ccncc2Nc2ncc3ccc(-c4c(F)cc(C5(O)CCC5)cc4F)nn23)C[C@@H]1C]

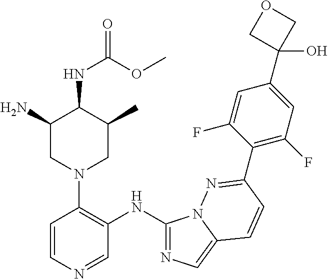 [SMILES: COC(=O)N[C@@H]1[C@H](N)CN(c2ccncc2Nc2ncc3ccc(-c4c(F)cc(C5(O)COC5)cc4F)nn23)C[C@@H]1C]

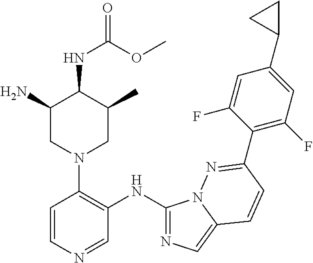 [SMILES: COC(=O)N[C@@H]1[C@H](N)CN(c2ccncc2Nc2ncc3ccc(-c4c(F)cc(C5CC5)cc4F)nn23)C[C@@H]1C]

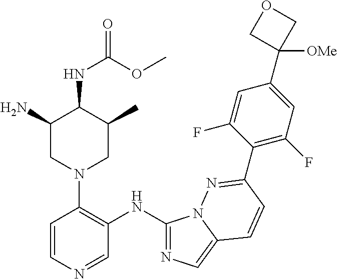 [SMILES: COC(=O)N[C@@H]1[C@H](N)CN(c2ccncc2Nc2ncc3ccc(-c4c(F)cc(C5(OC)COC5)cc4F)nn23)C[C@@H]1C]

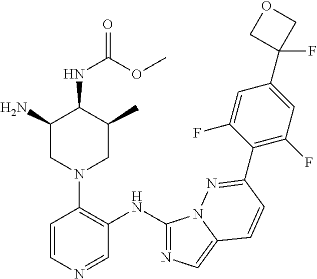 [SMILES: COC(=O)N[C@@H]1[C@H](N)CN(c2ccncc2Nc2ncc3ccc(-c4c(F)cc(C5(F)COC5)cc4F)nn23)C[C@@H]1C]

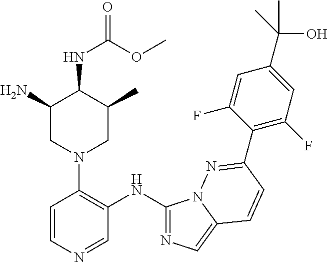 [SMILES: COC(=O)N[C@@H]1[C@H](N)CN(c2ccncc2Nc2ncc3ccc(-c4c(F)cc(C(C)(C)O)cc4F)nn23)C[C@@H]1C]

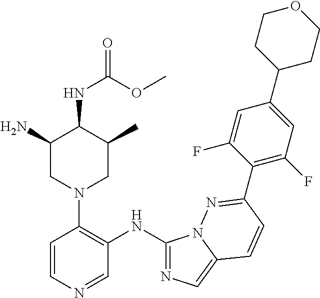 [SMILES: COC(=O)N[C@@H]1[C@H](N)CN(c2ccncc2Nc2ncc3ccc(-c4c(F)cc(C5CCOCC5)cc4F)nn23)C[C@@H]1C]

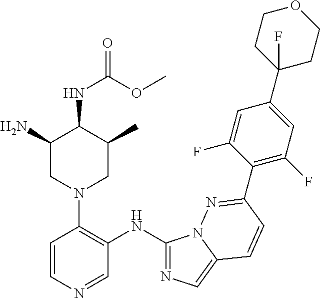

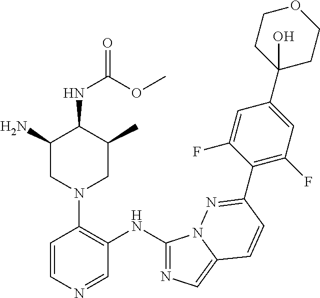

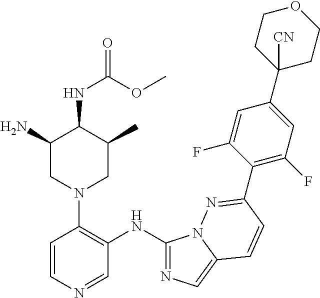

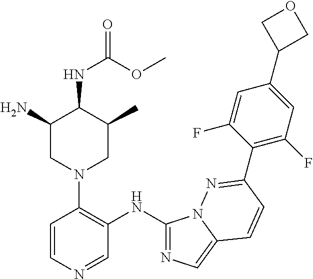 [SMILES: COC(=O)N[C@@H]1[C@H](N)CN(c2ccncc2Nc2ncc3ccc(-c4c(F)cc(C5COC5)cc4F)nn23)C[C@@H]1C]

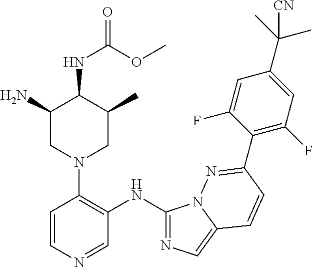 [SMILES: COC(=O)N[C@@H]1[C@H](N)CN(c2ccncc2Nc2ncc3ccc(-c4c(F)cc(C(C)(C)C#N)cc4F)nn23)C[C@@H]1C]

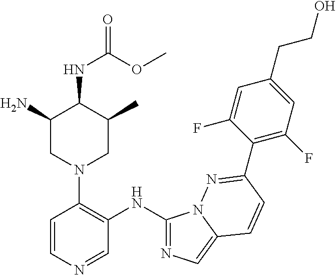 [SMILES: COC(=O)N[C@@H]1[C@H](N)CN(c2ccncc2Nc2ncc3ccc(-c4c(F)cc(CCO)cc4F)nn23)C[C@@H]1C]

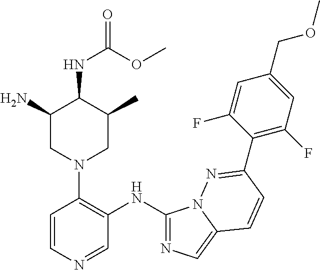 [SMILES: COCc1cc(F)c(-c2ccc3cnc(Nc4cnccc4N4C[C@@H](N)[C@@H](NC(=O)OC)[C@@H](C)C4)n3n2)c(F)c1]

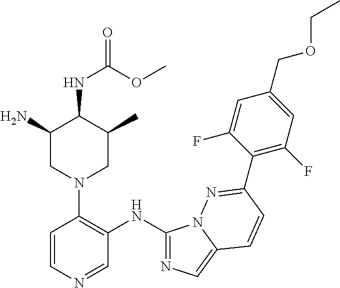 [SMILES: CCOCc1cc(F)c(-c2ccc3cnc(Nc4cnccc4N4C[C@@H](N)[C@@H](NC(=O)OC)[C@@H](C)C4)n3n2)c(F)c1]

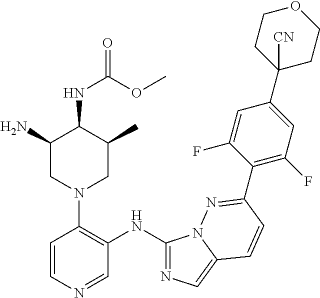

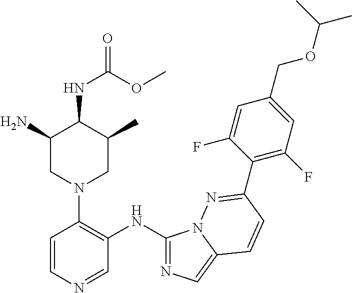 [SMILES: COC(=O)N[C@@H]1[C@H](N)CN(c2ccncc2Nc2ncc3ccc(-c4c(F)cc(COC(C)C)cc4F)nn23)C[C@@H]1C]

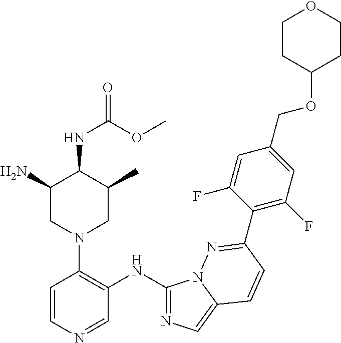 [SMILES: COC(=O)N[C@@H]1[C@H](N)CN(c2ccncc2Nc2ncc3ccc(-c4c(F)cc(COC5CCOCC5)cc4F)nn23)C[C@@H]1C]

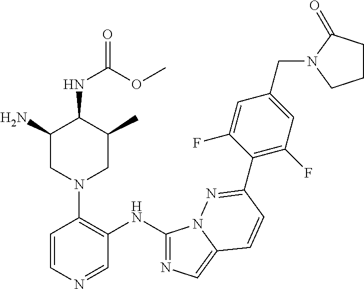 [SMILES: COC(=O)N[C@@H]1[C@H](N)CN(c2ccncc2Nc2ncc3ccc(-c4c(F)cc(CN5CCCC5=O)cc4F)nn23)C[C@@H]1C]

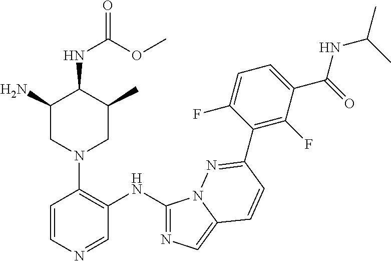 [SMILES: COC(=O)N[C@@H]1[C@H](N)CN(c2ccncc2Nc2ncc3ccc(-c4c(F)ccc(C(=O)NC(C)C)c4F)nn23)C[C@@H]1C]

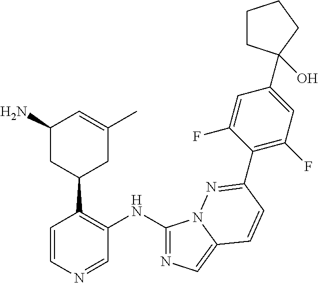 [SMILES: CC1=C[C@H](N)C[C@H](c2ccncc2Nc2ncc3ccc(-c4c(F)cc(C5(O)CCCC5)cc4F)nn23)C1]

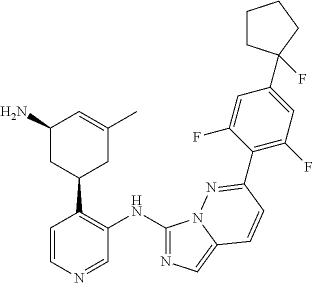 [SMILES: CC1=C[C@H](N)C[C@H](c2ccncc2Nc2ncc3ccc(-c4c(F)cc(C5(F)CCCC5)cc4F)nn23)C1]

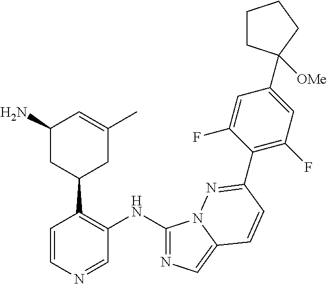 [SMILES: COC1(c2cc(F)c(-c3ccc4cnc(Nc5cnccc5[C@@H]5CC(C)=C[C@H](N)C5)n4n3)c(F)c2)CCCC1]

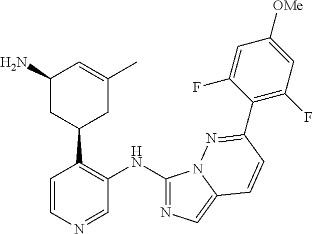 [SMILES: COc1cc(F)c(-c2ccc3cnc(Nc4cnccc4[C@@H]4CC(C)=C[C@H](N)C4)n3n2)c(F)c1]

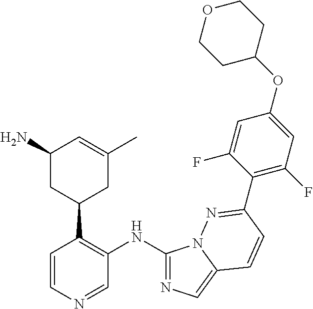 [SMILES: CC1=C[C@H](N)C[C@H](c2ccncc2Nc2ncc3ccc(-c4c(F)cc(OC5CCOCC5)cc4F)nn23)C1]

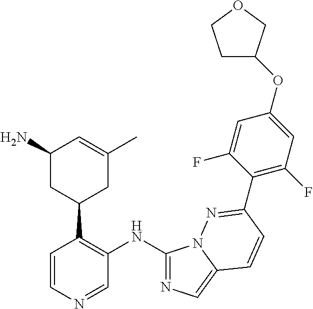 [SMILES: CC1=C[C@H](N)C[C@H](c2ccncc2Nc2ncc3ccc(-c4c(F)cc(OC5CCOC5)cc4F)nn23)C1]

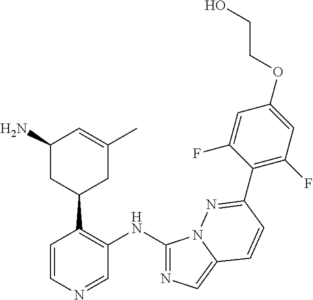 [SMILES: CC1=C[C@H](N)C[C@H](c2ccncc2Nc2ncc3ccc(-c4c(F)cc(OCCO)cc4F)nn23)C1]

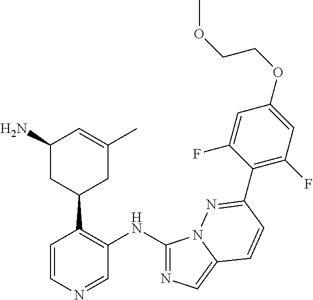 [SMILES: COCCOc1cc(F)c(-c2ccc3cnc(Nc4cnccc4[C@@H]4CC(C)=C[C@H](N)C4)n3n2)c(F)c1]

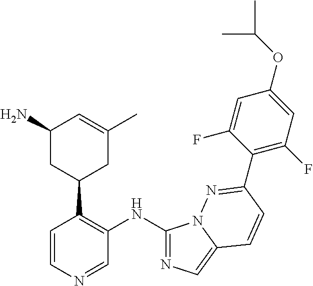 [SMILES: CC1=C[C@H](N)C[C@H](c2ccncc2Nc2ncc3ccc(-c4c(F)cc(OC(C)C)cc4F)nn23)C1]

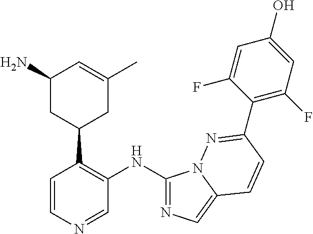 [SMILES: CC1=C[C@H](N)C[C@H](c2ccncc2Nc2ncc3ccc(-c4c(F)cc(O)cc4F)nn23)C1]

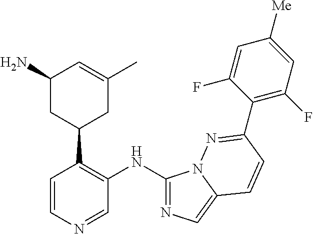 [SMILES: CC1=C[C@H](N)C[C@H](c2ccncc2Nc2ncc3ccc(-c4c(F)cc(C)cc4F)nn23)C1]

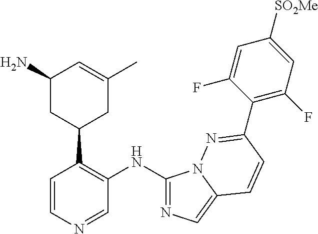 [SMILES: CC1=C[C@H](N)C[C@H](c2ccncc2Nc2ncc3ccc(-c4c(F)cc(S(C)(=O)=O)cc4F)nn23)C1]

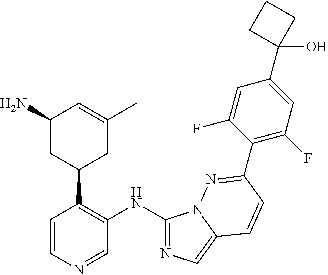 [SMILES: CC1=C[C@H](N)C[C@H](c2ccncc2Nc2ncc3ccc(-c4c(F)cc(C5(O)CCC5)cc4F)nn23)C1]

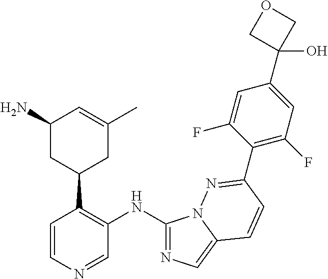 [SMILES: CC1=C[C@H](N)C[C@H](c2ccncc2Nc2ncc3ccc(-c4c(F)cc(C5(O)COC5)cc4F)nn23)C1]

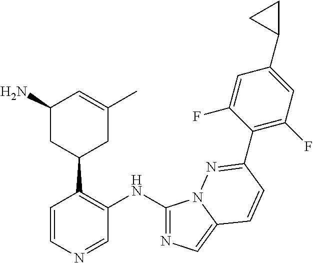 [SMILES: CC1=C[C@H](N)C[C@H](c2ccncc2Nc2ncc3ccc(-c4c(F)cc(C5CC5)cc4F)nn23)C1]

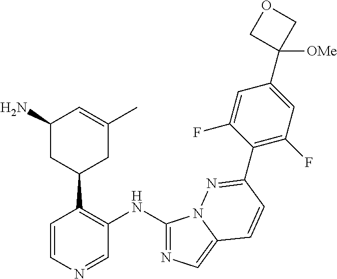 [SMILES: COC1(c2cc(F)c(-c3ccc4cnc(Nc5cnccc5[C@@H]5CC(C)=C[C@H](N)C5)n4n3)c(F)c2)COC1]

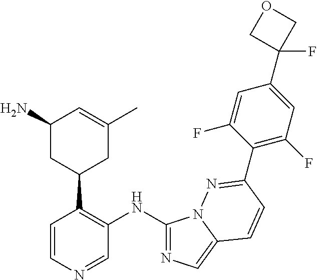 [SMILES: CC1=C[C@H](N)C[C@H](c2ccncc2Nc2ncc3ccc(-c4c(F)cc(C5(F)COC5)cc4F)nn23)C1]

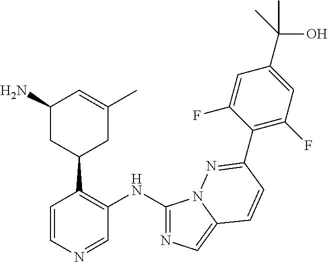 [SMILES: CC1=C[C@H](N)C[C@H](c2ccncc2Nc2ncc3ccc(-c4c(F)cc(C(C)(C)O)cc4F)nn23)C1]

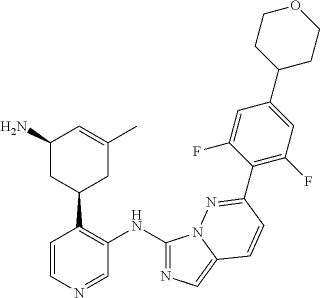 [SMILES: CC1=C[C@H](N)C[C@H](c2ccncc2Nc2ncc3ccc(-c4c(F)cc(C5CCOCC5)cc4F)nn23)C1]

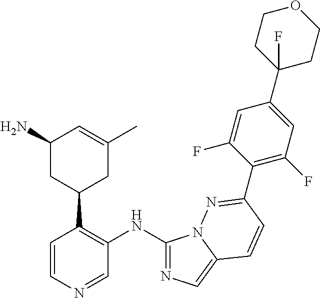

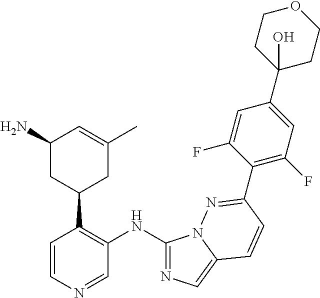

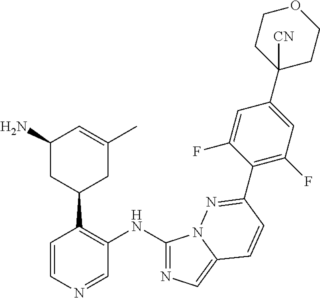

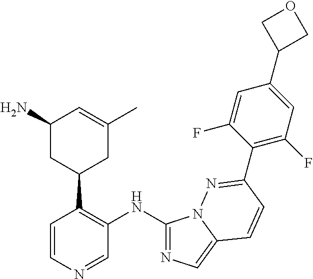 [SMILES: CC1=C[C@H](N)C[C@H](c2ccncc2Nc2ncc3ccc(-c4c(F)cc(C5COC5)cc4F)nn23)C1]

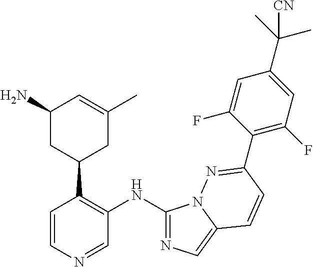 [SMILES: CC1=C[C@H](N)C[C@H](c2ccncc2Nc2ncc3ccc(-c4c(F)cc(C(C)(C)C#N)cc4F)nn23)C1]

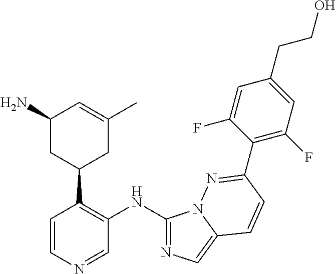 [SMILES: CC1=C[C@H](N)C[C@H](c2ccncc2Nc2ncc3ccc(-c4c(F)cc(CCO)cc4F)nn23)C1]

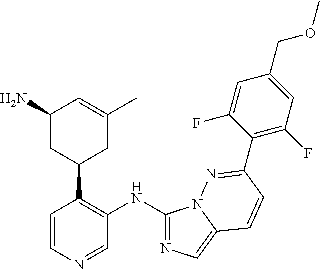 [SMILES: COCc1cc(F)c(-c2ccc3cnc(Nc4cnccc4[C@@H]4CC(C)=C[C@H](N)C4)n3n2)c(F)c1]

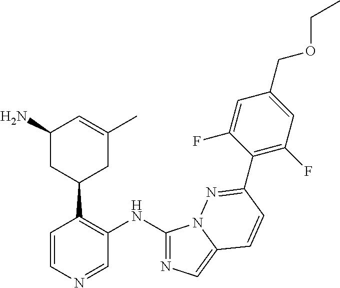 [SMILES: CCOCc1cc(F)c(-c2ccc3cnc(Nc4cnccc4[C@@H]4CC(C)=C[C@H](N)C4)n3n2)c(F)c1]

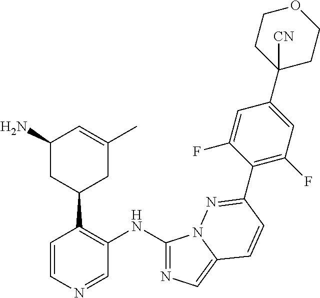

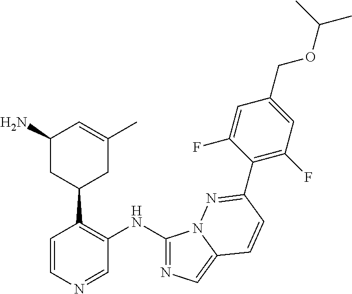 [SMILES: CC1=C[C@H](N)C[C@H](c2ccncc2Nc2ncc3ccc(-c4c(F)cc(COC(C)C)cc4F)nn23)C1]

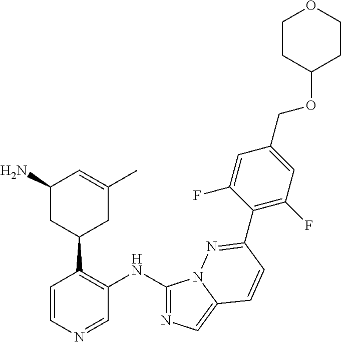 [SMILES: CC1=C[C@H](N)C[C@H](c2ccncc2Nc2ncc3ccc(-c4c(F)cc(COC5CCOCC5)cc4F)nn23)C1]

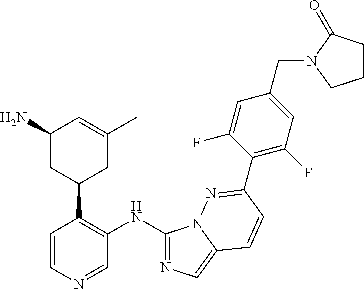 [SMILES: CC1=C[C@H](N)C[C@H](c2ccncc2Nc2ncc3ccc(-c4c(F)cc(CN5CCCC5=O)cc4F)nn23)C1]

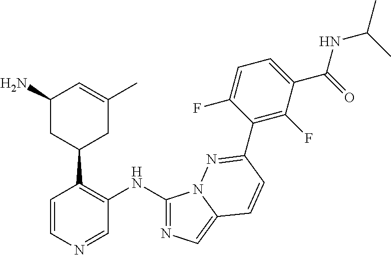 [SMILES: CC1=C[C@H](N)C[C@H](c2ccncc2Nc2ncc3ccc(-c4c(F)ccc(C(=O)NC(C)C)c4F)nn23)C1]

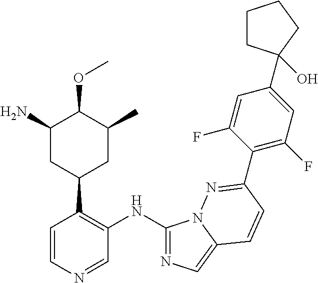 [SMILES: CO[C@@H]1[C@H](N)C[C@H](c2ccncc2Nc2ncc3ccc(-c4c(F)cc(C5(O)CCCC5)cc4F)nn23)C[C@@H]1C]

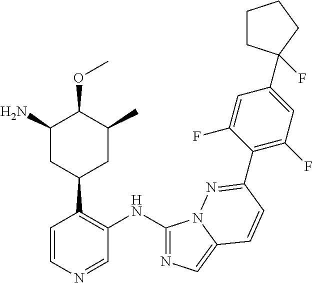 [SMILES: CO[C@@H]1[C@H](N)C[C@H](c2ccncc2Nc2ncc3ccc(-c4c(F)cc(C5(F)CCCC5)cc4F)nn23)C[C@@H]1C]

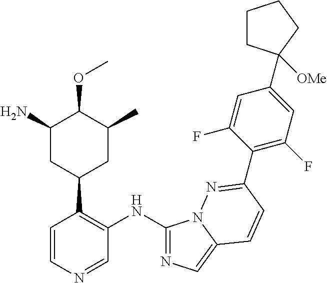 [SMILES: CO[C@@H]1[C@H](N)C[C@H](c2ccncc2Nc2ncc3ccc(-c4c(F)cc(C5(OC)CCCC5)cc4F)nn23)C[C@@H]1C]

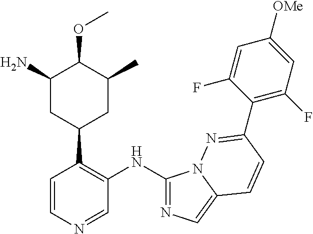 [SMILES: COc1cc(F)c(-c2ccc3cnc(Nc4cnccc4[C@H]4C[C@@H](N)[C@@H](OC)[C@@H](C)C4)n3n2)c(F)c1]

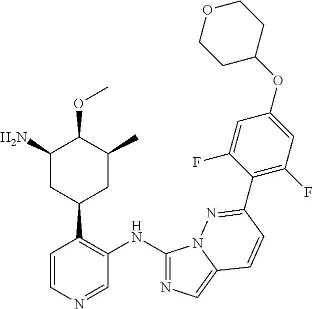 [SMILES: CO[C@@H]1[C@H](N)C[C@H](c2ccncc2Nc2ncc3ccc(-c4c(F)cc(OC5CCOCC5)cc4F)nn23)C[C@@H]1C]

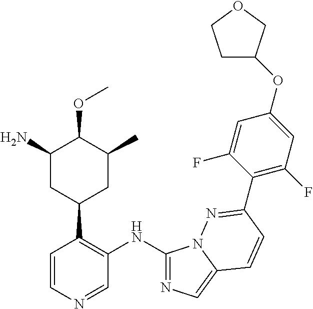 [SMILES: CO[C@@H]1[C@H](N)C[C@H](c2ccncc2Nc2ncc3ccc(-c4c(F)cc(OC5CCOC5)cc4F)nn23)C[C@@H]1C]

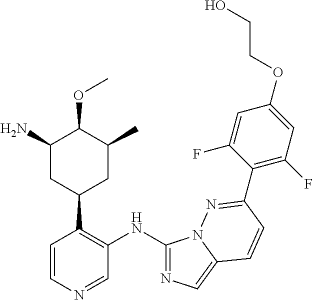 [SMILES: CO[C@@H]1[C@H](N)C[C@H](c2ccncc2Nc2ncc3ccc(-c4c(F)cc(OCCO)cc4F)nn23)C[C@@H]1C]

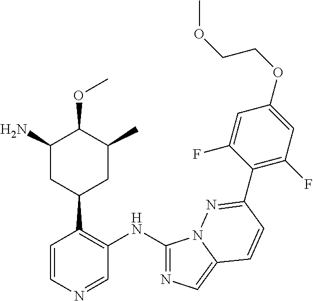 [SMILES: COCCOc1cc(F)c(-c2ccc3cnc(Nc4cnccc4[C@H]4C[C@@H](N)[C@@H](OC)[C@@H](C)C4)n3n2)c(F)c1]

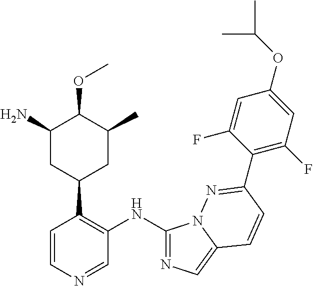 [SMILES: CO[C@@H]1[C@H](N)C[C@H](c2ccncc2Nc2ncc3ccc(-c4c(F)cc(OC(C)C)cc4F)nn23)C[C@@H]1C]

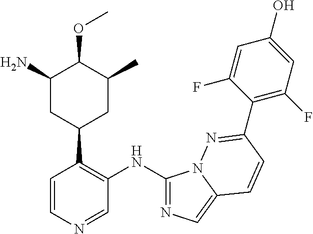 [SMILES: CO[C@@H]1[C@H](N)C[C@H](c2ccncc2Nc2ncc3ccc(-c4c(F)cc(O)cc4F)nn23)C[C@@H]1C]

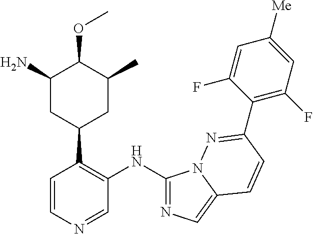 [SMILES: CO[C@@H]1[C@H](N)C[C@H](c2ccncc2Nc2ncc3ccc(-c4c(F)cc(C)cc4F)nn23)C[C@@H]1C]

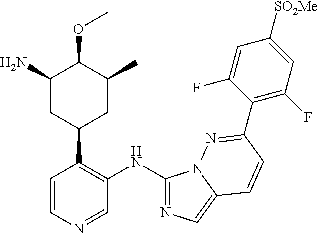 [SMILES: CO[C@@H]1[C@H](N)C[C@H](c2ccncc2Nc2ncc3ccc(-c4c(F)cc(S(C)(=O)=O)cc4F)nn23)C[C@@H]1C]

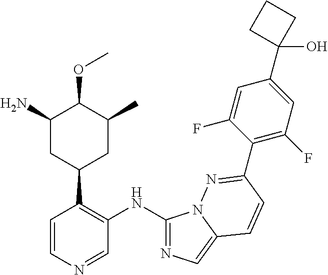 [SMILES: CO[C@@H]1[C@H](N)C[C@H](c2ccncc2Nc2ncc3ccc(-c4c(F)cc(C5(O)CCC5)cc4F)nn23)C[C@@H]1C]

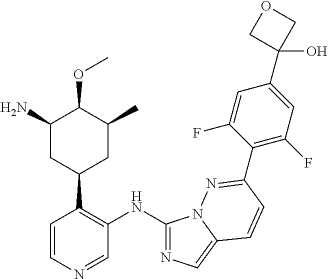 [SMILES: CO[C@@H]1[C@H](N)C[C@H](c2ccncc2Nc2ncc3ccc(-c4c(F)cc(C5(O)COC5)cc4F)nn23)C[C@@H]1C]

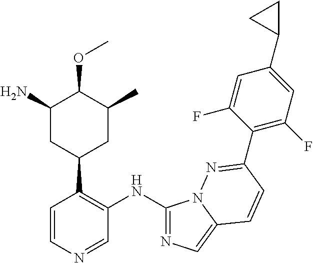 [SMILES: CO[C@@H]1[C@H](N)C[C@H](c2ccncc2Nc2ncc3ccc(-c4c(F)cc(C5CC5)cc4F)nn23)C[C@@H]1C]

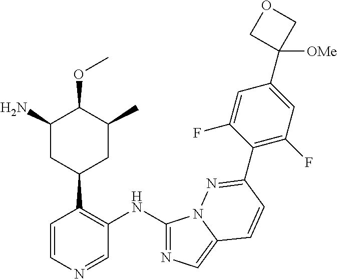 [SMILES: CO[C@@H]1[C@H](N)C[C@H](c2ccncc2Nc2ncc3ccc(-c4c(F)cc(C5(OC)COC5)cc4F)nn23)C[C@@H]1C]

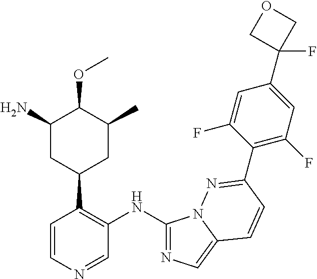 [SMILES: CO[C@@H]1[C@H](N)C[C@H](c2ccncc2Nc2ncc3ccc(-c4c(F)cc(C5(F)COC5)cc4F)nn23)C[C@@H]1C]

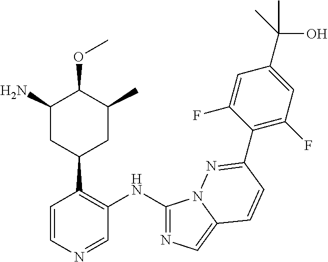 [SMILES: CO[C@@H]1[C@H](N)C[C@H](c2ccncc2Nc2ncc3ccc(-c4c(F)cc(C(C)(C)O)cc4F)nn23)C[C@@H]1C]

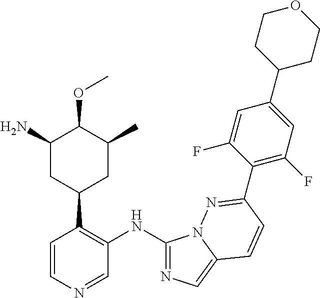 [SMILES: CO[C@@H]1[C@H](N)C[C@H](c2ccncc2Nc2ncc3ccc(-c4c(F)cc(C5CCOCC5)cc4F)nn23)C[C@@H]1C]

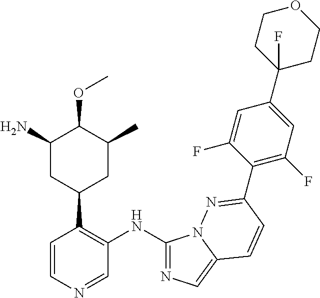

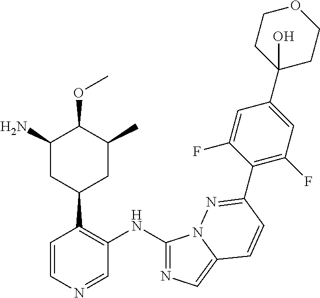

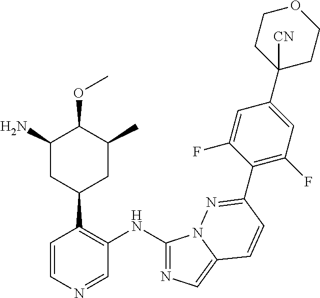

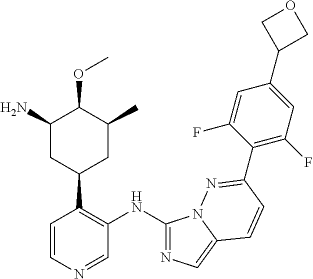 [SMILES: CO[C@@H]1[C@H](N)C[C@H](c2ccncc2Nc2ncc3ccc(-c4c(F)cc(C5COC5)cc4F)nn23)C[C@@H]1C]

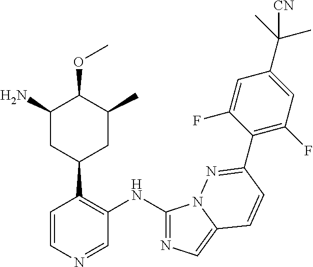 [SMILES: CO[C@@H]1[C@H](N)C[C@H](c2ccncc2Nc2ncc3ccc(-c4c(F)cc(C(C)(C)C#N)cc4F)nn23)C[C@@H]1C]

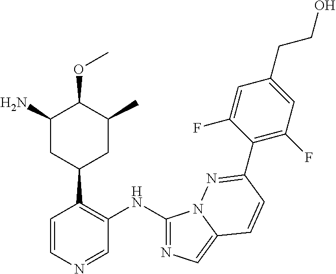 [SMILES: CO[C@@H]1[C@H](N)C[C@H](c2ccncc2Nc2ncc3ccc(-c4c(F)cc(CCO)cc4F)nn23)C[C@@H]1C]

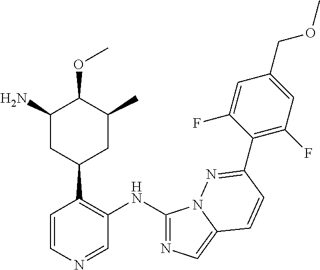 [SMILES: COCc1cc(F)c(-c2ccc3cnc(Nc4cnccc4[C@H]4C[C@@H](N)[C@@H](OC)[C@@H](C)C4)n3n2)c(F)c1]

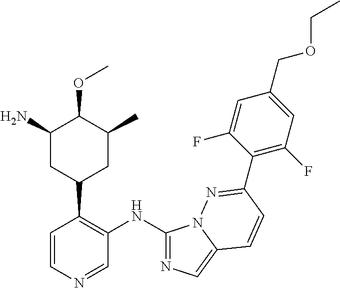

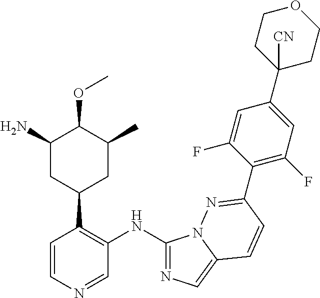

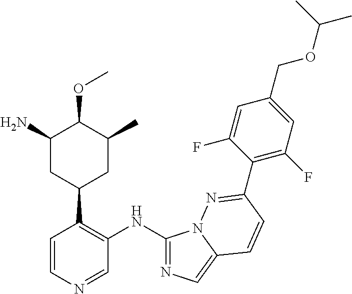 [SMILES: CO[C@@H]1[C@H](N)C[C@H](c2ccncc2Nc2ncc3ccc(-c4c(F)cc(COC(C)C)cc4F)nn23)C[C@@H]1C]

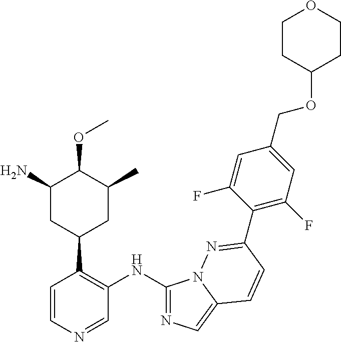 [SMILES: CO[C@@H]1[C@H](N)C[C@H](c2ccncc2Nc2ncc3ccc(-c4c(F)cc(COC5CCOCC5)cc4F)nn23)C[C@@H]1C]

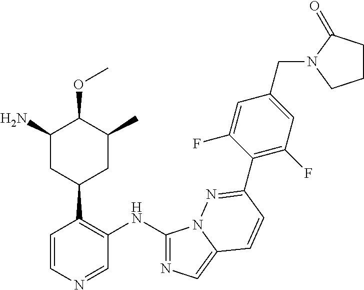 [SMILES: CO[C@@H]1[C@H](N)C[C@H](c2ccncc2Nc2ncc3ccc(-c4c(F)cc(CN5CCCC5=O)cc4F)nn23)C[C@@H]1C]

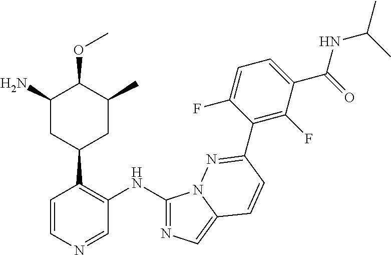 [SMILES: CO[C@@H]1[C@H](N)C[C@H](c2ccncc2Nc2ncc3ccc(-c4c(F)ccc(C(=O)NC(C)C)c4F)nn23)C[C@@H]1C]

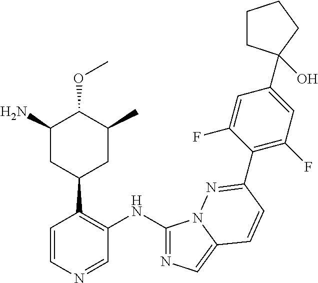 [SMILES: CO[C@H]1[C@H](N)C[C@H](c2ccncc2Nc2ncc3ccc(-c4c(F)cc(C5(O)CCCC5)cc4F)nn23)C[C@@H]1C]

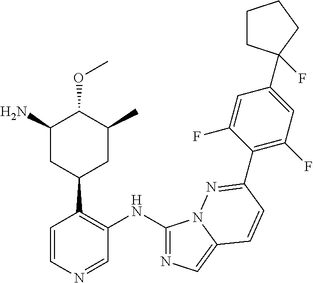 [SMILES: CO[C@H]1[C@H](N)C[C@H](c2ccncc2Nc2ncc3ccc(-c4c(F)cc(C5(F)CCCC5)cc4F)nn23)C[C@@H]1C]

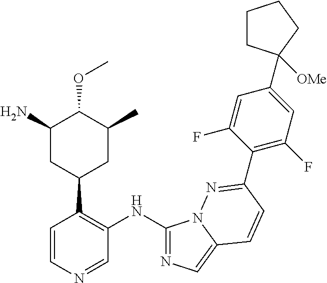 [SMILES: CO[C@H]1[C@H](N)C[C@H](c2ccncc2Nc2ncc3ccc(-c4c(F)cc(C5(OC)CCCC5)cc4F)nn23)C[C@@H]1C]

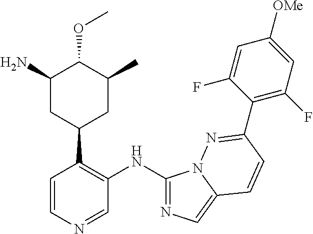 [SMILES: COc1cc(F)c(-c2ccc3cnc(Nc4cnccc4[C@H]4C[C@@H](N)[C@H](OC)[C@@H](C)C4)n3n2)c(F)c1]

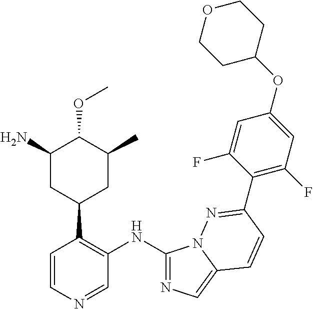 [SMILES: CO[C@H]1[C@H](N)C[C@H](c2ccncc2Nc2ncc3ccc(-c4c(F)cc(OC5CCOCC5)cc4F)nn23)C[C@@H]1C]

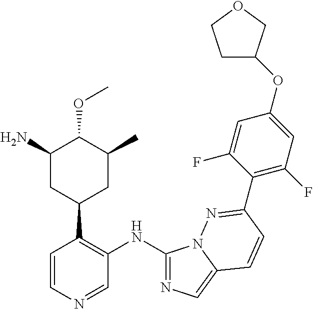 [SMILES: CO[C@H]1[C@H](N)C[C@H](c2ccncc2Nc2ncc3ccc(-c4c(F)cc(OC5CCOC5)cc4F)nn23)C[C@@H]1C]

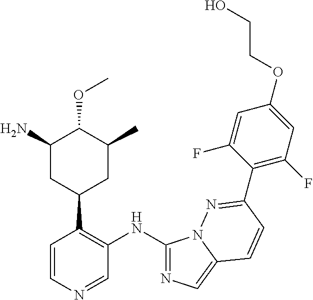 [SMILES: CO[C@H]1[C@H](N)C[C@H](c2ccncc2Nc2ncc3ccc(-c4c(F)cc(OCCO)cc4F)nn23)C[C@@H]1C]

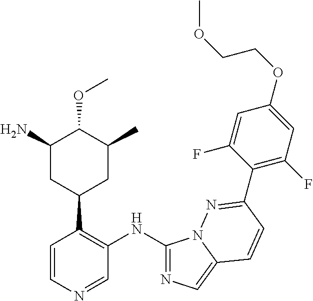 [SMILES: COCCOc1cc(F)c(-c2ccc3cnc(Nc4cnccc4[C@H]4C[C@@H](N)[C@H](OC)[C@@H](C)C4)n3n2)c(F)c1]

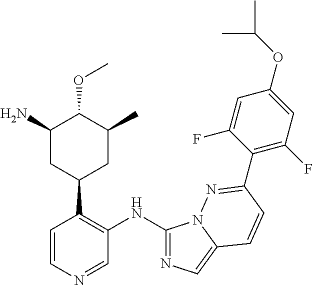 [SMILES: CO[C@H]1[C@H](N)C[C@H](c2ccncc2Nc2ncc3ccc(-c4c(F)cc(OC(C)C)cc4F)nn23)C[C@@H]1C]

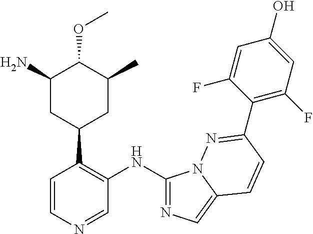 [SMILES: CO[C@H]1[C@H](N)C[C@H](c2ccncc2Nc2ncc3ccc(-c4c(F)cc(O)cc4F)nn23)C[C@@H]1C]

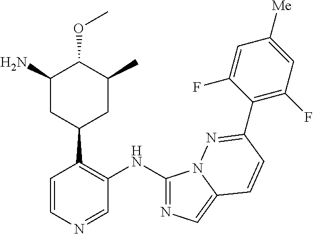 [SMILES: CO[C@H]1[C@H](N)C[C@H](c2ccncc2Nc2ncc3ccc(-c4c(F)cc(C)cc4F)nn23)C[C@@H]1C]

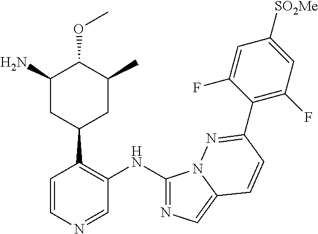 [SMILES: CO[C@H]1[C@H](N)C[C@H](c2ccncc2Nc2ncc3ccc(-c4c(F)cc(S(C)(=O)=O)cc4F)nn23)C[C@@H]1C]

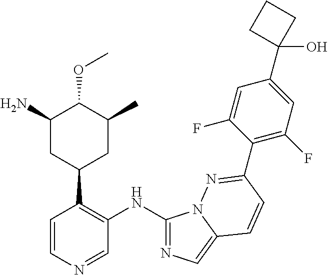 [SMILES: CO[C@H]1[C@H](N)C[C@H](c2ccncc2Nc2ncc3ccc(-c4c(F)cc(C5(O)CCC5)cc4F)nn23)C[C@@H]1C]

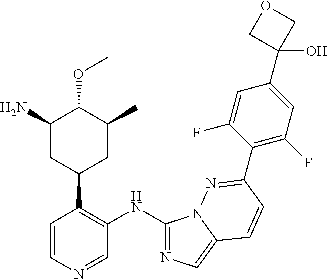 [SMILES: CO[C@H]1[C@H](N)C[C@H](c2ccncc2Nc2ncc3ccc(-c4c(F)cc(C5(O)COC5)cc4F)nn23)C[C@@H]1C]

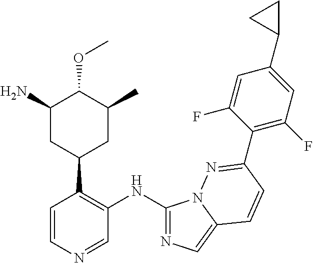 [SMILES: CO[C@H]1[C@H](N)C[C@H](c2ccncc2Nc2ncc3ccc(-c4c(F)cc(C5CC5)cc4F)nn23)C[C@@H]1C]

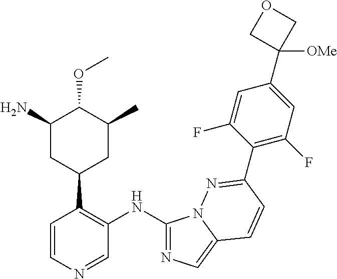 [SMILES: CO[C@H]1[C@H](N)C[C@H](c2ccncc2Nc2ncc3ccc(-c4c(F)cc(C5(OC)COC5)cc4F)nn23)C[C@@H]1C]

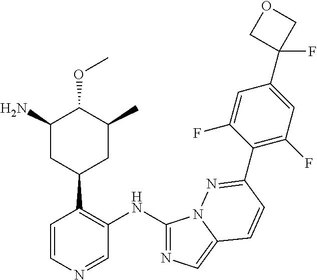 [SMILES: CO[C@H]1[C@H](N)C[C@H](c2ccncc2Nc2ncc3ccc(-c4c(F)cc(C5(F)COC5)cc4F)nn23)C[C@@H]1C]

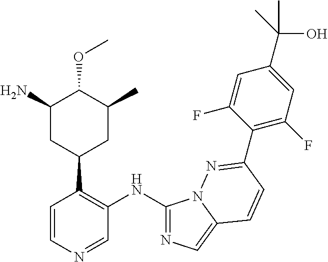 [SMILES: CO[C@H]1[C@H](N)C[C@H](c2ccncc2Nc2ncc3ccc(-c4c(F)cc(C(C)(C)O)cc4F)nn23)C[C@@H]1C]

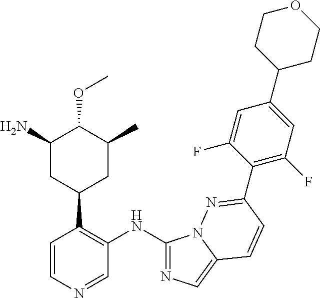 [SMILES: CO[C@H]1[C@H](N)C[C@H](c2ccncc2Nc2ncc3ccc(-c4c(F)cc(C5CCOCC5)cc4F)nn23)C[C@@H]1C]

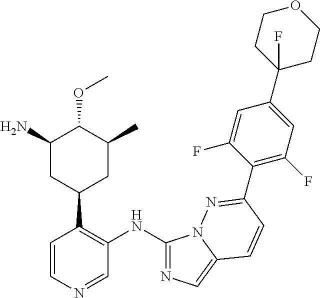

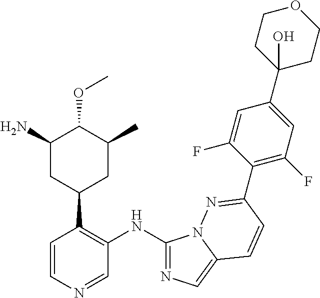

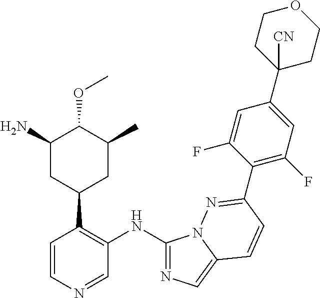

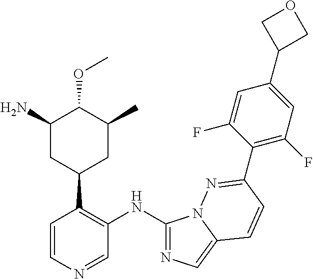 [SMILES: CO[C@H]1[C@H](N)C[C@H](c2ccncc2Nc2ncc3ccc(-c4c(F)cc(C5COC5)cc4F)nn23)C[C@@H]1C]

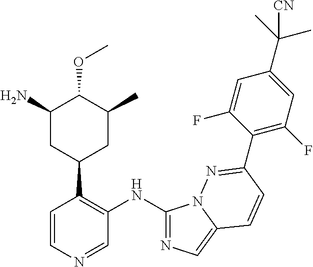 [SMILES: CO[C@H]1[C@H](N)C[C@H](c2ccncc2Nc2ncc3ccc(-c4c(F)cc(C(C)(C)C#N)cc4F)nn23)C[C@@H]1C]

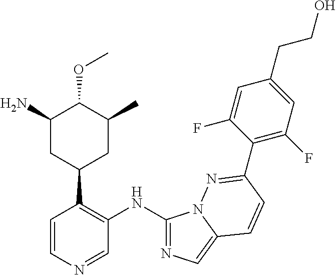 [SMILES: CO[C@H]1[C@H](N)C[C@H](c2ccncc2Nc2ncc3ccc(-c4c(F)cc(CCO)cc4F)nn23)C[C@@H]1C]

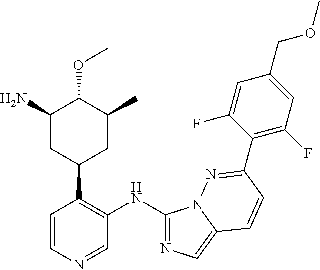 [SMILES: COCc1cc(F)c(-c2ccc3cnc(Nc4cnccc4[C@H]4C[C@@H](N)[C@H](OC)[C@@H](C)C4)n3n2)c(F)c1]

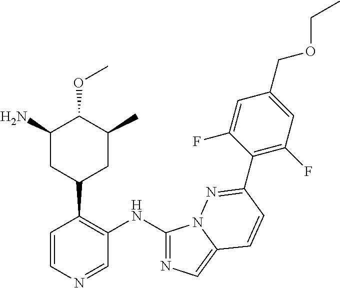

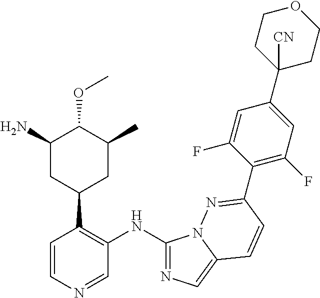

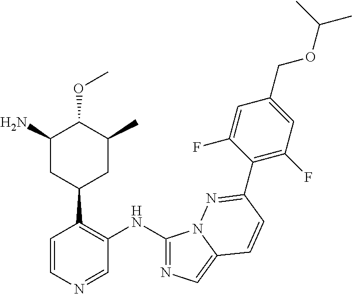 [SMILES: CO[C@H]1[C@H](N)C[C@H](c2ccncc2Nc2ncc3ccc(-c4c(F)cc(COC(C)C)cc4F)nn23)C[C@@H]1C]

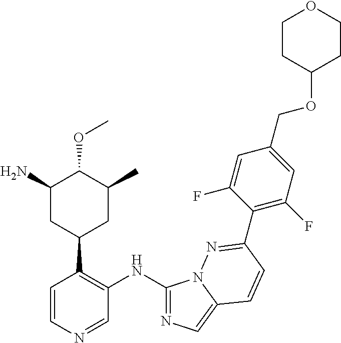 [SMILES: CO[C@H]1[C@H](N)C[C@H](c2ccncc2Nc2ncc3ccc(-c4c(F)cc(COC5CCOCC5)cc4F)nn23)C[C@@H]1C]

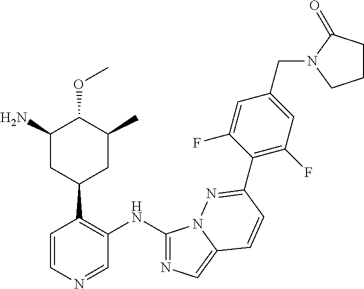 [SMILES: CO[C@H]1[C@H](N)C[C@H](c2ccncc2Nc2ncc3ccc(-c4c(F)cc(CN5CCCC5=O)cc4F)nn23)C[C@@H]1C]

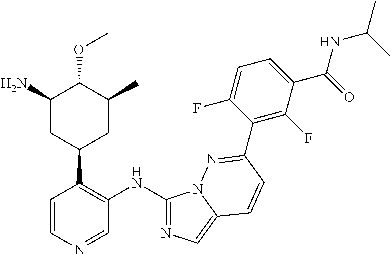 [SMILES: CO[C@H]1[C@H](N)C[C@H](c2ccncc2Nc2ncc3ccc(-c4c(F)ccc(C(=O)NC(C)C)c4F)nn23)C[C@@H]1C]

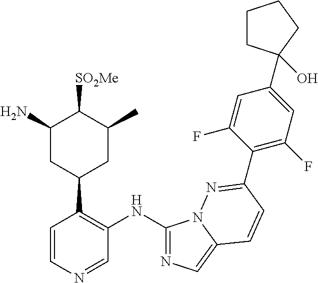 [SMILES: C[C@H]1C[C@@H](c2ccncc2Nc2ncc3ccc(-c4c(F)cc(C5(O)CCCC5)cc4F)nn23)C[C@@H](N)[C@H]1S(C)(=O)=O]

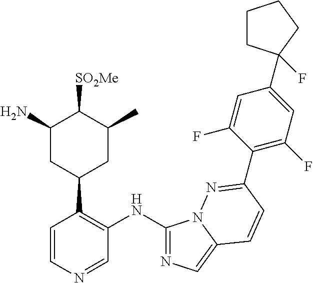 [SMILES: C[C@H]1C[C@@H](c2ccncc2Nc2ncc3ccc(-c4c(F)cc(C5(F)CCCC5)cc4F)nn23)C[C@@H](N)[C@H]1S(C)(=O)=O]

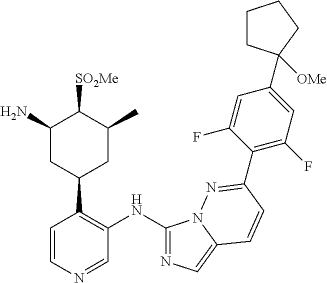 [SMILES: COC1(c2cc(F)c(-c3ccc4cnc(Nc5cnccc5[C@H]5C[C@@H](N)[C@@H](S(C)(=O)=O)[C@@H](C)C5)n4n3)c(F)c2)CCCC1]

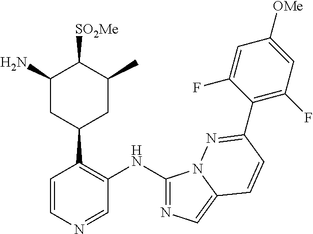 [SMILES: COc1cc(F)c(-c2ccc3cnc(Nc4cnccc4[C@H]4C[C@@H](N)[C@@H](S(C)(=O)=O)[C@@H](C)C4)n3n2)c(F)c1]

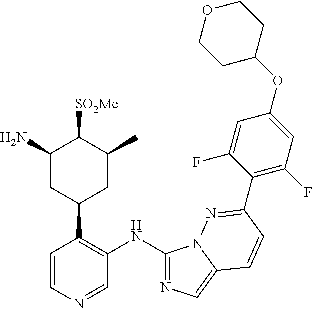 [SMILES: C[C@H]1C[C@@H](c2ccncc2Nc2ncc3ccc(-c4c(F)cc(OC5CCOCC5)cc4F)nn23)C[C@@H](N)[C@H]1S(C)(=O)=O]

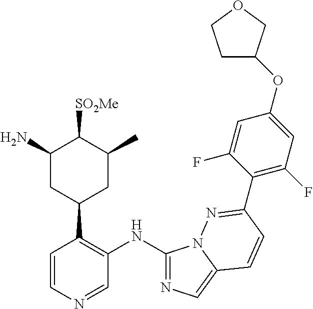 [SMILES: C[C@H]1C[C@@H](c2ccncc2Nc2ncc3ccc(-c4c(F)cc(OC5CCOC5)cc4F)nn23)C[C@@H](N)[C@H]1S(C)(=O)=O]

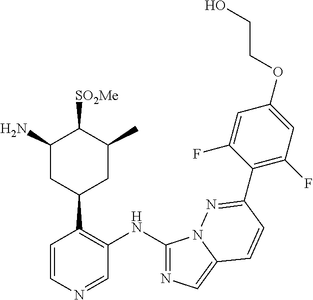 [SMILES: C[C@H]1C[C@@H](c2ccncc2Nc2ncc3ccc(-c4c(F)cc(OCCO)cc4F)nn23)C[C@@H](N)[C@H]1S(C)(=O)=O]

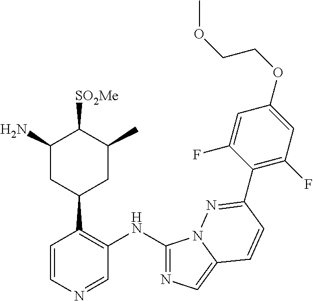 [SMILES: COCCOc1cc(F)c(-c2ccc3cnc(Nc4cnccc4[C@H]4C[C@@H](N)[C@@H](S(C)(=O)=O)[C@@H](C)C4)n3n2)c(F)c1]

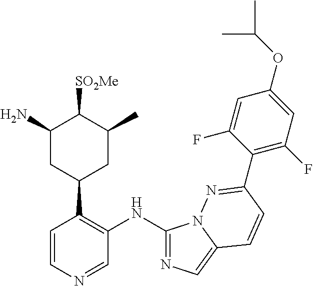 [SMILES: CC(C)Oc1cc(F)c(-c2ccc3cnc(Nc4cnccc4[C@H]4C[C@@H](N)[C@@H](S(C)(=O)=O)[C@@H](C)C4)n3n2)c(F)c1]

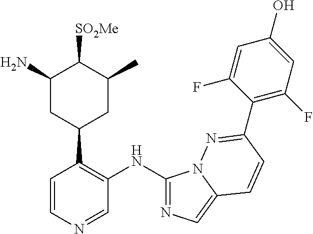 [SMILES: C[C@H]1C[C@@H](c2ccncc2Nc2ncc3ccc(-c4c(F)cc(O)cc4F)nn23)C[C@@H](N)[C@H]1S(C)(=O)=O]

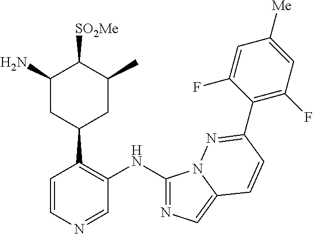 [SMILES: Cc1cc(F)c(-c2ccc3cnc(Nc4cnccc4[C@H]4C[C@@H](N)[C@@H](S(C)(=O)=O)[C@@H](C)C4)n3n2)c(F)c1]

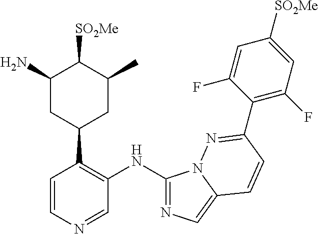 [SMILES: C[C@H]1C[C@@H](c2ccncc2Nc2ncc3ccc(-c4c(F)cc(S(C)(=O)=O)cc4F)nn23)C[C@@H](N)[C@H]1S(C)(=O)=O]

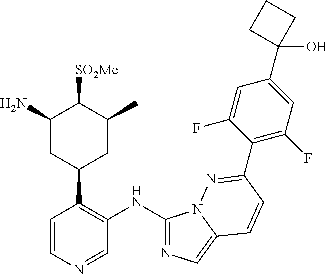 [SMILES: C[C@H]1C[C@@H](c2ccncc2Nc2ncc3ccc(-c4c(F)cc(C5(O)CCC5)cc4F)nn23)C[C@@H](N)[C@H]1S(C)(=O)=O]

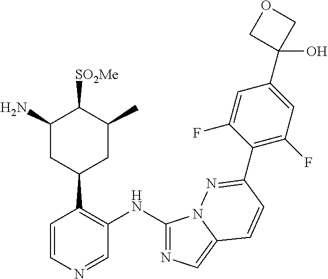 [SMILES: C[C@H]1C[C@@H](c2ccncc2Nc2ncc3ccc(-c4c(F)cc(C5(O)COC5)cc4F)nn23)C[C@@H](N)[C@H]1S(C)(=O)=O]

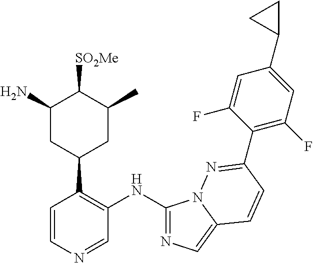 [SMILES: C[C@H]1C[C@@H](c2ccncc2Nc2ncc3ccc(-c4c(F)cc(C5CC5)cc4F)nn23)C[C@@H](N)[C@H]1S(C)(=O)=O]

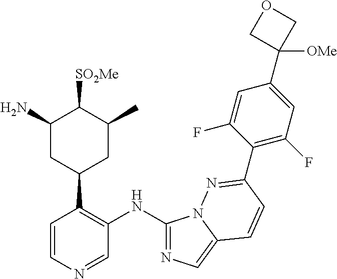 [SMILES: COC1(c2cc(F)c(-c3ccc4cnc(Nc5cnccc5[C@H]5C[C@@H](N)[C@@H](S(C)(=O)=O)[C@@H](C)C5)n4n3)c(F)c2)COC1]

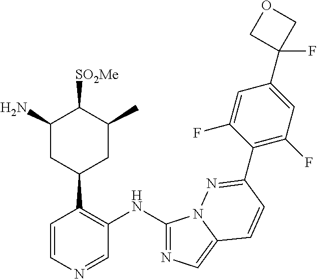 [SMILES: C[C@H]1C[C@@H](c2ccncc2Nc2ncc3ccc(-c4c(F)cc(C5(F)COC5)cc4F)nn23)C[C@@H](N)[C@H]1S(C)(=O)=O]

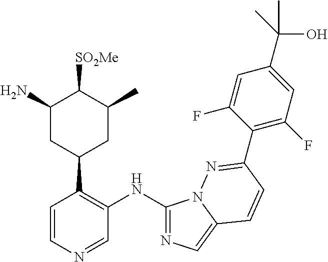 [SMILES: C[C@H]1C[C@@H](c2ccncc2Nc2ncc3ccc(-c4c(F)cc(C(C)(C)O)cc4F)nn23)C[C@@H](N)[C@H]1S(C)(=O)=O]

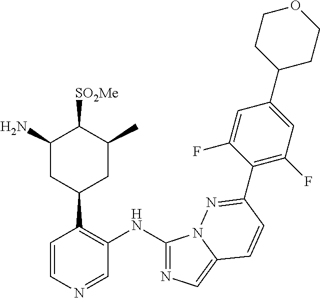 [SMILES: C[C@H]1C[C@@H](c2ccncc2Nc2ncc3ccc(-c4c(F)cc(C5CCOCC5)cc4F)nn23)C[C@@H](N)[C@H]1S(C)(=O)=O]

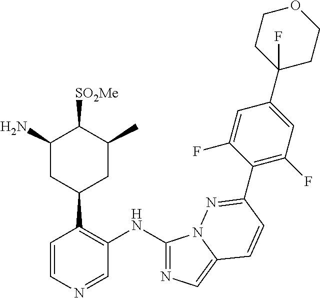

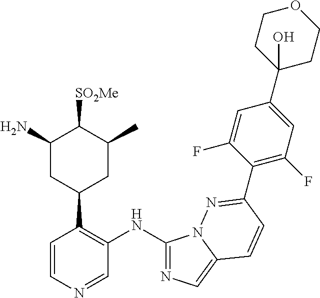

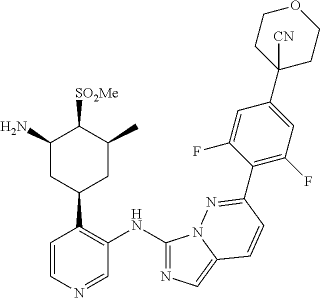

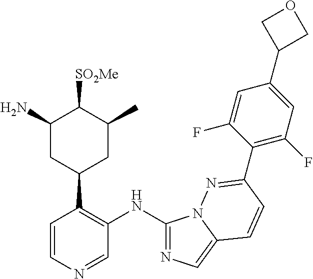 [SMILES: C[C@H]1C[C@@H](c2ccncc2Nc2ncc3ccc(-c4c(F)cc(C5COC5)cc4F)nn23)C[C@@H](N)[C@H]1S(C)(=O)=O]

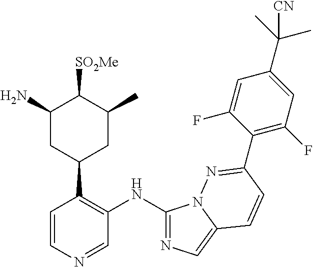 [SMILES: C[C@H]1C[C@@H](c2ccncc2Nc2ncc3ccc(-c4c(F)cc(C(C)(C)C#N)cc4F)nn23)C[C@@H](N)[C@H]1S(C)(=O)=O]

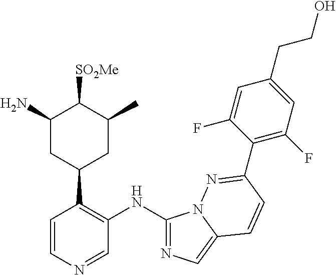 [SMILES: C[C@H]1C[C@@H](c2ccncc2Nc2ncc3ccc(-c4c(F)cc(CCO)cc4F)nn23)C[C@@H](N)[C@H]1S(C)(=O)=O]

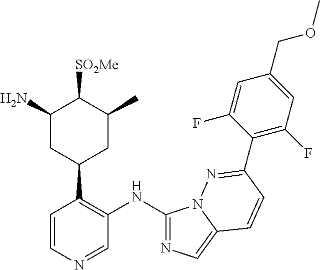 [SMILES: COCc1cc(F)c(-c2ccc3cnc(Nc4cnccc4[C@H]4C[C@@H](N)[C@@H](S(C)(=O)=O)[C@@H](C)C4)n3n2)c(F)c1]

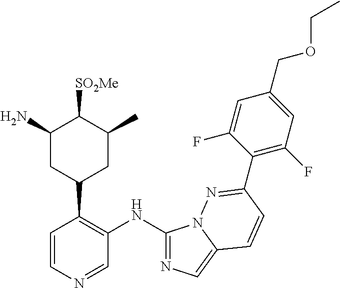

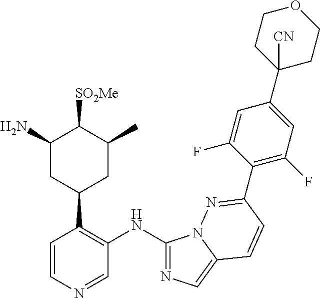

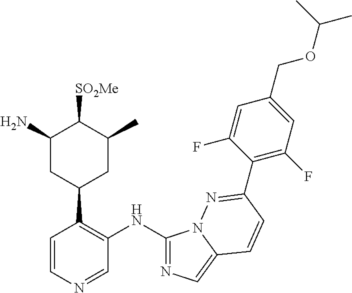 [SMILES: CC(C)OCc1cc(F)c(-c2ccc3cnc(Nc4cnccc4[C@H]4C[C@@H](N)[C@@H](S(C)(=O)=O)[C@@H](C)C4)n3n2)c(F)c1]

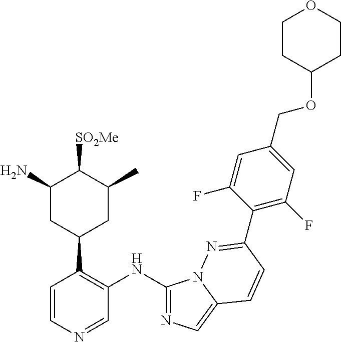 [SMILES: C[C@H]1C[C@@H](c2ccncc2Nc2ncc3ccc(-c4c(F)cc(COC5CCOCC5)cc4F)nn23)C[C@@H](N)[C@H]1S(C)(=O)=O]

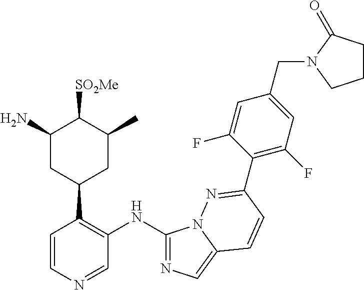 [SMILES: C[C@H]1C[C@@H](c2ccncc2Nc2ncc3ccc(-c4c(F)cc(CN5CCCC5=O)cc4F)nn23)C[C@@H](N)[C@H]1S(C)(=O)=O]

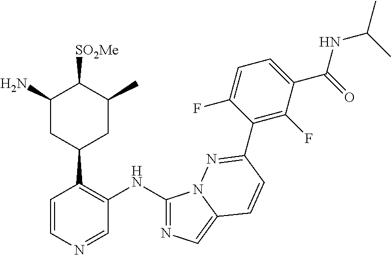 [SMILES: CC(C)NC(=O)c1ccc(F)c(-c2ccc3cnc(Nc4cnccc4[C@H]4C[C@@H](N)[C@@H](S(C)(=O)=O)[C@@H](C)C4)n3n2)c1F]

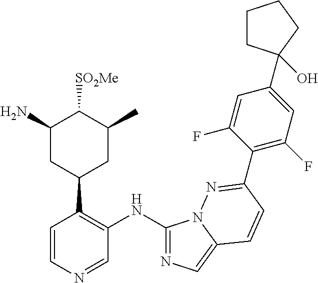 [SMILES: C[C@H]1C[C@@H](c2ccncc2Nc2ncc3ccc(-c4c(F)cc(C5(O)CCCC5)cc4F)nn23)C[C@@H](N)[C@@H]1S(C)(=O)=O]

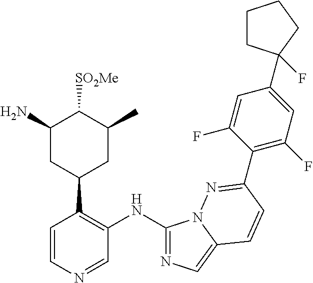 [SMILES: C[C@H]1C[C@@H](c2ccncc2Nc2ncc3ccc(-c4c(F)cc(C5(F)CCCC5)cc4F)nn23)C[C@@H](N)[C@@H]1S(C)(=O)=O]

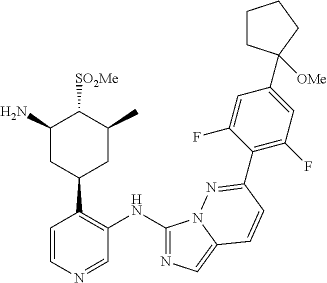 [SMILES: COC1(c2cc(F)c(-c3ccc4cnc(Nc5cnccc5[C@H]5C[C@@H](N)[C@H](S(C)(=O)=O)[C@@H](C)C5)n4n3)c(F)c2)CCCC1]

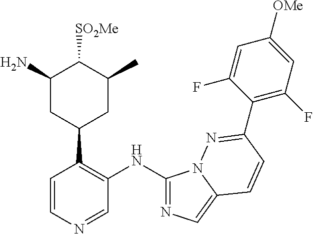 [SMILES: COc1cc(F)c(-c2ccc3cnc(Nc4cnccc4[C@H]4C[C@@H](N)[C@H](S(C)(=O)=O)[C@@H](C)C4)n3n2)c(F)c1]

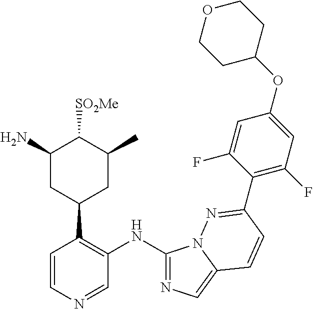 [SMILES: C[C@H]1C[C@@H](c2ccncc2Nc2ncc3ccc(-c4c(F)cc(OC5CCOCC5)cc4F)nn23)C[C@@H](N)[C@@H]1S(C)(=O)=O]

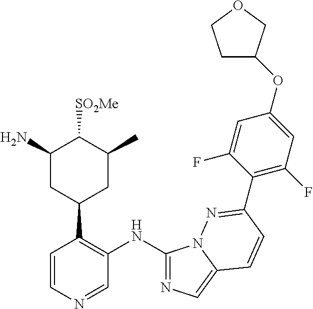 [SMILES: C[C@H]1C[C@@H](c2ccncc2Nc2ncc3ccc(-c4c(F)cc(OC5CCOC5)cc4F)nn23)C[C@@H](N)[C@@H]1S(C)(=O)=O]

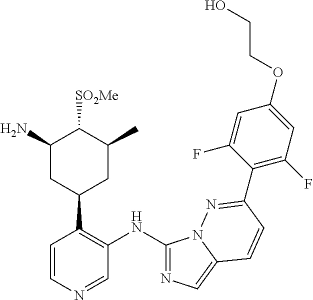 [SMILES: C[C@H]1C[C@@H](c2ccncc2Nc2ncc3ccc(-c4c(F)cc(OCCO)cc4F)nn23)C[C@@H](N)[C@@H]1S(C)(=O)=O]

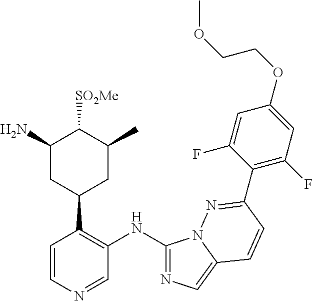 [SMILES: COCCOc1cc(F)c(-c2ccc3cnc(Nc4cnccc4[C@H]4C[C@@H](N)[C@H](S(C)(=O)=O)[C@@H](C)C4)n3n2)c(F)c1]

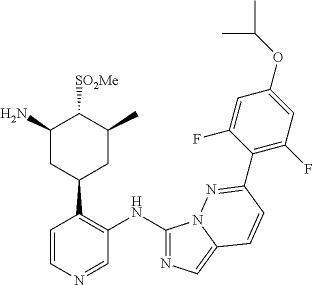 [SMILES: CC(C)Oc1cc(F)c(-c2ccc3cnc(Nc4cnccc4[C@H]4C[C@@H](N)[C@H](S(C)(=O)=O)[C@@H](C)C4)n3n2)c(F)c1]

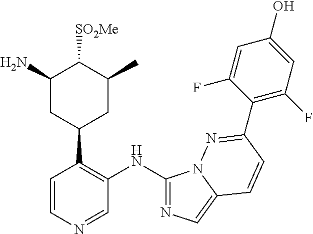 [SMILES: C[C@H]1C[C@@H](c2ccncc2Nc2ncc3ccc(-c4c(F)cc(O)cc4F)nn23)C[C@@H](N)[C@@H]1S(C)(=O)=O]

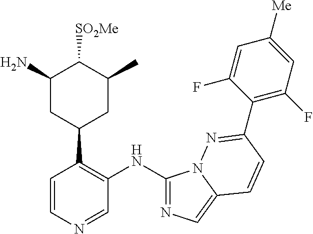 [SMILES: Cc1cc(F)c(-c2ccc3cnc(Nc4cnccc4[C@H]4C[C@@H](N)[C@H](S(C)(=O)=O)[C@@H](C)C4)n3n2)c(F)c1]

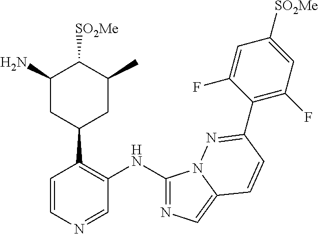 [SMILES: C[C@H]1C[C@@H](c2ccncc2Nc2ncc3ccc(-c4c(F)cc(S(C)(=O)=O)cc4F)nn23)C[C@@H](N)[C@@H]1S(C)(=O)=O]

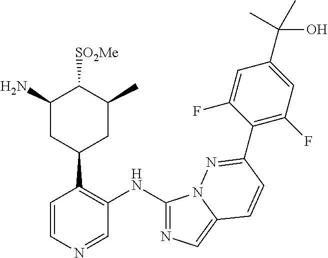 [SMILES: C[C@H]1C[C@@H](c2ccncc2Nc2ncc3ccc(-c4c(F)cc(C(C)(C)O)cc4F)nn23)C[C@@H](N)[C@@H]1S(C)(=O)=O]

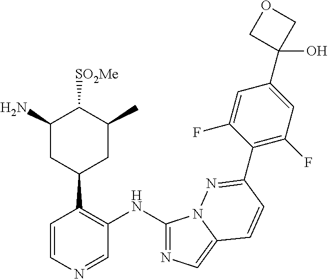 [SMILES: C[C@H]1C[C@@H](c2ccncc2Nc2ncc3ccc(-c4c(F)cc(C5(O)COC5)cc4F)nn23)C[C@@H](N)[C@@H]1S(C)(=O)=O]

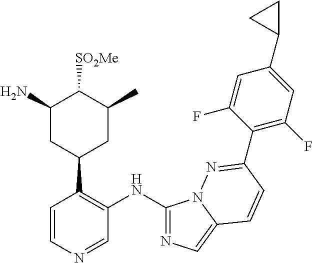 [SMILES: C[C@H]1C[C@@H](c2ccncc2Nc2ncc3ccc(-c4c(F)cc(C5CC5)cc4F)nn23)C[C@@H](N)[C@@H]1S(C)(=O)=O]

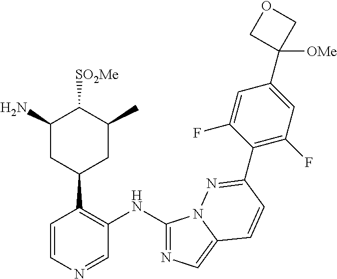 [SMILES: COC1(c2cc(F)c(-c3ccc4cnc(Nc5cnccc5[C@H]5C[C@@H](N)[C@H](S(C)(=O)=O)[C@@H](C)C5)n4n3)c(F)c2)COC1]

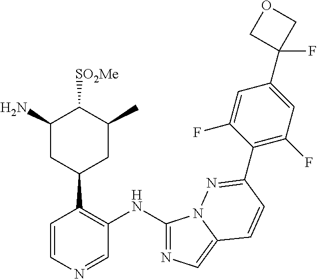 [SMILES: C[C@H]1C[C@@H](c2ccncc2Nc2ncc3ccc(-c4c(F)cc(C5(F)COC5)cc4F)nn23)C[C@@H](N)[C@@H]1S(C)(=O)=O]

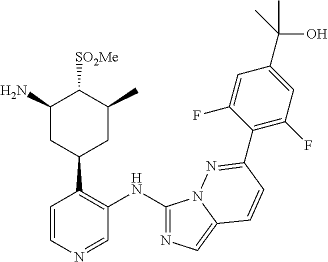 [SMILES: C[C@H]1C[C@@H](c2ccncc2Nc2ncc3ccc(-c4c(F)cc(C(C)(C)O)cc4F)nn23)C[C@@H](N)[C@@H]1S(C)(=O)=O]

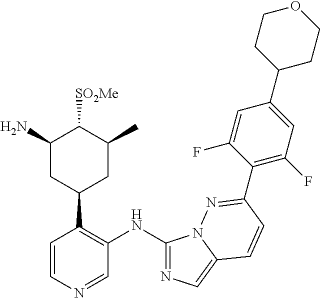 [SMILES: C[C@H]1C[C@@H](c2ccncc2Nc2ncc3ccc(-c4c(F)cc(C5CCOCC5)cc4F)nn23)C[C@@H](N)[C@@H]1S(C)(=O)=O]

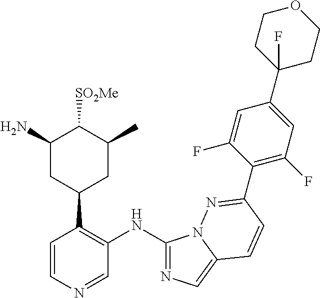

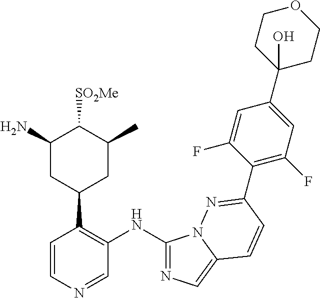

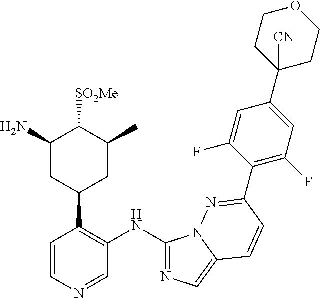

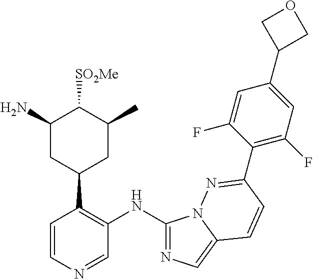 [SMILES: C[C@H]1C[C@@H](c2ccncc2Nc2ncc3ccc(-c4c(F)cc(C5COC5)cc4F)nn23)C[C@@H](N)[C@@H]1S(C)(=O)=O]

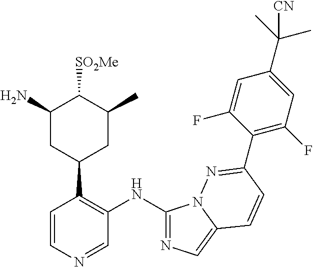 [SMILES: C[C@H]1C[C@@H](c2ccncc2Nc2ncc3ccc(-c4c(F)cc(C(C)(C)C#N)cc4F)nn23)C[C@@H](N)[C@@H]1S(C)(=O)=O]

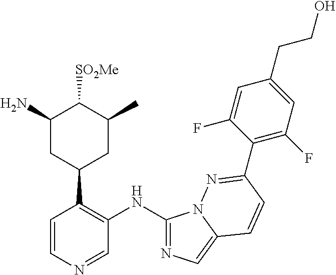 [SMILES: C[C@H]1C[C@@H](c2ccncc2Nc2ncc3ccc(-c4c(F)cc(CCO)cc4F)nn23)C[C@@H](N)[C@@H]1S(C)(=O)=O]

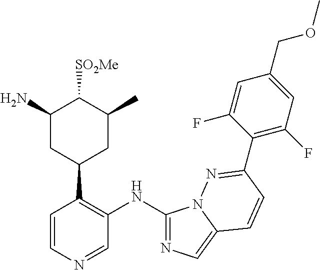 [SMILES: COCc1cc(F)c(-c2ccc3cnc(Nc4cnccc4[C@H]4C[C@@H](N)[C@H](S(C)(=O)=O)[C@@H](C)C4)n3n2)c(F)c1]

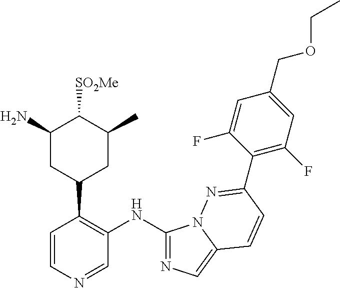

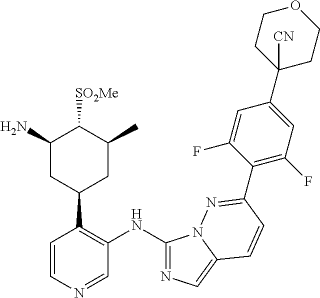

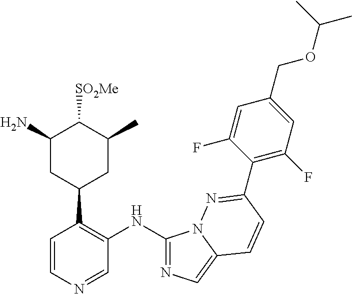 [SMILES: CC(C)OCc1cc(F)c(-c2ccc3cnc(Nc4cnccc4[C@H]4C[C@@H](N)[C@H](S(C)(=O)=O)[C@@H](C)C4)n3n2)c(F)c1]

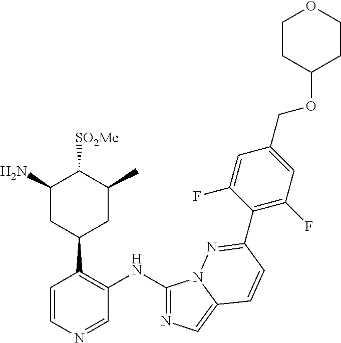 [SMILES: C[C@H]1C[C@@H](c2ccncc2Nc2ncc3ccc(-c4c(F)cc(COC5CCOCC5)cc4F)nn23)C[C@@H](N)[C@@H]1S(C)(=O)=O]

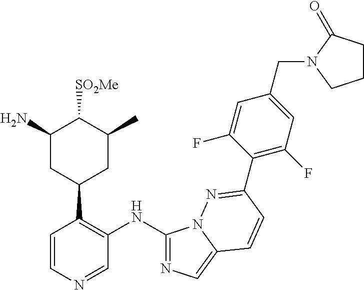 [SMILES: C[C@H]1C[C@@H](c2ccncc2Nc2ncc3ccc(-c4c(F)cc(CN5CCCC5=O)cc4F)nn23)C[C@@H](N)[C@@H]1S(C)(=O)=O]

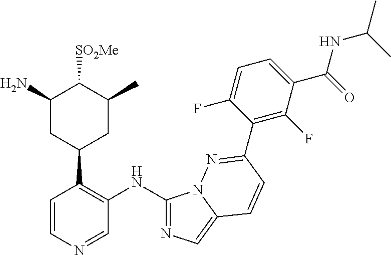 [SMILES: CC(C)NC(=O)c1ccc(F)c(-c2ccc3cnc(Nc4cnccc4[C@H]4C[C@@H](N)[C@H](S(C)(=O)=O)[C@@H](C)C4)n3n2)c1F]

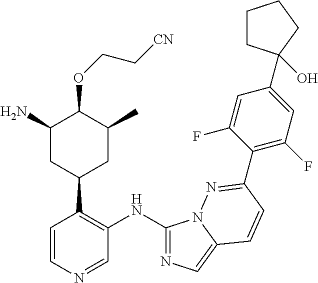 [SMILES: C[C@H]1C[C@@H](c2ccncc2Nc2ncc3ccc(-c4c(F)cc(C5(O)CCCC5)cc4F)nn23)C[C@@H](N)[C@H]1OCCC#N]

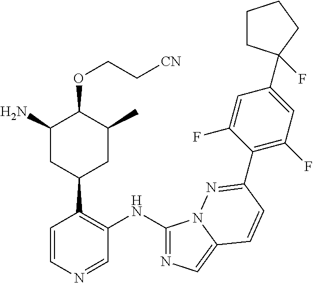 [SMILES: C[C@H]1C[C@@H](c2ccncc2Nc2ncc3ccc(-c4c(F)cc(C5(F)CCCC5)cc4F)nn23)C[C@@H](N)[C@H]1OCCC#N]

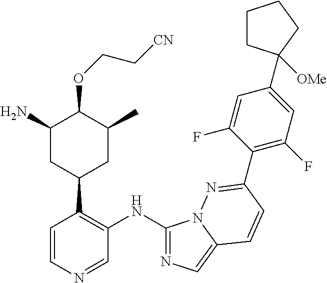 [SMILES: COC1(c2cc(F)c(-c3ccc4cnc(Nc5cnccc5[C@H]5C[C@@H](N)[C@@H](OCCC#N)[C@@H](C)C5)n4n3)c(F)c2)CCCC1]

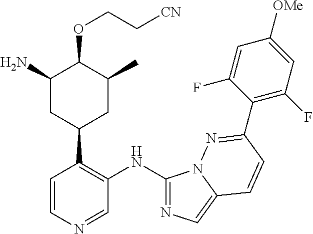 [SMILES: COc1cc(F)c(-c2ccc3cnc(Nc4cnccc4[C@H]4C[C@@H](N)[C@@H](OCCC#N)[C@@H](C)C4)n3n2)c(F)c1]

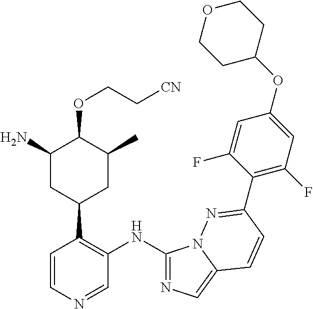 [SMILES: C[C@H]1C[C@@H](c2ccncc2Nc2ncc3ccc(-c4c(F)cc(OC5CCOCC5)cc4F)nn23)C[C@@H](N)[C@H]1OCCC#N]

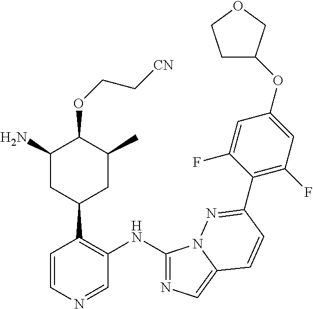 [SMILES: C[C@H]1C[C@@H](c2ccncc2Nc2ncc3ccc(-c4c(F)cc(OC5CCOC5)cc4F)nn23)C[C@@H](N)[C@H]1OCCC#N]

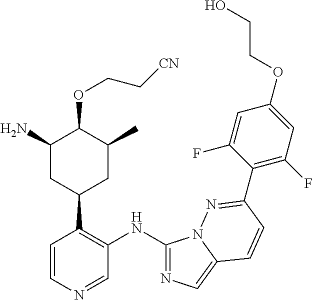 [SMILES: C[C@H]1C[C@@H](c2ccncc2Nc2ncc3ccc(-c4c(F)cc(OCCO)cc4F)nn23)C[C@@H](N)[C@H]1OCCC#N]

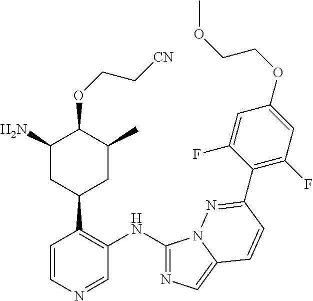 [SMILES: COCCOc1cc(F)c(-c2ccc3cnc(Nc4cnccc4[C@H]4C[C@@H](N)[C@@H](OCCC#N)[C@@H](C)C4)n3n2)c(F)c1]

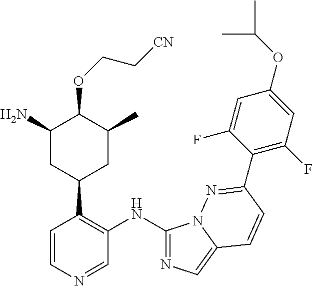 [SMILES: CC(C)Oc1cc(F)c(-c2ccc3cnc(Nc4cnccc4[C@H]4C[C@@H](N)[C@@H](OCCC#N)[C@@H](C)C4)n3n2)c(F)c1]

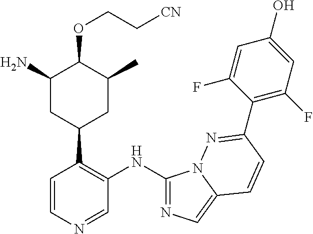 [SMILES: C[C@H]1C[C@@H](c2ccncc2Nc2ncc3ccc(-c4c(F)cc(O)cc4F)nn23)C[C@@H](N)[C@H]1OCCC#N]

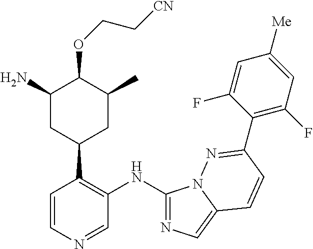 [SMILES: Cc1cc(F)c(-c2ccc3cnc(Nc4cnccc4[C@H]4C[C@@H](N)[C@@H](OCCC#N)[C@@H](C)C4)n3n2)c(F)c1]

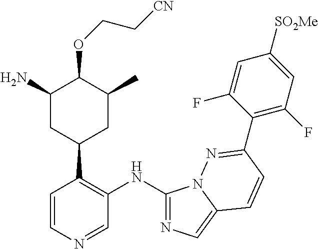 [SMILES: C[C@H]1C[C@@H](c2ccncc2Nc2ncc3ccc(-c4c(F)cc(S(C)(=O)=O)cc4F)nn23)C[C@@H](N)[C@H]1OCCC#N]

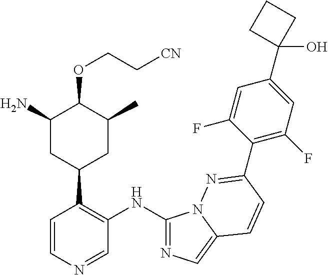 [SMILES: C[C@H]1C[C@@H](c2ccncc2Nc2ncc3ccc(-c4c(F)cc(C5(O)CCC5)cc4F)nn23)C[C@@H](N)[C@H]1OCCC#N]

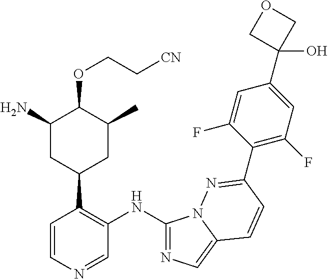 [SMILES: C[C@H]1C[C@@H](c2ccncc2Nc2ncc3ccc(-c4c(F)cc(C5(O)COC5)cc4F)nn23)C[C@@H](N)[C@H]1OCCC#N]

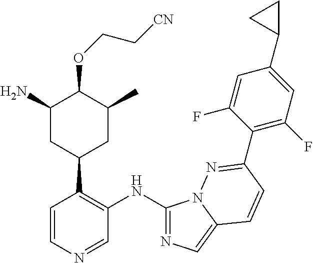 [SMILES: C[C@H]1C[C@@H](c2ccncc2Nc2ncc3ccc(-c4c(F)cc(C5CC5)cc4F)nn23)C[C@@H](N)[C@H]1OCCC#N]

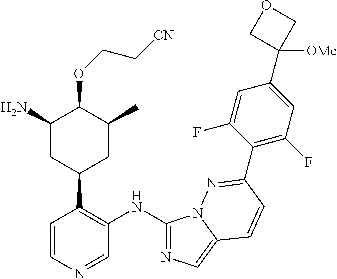 [SMILES: COC1(c2cc(F)c(-c3ccc4cnc(Nc5cnccc5[C@H]5C[C@@H](N)[C@@H](OCCC#N)[C@@H](C)C5)n4n3)c(F)c2)COC1]

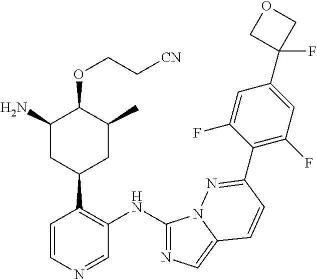 [SMILES: C[C@H]1C[C@@H](c2ccncc2Nc2ncc3ccc(-c4c(F)cc(C5(F)COC5)cc4F)nn23)C[C@@H](N)[C@H]1OCCC#N]

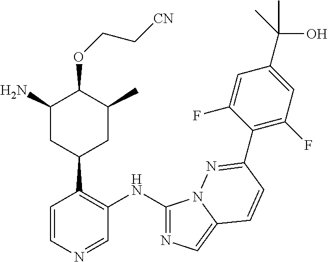 [SMILES: C[C@H]1C[C@@H](c2ccncc2Nc2ncc3ccc(-c4c(F)cc(C(C)(C)O)cc4F)nn23)C[C@@H](N)[C@H]1OCCC#N]

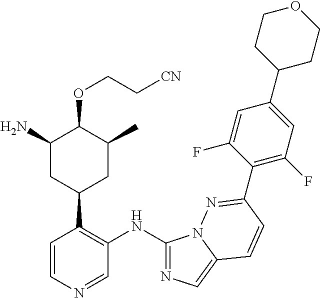 [SMILES: C[C@H]1C[C@@H](c2ccncc2Nc2ncc3ccc(-c4c(F)cc(C5CCOCC5)cc4F)nn23)C[C@@H](N)[C@H]1OCCC#N]

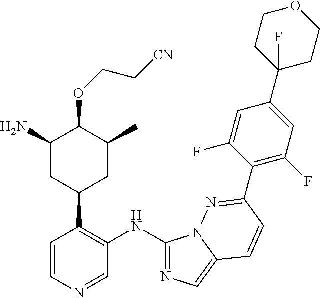

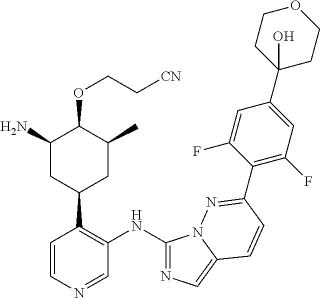

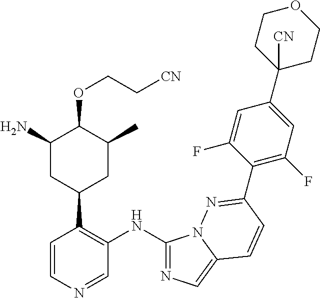

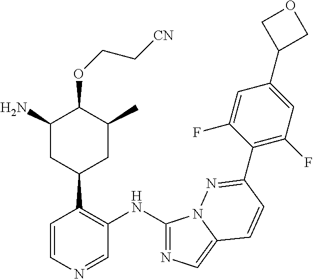 [SMILES: C[C@H]1C[C@@H](c2ccncc2Nc2ncc3ccc(-c4c(F)cc(C5COC5)cc4F)nn23)C[C@@H](N)[C@H]1OCCC#N]

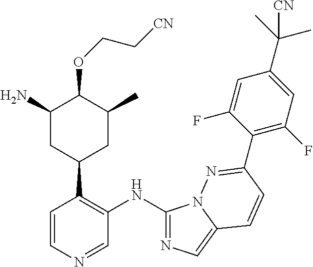 [SMILES: C[C@H]1C[C@@H](c2ccncc2Nc2ncc3ccc(-c4c(F)cc(C(C)(C)C#N)cc4F)nn23)C[C@@H](N)[C@H]1OCCC#N]

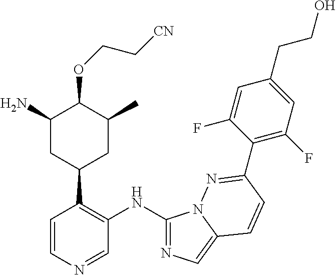 [SMILES: C[C@H]1C[C@@H](c2ccncc2Nc2ncc3ccc(-c4c(F)cc(CCO)cc4F)nn23)C[C@@H](N)[C@H]1OCCC#N]

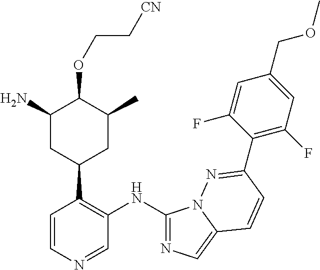 [SMILES: COCc1cc(F)c(-c2ccc3cnc(Nc4cnccc4[C@H]4C[C@@H](N)[C@@H](OCCC#N)[C@@H](C)C4)n3n2)c(F)c1]

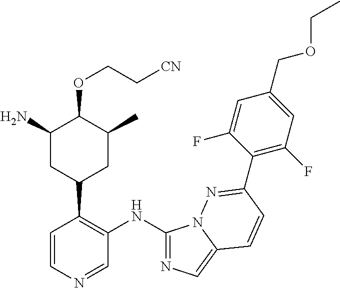

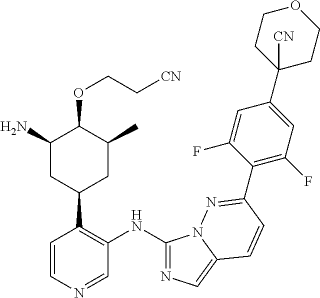

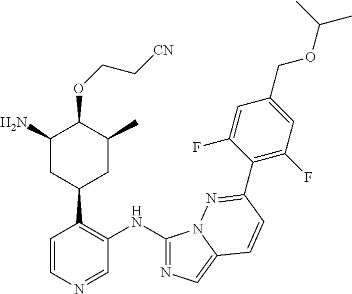 [SMILES: CC(C)OCc1cc(F)c(-c2ccc3cnc(Nc4cnccc4[C@H]4C[C@@H](N)[C@@H](OCCC#N)[C@@H](C)C4)n3n2)c(F)c1]

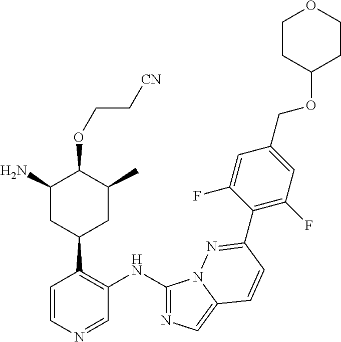 [SMILES: C[C@H]1C[C@@H](c2ccncc2Nc2ncc3ccc(-c4c(F)cc(COC5CCOCC5)cc4F)nn23)C[C@@H](N)[C@H]1OCCC#N]

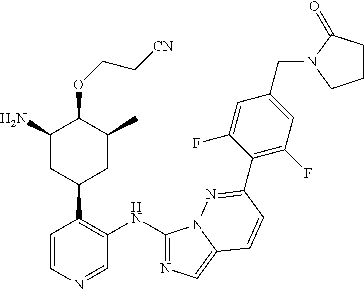 [SMILES: C[C@H]1C[C@@H](c2ccncc2Nc2ncc3ccc(-c4c(F)cc(CN5CCCC5=O)cc4F)nn23)C[C@@H](N)[C@H]1OCCC#N]

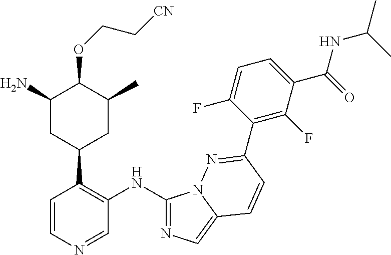 [SMILES: CC(C)NC(=O)c1ccc(F)c(-c2ccc3cnc(Nc4cnccc4[C@H]4C[C@@H](N)[C@@H](OCCC#N)[C@@H](C)C4)n3n2)c1F]

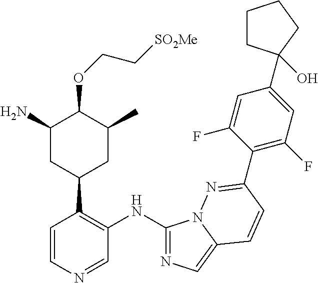 [SMILES: C[C@H]1C[C@@H](c2ccncc2Nc2ncc3ccc(-c4c(F)cc(C5(O)CCCC5)cc4F)nn23)C[C@@H](N)[C@H]1OCCS(C)(=O)=O]

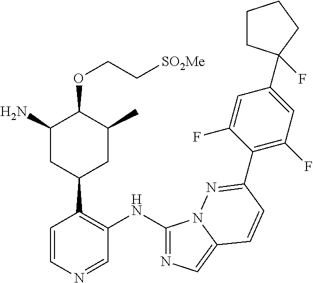 [SMILES: C[C@H]1C[C@@H](c2ccncc2Nc2ncc3ccc(-c4c(F)cc(C5(F)CCCC5)cc4F)nn23)C[C@@H](N)[C@H]1OCCS(C)(=O)=O]

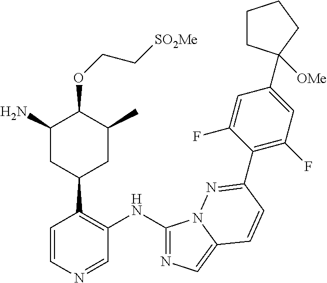 [SMILES: COC1(c2cc(F)c(-c3ccc4cnc(Nc5cnccc5[C@H]5C[C@@H](N)[C@@H](OCCS(C)(=O)=O)[C@@H](C)C5)n4n3)c(F)c2)CCCC1]

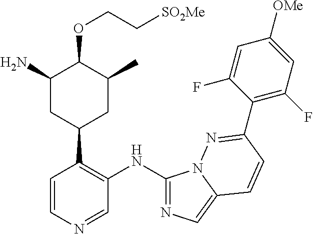 [SMILES: COc1cc(F)c(-c2ccc3cnc(Nc4cnccc4[C@H]4C[C@@H](N)[C@@H](OCCS(C)(=O)=O)[C@@H](C)C4)n3n2)c(F)c1]

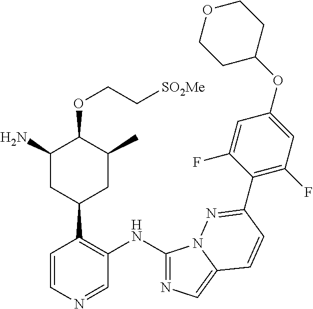 [SMILES: C[C@H]1C[C@@H](c2ccncc2Nc2ncc3ccc(-c4c(F)cc(OC5CCOCC5)cc4F)nn23)C[C@@H](N)[C@H]1OCCS(C)(=O)=O]

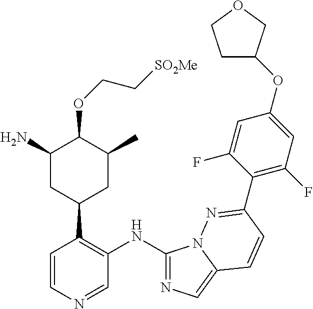 [SMILES: C[C@H]1C[C@@H](c2ccncc2Nc2ncc3ccc(-c4c(F)cc(OC5CCOC5)cc4F)nn23)C[C@@H](N)[C@H]1OCCS(C)(=O)=O]

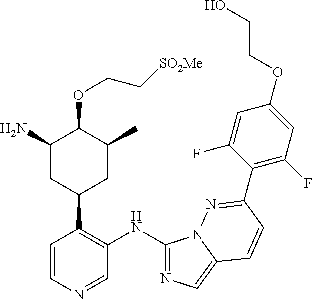 [SMILES: C[C@H]1C[C@@H](c2ccncc2Nc2ncc3ccc(-c4c(F)cc(OCCO)cc4F)nn23)C[C@@H](N)[C@H]1OCCS(C)(=O)=O]

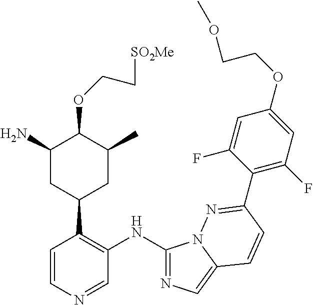 [SMILES: COCCOc1cc(F)c(-c2ccc3cnc(Nc4cnccc4[C@H]4C[C@@H](N)[C@@H](OCCS(C)(=O)=O)[C@@H](C)C4)n3n2)c(F)c1]

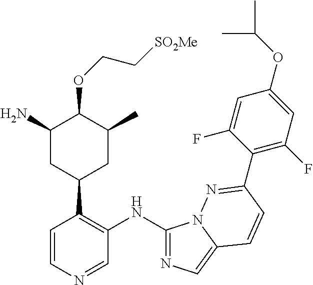 [SMILES: CC(C)Oc1cc(F)c(-c2ccc3cnc(Nc4cnccc4[C@H]4C[C@@H](N)[C@@H](OCCS(C)(=O)=O)[C@@H](C)C4)n3n2)c(F)c1]

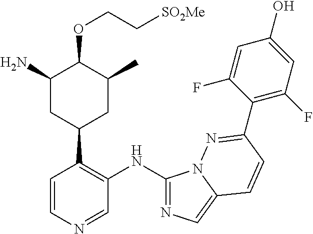 [SMILES: C[C@H]1C[C@@H](c2ccncc2Nc2ncc3ccc(-c4c(F)cc(O)cc4F)nn23)C[C@@H](N)[C@H]1OCCS(C)(=O)=O]

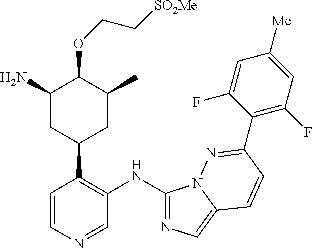 [SMILES: Cc1cc(F)c(-c2ccc3cnc(Nc4cnccc4[C@H]4C[C@@H](N)[C@@H](OCCS(C)(=O)=O)[C@@H](C)C4)n3n2)c(F)c1]

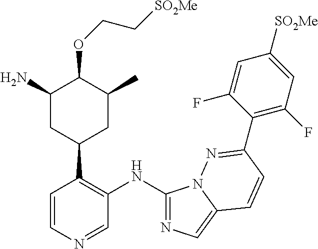 [SMILES: C[C@H]1C[C@@H](c2ccncc2Nc2ncc3ccc(-c4c(F)cc(S(C)(=O)=O)cc4F)nn23)C[C@@H](N)[C@H]1OCCS(C)(=O)=O]

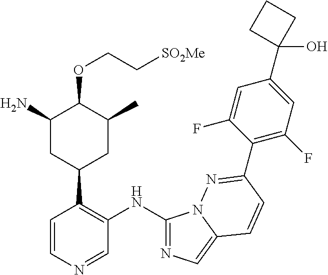 [SMILES: C[C@H]1C[C@@H](c2ccncc2Nc2ncc3ccc(-c4c(F)cc(C5(O)CCC5)cc4F)nn23)C[C@@H](N)[C@H]1OCCS(C)(=O)=O]

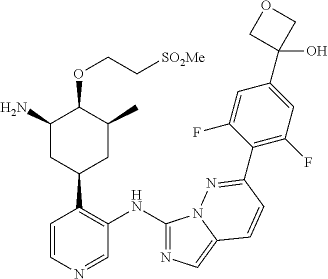 [SMILES: C[C@H]1C[C@@H](c2ccncc2Nc2ncc3ccc(-c4c(F)cc(C5(O)COC5)cc4F)nn23)C[C@@H](N)[C@H]1OCCS(C)(=O)=O]

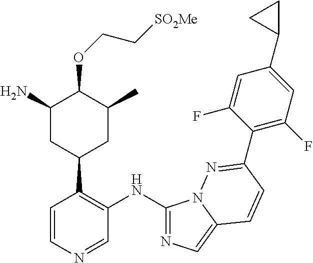 [SMILES: C[C@H]1C[C@@H](c2ccncc2Nc2ncc3ccc(-c4c(F)cc(C5CC5)cc4F)nn23)C[C@@H](N)[C@H]1OCCS(C)(=O)=O]

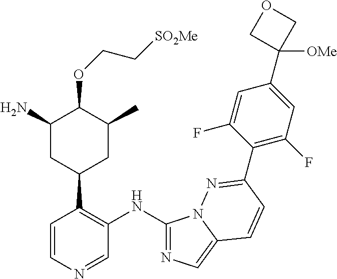 [SMILES: COC1(c2cc(F)c(-c3ccc4cnc(Nc5cnccc5[C@H]5C[C@@H](N)[C@@H](OCCS(C)(=O)=O)[C@@H](C)C5)n4n3)c(F)c2)COC1]

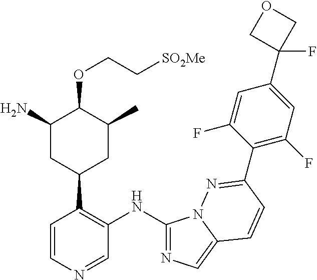 [SMILES: C[C@H]1C[C@@H](c2ccncc2Nc2ncc3ccc(-c4c(F)cc(C5(F)COC5)cc4F)nn23)C[C@@H](N)[C@H]1OCCS(C)(=O)=O]

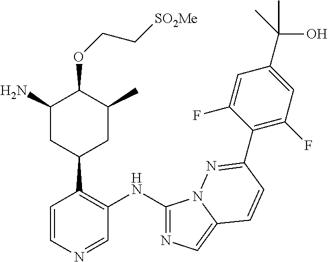 [SMILES: C[C@H]1C[C@@H](c2ccncc2Nc2ncc3ccc(-c4c(F)cc(C(C)(C)O)cc4F)nn23)C[C@@H](N)[C@H]1OCCS(C)(=O)=O]

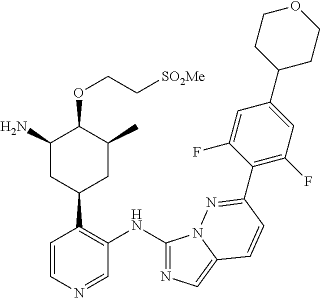 [SMILES: C[C@H]1C[C@@H](c2ccncc2Nc2ncc3ccc(-c4c(F)cc(C5CCOCC5)cc4F)nn23)C[C@@H](N)[C@H]1OCCS(C)(=O)=O]

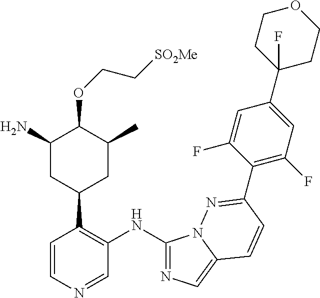

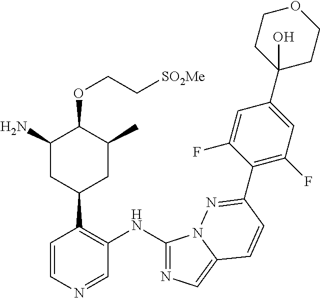

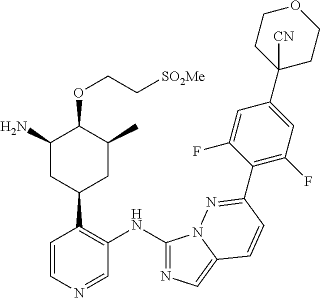

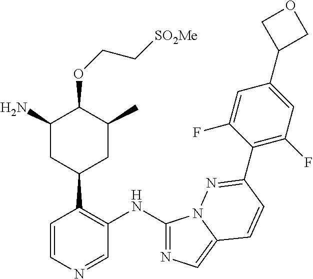 [SMILES: C[C@H]1C[C@@H](c2ccncc2Nc2ncc3ccc(-c4c(F)cc(C5COC5)cc4F)nn23)C[C@@H](N)[C@H]1OCCS(C)(=O)=O]

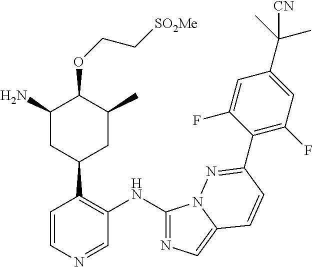 [SMILES: C[C@H]1C[C@@H](c2ccncc2Nc2ncc3ccc(-c4c(F)cc(C(C)(C)C#N)cc4F)nn23)C[C@@H](N)[C@H]1OCCS(C)(=O)=O]

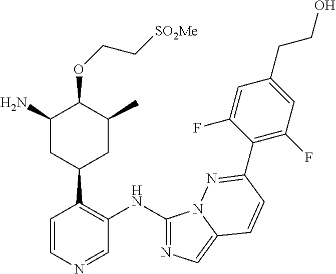 [SMILES: C[C@H]1C[C@@H](c2ccncc2Nc2ncc3ccc(-c4c(F)cc(CCO)cc4F)nn23)C[C@@H](N)[C@H]1OCCS(C)(=O)=O]

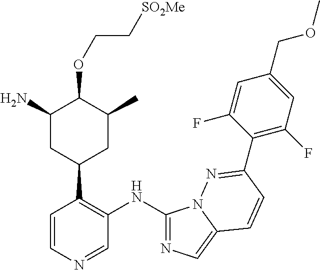 [SMILES: COCc1cc(F)c(-c2ccc3cnc(Nc4cnccc4[C@H]4C[C@@H](N)[C@@H](OCCS(C)(=O)=O)[C@@H](C)C4)n3n2)c(F)c1]

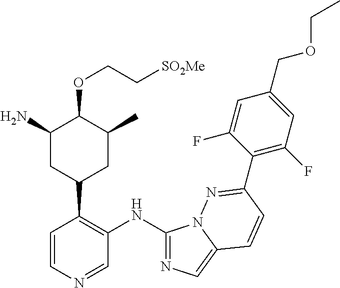

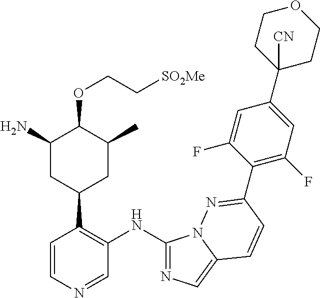

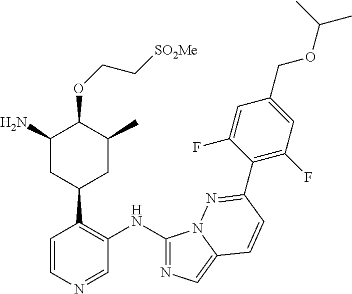 [SMILES: CC(C)OCc1cc(F)c(-c2ccc3cnc(Nc4cnccc4[C@H]4C[C@@H](N)[C@@H](OCCS(C)(=O)=O)[C@@H](C)C4)n3n2)c(F)c1]

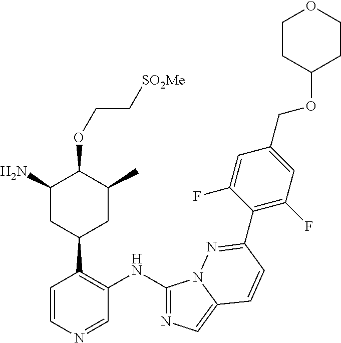 [SMILES: C[C@H]1C[C@@H](c2ccncc2Nc2ncc3ccc(-c4c(F)cc(COC5CCOCC5)cc4F)nn23)C[C@@H](N)[C@H]1OCCS(C)(=O)=O]

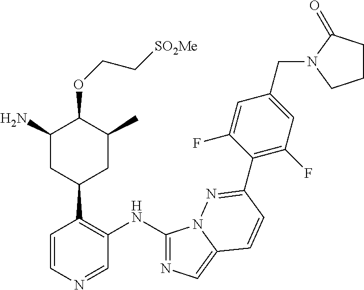 [SMILES: C[C@H]1C[C@@H](c2ccncc2Nc2ncc3ccc(-c4c(F)cc(CN5CCCC5=O)cc4F)nn23)C[C@@H](N)[C@H]1OCCS(C)(=O)=O]

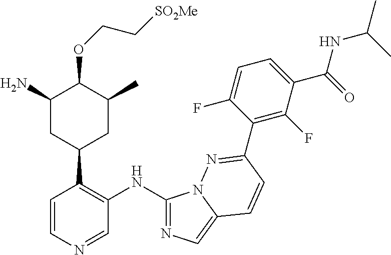 [SMILES: CC(C)NC(=O)c1ccc(F)c(-c2ccc3cnc(Nc4cnccc4[C@H]4C[C@@H](N)[C@@H](OCCS(C)(=O)=O)[C@@H](C)C4)n3n2)c1F]

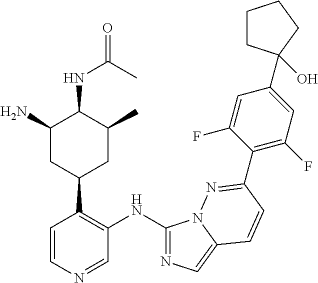 [SMILES: CC(=O)N[C@@H]1[C@H](N)C[C@H](c2ccncc2Nc2ncc3ccc(-c4c(F)cc(C5(O)CCCC5)cc4F)nn23)C[C@@H]1C]

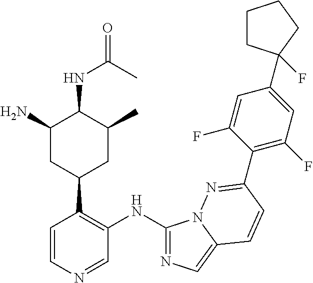 [SMILES: CC(=O)N[C@@H]1[C@H](N)C[C@H](c2ccncc2Nc2ncc3ccc(-c4c(F)cc(C5(F)CCCC5)cc4F)nn23)C[C@@H]1C]

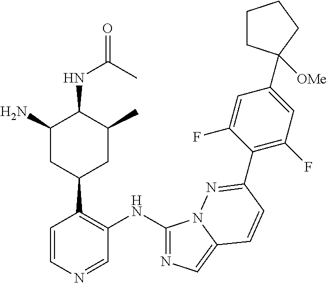 [SMILES: COC1(c2cc(F)c(-c3ccc4cnc(Nc5cnccc5[C@H]5C[C@@H](N)[C@@H](NC(C)=O)[C@@H](C)C5)n4n3)c(F)c2)CCCC1]

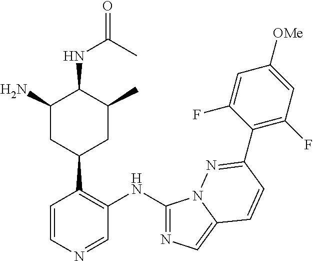 [SMILES: COc1cc(F)c(-c2ccc3cnc(Nc4cnccc4[C@H]4C[C@@H](N)[C@@H](NC(C)=O)[C@@H](C)C4)n3n2)c(F)c1]

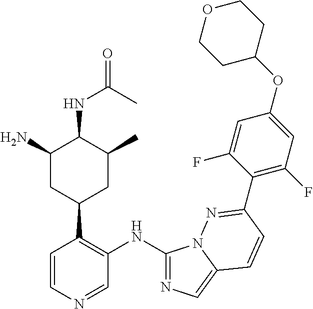 [SMILES: CC(=O)N[C@@H]1[C@H](N)C[C@H](c2ccncc2Nc2ncc3ccc(-c4c(F)cc(OC5CCOCC5)cc4F)nn23)C[C@@H]1C]

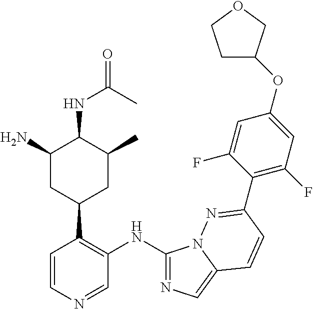 [SMILES: CC(=O)N[C@@H]1[C@H](N)C[C@H](c2ccncc2Nc2ncc3ccc(-c4c(F)cc(OC5CCOC5)cc4F)nn23)C[C@@H]1C]

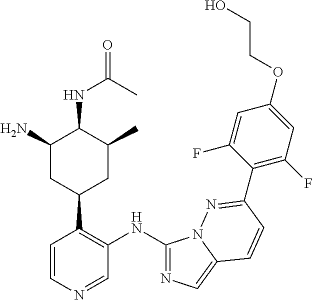 [SMILES: CC(=O)N[C@@H]1[C@H](N)C[C@H](c2ccncc2Nc2ncc3ccc(-c4c(F)cc(OCCO)cc4F)nn23)C[C@@H]1C]

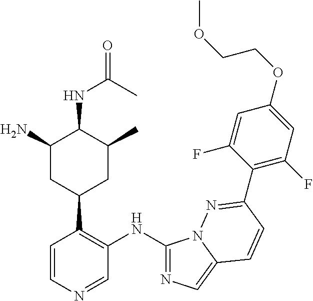 [SMILES: COCCOc1cc(F)c(-c2ccc3cnc(Nc4cnccc4[C@H]4C[C@@H](N)[C@@H](NC(C)=O)[C@@H](C)C4)n3n2)c(F)c1]

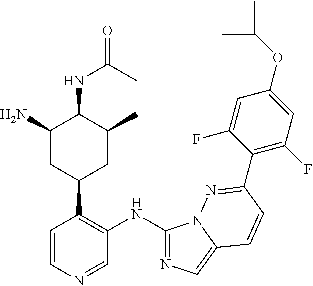 [SMILES: CC(=O)N[C@@H]1[C@H](N)C[C@H](c2ccncc2Nc2ncc3ccc(-c4c(F)cc(OC(C)C)cc4F)nn23)C[C@@H]1C]

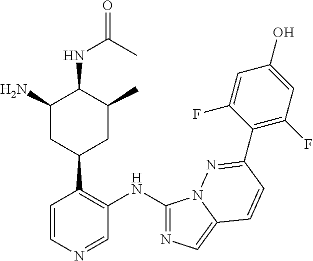 [SMILES: CC(=O)N[C@@H]1[C@H](N)C[C@H](c2ccncc2Nc2ncc3ccc(-c4c(F)cc(O)cc4F)nn23)C[C@@H]1C]

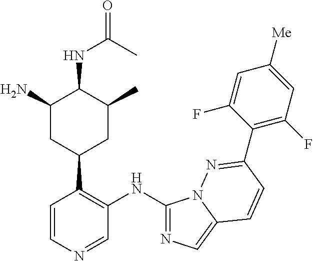 [SMILES: CC(=O)N[C@@H]1[C@H](N)C[C@H](c2ccncc2Nc2ncc3ccc(-c4c(F)cc(C)cc4F)nn23)C[C@@H]1C]

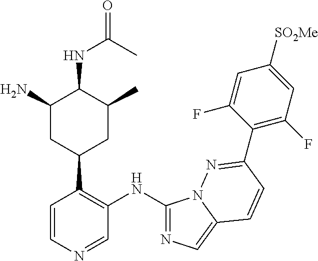 [SMILES: CC(=O)N[C@@H]1[C@H](N)C[C@H](c2ccncc2Nc2ncc3ccc(-c4c(F)cc(S(C)(=O)=O)cc4F)nn23)C[C@@H]1C]

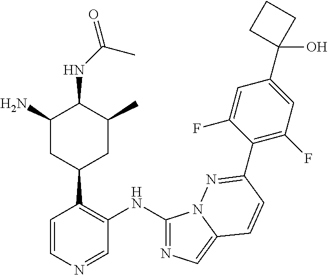 [SMILES: CC(=O)N[C@@H]1[C@H](N)C[C@H](c2ccncc2Nc2ncc3ccc(-c4c(F)cc(C5(O)CCC5)cc4F)nn23)C[C@@H]1C]

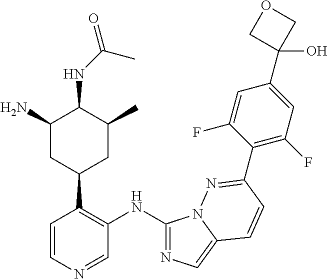 [SMILES: CC(=O)N[C@@H]1[C@H](N)C[C@H](c2ccncc2Nc2ncc3ccc(-c4c(F)cc(C5(O)COC5)cc4F)nn23)C[C@@H]1C]

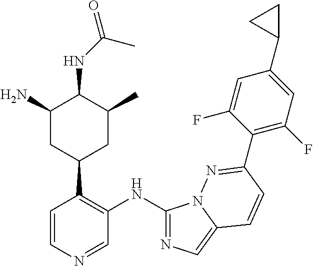 [SMILES: CC(=O)N[C@@H]1[C@H](N)C[C@H](c2ccncc2Nc2ncc3ccc(-c4c(F)cc(C5CC5)cc4F)nn23)C[C@@H]1C]

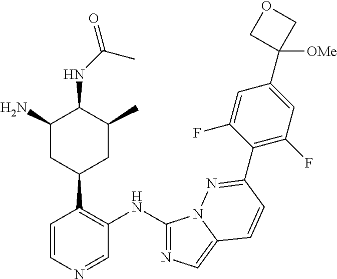 [SMILES: COC1(c2cc(F)c(-c3ccc4cnc(Nc5cnccc5[C@H]5C[C@@H](N)[C@@H](NC(C)=O)[C@@H](C)C5)n4n3)c(F)c2)COC1]

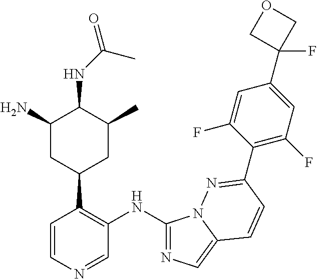 [SMILES: CC(=O)N[C@@H]1[C@H](N)C[C@H](c2ccncc2Nc2ncc3ccc(-c4c(F)cc(C5(F)COC5)cc4F)nn23)C[C@@H]1C]

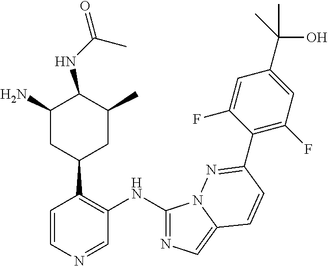 [SMILES: CC(=O)N[C@@H]1[C@H](N)C[C@H](c2ccncc2Nc2ncc3ccc(-c4c(F)cc(C(C)(C)O)cc4F)nn23)C[C@@H]1C]

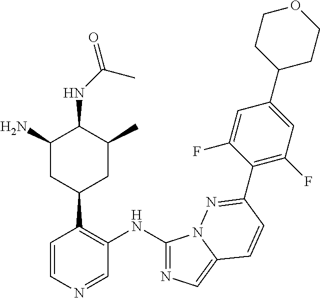 [SMILES: CC(=O)N[C@@H]1[C@H](N)C[C@H](c2ccncc2Nc2ncc3ccc(-c4c(F)cc(C5CCOCC5)cc4F)nn23)C[C@@H]1C]

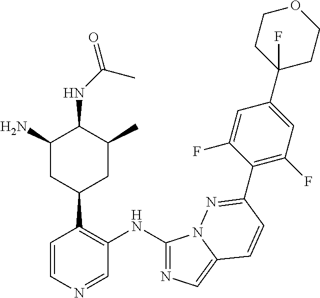

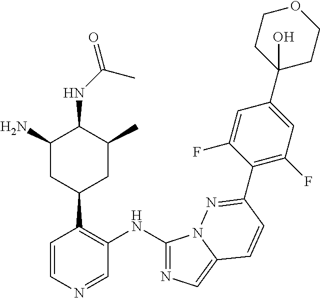

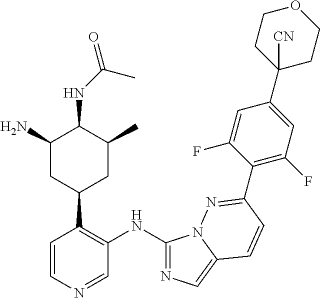

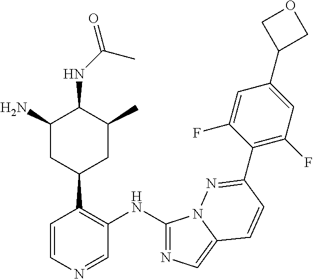 [SMILES: CC(=O)N[C@@H]1[C@H](N)C[C@H](c2ccncc2Nc2ncc3ccc(-c4c(F)cc(C5COC5)cc4F)nn23)C[C@@H]1C]

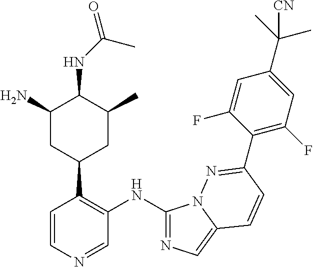 [SMILES: CC(=O)N[C@@H]1[C@H](N)C[C@H](c2ccncc2Nc2ncc3ccc(-c4c(F)cc(C(C)(C)C#N)cc4F)nn23)C[C@@H]1C]

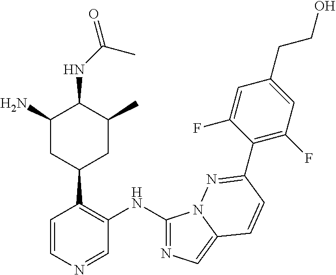 [SMILES: CC(=O)N[C@@H]1[C@H](N)C[C@H](c2ccncc2Nc2ncc3ccc(-c4c(F)cc(CCO)cc4F)nn23)C[C@@H]1C]

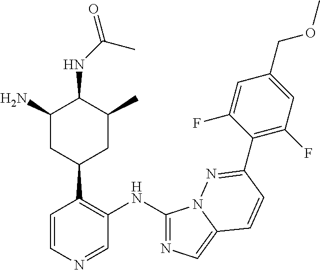 [SMILES: COCc1cc(F)c(-c2ccc3cnc(Nc4cnccc4[C@H]4C[C@@H](N)[C@@H](NC(C)=O)[C@@H](C)C4)n3n2)c(F)c1]

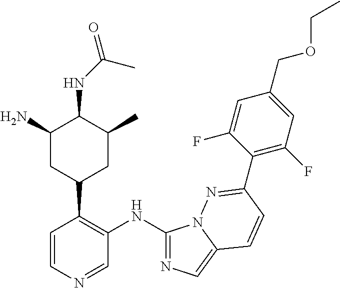

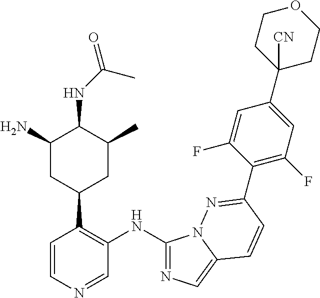

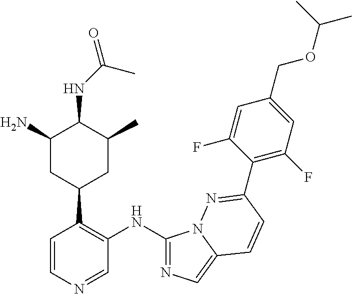 [SMILES: CC(=O)N[C@@H]1[C@H](N)C[C@H](c2ccncc2Nc2ncc3ccc(-c4c(F)cc(COC(C)C)cc4F)nn23)C[C@@H]1C]

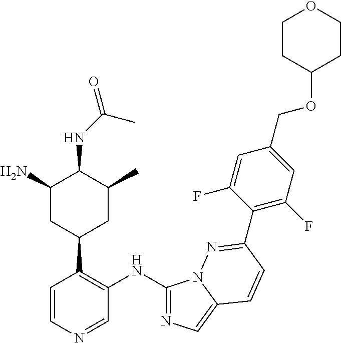 [SMILES: CC(=O)N[C@@H]1[C@H](N)C[C@H](c2ccncc2Nc2ncc3ccc(-c4c(F)cc(COC5CCOCC5)cc4F)nn23)C[C@@H]1C]

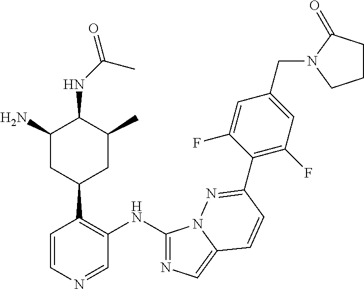 [SMILES: CC(=O)N[C@@H]1[C@H](N)C[C@H](c2ccncc2Nc2ncc3ccc(-c4c(F)cc(CN5CCCC5=O)cc4F)nn23)C[C@@H]1C]

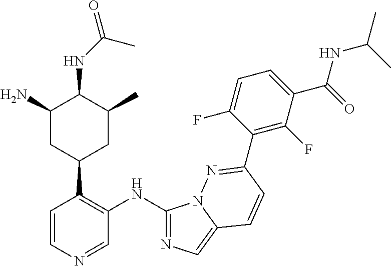 [SMILES: CC(=O)N[C@@H]1[C@H](N)C[C@H](c2ccncc2Nc2ncc3ccc(-c4c(F)ccc(C(=O)NC(C)C)c4F)nn23)C[C@@H]1C]

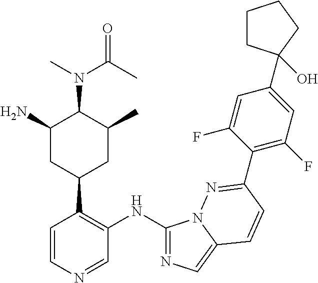 [SMILES: CC(=O)N(C)[C@@H]1[C@H](N)C[C@H](c2ccncc2Nc2ncc3ccc(-c4c(F)cc(C5(O)CCCC5)cc4F)nn23)C[C@@H]1C]

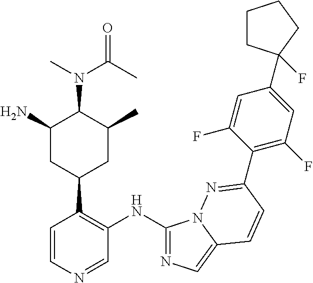 [SMILES: CC(=O)N(C)[C@@H]1[C@H](N)C[C@H](c2ccncc2Nc2ncc3ccc(-c4c(F)cc(C5(F)CCCC5)cc4F)nn23)C[C@@H]1C]

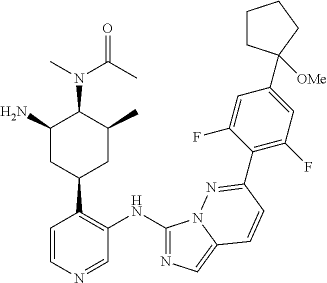 [SMILES: COC1(c2cc(F)c(-c3ccc4cnc(Nc5cnccc5[C@H]5C[C@@H](N)[C@@H](N(C)C(C)=O)[C@@H](C)C5)n4n3)c(F)c2)CCCC1]

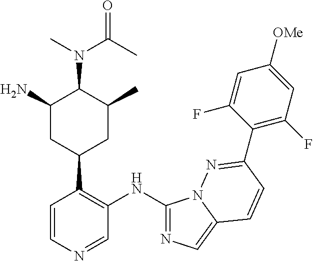 [SMILES: COc1cc(F)c(-c2ccc3cnc(Nc4cnccc4[C@H]4C[C@@H](N)[C@@H](N(C)C(C)=O)[C@@H](C)C4)n3n2)c(F)c1]

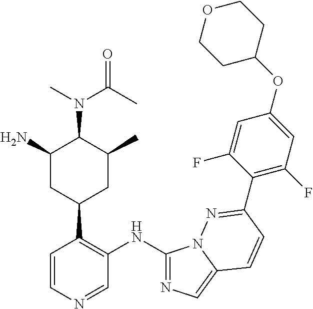 [SMILES: CC(=O)N(C)[C@@H]1[C@H](N)C[C@H](c2ccncc2Nc2ncc3ccc(-c4c(F)cc(OC5CCOCC5)cc4F)nn23)C[C@@H]1C]

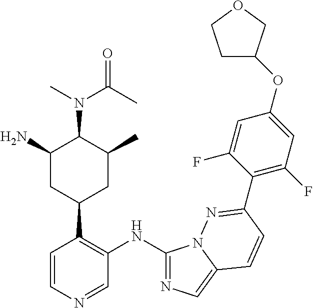 [SMILES: CC(=O)N(C)[C@@H]1[C@H](N)C[C@H](c2ccncc2Nc2ncc3ccc(-c4c(F)cc(OC5CCOC5)cc4F)nn23)C[C@@H]1C]

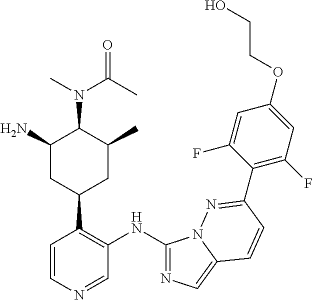 [SMILES: CC(=O)N(C)[C@@H]1[C@H](N)C[C@H](c2ccncc2Nc2ncc3ccc(-c4c(F)cc(OCCO)cc4F)nn23)C[C@@H]1C]

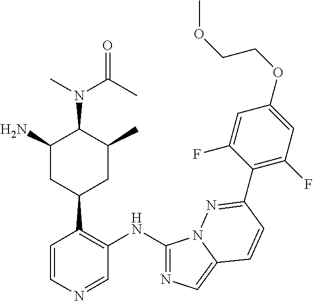 [SMILES: COCCOc1cc(F)c(-c2ccc3cnc(Nc4cnccc4[C@H]4C[C@@H](N)[C@@H](N(C)C(C)=O)[C@@H](C)C4)n3n2)c(F)c1]

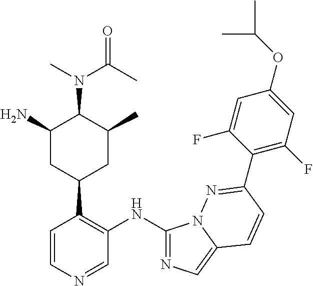 [SMILES: CC(=O)N(C)[C@@H]1[C@H](N)C[C@H](c2ccncc2Nc2ncc3ccc(-c4c(F)cc(OC(C)C)cc4F)nn23)C[C@@H]1C]

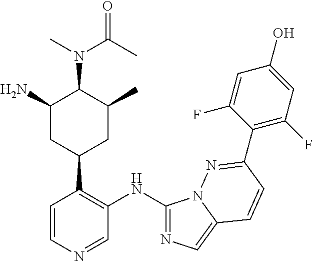 [SMILES: CC(=O)N(C)[C@@H]1[C@H](N)C[C@H](c2ccncc2Nc2ncc3ccc(-c4c(F)cc(O)cc4F)nn23)C[C@@H]1C]

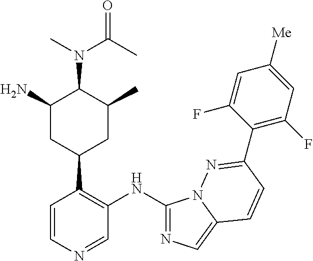 [SMILES: CC(=O)N(C)[C@@H]1[C@H](N)C[C@H](c2ccncc2Nc2ncc3ccc(-c4c(F)cc(C)cc4F)nn23)C[C@@H]1C]

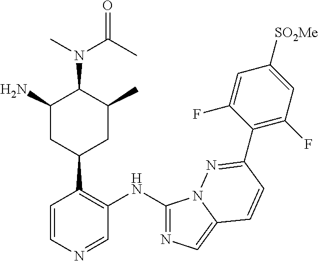 [SMILES: CC(=O)N(C)[C@@H]1[C@H](N)C[C@H](c2ccncc2Nc2ncc3ccc(-c4c(F)cc(S(C)(=O)=O)cc4F)nn23)C[C@@H]1C]

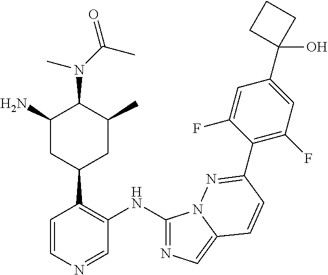 [SMILES: CC(=O)N(C)[C@@H]1[C@H](N)C[C@H](c2ccncc2Nc2ncc3ccc(-c4c(F)cc(C5(O)CCC5)cc4F)nn23)C[C@@H]1C]

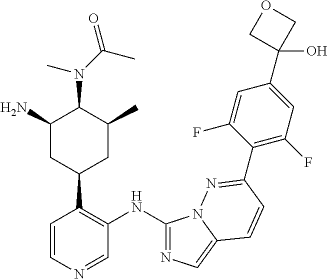 [SMILES: CC(=O)N(C)[C@@H]1[C@H](N)C[C@H](c2ccncc2Nc2ncc3ccc(-c4c(F)cc(C5(O)COC5)cc4F)nn23)C[C@@H]1C]

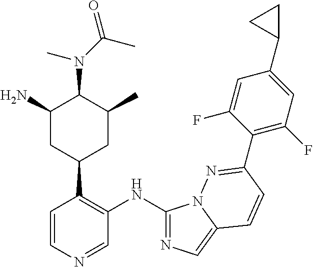 [SMILES: CC(=O)N(C)[C@@H]1[C@H](N)C[C@H](c2ccncc2Nc2ncc3ccc(-c4c(F)cc(C5CC5)cc4F)nn23)C[C@@H]1C]

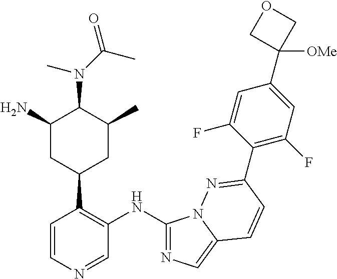 [SMILES: COC1(c2cc(F)c(-c3ccc4cnc(Nc5cnccc5[C@H]5C[C@@H](N)[C@@H](N(C)C(C)=O)[C@@H](C)C5)n4n3)c(F)c2)COC1]

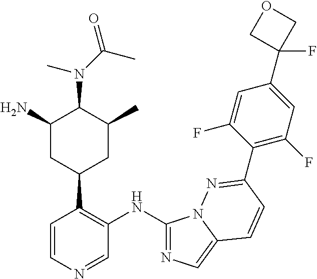 [SMILES: CC(=O)N(C)[C@@H]1[C@H](N)C[C@H](c2ccncc2Nc2ncc3ccc(-c4c(F)cc(C5(F)COC5)cc4F)nn23)C[C@@H]1C]

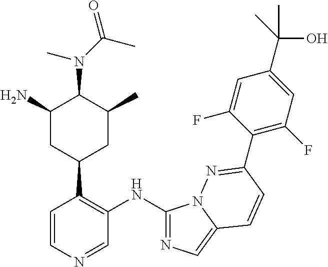 [SMILES: CC(=O)N(C)[C@@H]1[C@H](N)C[C@H](c2ccncc2Nc2ncc3ccc(-c4c(F)cc(C(C)(C)O)cc4F)nn23)C[C@@H]1C]

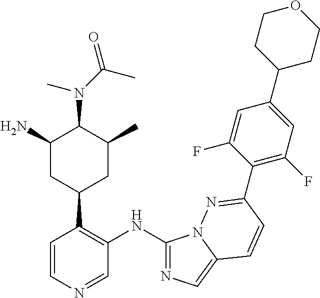 [SMILES: CC(=O)N(C)[C@@H]1[C@H](N)C[C@H](c2ccncc2Nc2ncc3ccc(-c4c(F)cc(C5CCOCC5)cc4F)nn23)C[C@@H]1C]

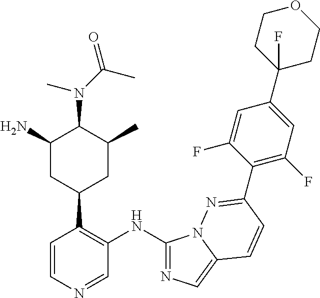

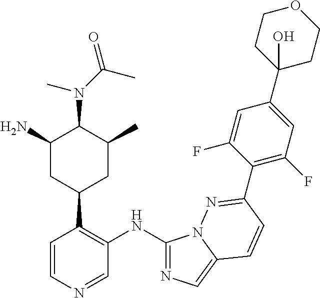

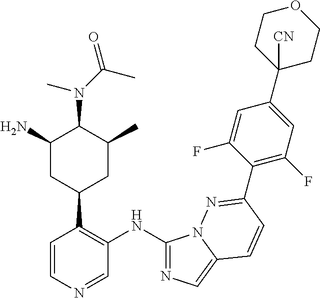

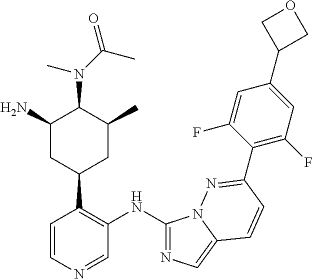 [SMILES: CC(=O)N(C)[C@@H]1[C@H](N)C[C@H](c2ccncc2Nc2ncc3ccc(-c4c(F)cc(C5COC5)cc4F)nn23)C[C@@H]1C]

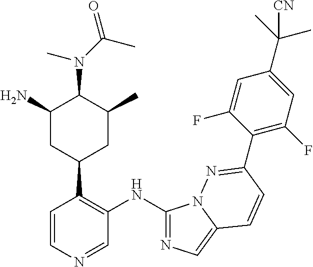 [SMILES: CC(=O)N(C)[C@@H]1[C@H](N)C[C@H](c2ccncc2Nc2ncc3ccc(-c4c(F)cc(C(C)(C)C#N)cc4F)nn23)C[C@@H]1C]

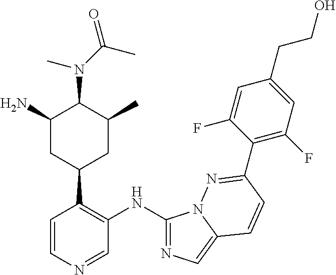 [SMILES: CC(=O)N(C)[C@@H]1[C@H](N)C[C@H](c2ccncc2Nc2ncc3ccc(-c4c(F)cc(CCO)cc4F)nn23)C[C@@H]1C]

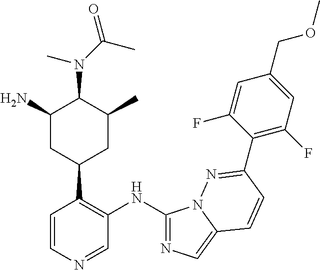 [SMILES: COCc1cc(F)c(-c2ccc3cnc(Nc4cnccc4[C@H]4C[C@@H](N)[C@@H](N(C)C(C)=O)[C@@H](C)C4)n3n2)c(F)c1]

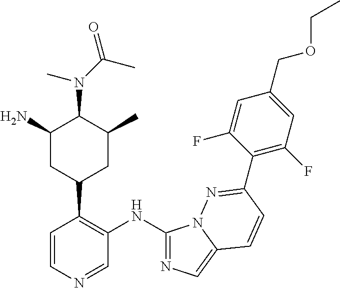

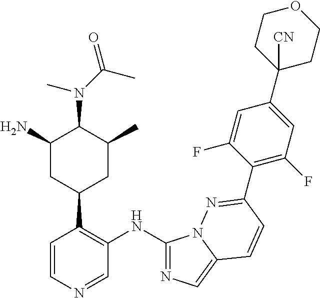

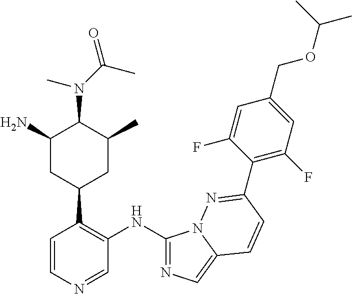 [SMILES: CC(=O)N(C)[C@@H]1[C@H](N)C[C@H](c2ccncc2Nc2ncc3ccc(-c4c(F)cc(COC(C)C)cc4F)nn23)C[C@@H]1C]

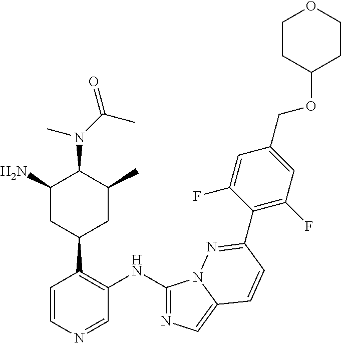 [SMILES: CC(=O)N(C)[C@@H]1[C@H](N)C[C@H](c2ccncc2Nc2ncc3ccc(-c4c(F)cc(COC5CCOCC5)cc4F)nn23)C[C@@H]1C]

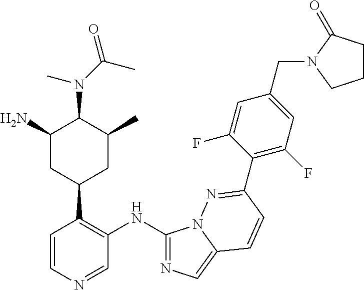 [SMILES: CC(=O)N(C)[C@@H]1[C@H](N)C[C@H](c2ccncc2Nc2ncc3ccc(-c4c(F)cc(CN5CCCC5=O)cc4F)nn23)C[C@@H]1C]

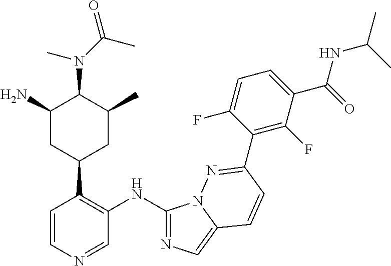 [SMILES: CC(=O)N(C)[C@@H]1[C@H](N)C[C@H](c2ccncc2Nc2ncc3ccc(-c4c(F)ccc(C(=O)NC(C)C)c4F)nn23)C[C@@H]1C]

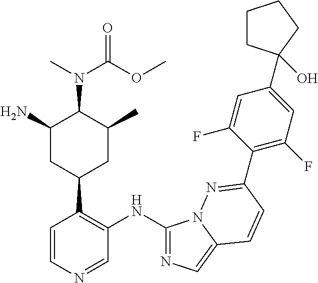 [SMILES: COC(=O)N(C)[C@@H]1[C@H](N)C[C@H](c2ccncc2Nc2ncc3ccc(-c4c(F)cc(C5(O)CCCC5)cc4F)nn23)C[C@@H]1C]

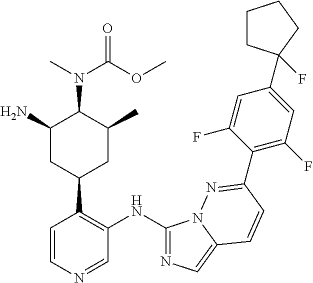 [SMILES: COC(=O)N(C)[C@@H]1[C@H](N)C[C@H](c2ccncc2Nc2ncc3ccc(-c4c(F)cc(C5(F)CCCC5)cc4F)nn23)C[C@@H]1C]

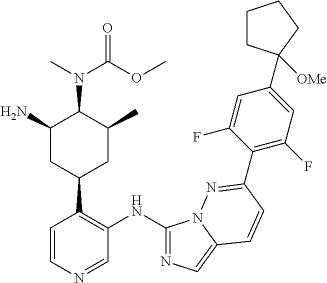 [SMILES: COC(=O)N(C)[C@@H]1[C@H](N)C[C@H](c2ccncc2Nc2ncc3ccc(-c4c(F)cc(C5(OC)CCCC5)cc4F)nn23)C[C@@H]1C]

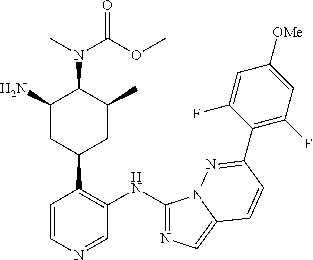 [SMILES: COC(=O)N(C)[C@@H]1[C@H](N)C[C@H](c2ccncc2Nc2ncc3ccc(-c4c(F)cc(OC)cc4F)nn23)C[C@@H]1C]

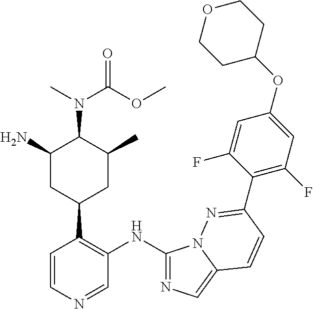 [SMILES: COC(=O)N(C)[C@@H]1[C@H](N)C[C@H](c2ccncc2Nc2ncc3ccc(-c4c(F)cc(OC5CCOCC5)cc4F)nn23)C[C@@H]1C]

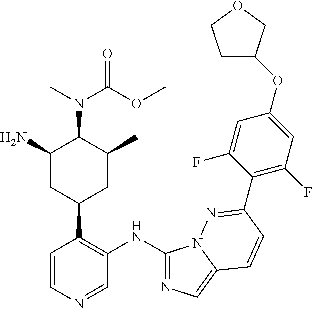 [SMILES: COC(=O)N(C)[C@@H]1[C@H](N)C[C@H](c2ccncc2Nc2ncc3ccc(-c4c(F)cc(OC5CCOC5)cc4F)nn23)C[C@@H]1C]

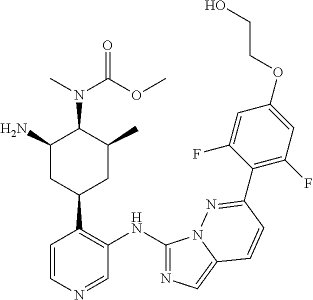 [SMILES: COC(=O)N(C)[C@@H]1[C@H](N)C[C@H](c2ccncc2Nc2ncc3ccc(-c4c(F)cc(OCCO)cc4F)nn23)C[C@@H]1C]

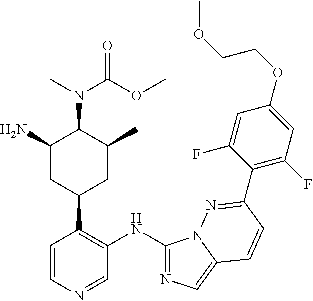 [SMILES: COCCOc1cc(F)c(-c2ccc3cnc(Nc4cnccc4[C@H]4C[C@@H](N)[C@@H](N(C)C(=O)OC)[C@@H](C)C4)n3n2)c(F)c1]

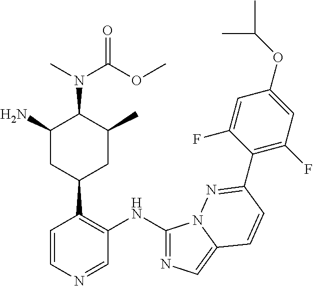 [SMILES: COC(=O)N(C)[C@@H]1[C@H](N)C[C@H](c2ccncc2Nc2ncc3ccc(-c4c(F)cc(OC(C)C)cc4F)nn23)C[C@@H]1C]

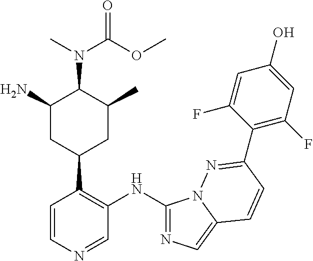 [SMILES: COC(=O)N(C)[C@@H]1[C@H](N)C[C@H](c2ccncc2Nc2ncc3ccc(-c4c(F)cc(O)cc4F)nn23)C[C@@H]1C]

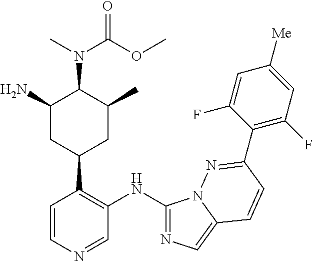 [SMILES: COC(=O)N(C)[C@@H]1[C@H](N)C[C@H](c2ccncc2Nc2ncc3ccc(-c4c(F)cc(C)cc4F)nn23)C[C@@H]1C]

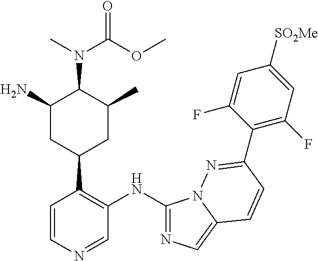 [SMILES: COC(=O)N(C)[C@@H]1[C@H](N)C[C@H](c2ccncc2Nc2ncc3ccc(-c4c(F)cc(S(C)(=O)=O)cc4F)nn23)C[C@@H]1C]

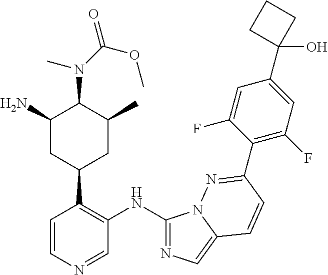 [SMILES: COC(=O)N(C)[C@@H]1[C@H](N)C[C@H](c2ccncc2Nc2ncc3ccc(-c4c(F)cc(C5(O)CCC5)cc4F)nn23)C[C@@H]1C]

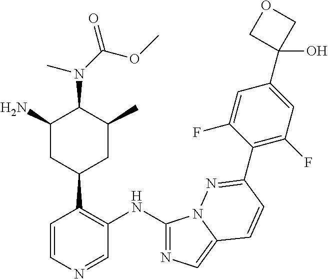 [SMILES: COC(=O)N(C)[C@@H]1[C@H](N)C[C@H](c2ccncc2Nc2ncc3ccc(-c4c(F)cc(C5(O)COC5)cc4F)nn23)C[C@@H]1C]

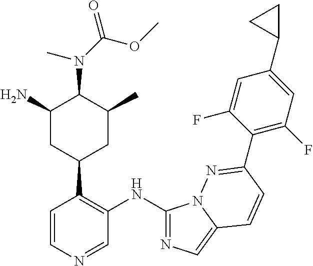 [SMILES: COC(=O)N(C)[C@@H]1[C@H](N)C[C@H](c2ccncc2Nc2ncc3ccc(-c4c(F)cc(C5CC5)cc4F)nn23)C[C@@H]1C]

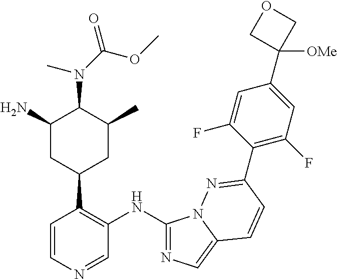 [SMILES: COC(=O)N(C)[C@@H]1[C@H](N)C[C@H](c2ccncc2Nc2ncc3ccc(-c4c(F)cc(C5(OC)COC5)cc4F)nn23)C[C@@H]1C]

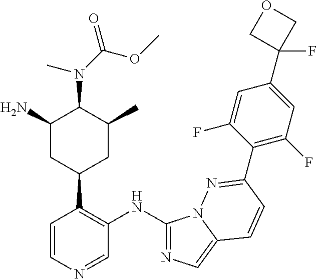 [SMILES: COC(=O)N(C)[C@@H]1[C@H](N)C[C@H](c2ccncc2Nc2ncc3ccc(-c4c(F)cc(C5(F)COC5)cc4F)nn23)C[C@@H]1C]

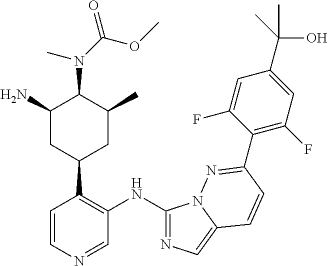 [SMILES: COC(=O)N(C)[C@@H]1[C@H](N)C[C@H](c2ccncc2Nc2ncc3ccc(-c4c(F)cc(C(C)(C)O)cc4F)nn23)C[C@@H]1C]

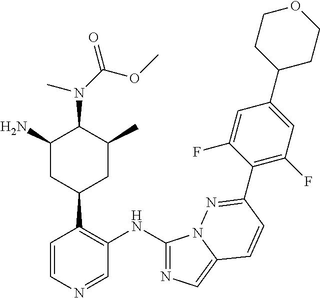 [SMILES: COC(=O)N(C)[C@@H]1[C@H](N)C[C@H](c2ccncc2Nc2ncc3ccc(-c4c(F)cc(C5CCOCC5)cc4F)nn23)C[C@@H]1C]

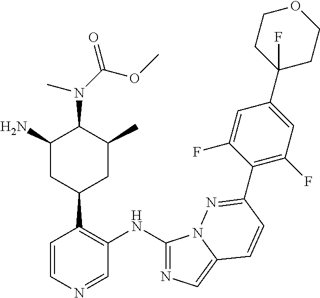

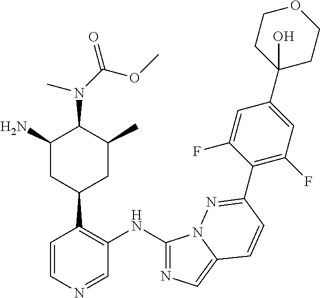

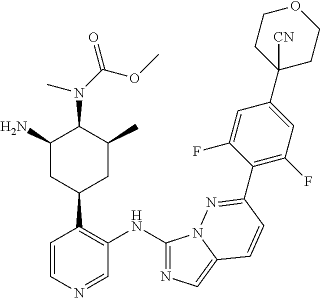

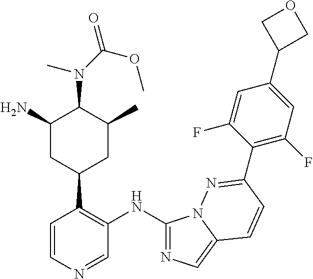 [SMILES: COC(=O)N(C)[C@@H]1[C@H](N)C[C@H](c2ccncc2Nc2ncc3ccc(-c4c(F)cc(C5COC5)cc4F)nn23)C[C@@H]1C]

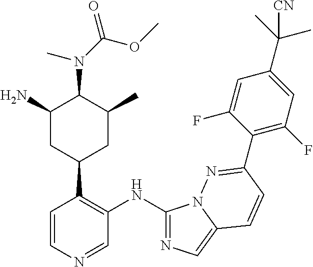 [SMILES: COC(=O)N(C)[C@@H]1[C@H](N)C[C@H](c2ccncc2Nc2ncc3ccc(-c4c(F)cc(C(C)(C)C#N)cc4F)nn23)C[C@@H]1C]

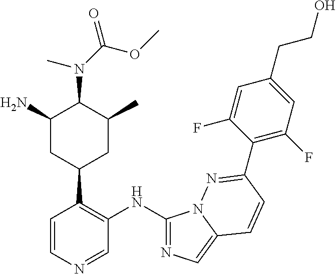 [SMILES: COC(=O)N(C)[C@@H]1[C@H](N)C[C@H](c2ccncc2Nc2ncc3ccc(-c4c(F)cc(CCO)cc4F)nn23)C[C@@H]1C]

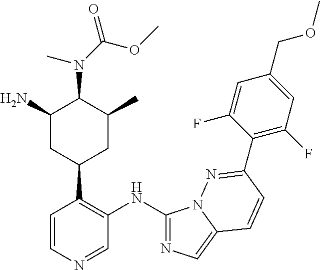 [SMILES: COCc1cc(F)c(-c2ccc3cnc(Nc4cnccc4[C@H]4C[C@@H](N)[C@@H](N(C)C(=O)OC)[C@@H](C)C4)n3n2)c(F)c1]

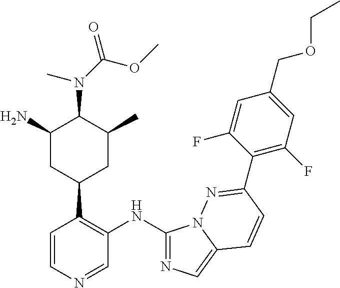 [SMILES: CCOCc1cc(F)c(-c2ccc3cnc(Nc4cnccc4[C@H]4C[C@@H](N)[C@@H](N(C)C(=O)OC)[C@@H](C)C4)n3n2)c(F)c1]

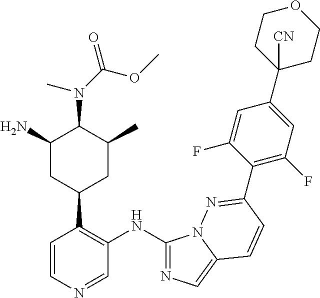

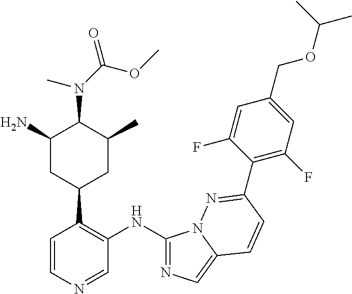 [SMILES: COC(=O)N(C)[C@@H]1[C@H](N)C[C@H](c2ccncc2Nc2ncc3ccc(-c4c(F)cc(COC(C)C)cc4F)nn23)C[C@@H]1C]

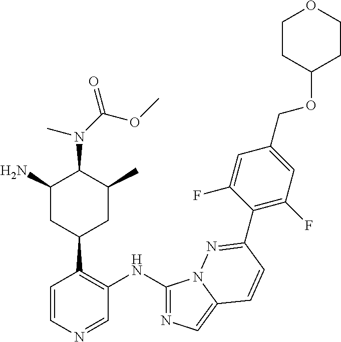 [SMILES: COC(=O)N(C)[C@@H]1[C@H](N)C[C@H](c2ccncc2Nc2ncc3ccc(-c4c(F)cc(COC5CCOCC5)cc4F)nn23)C[C@@H]1C]

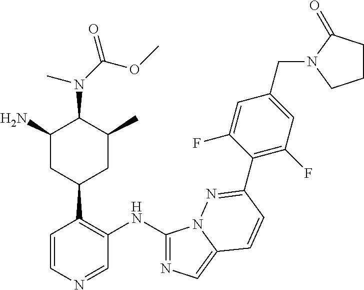 [SMILES: COC(=O)N(C)[C@@H]1[C@H](N)C[C@H](c2ccncc2Nc2ncc3ccc(-c4c(F)cc(CN5CCCC5=O)cc4F)nn23)C[C@@H]1C]

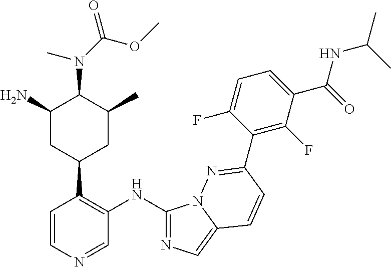 [SMILES: COC(=O)N(C)[C@@H]1[C@H](N)C[C@H](c2ccncc2Nc2ncc3ccc(-c4c(F)ccc(C(=O)NC(C)C)c4F)nn23)C[C@@H]1C]

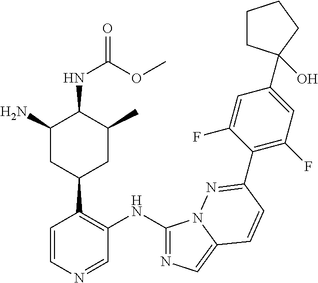 [SMILES: COC(=O)N[C@@H]1[C@H](N)C[C@H](c2ccncc2Nc2ncc3ccc(-c4c(F)cc(C5(O)CCCC5)cc4F)nn23)C[C@@H]1C]

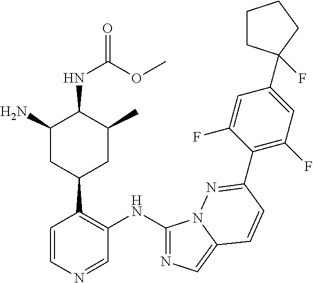 [SMILES: COC(=O)N[C@@H]1[C@H](N)C[C@H](c2ccncc2Nc2ncc3ccc(-c4c(F)cc(C5(F)CCCC5)cc4F)nn23)C[C@@H]1C]

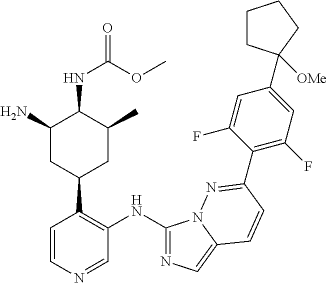 [SMILES: COC(=O)N[C@@H]1[C@H](N)C[C@H](c2ccncc2Nc2ncc3ccc(-c4c(F)cc(C5(OC)CCCC5)cc4F)nn23)C[C@@H]1C]

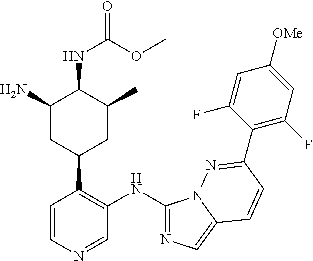 [SMILES: COC(=O)N[C@@H]1[C@H](N)C[C@H](c2ccncc2Nc2ncc3ccc(-c4c(F)cc(OC)cc4F)nn23)C[C@@H]1C]

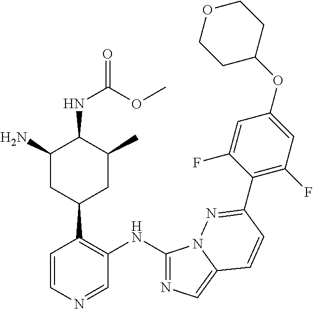 [SMILES: COC(=O)N[C@@H]1[C@H](N)C[C@H](c2ccncc2Nc2ncc3ccc(-c4c(F)cc(OC5CCOCC5)cc4F)nn23)C[C@@H]1C]

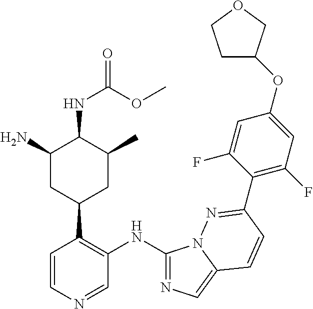 [SMILES: COC(=O)N[C@@H]1[C@H](N)C[C@H](c2ccncc2Nc2ncc3ccc(-c4c(F)cc(OC5CCOC5)cc4F)nn23)C[C@@H]1C]

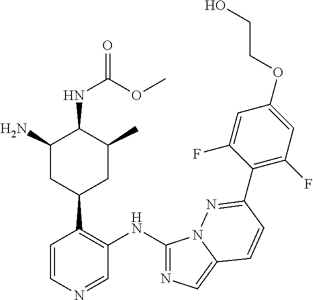 [SMILES: COC(=O)N[C@@H]1[C@H](N)C[C@H](c2ccncc2Nc2ncc3ccc(-c4c(F)cc(OCCO)cc4F)nn23)C[C@@H]1C]

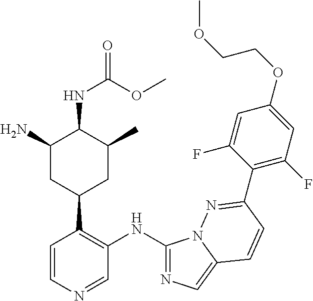 [SMILES: COCCOc1cc(F)c(-c2ccc3cnc(Nc4cnccc4[C@H]4C[C@@H](N)[C@@H](NC(=O)OC)[C@@H](C)C4)n3n2)c(F)c1]

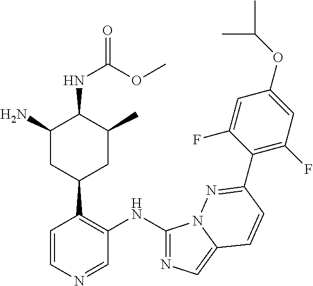 [SMILES: COC(=O)N[C@@H]1[C@H](N)C[C@H](c2ccncc2Nc2ncc3ccc(-c4c(F)cc(OC(C)C)cc4F)nn23)C[C@@H]1C]

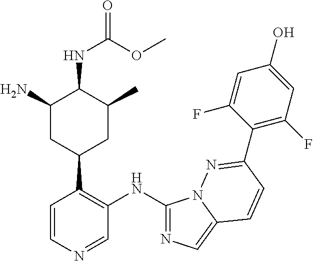 [SMILES: COC(=O)N[C@@H]1[C@H](N)C[C@H](c2ccncc2Nc2ncc3ccc(-c4c(F)cc(O)cc4F)nn23)C[C@@H]1C]

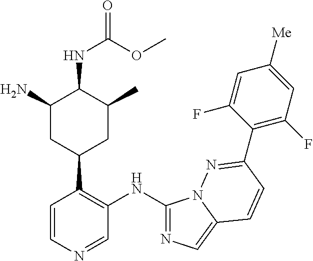 [SMILES: COC(=O)N[C@@H]1[C@H](N)C[C@H](c2ccncc2Nc2ncc3ccc(-c4c(F)cc(C)cc4F)nn23)C[C@@H]1C]

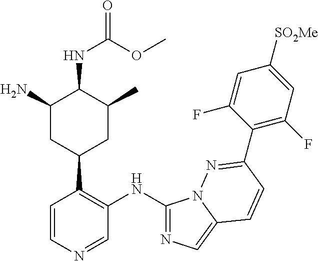 [SMILES: COC(=O)N[C@@H]1[C@H](N)C[C@H](c2ccncc2Nc2ncc3ccc(-c4c(F)cc(S(C)(=O)=O)cc4F)nn23)C[C@@H]1C]

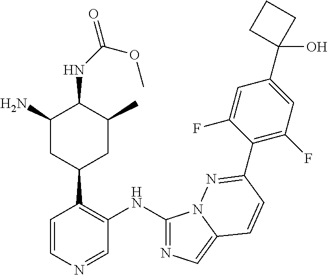 [SMILES: COC(=O)N[C@@H]1[C@H](N)C[C@H](c2ccncc2Nc2ncc3ccc(-c4c(F)cc(C5(O)CCC5)cc4F)nn23)C[C@@H]1C]

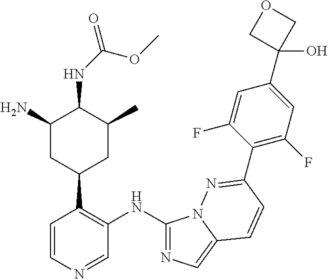 [SMILES: COC(=O)N[C@@H]1[C@H](N)C[C@H](c2ccncc2Nc2ncc3ccc(-c4c(F)cc(C5(O)COC5)cc4F)nn23)C[C@@H]1C]

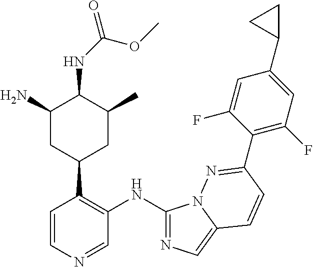 [SMILES: COC(=O)N[C@@H]1[C@H](N)C[C@H](c2ccncc2Nc2ncc3ccc(-c4c(F)cc(C5CC5)cc4F)nn23)C[C@@H]1C]

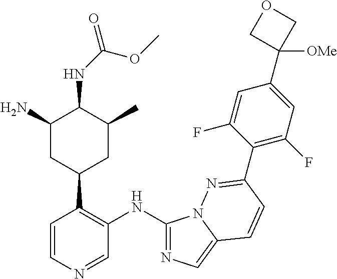 [SMILES: COC(=O)N[C@@H]1[C@H](N)C[C@H](c2ccncc2Nc2ncc3ccc(-c4c(F)cc(C5(OC)COC5)cc4F)nn23)C[C@@H]1C]

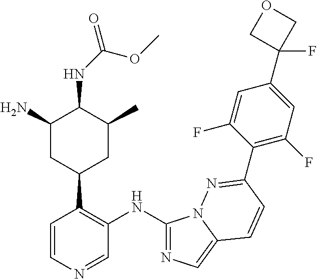 [SMILES: COC(=O)N[C@@H]1[C@H](N)C[C@H](c2ccncc2Nc2ncc3ccc(-c4c(F)cc(C5(F)COC5)cc4F)nn23)C[C@@H]1C]

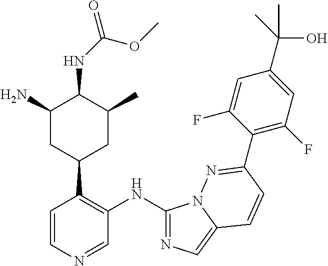 [SMILES: COC(=O)N[C@@H]1[C@H](N)C[C@H](c2ccncc2Nc2ncc3ccc(-c4c(F)cc(C(C)(C)O)cc4F)nn23)C[C@@H]1C]

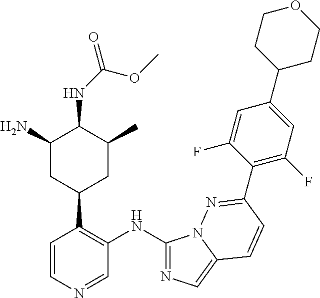 [SMILES: COC(=O)N[C@@H]1[C@H](N)C[C@H](c2ccncc2Nc2ncc3ccc(-c4c(F)cc(C5CCOCC5)cc4F)nn23)C[C@@H]1C]

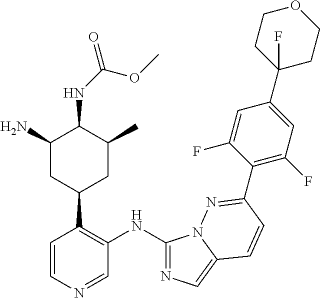

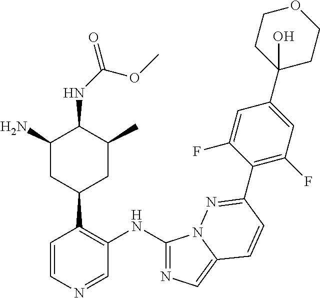

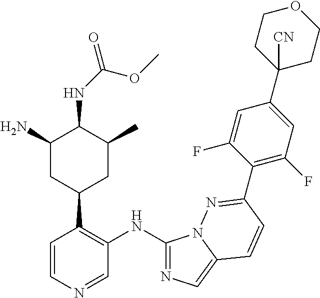

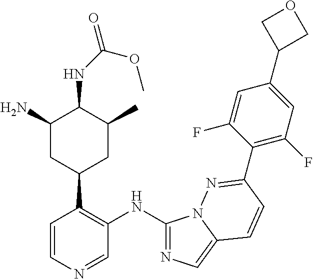 [SMILES: COC(=O)N[C@@H]1[C@H](N)C[C@H](c2ccncc2Nc2ncc3ccc(-c4c(F)cc(C5COC5)cc4F)nn23)C[C@@H]1C]

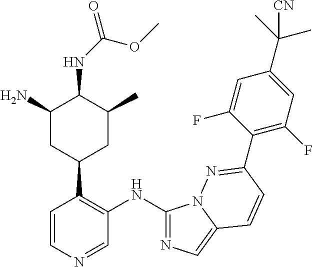 [SMILES: COC(=O)N[C@@H]1[C@H](N)C[C@H](c2ccncc2Nc2ncc3ccc(-c4c(F)cc(C(C)(C)C#N)cc4F)nn23)C[C@@H]1C]

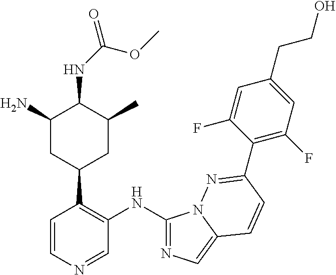 [SMILES: COC(=O)N[C@@H]1[C@H](N)C[C@H](c2ccncc2Nc2ncc3ccc(-c4c(F)cc(CCO)cc4F)nn23)C[C@@H]1C]

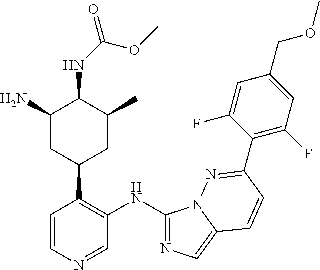 [SMILES: COCc1cc(F)c(-c2ccc3cnc(Nc4cnccc4[C@H]4C[C@@H](N)[C@@H](NC(=O)OC)[C@@H](C)C4)n3n2)c(F)c1]

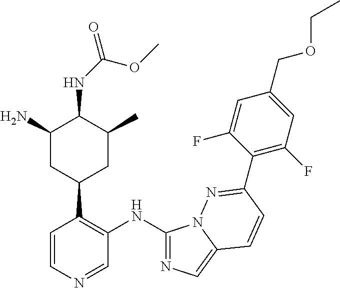 [SMILES: CCOCc1cc(F)c(-c2ccc3cnc(Nc4cnccc4[C@H]4C[C@@H](N)[C@@H](NC(=O)OC)[C@@H](C)C4)n3n2)c(F)c1]

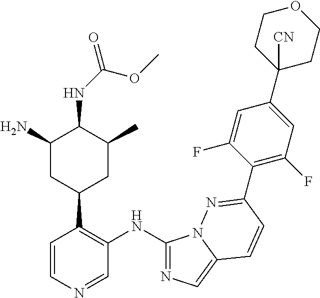

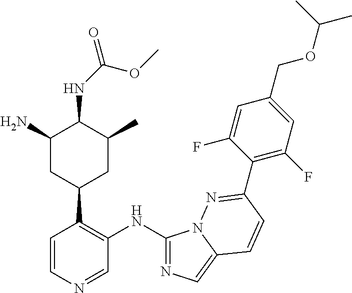 [SMILES: COC(=O)N[C@@H]1[C@H](N)C[C@H](c2ccncc2Nc2ncc3ccc(-c4c(F)cc(COC(C)C)cc4F)nn23)C[C@@H]1C]

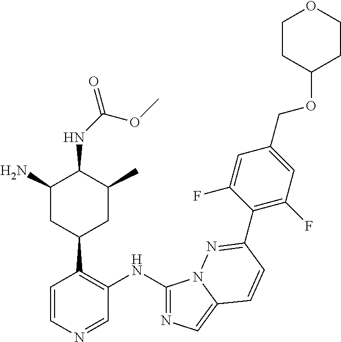 [SMILES: COC(=O)N[C@@H]1[C@H](N)C[C@H](c2ccncc2Nc2ncc3ccc(-c4c(F)cc(COC5CCOCC5)cc4F)nn23)C[C@@H]1C]

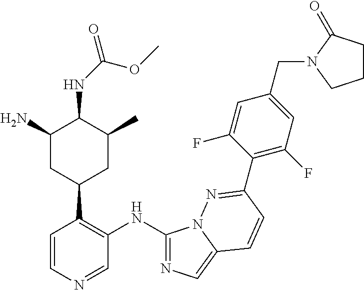 [SMILES: COC(=O)N[C@@H]1[C@H](N)C[C@H](c2ccncc2Nc2ncc3ccc(-c4c(F)cc(CN5CCCC5=O)cc4F)nn23)C[C@@H]1C]

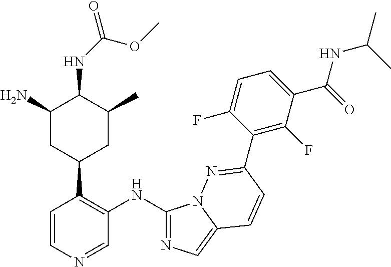 [SMILES: COC(=O)N[C@@H]1[C@H](N)C[C@H](c2ccncc2Nc2ncc3ccc(-c4c(F)ccc(C(=O)NC(C)C)c4F)nn23)C[C@@H]1C]

The invention provides compounds of Formula I wherein novel combinations of substituents on the cyclohexyl or piperidine ring and the phenyl/pyridinyl ring that provide advantageous biological activities. Advantages provided by preferred compounds include reduced drug-drug interactions due to reduction of time-dependent Cyp inhibition, or pharmacokinetic superiority based on improved clearance and/or metabolic properties.

These compounds may be used in racemic form, or the individual enantiomers may be used, or mixtures of the enantiomers or diastereomers may be used. Each enantiomer can be used, and preferably the compound to be used is the enantiomer that has greater activity as a Pim inhibitor on at least two of Pims 1, 2, and 3.

For purposes of the present invention, a therapeutically effective dose will generally be a total daily dose administered to a host in single dose, or divided doses administered within 24 hours, which may be in amounts, for example, of from 0.001 to 1000 mg/kg body weight daily, typically 0.01 to 100 mg/kg per day, and more preferred from 0.1 to 30 mg/kg body weight daily. Generally, daily dosage amounts of 1 to 4000 mg, or from 5 to 3000, or from 10 to 2000 mg, or from 100 to 2000 mg are anticipated for human subjects. Dosage unit compositions may contain such amounts or submultiples thereof to make up the daily dose.

The compounds of the present invention may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques. In preferred embodiments, the compound or composition of the invention is administered orally.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq. (1976).

While the compounds of the invention can be administered as the sole active pharmaceutical agent, they can also be used in combination with one or more other agents used in the treatment of cancer. The compounds of the present invention are also useful in combination with known therapeutic agents and anti-cancer agents, and combinations of the presently disclosed compounds with other anti-cancer or chemotherapeutic agents are within the scope of the invention. Examples of such agents can be found in Cancer Principles and Practice of Oncology, V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: MEK inhibitors, estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The compounds of the invention are also useful when co-administered with radiation therapy.

Therefore, in one embodiment of the invention, the compounds of the invention are also used in combination with known therapeutic or anticancer agents including, for example, estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

In certain presently preferred embodiments of the invention, representative therapeutic agents useful in combination with the compounds of the invention for the treatment of cancer include, for example, MEK inhibitors, irinotecan, topotecan, gemcitabine, 5-fluorouracil, cytarabine, daunorubicin, PI3 Kinase inhibitors, mTOR inhibitors, DNA synthesis inhibitors, leucovorin carboplatin, cisplatin, taxanes, tezacitabine, cyclophosphamide, vinca alkaloids, imatinib (Gleevec), anthracyclines, rituximab, trastuzumab, Revlimid, thalidomide, Velcade, dexamethasone, daunorubicin, cytaribine, clofarabine, Mylotarg, lenalidomide, bortezomib, as well as other cancer chemotherapeutic agents including targeted therapuetics.

The above compounds to be employed in combination with the compounds of the invention will be used in therapeutic amounts as indicated in the Physicians' Desk Reference (PDR) 47th Edition (1993), which is incorporated herein by reference, or such therapeutically useful amounts as would be known to one of ordinary skill in the art, or provided in prescribing materials such as a drug label for the additional therapeutic agent.

The compounds of the invention and the other anticancer agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions, which are given at the same time or different times, or the therapeutic agents, can be given as a single composition.

In one embodiment, the invention provides a method of inhibiting Pim1, Pim2 or Pim3 in a human or animal subject. The method includes administering an effective amount of a compound, or a pharmaceutically acceptable salt thereof, of any of the embodiments of compounds of Formula I to a subject in need thereof.

The present invention will be understood more readily by reference to the following examples, which are provided by way of illustration and are not intended to be limiting of the present invention.

### Synthetic Methods

The compounds of the invention can be obtained through procedures known to those skilled in the art. In particular, methods for making the bicyclic fused pyridazine portion of these compounds and attaching it to a suitable 4-substituted pyridin-3-ylamine group are described in, e.g., WO2012/148775. The specific substituted phenyl and pyridinyl rings corresponding to the ring containing Z in Formula I are also known in the art, and some are described herein.

Scheme 1 illustrates synthetic methods useful to prepare the fused bicyclic pyridazine ring system of Formula I where Z² is CH. Examples of this method and conditions for the reactions are known in the art, e.g., WO2012/148775. Arylation of a halopyridazine (1-a) can be done using Suzuki or Negishi arylation conditions to provide compounds of formula 1-b, where Ar is a suitably substituted phenyl group. The methyl group on the pyridazine can then be functionalized by free radical halogenation conditions, followed by nucleophilic displacement with azide to give 1-c. If functionality in the aryl moiety attached to the pyridazine is not stable to the radical chlorination conditions, performing the Suzuki or Negishi arylation on the corresponding (6-halopyridazin-3-yl)methanol and subsequent conversion of the primary alcohol to azide (via a mesylate or chloride) is another option for preparing compounds 1-c. Subsequent reaction or azido compounds 1-c with a trialkyphosphine (e.g., Me₃P) reduces the azide to a phosphine imine, which reacts readily with an isothiocyanate of formula 1-d to produce compounds of formula 1-f

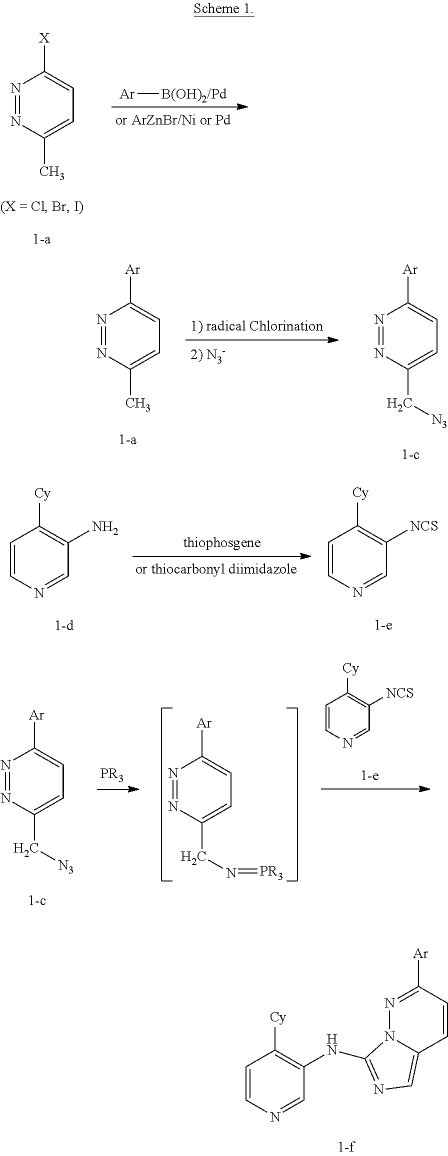

Methods to make the required aryl boronates and substituted aminopyridines (1-d) are well known in the art: see for example WO2012/004217, WO2012/120415, and WO2012/120428. General methods for converting the aminopyridines of formula 1-d into the requisite isothiocyanates (1-e) using thiophosgene or the alternative reagent thiocarbonyl diimidazole are known in the art, and are described in WO2012/148775.

Compounds of Formula I wherein Z² is N can be prepared by the method shown in Scheme 2, using a hydrazino-pyridazine:

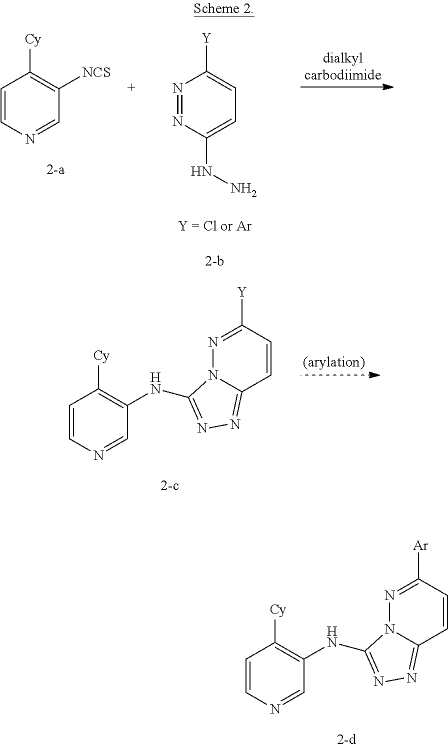

If Y═Cl in 2-b, Negishi or Suzuki arylation can be used to install the desired substituted phenyl group to arrive at 2-d.

### EXAMPLES

Referring to the examples that follow, embodiments of the compounds of Formula I can be synthesized using the methods described herein and other methods well known in the art.

The compounds and/or intermediates can be characterized by high performance liquid chromatography (HPLC) using a Waters Millenium chromatography system with a 2695 Separation Module (Milford, Mass.). The analytical columns referred to are reversed phase Phenomenex Luna C18-5μ, 4.6×50 mm, from Alltech (Deerfield, Ill.). A gradient elution can be used (flow 2.5 mL/min), typically starting with 5% acetonitrile/95% water and progressing to 100% acetonitrile over a period of 10 minutes. All solvents would contain 0.1% trifluoroacetic acid (TFA). Compounds can be detected by ultraviolet light (UV) absorption at either 220 or 254 nm. HPLC solvents can be obtained from Burdick and Jackson (Muskegan, Mich.), or Fisher Scientific (Pittsburgh, Pa.).

In some instances, where purity is reported herein, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well-known iodine vapor and other various staining techniques.

Mass spectrometric analyses reported herein were performed on one of three LCMS instruments: a Waters System (Alliance HT HPLC and a Micromass ZQ mass spectrometer; Column: Eclipse XDB-C18, 2.1×50 mm; gradient: 5-95% (or 35-95%, or 65-95% or 95-95%) acetonitrile in water with 0.05% TFA over a 4 min period; flow rate 0.8 mL/min; molecular weight range 200-1500; cone Voltage 20 V; column temperature 40° C.), another Waters System (ACQUITY UPLC system and a ZQ 2000 system; Column: ACQUITY UPLC HSS—C18, 1.8 um, 2.1×50 mm; gradient: 5-95% (or 35-95%, or 65-95% or 95-95%) acetonitrile in water with 0.05% TFA over a 1.3 min period; flow rate 1.2 mL/min; molecular weight range 150-850; cone Voltage 20 V; column temperature 50° C.) or a Hewlett Packard System (Series 1100 HPLC; Column: Eclipse XDB-C18, 2.1×50 mm; gradient: 5-95% acetonitrile in water with 0.05% TFA over a 4 min period; flow rate 0.8 mL/min; molecular weight range 150-850; cone Voltage 50 V; column temperature 30° C.). All masses were reported as those of the protonated parent ions.

Nuclear magnetic resonance (NMR) analyses described herein were performed on some of the compounds with a Varian 400 MHz NMR (Palo Alto, Calif.). The spectral reference was either TMS or the known chemical shift of the solvent.

Preparative separations described herein were are carried out using a Flash 40 chromatography system and KP-Sil, 60A (Biotage, Charlottesville, Va.), or by flash column chromatography using silica gel (230-400 mesh) packing material on ISCO or Analogix purification systems, or by HPLC using a Waters 2767 Sample Manager, C-18 reversed phase column, 30×50 mm, flow 75 mL/min. Typical solvents employed for the Flash 40 Biotage, ISCO or Analogixsystem for silica gel column chromatography are dichloromethane, methanol, ethyl acetate, hexane, n-heptanes, acetone, aqueous ammonia (or ammonium hydroxide), and triethyl amine. Typical solvents employed for the reverse phase HPLC are varying concentrations of acetonitrile and water with 0.1% trifluoroacetic acid.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

It is understood that the invention is not limited to the embodiments set forth herein for illustration, but embraces all such forms thereof as come within the scope of the above disclosure.

The examples below as well as throughout the application, the following abbreviations have the following meanings. If not defined, the terms have their generally accepted meanings

ABBREVIATIONS

| | |
|---|---|
| BINAP | 2,2′-Bis(diphenylphosphino)-1,1′-binaphthalene |
| Boc₂O | di-tert-butyl dicarbonate |
| Boc-OSu | N-(tert-Butoxycarbonyloxy)succinimide |
| DAST | (diethylamino)sulfurtrifluoride |
| DBU | 1,8-Diazabicyclo[5.4.0]undec-7-ene |
| DCM | Dichloromethane |
| DIAD | diisopropylazodicarboxylate |
| DIEA | diisopropylethylamine |
| DMA | Dimethylacetamide |
| DMAP | 4-dimethylaminopyridine |
| DME | 1,2-dimethoxyethane |
| DMF | N,N-dimethylformamide |
| DPPF | 1,1′-bis(diphenylphosphino)ferrocene |
| EDC | 1-(3-Dimethylaminopropyl)-3-ethylcarbodiimide |
| | hydrochloride |
| EtOAc | ethyl acetate |
| EtOH | Ethanol |
| HOAT | Hydroxyazabenzotriazole |
| K₂CO₃ | Potassium carbonate |
| Lawesson's | 2,4-bis(4-methoxyphenyl)-1,3,2,4- |
| reagent | dithiadiphosphetane-2,4-dithione |
| LiOH | Lithium hydroxide |
| MCPBA | Meta-chloroperbenzoic acid |
| MeCN | Acetonitrile |
| methylDAST | (dimethylamino)sulfurtrifluoride |
| MgSO₄ | Magnesium sulfate |
| MeOH | Methanol |
| MsCl | Methane sulfonyl chloride |
| Na₂CO₃ | sodium carbonate |
| NaCl | Sodium chloride |
| NaHCO_{₃} | sodium bicarbonate |
| NaHMDS | Sodium bis(trimethylsilyl)amide |
| NBS | N-bromosuccinimide |
| NMP | N-methyl-2-pyrrolidone |
| oxone | Potassium peroxymonosulfate |
| p-TSA | para-toluene sulfonic acid |
| Pd_{₂}(dba)_{₃} | Tris(dibenzylideneacetone)dipalladium(0) |
| Pd(PPh_{₃})_{₄} | Tetrakis(triphenylphospine)palladium(0) |
| Pd(dppf)Cl_{₂}- | Dichloro-(1,2-bis(diphenylphosphino)ethan)- |
| DCM | Palladium(II)-dichloromothethane adduct |
| RT or rt | room temperature |
| TBDMSCl | tert-butyldimethylsilylchloride |
| TEA | Triethylamine |
| THF | tetrahydrofuran |

### Synthesis of Intermediates

### Synthesis of (R)-tert-butyl 4-((1R,2R)-3-((R)-4-benzyl-2-oxooxazolidin-3-yl)-1-hydroxy-2-methyl-3-oxopropyl)-2,2-dimethyloxazolidine-3-carboxylate

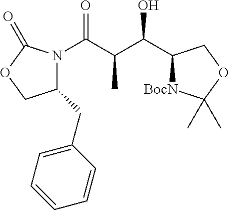 [SMILES: C[C@@H](C(=O)N1C(=O)OC[C@H]1Cc1ccccc1)[C@@H](O)[C@H]1COC(C)(C)N1C(=O)OC(C)(C)C]

To a solution of (R)-4-benzyl-3-propionyloxazolidin-2-one (1.0 equiv.) in DCM (0.13 M) was added TiCl₄ (1.0 equiv.) at −40° C. The mixture was stirred at −40° C. for 10 min (yellow suspension), then DIPEA (2.5 equiv.) was added (dark red solution) and stirred at 0° C. for 20 min. (R)-tert-butyl 4-formyl-2,2-dimethyloxazolidine-3-carboxylate (1.0 equiv.) in DCM (0.5 M) was then added dropwise and the resulting mixture was stirred for 1.5 hours. The reaction was quenched by the addition of aqueous ammonium chloride and the mixture was extracted with ethyl acetate. The organic phase was separated, washed with brine, dried with magnesium sulfate, filtered, and concentrated. The residue was purified via column chromatography eluting with ethyl acetate and hexanes (1:4) to give (R)-tert-butyl 4-((1R,2R)-3-((R)-4-benzyl-2-oxooxazolidin-3-yl)-1-hydroxy-2-methyl-3-oxopropyl)-2,2-dimethyloxazolidine-3-carboxylate as the major product (5:2) in 58% yield. LC/MS=363.3 (M+H-Boc), Rt=1.09 min.

### Synthesis of (R)-tert-butyl 4-((1R,2R)-3-((R)-4-benzyl-2-oxooxazolidin-3-yl)-1-(tert-butyldimethylsilyloxy)-2-methyl-3-oxopropyl)-2,2-dimethyloxazolidine-3-carboxylate

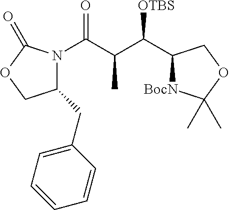 [SMILES: C[C@@H](C(=O)N1C(=O)OC[C@H]1Cc1ccccc1)[C@@H](O[Si](C)(C)C(C)(C)C)[C@H]1COC(C)(C)N1C(=O)OC(C)(C)C]

To a solution of (R)-tert-butyl 4-((1R,2R)-3-((R)-4-benzyl-2-oxooxazolidin-3-yl)-1-hydroxy-2-methyl-3-oxopropyl)-2,2-dimethyloxazolidine-3-carboxylate (1.0 equiv.) and lutidine (1.8 equiv.) in DCM (0.1M) was added TBSOTf (1.4 equiv.) at −40° C. The reaction mixture was stirred at −40° C. for 2 hours. The solution was diluted with ethyl acetate and washed with sat. NaHCO₃, sat. NaCl, dried with magnesium sulfate, filtered, and concentrated. The residue was purified by silica gel column chromatography eluting with ethyl acetate and hexanes (1:4) to give (R)-tert-butyl 4-((1R,2R)-3-((R)-4-benzyl-2-oxooxazolidin-3-yl)-1-(tert-butyldimethylsilyloxy)-2-methyl-3-oxopropyl)-2,2-dimethyloxazolidine-3-carboxylate as the major product (5:2) in 83% yield. LC/MS=577.3 (M+H), Rt=1.33 min (Frac 65%-95% method).

### Synthesis of (R)-tert-butyl 4-((1R,2S)-1-(tert-butyldimethylsilyloxy)-3-hydroxy-2-methylpropyl)-2,2-dimethyloxazolidine-3-carboxylate

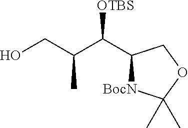 [SMILES: C[C@@H](CO)[C@@H](O[Si](C)(C)C(C)(C)C)[C@H]1COC(C)(C)N1C(=O)OC(C)(C)C]

To a solution of (R)-tert-butyl 4-((1R,2R)-3-((R)-4-benzyl-2-oxooxazolidin-3-yl)-1-(tert-butyldimethylsilyloxy)-2-methyl-3-oxopropyl)-2,2-dimethyloxazolidine-3-carboxylate (1.0 equiv.) and ethanol (3.0 equiv.) in THF (0.09 M) was added LiBH₄ (3.0 equiv.) at −30° C. The reaction mixture was allowed to warm up to 0° C. and stirred at that temperature for 3 hours. The solution was then diluted with diethyl ether and 1N NaOH was added. The resulting mixture was extracted with ethyl acetate, the organic layer was separated, washed with sat. NaCl, dried over magnesium sulfate, filtered, and concentrated. The residue was purified via silica gel column chromatography eluting with ethyl acetate and hexanes (1:4) to give (R)-tert-butyl 4-((1R,2S)-1-(tert-butyldimethylsilyloxy)-3-hydroxy-2-methylpropyl)-2,2-dimethyloxazolidine-3-carboxylate as the major product (5:2 ratio) in 71% yield. LC/MS=304.3 (M+H-Boc), Rt=0.95 min (Frac 65%-95% method).

### Synthesis of (R)-tert-butyl 4-((1R,2S)-3-azido-1-(tert-butyldimethylsilyloxy)-2-methylpropyl)-2,2-dimethyloxazolidine-3-carboxylate

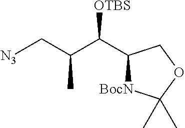 [SMILES: C[C@@H](CN=[N+]=[N-])[C@@H](O[Si](C)(C)C(C)(C)C)[C@H]1COC(C)(C)N1C(=O)OC(C)(C)C]

To a solution of (R)-tert-butyl 4-((1R,2S)-1-(tert-butyldimethylsilyloxy)-3-hydroxy-2-methylpropyl)-2,2-dimethyloxazolidine-3-carboxylate (1.0 equiv.), DIAD (2.0 equiv.), and PPh₃ (2.0 equiv.) in THF (0.18 M) was added DPPA (2.0 equiv., 1M solution in THF). The reaction mixture was stirred at room temperature overnight. Upon removal of the volatiles under vacuo, the residue was purified by silica gel column chromatography eluting with ethyl acetate and hexanes (1:6) to give (R)-tert-butyl 4-((1R,2S)-3-azido-1-(tert-butyldimethylsilyloxy)-2-methylpropyl)-2,2-dimethyloxazolidine-3-carboxylate as the major product (5:2) in 86% yield. LC/MS=329.3 (M+H-Boc), Rt=1.40 min (Frac 65%-95% method).

### Synthesis of tert-butyl (2R,3R,4S)-5-azido-3-(tert-butyldimethylsilyloxy)-1-hydroxy-4-methylpentan-2-ylcarbamate

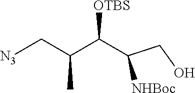 [SMILES: C[C@@H](CN=[N+]=[N-])[C@@H](O[Si](C)(C)C(C)(C)C)[C@@H](CO)NC(=O)OC(C)(C)C]

To a solution of (R)-tert-butyl 4-((1R,2S)-3-azido-1-(tert-butyldimethylsilyloxy)-2-methylpropyl)-2,2-dimethyloxazolidine-3-carboxylate (1.0 equiv.) in EtOH (0.1 M) was added PPTS (1.3 equiv.) and the mixture was refluxed for 2 days. The volatiles were removed under vacuo, the residue was dissolved in DCM (0.1 M) and DIEA (1.5 equiv.) and Boc₂O (1.0 equiv.) were added to the reaction mixture. The solution was stirred for 3 hours at room temperature. The solvents were removed under reduced pressure and the residue was diluted with ethyl acetate, washed with water, aqueous NaHSO₄, aqueous NaHCO₃, sat. NaCl, the organic phase was dried with magnesium sulfate, filtered, and concentrated. The residue was purified via silica gel column chromatography eluting with ethyl acetate and hexanes (1:3) to give tert-butyl (2R,3R,4S)-5-azido-3-(tert-butyldimethylsilyloxy)-1-hydroxy-4-methylpentan-2-ylcarbamate as the major isomer (5:2) in 70% yield. LC/MS=289.3 (M+H-Boc), Rt=0.76 min (Frac 65%-95% method).

### Synthesis of (2R,3R,4S)-5-azido-2-(tert-butoxycarbonylamino)-3-(tert-butyldimethylsilyloxy)-4-methylpentyl methanesulfonate

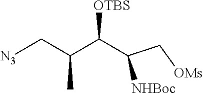 [SMILES: C[C@@H](CN=[N+]=[N-])[C@@H](O[Si](C)(C)C(C)(C)C)[C@@H](COS(C)(=O)=O)NC(=O)OC(C)(C)C]

To a solution of tert-butyl (2R,3R,4S)-5-azido-3-(tert-butyldimethylsilyloxy)-1-hydroxy-4-methylpentan-2-ylcarbamate (1.0 equiv.) in pyridine (0.2 M) was added MsCl (1.3 equiv.) followed by DMAP (catalytic amount) at 0° C. The mixture was stirred at that temperature for 1 hour. The solution was diluted with ether and ethyl acetate (4:1), washed with aq. NaHSO₄, sat. NaHCO₃, brine, dried over magnesium sulfate, filtered, and concentrated. The residue was purified by silica gel column chromatography eluting with ethyl acetate and hexanes (1:3) to give (2R,3R,4S)-5-azido-2-(tert-butoxycarbonylamino)-3-(tert-butyldimethylsilyloxy)-4-methylpentyl methanesulfonate as the major isomer (5:2) in 90% yield. LC/MS=367.3 (M+H-Boc), Rt=0.81 min (Frac 65%-95% method).

### Synthesis of tert-butyl (3R,4R,5S)-4-(tert-butyldimethylsilyloxy)-5-methylpiperidin-3-ylcarbamate

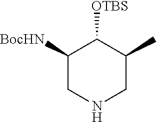 [SMILES: C[C@H]1CNC[C@@H](NC(=O)OC(C)(C)C)[C@@H]1O[Si](C)(C)C(C)(C)C]

A solution of (2R,3R,4S)-5-azido-2-(tert-butoxycarbonylamino)-3-(tert-butyldimethylsilyloxy)-4-methylpentyl methanesulfonate in MeOH (0.09 M) was degassed with nitrogen for 20 min. DIEA (2.5 equiv.) was added, followed by 10% Pd/C (0.1 equiv.). The reaction mixture was stirred under a hydrogen balloon for 2 hours. The solution was filtered and the filtrate was concentrated under vacuo to afford tert-butyl (3R,4R,5S)-4-(tert-butyldimethylsilyloxy)-5-methylpiperidin-3-ylcarbamate as the major isomer (5:2) in >99% yield. LC/MS=345.2 (M+H-Boc), Rt=0.95 and 0.99 min.

### Synthesis of tert-butyl (3R,4R,5S)-4-(tert-butyldimethylsilyloxy)-5-methyl-1-(3-nitropyridin-4-yl)piperidin-3-ylcarbamate

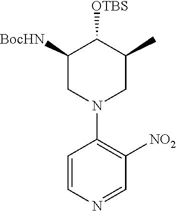 [SMILES: C[C@H]1CN(c2ccncc2[N+](=O)[O-])C[C@@H](NC(=O)OC(C)(C)C)[C@@H]1O[Si](C)(C)C(C)(C)C]

To a solution of tert-butyl (3R,4R,5S)-4-(tert-butyldimethylsilyloxy)-5-methylpiperidin-3-ylcarbamate (1.0 equiv.) in i-PrOH (0.09 M) was added DIEA (2.5 equiv.) and 4-chloro-3-nitropyridine (1.5 equiv.). The reaction mixture was stirred at 60° C. for 2 hours. The volatiles were removed under vacuo, the residue was diluted with ethyl acetate and washed with sat. NaCl. The organic phase was dried with magnesium sulfate, filtered, and concentrated. The crude material was purified by silica gel column chromatography eluting with ethyl acetate and hexanes (1:2) to give tert-butyl (3R,4R,5S)-4-(tert-butyldimethylsilyloxy)-5-methyl-1-(3-nitropyridin-4-yl)piperidin-3-ylcarbamate in 76% yield. LC/MS=467.3 (M+H), Rt=1.09 min.

### Synthesis of tert-butyl (3R,4R,5S)-1-(3-aminopyridin-4-yl)-4-(tert-butyldimethylsilyloxy)-5-methylpiperidin-3-ylcarbamate

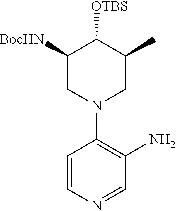 [SMILES: C[C@H]1CN(c2ccncc2N)C[C@@H](NC(=O)OC(C)(C)C)[C@@H]1O[Si](C)(C)C(C)(C)C]

A solution of tert-butyl (3R,4R,5S)-4-(tert-butyldimethylsilyloxy)-5-methyl-1-(3-nitropyridin-4-yl)piperidin-3-ylcarbamate (1.0 equiv.) in MeOH (0.05 M) was degassed with nitrogen for 20 min. 10% Pd/C (0.2 equiv.) was added to the mixture and the solution was stirred under a hydrogen balloon for 3 hours. The reaction was filtered and the filtrate was concentrated under reduced pressure to give tert-butyl (3R,4R,5S)-1-(3-aminopyridin-4-yl)-4-(tert-butyldimethylsilyloxy)-5-methylpiperidin-3-ylcarbamate as the desired product in 94% yield. LC/MS=437.4 (M+H), Rt=1.08 min. ¹H-NMR (300 MHz, CDCl₃): δ 8.01 (s, 1H), 7.95 (d, J=6.0 Hz, 1H), 6.76 (d, J=6.0 Hz, 1H), 4.44 (br s, 1H), 3.74 (br s, 2H), 3.59-3.55 (m, 1H), 3.25-3.13 (m, 2H), 2.47-2.35 (m, 2H), 1.89 (br s, 2H), 1.44 (s, 9H), 1.04 (d, J=6.0, 3H), 0.92 (s, 9H), 0.13 (d, J=9.0, 6H).

### Synthesis of tert-butyl ((3R,4R,5S)-4-((tert-butyldimethylsilyl)oxy)-1-(3-isothiocyanatopyridin-4-yl)-5-methylpiperidin-3-yl)carbamate

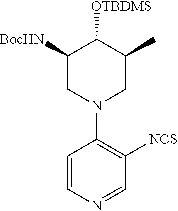

A solution of tert-butyl ((3R,4R,5S)-1-(3-aminopyridin-4-yl)-4-((tert-butyldimethylsilyl)oxy)-5-methylpiperidin-3-yl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((3R,4R,5S)-4-((tert-butyldimethylsilyl)oxy)-1-(3-isothiocyanatopyridin-4-yl)-5-methylpiperidin-3-yl)carbamate is obtained.

### Synthesis of tert-butyl tert-butoxycarbonyl(4-((3R,4R,5S)-3-((tert-butoxycarbonyl)amino)-4-((tert-butyldimethylsilyl)oxy)-5-methylpiperidin-1-yl)pyridin-3-yl)carbamate

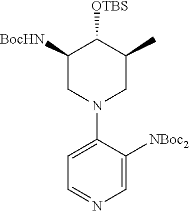

To a solution of tert-butyl (3R,4R,5S)-1-(3-aminopyridin-4-yl)-4-(tert-butyldimethylsilyloxy)-5-methylpiperidin-3-ylcarbamate (1.0 equiv.) in CH₂Cl₂ (0.50 M) at RT was added Boc₂O (6.0 equiv.), followed by DMAP (2.0 equiv.). The resulting mixture was stirred at RT for 16 hrs. The reaction mixture was then diluted with EtOAc and water. The organic layer was washed with Brine, dried over MgSO₄, concentrated and purified by flash column chromatography to yield tert-butyl tert-butoxycarbonyl(4-((3R,4R,5S)-3-((tert-butoxycarbonyl)amino)-4-((tert-butyldimethylsilyl)oxy)-5-methylpiperidin-1-yl)pyridin-3-yl)carbamate in 57% yield. LC/MS (m/z)=637.3 (MH⁺), Rₜ=1.17 min.

### Synthesis of tert-butyl tert-butoxycarbonyl(4-((3R,4R,5S)-3-((tert-butoxycarbonyl)amino)-4-hydroxy-5-methylpiperidin-1-yl)pyridin-3-yl)carbamate

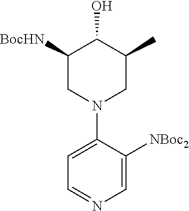

To a solution of tert-butyl tert-butoxycarbonyl(4-((3R,4R,5S)-3-((tert-butoxycarbonyl)amino)-4-((tert-butyldimethylsilyl)oxy)-5-methylpiperidin-1-yl)pyridin-3-yl)carbamate (1.0 equiv.) in THF (0.20 M) at RT was added TBAF (1.0 equiv.). The resulting mixture was stirred at rt for 4 hrs. The reaction mixture was then diluted with EtOAc and water. The organic layer was washed with Brine, dried over MgSO₄, concentrated and purified by flash column chromatography to yield tert-butyl tert-butoxycarbonyl(4-((3R,4R,5S)-3-((tert-butoxycarbonyl)amino)-4-hydroxy-5-methylpiperidin-1-yl)pyridin-3-yl)carbamate in 87% yield. LC/MS (m/z)=523.4 (MH⁺), Rₜ=0.72 min. ¹H NMR (400 MHz, <cdcl3>) δ ppm 1.03 (d, J=6.65 Hz, 3H), 1.34-1.51 (m, 54H), 1.72-1.87 (m, 1H), 2.05 (s, 1H), 2.46-2.57 (m, 1H), 2.69 (t, J=11.35 Hz, 1H), 2.78-2.94 (m, 1H), 3.00-3.14 (m, 1H), 3.45 (d, J=12.52 Hz, 1H), 3.53-3.76 (m, 1H), 4.63 (d, J=6.26 Hz, 1H), 6.88 (d, J=5.48 Hz, 3H), 8.13 (s, 3H), 8.26-8.36 (m, 3H).

### Synthesis of (3R,4R,5S)-1-(3-(bis(tert-butoxycarbonyl)amino)pyridin-4-yl)-3-((tert-butoxycarbonyl)amino)-5-methylpiperidin-4-yl methanesulfonate

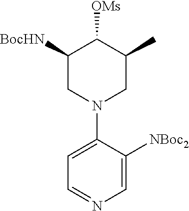

To a solution of tert-butyl tert-butoxycarbonyl(4-((3R,4R,5S)-3-((tert-butoxycarbonyl)amino)-4-hydroxy-5-methylpiperidin-1-yl)pyridin-3-yl)carbamate (1.0 equiv.) in DCM (0.20 M) was added TEA (1.7 equiv.), followed by MsCl (1.3 equiv.). The capped solution was stirred at rt for 90 mins. The reaction mixture was quenched with NaHCO_{3(sat.)}, and extracted with EtOAc. The organic layer was washed with NaCl_{(sat.)}, dried over MgSO₄, filtered, concentrated to yield (3R,4R,5S)-1-(3-(bis(tert-butoxycarbonyl)amino)pyridin-4-yl)-3-((tert-butoxycarbonyl)amino)-5-methylpiperidin-4-yl methanesulfonate in 99% yield. LC/MS (m/z)=601.3 (MH⁺), Rₜ=0.83 min.

### Synthesis of tert-butyl (4-((3R,4S,5S)-4-azido-3-((tert-butoxycarbonyl)amino)-5-methylpyridin-1-yl)pyridin-3-yl)(tert-butoxycarbonyl)carbamate

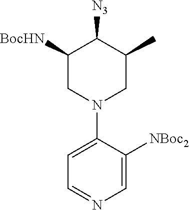

To a solution of (3R,4R,5S)-1-(3-(bis(tert-butoxycarbonyl)amino)pyridin-4-yl)-3-((tert-butoxycarbonyl)amino)-5-methylpiperidin-4-yl methanesulfonate (1.0 equiv.) in DMF (0.13 M) was added NaN₃ (5.0 equiv.). The solution was submerged in an 80° C. oil bath and left stirring under Ar for 24 hrs. The solution was cooled to rt and left stirring under Ar overnight. The solution was partitioned between EtOAc and H₂O. The organic layer was washed with Na₂CO_{3(sat.)}, NaCl_{(sat.)}, dried over MgSO₄, filtered, concentrated to yield tert-butyl (4-((3R,4S,5 S)-4-azido-3-((tert-butoxycarbonyl)amino)-5-methylpiperidin-1-yl)pyridin-3-yl)(tert-butoxycarbonyl)carbamate in 60% yield. LC/MS (m/z)=548.4 (MH⁺), Rₜ=0.94 min. ¹H NMR (400 MHz, <cdcl3>) δ ppm 0.97-1.13 (m, 3H), 1.33-1.52 (m, 30H), 2.03-2.19 (m, 1H), 2.66-2.87 (m, 2H), 3.16 (dd, J=12.72, 2.15 Hz, 1H), 3.22-3.32 (m, 1H), 3.81-4.01 (m, 2H), 4.78 (d, J=9.00 Hz, 1H), 6.76-6.88 (m, 1H), 8.05-8.18 (m, 1H), 8.26-8.37 (m, 1H).

### Synthesis of tert-butyl ((3R,4S,5S)-1-(3-aminopyridin-4-yl)-4-azido-5-methylpiperidin-3-yl)carbamate

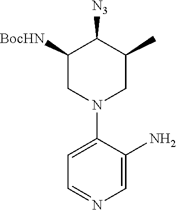 [SMILES: C[C@H]1CN(c2ccncc2N)C[C@@H](NC(=O)OC(C)(C)C)[C@H]1N=[N+]=[N-]]

A solution of 4 M HCl in dioxane (30.0 equiv.) was added to tert-butyl (4-((3R,4S,5S)-4-azido-3-((tert-butoxycarbonyl)amino)-5-methylpiperidin-1-yl)pyridin-3-yl)(tert-butoxycarbonyl)carbamate (1.0 equiv.). The solution started to go homogeneous for a few minutes, but then a ppt formed and the solution went very thick. After sitting at rt for 1 hour, the volatiles were removed in vacuo and the solid was pumped on for 5 minutes on the high vac. To the residue was added CH₂Cl₂ (0.11 M), TEA (5.0 equiv.) and Boc₂O (1.0 equiv.). The solution was left stirring at rt for 1 hr. The volatiles were removed in vacuo and the residue was partitioned between EtOAc and H₂O. The organic layer was washed with Na₂CO_{3(sat.)}, NaCl_{(sat.)}, dried over MgSO₄, filtered, concentrated and purified by ISCO SiO₂ chromatography to yield tert-butyl ((3R,4S,5S)-1-(3-aminopyridin-4-yl)-4-azido-5-methylpiperidin-3-yl)carbamate in 33% yield. LC/MS (m/z)=348.2 (MH⁺), Rₜ=0.70 min. ¹H NMR (400 MHz, CHLOROFORM-d) δ ppm 1.02-1.18 (m, 3H), 1.36-1.54 (m, 10H), 2.19 (qd, J=6.91, 3.91 Hz, 1H), 2.57 (q, J=10.96 Hz, 2H), 2.96 (d, J=9.00 Hz, 1H), 3.20 (dd, J=11.15, 3.72 Hz, 1H), 3.55-3.73 (m, 2H), 3.90 (br. s., 1H), 4.01 (br. s., 1H), 4.81 (d, J=8.61 Hz, 1H), 6.72-6.83 (m, 1H), 7.96 (d, J=5.09 Hz, 1H), 8.02 (s, 1H).

### Synthesis of di-tert-butyl (4-((3R,4S,5S)-3-((tert-butoxycarbonyl)amino)-5-methyl-4-(methylamino)piperidin-1-yl)pyridin-3-yl)iminodicarbonate

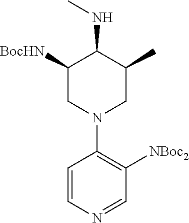

To a solution of di-tert-butyl 4-((3R,4S,5S)-4-azido-3-(tert-butoxycarbonylamino)-5-methylpiperidin-1-yl)pyridin-3-yliminodicarbonate (1.0 equiv.) in DCM (0.14 M) at rt was added PMe₃ (2.0 equiv.). After stirring at rt for 2 hr, PARAFORMALDEHYDE (5.0 equiv.) was added and the mixture was stirred at rt for another 2.5 hrs. The reaction was added MeOH (0.14 M), cooled to 0° C. and added NaBH₄ (5.0 equiv.). After 30 min at rt, the reaction was quenched with sat. NaHCO₃ and extract with EtOAc to yield di-tert-butyl 4-((3R,4S,5 S)-3-(tert-butoxycarbonylamino)-5-methyl-4-(methylamino)piperidin-1-yl)pyridin-3-yliminodicarbonate in 85% yield. LC/MS (m/z)=536.3 (MH⁺), Rₜ=0.61 min.

### Synthesis of methyl ((3R,4S,5S)-1-(3-aminopyridin-4-yl)-3-((tert-butoxycarbonyl)amino)-5-methylpiperidin-4-yl)(methyl)carbamate

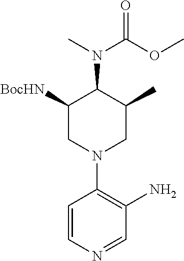 [SMILES: COC(=O)N(C)[C@H]1[C@@H](C)CN(c2ccncc2N)C[C@H]1NC(=O)OC(C)(C)C]

To a solution of di-tert-butyl 4-((3R,4S,5S)-3-(tert-butoxycarbonylamino)-5-methyl-4-(methylamino)piperidin-1-yl)pyridin-3-yliminodicarbonate (1.0 equiv.) in DCM (0.10 M) was added DIEA (3.0 equiv.) the reaction mixture was then cooled to 0° C. To this solution was added methyl chloroformate (1.2 equiv.). The resulting mixture was at RT for 50 min. The reaction mixture was quenched with NaHCO₃ and diluted with EtOAc. The aqeuous layer was separated and extracted with EtOAc, the combined organics were then dried over MgSO₄ and concentrated in vaccuo. 4 M HCl (43.0 equiv.) in dioxane was added to the residue. After 1 hr, the volatile was removed in vacuo. To the solution of the residue in DCM (0.10 M) at 0° C. was added DIEA (3.0 equiv.) and BocOSu (1.0 equiv.). After 60 min at rt, The reaction mixture was quenched with NaHCO₃ and diluted with EtOAc. The aqueous layer was separated and extracted with EtOAc, the combined organics were then dried over MgSO₄ and concentrated in vaccuo to yield a yellow residue, which was purified by ISCO SiO₂ chromatography to yield methyl ((3R,4S,5S)-1-(3-aminopyridin-4-yl)-3-((tert-butoxycarbonyl)amino)-5-methylpiperidin-4-yl)(methyl)carbamate in 44% yield. LC/MS (m/z)=394.2 (MH⁺), Rₜ=0.59 min. ¹H NMR (400 MHz, <cdcl3>) δ ppm 1.06 (d, J=7.04 Hz, 3H), 1.40-1.52 (m, 10H), 2.28-2.42 (m, 1H), 2.89 (d, J=4.70 Hz, 2H), 3.08 (dd, J=11.93 Hz, 4.50 Hz, 1H), 3.15 (s, 3H), 3.47 (dd, J=11.15, 4.11 Hz, 1H), 3.67-3.79 (m, 5H), 4.13-4.23 (m, 1H), 4.56-4.77 (m, 1H), 6.80 (d, J=5.09 Hz, 1H), 7.97 (d, J=5.48 Hz, 1H), 8.04 (s, 1H).

### Synthesis of methyl ((3R,4S,5S)-3-((tert-butoxycarbonyl)amino)-1-(3-isothiocyanatopyridin-4-yl)-5-methylpiperidin-4-yl)(methyl)carbamate

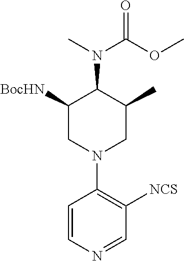

A solution of methyl ((3R,4S,5S)-1-(3-aminopyridin-4-yl)-3-((tert-butoxycarbonyl)amino)-5-methylpiperidin-4-yl)(methyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, methyl ((3R,4S,5S)-3-((tert-butoxycarbonyl)amino)-1-(3-isothiocyanatopyridin-4-yl)-5-methylpiperidin-4-yl)(methyl)carbamate is obtained.

### Synthesis of tert-butyl ((3R,4S,5S)-1-(3-aminopyridin-4-yl)-5-methyl-4-(N-methylacetamido)piperidin-3-yl)carbamate

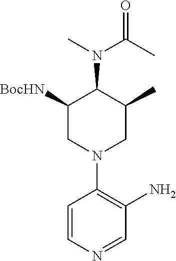 [SMILES: CC(=O)N(C)[C@H]1[C@@H](C)CN(c2ccncc2N)C[C@H]1NC(=O)OC(C)(C)C]

To a solution of di-tert-butyl 4-((3R,4S,5S)-3-(tert-butoxycarbonylamino)-5-methyl-4-(methylamino)piperidin-1-yl)pyridin-3-yliminodicarbonate (1.0 equiv.) in DCM (0.10 M) was added DIEA (3.0 equiv.) the reaction mixture was then cooled to 0° C. To this solution was added acetic anhydride (1.2 equiv.). The resulting mixture was at RT for 50 min. The reaction mixture was quenched with NaHCO₃ and diluted with EtOAc. The aqeuous layer was separated and extracted with EtOAc, the combined organics were then dried over MgSO₄ and concentrated in vacuo. 4 M HCl (43.0 equiv.) in Dioxane was added to the residue. After 1 hr, the volatile was removed in vacuo. To the solution of the residue in DCM (0.10 M) at 0° C. was added DIEA (3.0 equiv.) and BocOSu (1.0 equiv.). After 60 min at rt, The reaction mixture was quenched with NaHCO₃ and diluted with EtOAc. The aqeuous layer was separated and extracted with EtOAc, the combined organics were then dried over MgSO₄ and concentrated in vaccuo to yield a yellow residue, which was purified by SiO₂ chromatography to yield tert-butyl ((3R,4S,5S)-1-(3-aminopyridin-4-yl)-5-methyl-4-(N-methylacetamido)piperidin-3-yl)carbamate in 60% yield. LC/MS (m/z)=378.2 (MH⁺), Rₜ=0.50 min. ¹H NMR (400 MHz, <cdcl3>) δ ppm 1.06 (d, J=7.83 Hz, 3H), 1.39-1.50 (m, 9H), 2.20 (br. s., 3H), 2.34-2.48 (m, 1H), 2.84-3.28 (m, 6H), 3.77 (d, J=18.00 Hz, 2H), 4.19-4.62 (m, 1H), 6.82 (d, J=5.09 Hz, 1H), 7.97 (br. s., 1H), 8.05 (br. s., 1H).

### Synthesis of tert-butyl ((3R,4S,5S)-1-(3-isothiocyanatopyridin-4-yl)-5-methyl-4-(N-methylacetamido)piperidin-3-yl)carbamate

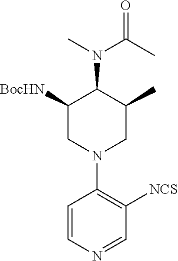

A solution of tert-butyl ((3R,4S,5S)-1-(3-aminopyridin-4-yl)-5-methyl-4-(N-methylacetamido)piperidin-3-yl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((3R,4S,5S)-1-(3-isothiocyanatopyridin-4-yl)-5-methyl-4-(N-methylacetamido)piperidin-3-yl)carbamate is obtained.

### Synthesis of 3-(4,4,5,5-tetramethyl-1,3,2-dioxaborolan-2-yl)cyclohex-2-enone

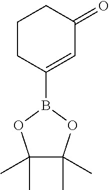 [SMILES: CC1(C)OB(C2=CC(=O)CCC2)OC1(C)C]

To a solution of 3-oxocyclohex-1-enyl trifluoromethanesulfonate (1.0 equiv.) in degassed dioxane (0.3 M) was added bis(pinacolato)diboron (2.0 equiv.), KOAc (3.0 equiv.), and Pd(dppf)Cl₂-DCM (0.05 equiv.). The reaction was heated to 80° C. for 2 h, then filtered. The dioxane solution was used for the next step without further purification. LC/MS=140.9 (M+H of boronic acid).

### Synthesis of 3-(3-nitropyridin-4-yl)cyclohex-2-enone

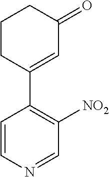 [SMILES: O=C1C=C(c2ccncc2[N+](=O)[O-])CCC1]

To a solution of 3-(4,4,5,5-tetramethyl-1,3,2-dioxaborolan-2-yl)cyclohex-2-enone (1.0 equiv.) in degassed dioxane and 2M Na₂CO₃ was added 4-chloro-3-nitropyridine (1.2 equiv.) and Pd(dppf)Cl₂-DCM (0.05 equiv.). The reaction was heated in an oil bath to 110° C. for 2 hours. Cooled to room temperature, then diluted with EtOAc, added H₂O—dark solution, lots of emulsions. Filtered to get rid of the solids, then extracted the organic phase, dried with Na₂SO₄, and concentrated. The crude was purified via silica gel chromatography eluting with ethyl acetate and hexanes (1:1) to yield 3-(3-nitropyridin-4-yl)cyclohex-2-enone (55%, 2 steps). LC/MS=219 (M+H), LC=2.294 min.

### Synthesis of 3-(3-nitropyridin-4-yl)cyclohex-2-enol

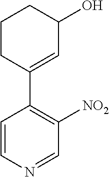 [SMILES: O=[N+]([O-])c1cnccc1C1=CC(O)CCC1]

To a solution of 3-(3-nitropyridin-4-yl)cyclohex-2-enone (1.0 equiv.) was added EtOH (0.2 M) and CeCl₃-7H₂O (1.3 equiv.). The reaction was cooled to 0° C., then NaBH₄ (1.3 equiv.) was added in portions. Stirred for 2 h at 0° C., then quenched by adding water, concentrated to remove the EtOH, added EtOAc, extracted the organics, dried with brine, then Na₂SO₄, and concentrated to yield 3-(3-nitropyridin-4-yl)cyclohex-2-enol (99%). LC/MS=221.1 (M+H), LC=2.235 min.

### Synthesis of 2-(3-(3-nitropyridin-4-yl)cyclohex-2-enyl)isoindoline-1,3-dione

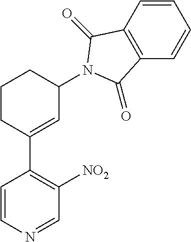 [SMILES: O=C1c2ccccc2C(=O)N1C1C=C(c2ccncc2[N+](=O)[O-])CCC1]

To a solution of 3-(3-nitropyridin-4-yl)cyclohex-2-enol (1.0 equiv.), triphenylphosphine (1.5 equiv.) and phthalimide (1.5 equiv.) in THF (0.3 M) at 0° C. was added (E)-di-tert-butyl diazene-1,2-dicarboxylate (1.5 equiv.) dropwise. The reaction was stirred at 0° C. for 2 hours. Concentrated to dryness under vacuo, then purified the crude via silica gel column chromatography eluting with EtOAc and hexanes (1:1 with 5% methanol) to afford the 2-(3-(3-nitropyridin-4-yl)cyclohex-2-enyl)isoindoline-1,3-dione (63% yield). LC/MS=350.3 (M+H), LC=3.936 min.

### Synthesis of 2-(3-(3-aminopyridin-4-yl)cyclohex-2-enyl)isoindoline-1,3-dione

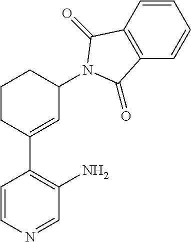 [SMILES: Nc1cnccc1C1=CC(N2C(=O)c3ccccc3C2=O)CCC1]

To a solution of 2-(3-(3-nitropyridin-4-yl)cyclohex-2-enyl)isoindoline-1,3-dione (1.0 equiv.) in AcOH (0.38 M) was added Fe (6.0 equiv.) and the reaction was stirred at room temperature for 2 h. Filtered, then washed with methanol and concentrated the filtrate. To the crude was added ethyl acetate and saturated NaHCO₃, the organics were dried with Na₂SO₄, and concentrated to give 2-(3-(3-aminopyridin-4-yl)cyclohex-2-enyl)isoindoline-1,3-dione as a yellow thick gum in 96% yield. LC/MS=320.0 (M+H), LC=2.410 min.

### Synthesis of 2-(3-(3-aminopyridin-4-yl)cyclohexyl)isoindoline-1,3-dione

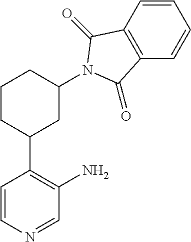 [SMILES: Nc1cnccc1C1CCCC(N2C(=O)c3ccccc3C2=O)C1]

To a solution of 2-(3-(3-nitropyridin-4-yl)cyclohex-2-enyl)isoindoline-1,3-dione (1.0 equiv.) in acetic acid (0.1 M) was added 10% Pd/C (0.2 equiv.) and the reaction was stirred under a H₂ balloon. After 3 days, the reaction was filtered through Celite, washed with ethyl acetate and methanol, the filtrate was diluted with ethyl acetate and washed twice with sat. 2M Na₂CO₃. The organic phase was dried with magnesium sulfate, filtered and concentrated. The crude material was triturated with hexanes and ethyl acetate to afford 2-(3-(3-aminopyridin-4-yl)cyclohexyl)isoindoline-1,3-dione in 73% yield. LC/MS=322.2 (M+H), Rt=0.64 min.

### Synthesis of 2-((1S,3R)-3-(3-isothiocyanatopyridin-4-yl)cyclohexyl)isoindoline-1,3-dione

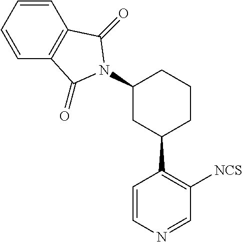

A solution of 2-((1S,3R)-3-(3-aminopyridin-4-yl)cyclohexyl)isoindoline-1,3-dione (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, 2-((1S,3R)-3-(3-isothiocyanatopyridin-4-yl)cyclohexyl)isoindoline-1,3-dione is obtained.

### Synthesis of 5-methyl-3-oxocyclohex-1-enyltrifluoromethanesulfonate

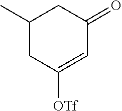 [SMILES: CC1CC(=O)C=C(OS(=O)(=O)C(F)(F)F)C1]

To a solution of 5-methylcyclohexane-1,3-dione (1.0 equiv.) in DCM (0.5M) was added Na₂CO₃ (1.1 equiv.) and cooled to 0° C. Added Tf₂O (1.0 equiv.) in DCM (5.0 M) dropwise over 1 hr at 0° C. under a nitrogen atmosphere. Upon addition, the reaction was stirred for 1 hr at room temperature (dark red solution). The solution was filtered and the filtrate was quenched by careful addition of saturated NaHCO₃ with vigorous stirring until pH=7. The solution was transferred to a separatory funnel and the layers were separated. The organic layer was washed with brine, dried with Na₂SO₄, filtered, concentrated under vacuo and dried under high vacuum for 15 min to yield 5-methyl-3-oxocyclohex-1-enyl trifluoromethanesulfonate as light yellow oil in 78% yield. The triflate decomposes upon storage and should be used immediately for the next reaction. LC/MS=259.1/300.1 (M+H and M+CH₃CN); Rt=0.86 min, LC=3.84 min. ¹H-NMR (400 MHz, CDCl₃) δ ppm: 6.05 (s, 1H), 2.70 (dd, J=17.2, 4.3, 1H), 2.53 (dd, J=16.6, 3.7, 1H), 2.48-2.31 (m, 2H), 2.16 (dd, J=16.4, 11.7, 1H), 1.16 (d, J=5.9, 3H).

### Synthesis of 5-methyl-3-(4,4,5,5-tetramethyl-1,3,2-dioxaborolan-2-yl)cyclohex-2-enone

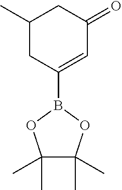 [SMILES: CC1CC(=O)C=C(B2OC(C)(C)C(C)(C)O2)C1]

To a solution of 5-methyl-3-oxocyclohex-1-enyl trifluoromethanesulfonate (1.0 equiv.) in degassed dioxane (0.7 M) was added bis(pinacolato)diboron (2.0 equiv.), KOAc (3.0 equiv.), and Pd(dppf)Cl₂-DCM (0.03 equiv.). The reaction was heated to 80° C. for 10 h then cooled to room temperature and filtered through a coarse frit glass funnel. The cake was rinsed with more dioxane and the filtrate solution was used for the next step without further purification. LC/MS=155.1 (M+H of boronic acid); Rt=0.41 min, LC=1.37 min.

### Synthesis of 5-methyl-3-(3-nitropyridin-4-yl)cyclohex-2-enone

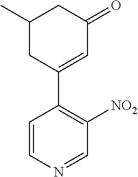 [SMILES: CC1CC(=O)C=C(c2ccncc2[N+](=O)[O-])C1]

To a solution of 5-methyl-3-(4,4,5,5-tetramethyl-1,3,2-dioxaborolan-2-yl)cyclohex-2-enone (1.0 equiv.) in degassed dioxane (0.5 M) and 2M Na₂CO₃ (2 equiv.) was added 4-chloro-3-nitropyridine (1.3 equiv.) and Pd(dppf)Cl₂-DCM (0.05 equiv.). The reaction was placed under a reflux condenser and heated in an oil bath to 110° C. for 1 h. Cooled to room temperature, filtered through a pad of Celite, washed the pad with ethyl acetate and concentrated the filtrate under vacuo. The residue was further pumped at 80° C. on a rotary evaporator for one hour to remove boronate by-products (M+H=101) via sublimation. The residue was partitioned between brine and ethyl acetate, and the layers were separated, the aqueous phase was further extracted with ethyl acetate (4×), the organics were combined, dried over sodium sulfate, filtered, and concentrated. The crude was purified via silica gel chromatography loading in DCM and eluting with 2-50% ethyl acetate and hexanes. The pure fractions were concentrated in vacuo to yield an orange oil. The oil was placed under high vacuum (˜500 mtorr) with seed crystals overnight to yield an orange solid. The solid was further purified via trituration in hexanes to yield 5-methyl-3-(3-nitropyridin-4-yl) cyclohex-2-enone (48% 2 steps). LC/MS=233.2 (M+H); Rt=0.69 min, LC=2.70 min. ¹H-NMR (400 MHz, CdCl₃) δ ppm: 9.31 (s, 1H), 8.88 (d, J=5.1, 1H), 7.30 (d, J=5.1, 1H), 6.00 (d, J=2.4, 1H), 2.62 (dd, J=16.4, 3.5, 1H), 2.53-2.34 (m, 3H), 2.23 (dd, J=16.1, 11.7, 1H), 1.16 (d, J=6.3, 3H).

### Synthesis of cis-(+/−)-5-methyl-3-(3-nitropyridin-4-yl)cyclohex-2-enol

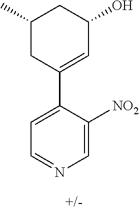 [SMILES: C[C@H]1CC(c2ccncc2[N+](=O)[O-])=C[C@@H](O)C1]

To a solution of 5-methyl-3-(3-nitropyridin-4-yl)cyclohex-2-enone (1.0 equiv.) in EtOH (0.3 M) was added CeCl₃-7H₂O (1.2 equiv.). The reaction was cooled to 0° C., then NaBH₄ (1.2 equiv.) was added in portions. Stirred for 1 h at 0° C., then quenched by adding water, concentrated to remove the EtOH, added EtOAc, extracted the organics, washed with brine, then dried with Na₂SO₄, filtered and concentrated to yield cis-(+/−)-5-methyl-3-(3-nitropyridin-4-yl)cyclohex-2-enol (94%). LC/MS=235.2 (M+H), LC=2.62 min.

### Synthesis of cis-(+/−)-5-methyl-3-(3-nitropyridin-4-yl)cyclohex-2-enol

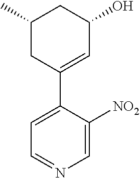 [SMILES: C[C@H]1CC(c2ccncc2[N+](=O)[O-])=C[C@@H](O)C1]

To a solution of 5-methyl-3-(3-nitropyridin-4-yl)cyclohex-2-enone (1.0 equiv.) in EtOH (0.3 M) was added CeCl₃-7H₂O (1.2 equiv.). The reaction was cooled to 0° C., then NaBH₄ (1.2 equiv.) was added in portions. Stirred for 1 h at 0° C., then quenched by adding water, concentrated to remove the EtOH, added EtOAc, extracted the organics, washed with brine, then dried with Na₂SO₄, filtered and concentrated to yield cis-(+/−)-5-methyl-3-(3-nitropyridin-4-yl)cyclohex-2-enol (94%). LC/MS=235.2 (M+H), LC=2.62 min.

### Synthesis of 4-(3-(tert-butyldimethylsilyloxy)-5-methylcyclohex-1-enyl)-3-nitropyridine

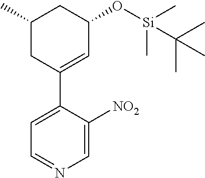 [SMILES: C[C@H]1CC(c2ccncc2[N+](=O)[O-])=C[C@@H](O[Si](C)(C)C(C)(C)C)C1]

To a solution of 5-methyl-3-(3-nitropyridin-4-yl)cyclohex-2-enol (1.0 equiv.) in DMF (0.5 M) was added imidazole (4.0 equiv.) and TBDMSCl (2.5 equiv.). After stirring for 18 hours the solution was portioned between EtOAc and H₂O and separated. After washing further with H₂O (3×) and NaCl (sat.), drying over MgSO₄, filtering and removal of solvents, 4-(3-(tert-butyldimethylsilyloxy)-5-methylcyclohex-1-enyl)-3-nitropyridine was obtained (85%). LC/MS=349.2 (M+H), LC=5.99 min.

### Synthesis of 4-(3-(tert-butyldimethylsilyloxy)-5-methylcyclohex-1-enyl)pyridin-3-amine

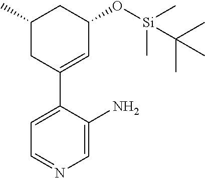 [SMILES: C[C@H]1CC(c2ccncc2N)=C[C@@H](O[Si](C)(C)C(C)(C)C)C1]

A heterogeneous solution of 4-(3-(tert-butyldimethylsilyloxy)-5-methylcyclohex-1-enyl)-3-nitropyridine (1.0 eq.) and iron (6.0 eq) in acetic acid, at a concentration of 0.4 M, was stirred vigorously for 2 hours. The mixture was then passed through a celite pad, eluting with MeOH. Upon removal of the volatiles in vacuo, the residue was dissolved in EtOAc, washed with Na₂CO_{3(sat.)}, NaCl_{(sat.)}, was dried over MgSO₄, was filtered and the volatiles were removed in vacuo yielding 4-(3-(tert-butyldimethylsilyloxy)-5-methylcyclohex-1-enyl)pyridin-3-amine (78%). LCMS (m/z): 319.3 (MH⁺); LC Rₜ=3.77 min.

### Synthesis of 4-(3-(tert-butyldimethylsilyloxy)-5-methylcyclohexyl)pyridin-3-amine

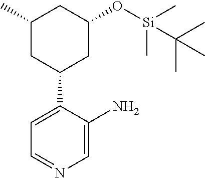 [SMILES: C[C@@H]1C[C@H](O[Si](C)(C)C(C)(C)C)C[C@H](c2ccncc2N)C1]

To a solution of 4-(3-(tert-butyldimethylsilyloxy)-5-methylcyclohex-1-enyl)-3-nitropyridine (1.0 equiv.) in methanol, at a concentration of 0.1 M, was added 10% palladium on carbon (0.1 eq.). The resultant heterogeneous solution was put under an atmosphere of hydrogen and was stirred for 15 hours. At this time the mixture was filtered through a pad of celite eluting with methanol. The volatiles were removed in vacuo yielding 4-(3-(tert-butyldimethylsilyloxy)-5-methylcyclohexyl)pyridin-3-amine (90%). LCMS (m/z): 321.3 (MH⁺); LC Rₜ=3.85 min.

### Synthesis of cis (+/−) benzyl 4-3-(tert-butyldimethylsilyloxy)-5-methylcyclohexyl)pyridin-3-ylcarbamate

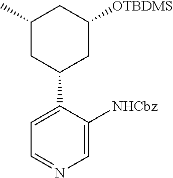 [SMILES: C[C@@H]1C[C@H](O[Si](C)(C)C(C)(C)C)C[C@H](c2ccncc2NC(=O)OCc2ccccc2)C1]

To a solution of cis-(+/−)-4-(3-(tert-butyldimethylsilyloxy)-5-methylcyclohexyl)pyridin-3-amine in dichloromethane at a concentration of 0.5 M was added benzyl 2,5-dioxopyrrolidin-1-yl carbonate (1.1 equiv.) and DMAP (0.05 equiv.). After stirring for 16 hours at rt, additional benzyl 2,5-dioxopyrrolidin-1-yl carbonate (0.55 equiv.) and DMAP (0.03 equiv.) were added. After stirring for an additional 24 hours at rt, additional benzyl 2,5-dioxopyrrolidin-1-yl carbonate (0.1 equiv.) and DMAP (0.03 equiv.) were added. After stirring for 18 more hours the solution was partitioned between EtOAc and Na₂CO_{3(sat.)} and separated. Upon further washing with Na₂CO_{3(sat.)} (2×) and NaCl_{(sat.)}, drying over MgSO₄, filtering and removal of solvents, cis (+/−) benzyl 4-3-(tert-butyldimethylsilyloxy)-5-methylcyclohexyl)pyridin-3-ylcarbamate was obtained. The crude material was used as is. LC/MS=455.3 (M+H), LC=4.39 min.

### Synthesis of cis-(+/−)benzyl 4-(3-hydroxy-5-methylcyclohexyl)pyridin-3-ylcarbamate

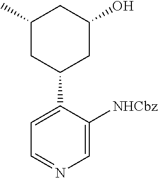 [SMILES: C[C@@H]1C[C@H](O)C[C@H](c2ccncc2NC(=O)OCc2ccccc2)C1]

A solution of cis (+/−) benzyl 4-3-(tert-butyldimethylsilyloxy)-5-methylcyclohexyl)pyridin-3-ylcarbamate in 1:2:1 6N HCl/THF/MeOH at a concentration of 0.1 M was stirred at rt for 6 hours. The pH was than adjusted to pH=7 by addition of 6N NaOH and the volatiles were removed in vacuo. The aqueous layer was extracted with EtOAc and the organic was washed with NaCl_{(sat.)}, dried over MgSO₄, filtered and upon removal of the volatiles in vacuo, cis-(+/−)benzyl 4-(3-hydroxy-5-methylcyclohexyl)pyridin-3-ylcarbamate was obtained. The crude material was used as is. LC/MS=341.2 (M+H), LC=2.38 min.

### Synthesis of cis (+/−)-benzyl 4-(3-methyl-5-oxocyclohexyl)pyridin-3-ylcarbamate

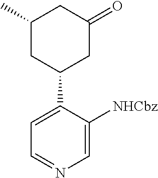 [SMILES: C[C@@H]1CC(=O)C[C@H](c2ccncc2NC(=O)OCc2ccccc2)C1]

To a 0° C. solution of cis-(+/−)-benzyl 4-(3-hydroxy-5-methylcyclohexyl)pyridin-3-ylcarbamate in wet CH₂Cl₂ at a concentration of 0.16 M was added Dess-Martin Periodinane (1.5 equiv.) and the solution was stirred for 18 hours as it warmed to rt. The solution was partitioned between EtOAc and 1:1 10% Na₂S₂O₃/NaHCO_{3(sat.)} and separated. Upon further washing with 1:1 10% Na₂S₂O₃/NaHCO_{3(sat.)} (2×) and NaCl_{(sat.)}, drying over MgSO₄, filtering, removal of solvents and purification by silica gel chromatography (75-100% EtOAc/hexanes), cis-(+/−)-benzyl-4-(3-methyl-5-oxocyclohexyl)pyridin-3-ylcarbamate was obtained as a white solid (53%, 5 steps). LC/MS=339.2 (M+H).

### Synthesis of cis-(+/−)-benzyl 4-(-3-(benzylamino)-5-methylcyclohexyl)pyridin-3-ylcarbamate

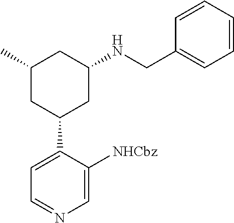 [SMILES: C[C@@H]1C[C@H](NCc2ccccc2)C[C@H](c2ccncc2NC(=O)OCc2ccccc2)C1]

A solution of cis-(+/−)-benzyl-4-(3-methyl-5-oxocyclohexyl)pyridin-3-ylcarbamate (1.0 equiv) and benzylamine (3.0 equiv) in MeOH, at a concentration of 0.25 M, was stirred at rt for 2 hours. Upon cooling in a −78° C. bath, LiBH₄ (1.1 equiv, 2.0 M in THF) was added and the solution was allowed to warm to rt with stirring over 16 hours. The solution was partitioned between EtOAc and NaHCO_{3(sat.)}, separated, washed further with NaHCO_{3(sat.)} and NaCl_{(sat.)}, dried over MgSO₄, filtered and after removal of volatiles in vacuo, cis-(+/−)-benzyl 4-(-3-(benzylamino)-5-methylcyclohexyl)pyridin-3-ylcarbamate was obtained as a 4:1 mixture of isomers, with the all cis as predominant LC/MS=430.3 (M+H), LC=0.62 min.

### Synthesis of cis (+/−)-tert-butyl (-3-(3-aminopyridin-4-yl)-5-methylcyclohexylcarbamate

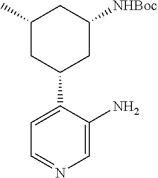 [SMILES: C[C@@H]1C[C@H](NC(=O)OC(C)(C)C)C[C@H](c2ccncc2N)C1]

To a solution of cis-(+/−)-benzyl 4-(-3-(benzylamino)-5-methylcyclohexyl)pyridin-3-ylcarbamate was (1.0 equiv.) in methanol, at a concentration of 0.07 M, was added 20% palladium hydroxide on carbon (0.2 eq.). The resultant heterogeneous solution was put under an atmosphere of hydrogen and was stirred for 14 hours. At this time the reaction was purged with Ar, Boc₂O (1.0 equiv.) was added and the solution was stirred for 8 hours. Additional Boc₂O (1.0 equiv.) was added and the solution was stirred for 16 more hours. At this time the mixture was filtered through a pad of celite eluting with methanol. Upon removal of volatiles in vacuo, purification by silica gel chromatography (2.5-2.5 MeOH/CH₂Cl₂ with 0.1% DIEA) and recrystallization from 10% EtOAc/hexanes yielded cis (+/−)-tert-butyl (-3-(3-aminopyridin-4-yl)-5-methylcyclohexylcarbamate (49%). LCMS (m/z): 306.3 (MH⁺), LC Rₜ=2.59 min. Pure enantiomers could be obtained by chiral chromatography.

### Synthesis of tert-butyl ((1S,3R,5S)-3-(3-isothiocyanatopyridin-4-yl)-5-methylcyclohexyl)carbamate

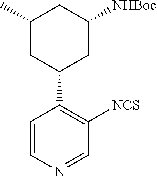

A solution of tert-butyl ((1 S,3R,5S)-3-(3-aminopyridin-4-yl)-5-methylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1S,3R,5S)-3-(3-isothiocyanatopyridin-4-yl)-5-methylcyclohexyl)carbamate is obtained.

### Synthesis of (+/−)-4-(5-methylcyclohexa-1,3-dienyl)-3-nitropyridine

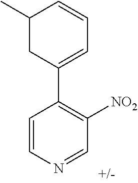 [SMILES: CC1C=CC=C(c2ccncc2[N+](=O)[O-])C1]

To a solution of (+/−)-5-methyl-3-(3-nitropyridin-4-yl)cyclohex-2-enol (1.0 equiv.) in dioxane (0.1M) was added p-TSA (1.0 equiv.), and the reaction was stirred at 100° C. for 3 h. The solution was cooled to room temperature, then passed through a pad of neutral alumina eluting with EtOAc to yield (+/−)-4-(5-methylcyclohexa-1,3-dienyl)-3-nitropyridine as a yellow oil in 68% yield. LC/MS=217.1 (M+H), LC=3.908 min.

### Synthesis of (+/−)-6-bromo-5-methyl-3-(3-nitropyridin-4-yl)cyclohex-2-enol

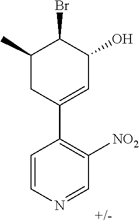 [SMILES: C[C@@H]1CC(c2ccncc2[N+](=O)[O-])=C[C@@H](O)[C@@H]1Br]

To a solution of 4-(5-methylcyclohexa-1,3-dienyl)-3-nitropyridine (1.0 equiv.) in THF and water (1:1, 0.13 M) was added NBS (1.5 equiv.) and the reaction was stirred at room temperature for 30 min. Upon completion, ethyl acetate and water were added to the reaction, the organic phase was dried with brine, then sodium sulfate, filtered, and concentrated. The crude material was purified via silica gel column chromatography eluting with ethyl acetate and hexanes (1:1) to give (+/−)-6-bromo-5-methyl-3-(3-nitropyridin-4-yl)cyclohex-2-enol as a yellow oil in 80% yield. LC/MS=315.0/313.0 (M+H), LC=2.966 min.

### Synthesis of (+/−)-2-azido-6-methyl-4-(3-nitropyridin-4-yl)cyclohex-3-enol

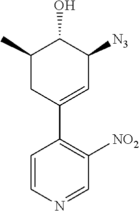 [SMILES: C[C@@H]1CC(c2ccncc2[N+](=O)[O-])=C[C@H](N=[N+]=[N-])[C@H]1O]

To a solution of (+/−)-6-bromo-5-methyl-3-(3-nitropyridin-4-yl)cyclohex-2-enol (1.0 equiv.) in THF (0.1 M) was added potassium tert-butoxide (1.5 equiv.). The reaction turned from orange to black almost immediately. By TLC, the formation of product is clean in 30 min. Quenched by adding saturated ammonium chloride and ethyl acetate. The organic phase was dried with brine, then sodium sulfate, filtered, and concentrated. The crude product was dissolved in ethanol and water (3:1, 0.1 M), and ammonium chloride (2.0 equiv) and sodium azide (2.0 equiv.) were added. The dark orange reaction was stirred at room temperature overnight. The conversion to product is clean as indicated by LC/MS. The reaction was concentrated to remove the ethanol, ethyl acetate and water were added, and the organic phase was dried with sodium sulfate, filtered, and concentrated. The crude material was purified via silica gel column chromatography eluting with ethyl acetate and hexanes (1:1) to give (+/−)-2-azido-6-methyl-4-(3-nitropyridin-4-yl)cyclohex-3-enol in 55% yield. LC/MS=276.0 (M+H), LC=2.803 min.

### Synthesis of (+/−)-tert-butyl 6-hydroxy-5-methyl-3-(3-nitropyridin-4-yl)cyclohex-2-enylcarbamate

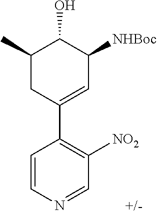 [SMILES: C[C@@H]1CC(c2ccncc2[N+](=O)[O-])=C[C@H](NC(=O)OC(C)(C)C)[C@H]1O]

To a solution of (+/−)-2-azido-6-methyl-4-(3-nitropyridin-4-yl)cyclohex-3-enol (1.0 equiv.) in pyridine and ammonium hydroxide (8:1, 0.08 M) was added trimethylphosphine (3.0 equiv.) and the brown solution was stirred at room temperature for 2 h. Upon completion, EtOH was added and the solution was concentrated in vacuo. More ethanol was added and the reaction was concentrated again. Dioxane and sat. NaHCO₃ (1:1, 0.08 M) were added to the crude, followed by Boc₂O (1.0 equiv.). Stirred the reaction mixture at room temperature for 2 h, then added water and ethyl acetate. The organic phase was dried with MgSO₄, and concentrated. The crude product was purified via silica gel column chromatography eluting with ethyl acetate and hexanes (1:1) to afford (+/−)-tert-butyl 6-hydroxy-5-methyl-3-(3-nitropyridin-4-yl)cyclohex-2-enylcarbamate (59%). LC/MS=350.1 (M+H), Rt: 0.76 min.

### Synthesis of (+/−)-2-(tert-butoxycarbonylamino)-6-methyl-4-(3-nitropyridin-4-yl)cyclohex-3-enyl acetate

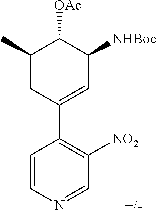 [SMILES: CC(=O)O[C@H]1[C@H](C)CC(c2ccncc2[N+](=O)[O-])=C[C@@H]1NC(=O)OC(C)(C)C]

To a solution of (+/−)-tert-butyl 6-hydroxy-5-methyl-3-(3-nitropyridin-4-yl)cyclohex-2-enylcarbamate (1.0 equiv.) in pyridine (0.1 M) was added Ac₂O (2.0 equiv.) and the reaction was stirred at room temperature overnight. Upon completion, the reaction was concentrated to dryness, then worked-up with ethyl acetate and water. The organic phase was dried with brine, then sodium sulfate, filtered, and concentrated to give (+/−)-2-(tert-butoxycarbonylamino)-6-methyl-4-(3-nitropyridin-4-yl)cyclohex-3-enyl acetate in 94% yield. LC/MS=392.2 (M+H), Rt=0.94 min.

### Synthesis of (1S,2S,4S,6R)-4-(3-aminopyridin-4-yl)-2-((tert-butoxycarbonyl)amino)-6-methylcyclohexyl acetate and (1R,2R,4R,6S)-4-(3-aminopyridin-4-yl)-2-((tert-butoxycarbonyl)amino)-6-methylcyclohexyl acetate

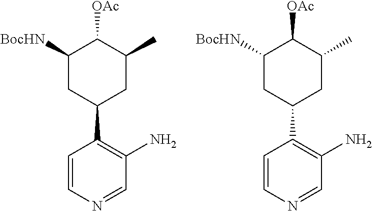 [SMILES: CC(=O)O[C@@H]1[C@@H](C)C[C@@H](c2ccncc2N)C[C@H]1NC(=O)OC(C)(C)C ; CC(=O)O[C@H]1[C@H](C)C[C@H](c2ccncc2N)C[C@@H]1NC(=O)OC(C)(C)C]

To a degassed solution of (+/−)-2-(tert-butoxycarbonylamino)-6-methyl-4-(3-nitropyridin-4-yl)cyclohex-3-enyl acetate (1.0 equiv.) in MeOH and EtOAc (1:1, 0.1 M) was added 10% Pd/C (0.1 equiv.) and the reaction was stirred at room temperature under a hydrogen balloon for 3 days. Upon completion, the solution was filtered through a pad of Celite, the pad was washed with ethyl acetate and the filtrate was concentrated. The crude material contained about 10% of the undesired isomer. The crude was dissolved in ethyl acetate (˜20%) and hexanes and heated until all dissolved. The solution was allowed to sit at room temperature for 2 days. The precipitate was then collected to give (+/−)-4-(3-aminopyridin-4-yl)-2-(tert-butoxycarbonylamino)-6-methylcyclohexyl acetate as the pure product in 59% yield. LC/MS=364.3 (M+H), Rt=0.63 min. The racemic material was resolved using an AD-H chiral column (20% i-PrOH/80% n-heptanes, 20 mL/min flow rate) to (1S,2S,4S,6R)-4-(3-aminopyridin-4-yl)-2-((tert-butoxycarbonyl)amino)-6-methylcyclohexyl acetate (peak#1, Rₜ=3.76 min on AD-H chiral analytical column, 20% i-PrOH/80% n-heptanes, 1 mL/min) and (1R,2R,4R,6S)-4-(3-aminopyridin-4-yl)-2-((tert-butoxycarbonyl)amino)-6-methylcyclohexyl acetate (peak#2, Rₜ=6.79 min on AD-H chiral analytical column, 20% i-PrOH/80% n-heptanes, 1 mL/min).

### Synthesis of (1R,2R,4R,6S)-2-((tert-butoxycarbonyl)amino)-4-(3-isothiocyanatopyridin-4-yl)-6-methylcyclohexyl acetate

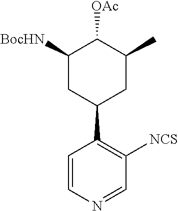

A solution of (1R,2R,4R,6S)-4-(3-aminopyridin-4-yl)-2-((tert-butoxycarbonyl)amino)-6-methylcyclohexyl acetate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, (1R,2R,4R,6S)-2-((tert-butoxycarbonyl)amino)-4-(3-isothiocyanatopyridin-4-yl)-6-methylcyclohexyl acetate is obtained.

### Synthesis of 2-(tert-butoxycarbonylamino)-6-methyl-4-(3-nitropyridin-4-yl)cyclohex-3-enyl methanesulfonate

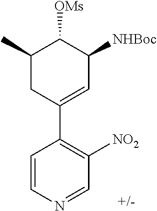 [SMILES: C[C@@H]1CC(c2ccncc2[N+](=O)[O-])=C[C@H](NC(=O)OC(C)(C)C)[C@H]1OS(C)(=O)=O]

To a solution of tert-butyl 6-hydroxy-5-methyl-3-(3-nitropyridin-4-yl)cyclohex-2-enylcarbamate (1.0 equiv.) in DCM (0.09 M) was added triethylamine (1.5 equiv.) and the reaction was cooled to 0° C. MsCl (1.2 equiv.) was added to the reaction and stirred for 3 h. Another 1.0 equiv. of MsCl was added to the reaction and stirred for another 2 h. Worked up the reaction by adding water, the organic phase was dried with brine, sodium sulfate, and concentrated. The crude product was purified via silica gel column chromatography eluting with ethyl acetate and hexanes (1:1) to afford 2-(tert-butoxycarbonylamino)-6-methyl-4-(3-nitropyridin-4-yl)cyclohex-3-enyl methanesulfonate as a white foam in 65% yield. LC/MS=428.2 (M+H), LC: 3.542 min.

### Synthesis of (+/−)-tert-butyl 7-methyl-5-(3-nitropyridin-4-yl)-2-oxo-3a,6,7,7a-tetrahydrobenzo[d]oxazole-3(2H)-carboxylate

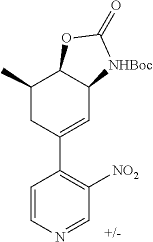

A solution of (+/−)-2-(tert-butoxycarbonylamino)-6-methyl-4-(3-nitropyridin-4-yl)cyclohex-3-enyl methanesulfonate (1.0 equiv.) in pyridine (0.2 M) was heated in the microwave at 110° C. for 10 min. The orange reaction was then concentrated under vacuo, the crude was dissolved in ethyl acetate and water, the organic phase was dried with sodium sulfate and concentrated under vacuo. The crude material was dissolved in DCM (0.2 M), triethylamine (1.8 equiv.) was added, followed by Boc₂O (1.2 equiv.). The reaction was stirred for 40 min, then concentrated to dryness. The crude material was purified via silica gel column chromatography eluting with hexane and ethyl acetate (1:1) to afford (+/−)-tert-butyl 7-methyl-5-(3-nitropyridin-4-yl)-2-oxo-3a,6,7,7a-tetrahydrobenzo[d]oxazole-3(2H)-carboxylate as a white foam in 66% yield. LC/MS=376.0 (M+H), LC: 3.424 min.

### Synthesis of (+/−)-tert-butyl 5-(3-aminopyridin-4-yl)-7-methyl-2-oxohexahydrobenzo[d]oxazole-3(2H)-carboxylate

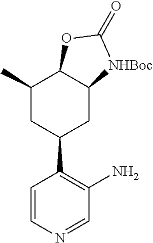

To a degassed solution of (+/−)-tert-butyl 7-methyl-5-(3-nitropyridin-4-yl)-2-oxo-3a,6,7,7a-tetrahydrobenzo[d]oxazole-3(2H)-carboxylate (1.0 equiv.) in MeOH and EtOAc (1:1, 0.1 M) was added 10% Pd/C (0.1 equiv.). The reaction was stirred under a hydrogen balloon overnight. Upon completion, the solution was filtered through a pad of Celite and the pad was washed with ethyl acetate. The filtrate was concentrated under vacuo to give (+/−)-tert-butyl 5-(3-aminopyridin-4-yl)-7-methyl-2-oxohexahydrobenzo[d]oxazole-3(2H)-carboxylate as the desired product as a yellow foam in 93% yield. LC/MS=348.1 (M+H), Rt=055 min.

### Synthesis of (3aR,5R,7S,7aS)-tert-butyl 5-(3-isothiocyanatopyridin-4-yl)-7-methyl-2-oxohexahydrobenzo[d]oxazole-3(2H)-carboxylate

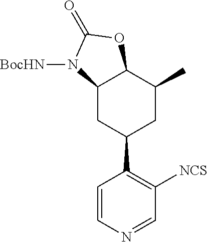

A solution of (3 aR,5R,7S,7aS)-tert-butyl 5-(3-aminopyridin-4-yl)-7-methyl-2-oxohexahydrobenzo[d]oxazole-3(2H)-carboxylate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, (3aR,5R,7S,7aS)-tert-butyl 5-(3-isothiocyanatopyridin-4-yl)-7-methyl-2-oxohexahydrobenzo[d]oxazole-3(2H)-carboxylate is obtained.

### Synthesis of (+/−)-tert-butyl ((1R,2R,3S,5R)-5-(3-((tert-butoxycarbonyl)amino)pyridin-4-yl)-2-hydroxy-3-methylcyclohexyl)carbamate

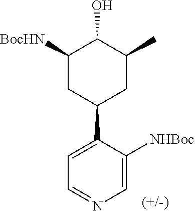 [SMILES: C[C@H]1C[C@@H](c2ccncc2NC(=O)OC(C)(C)C)C[C@@H](NC(=O)OC(C)(C)C)[C@@H]1O]

A solution of (+/−)-(1R,2R,4R,6S)-4-(3-aminopyridin-4-yl)-2-(tert-butoxycarbonylamino)-6-methylcyclohexyl acetate (1.0 equiv.) and Boc₂O (2.1 equiv.) in dioxane (0.34 M) was submerged in an 120° C. oil bath, fitted with a condenser and left stirring under Ar for 6 hrs. The reaction was cooled to rt and the volatiles were removed in vacuo. The residue was dissolved in EtOH (0.34 M), K₂CO₃ (10.0 equiv.) was added, a refluxing head was attached and the heterogeneous solution was submerged in an 50° C. oil bath and left stirring for 24 hrs. The reaction was cooled to rt. The volatiles were removed in vacuo and the residue was partitioned between EtOAc and H₂O. The organic layer was washed with Na₂CO_{3(sat.)}, NaCl_{(sat.)}, dried over MgSO₄, filtered, and concentrated. The residue was dissolved in CH₂Cl₂/Heptane and left standing. The solid that form was sonciated, filtered, rinsed with CH₂Cl₂ and pumped on to yield (+/−)-tert-butyl ((1R,2R,3S,5R)-5-(3-((tert-butoxycarbonyl)amino)pyridin-4-yl)-2-hydroxy-3-methylcyclohexyl)carbamate in 85% yield. LC/MS (m/z)=422.3 (MH⁺), Rₜ=0.65 min.

### Synthesis of (+/−)-(1R,2R,4R,6S)-2-(tert-butoxycarbonylamino)-4-(3-(tert-butoxycarbonylamino)pyridin-4-yl)-6-methylcyclohexyl methanesulfonate

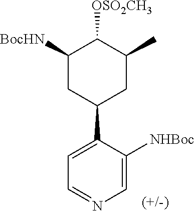

To a solution of (+/−)-tert-butyl ((1R,2R,3S,5R)-5-(3-((tert-butoxycarbonyl)amino)pyridin-4-yl)-2-hydroxy-3-methylcyclohexyl)carbamate (1.0 equiv.) in pyridine (0.17 M) was added MsCl (5.0 equiv.). The capped solution was stirred for 5 minutes and then the homogeneous solution was left standing at rt for 16 hrs. The volatiles were removed in vacuo and the residue was partitioned between EtOAc and H₂O. The organic layer was washed with H₂O, 10% CuSO₄, H₂O, Na₂CO_{3(sat.)}, NaCl_{(sat.)}, dried over MgSO₄, filtered, concentrated and purified by ISCO SiO₂ chromatography to yield (+/−)-(1R,2R,4R,6S)-2-(tert-butoxycarbonylamino)-4-(3-(tert-butoxycarbonylamino)pyridin-4-yl)-6-methylcyclohexyl methanesulfonate in 59% yield. LC/MS (m/z)=500.3 (MH⁺), Rₜ=0.74 min.

### Synthesis of (+/−)S-((1S,2R,4R,6S)-2-((tert-butoxycarbonyl)amino)-4-(3-((tert-butoxycarbonyl)amino)pyridin-4-yl)-6-methylcyclohexyl) ethanethioate

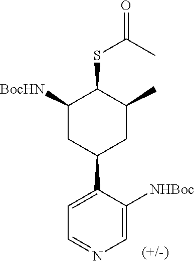 [SMILES: CC(=O)S[C@H]1[C@@H](C)C[C@@H](c2ccncc2NC(=O)OC(C)(C)C)C[C@H]1NC(=O)OC(C)(C)C]

To a solution of (+/−)-(1R,2R,4R,6S)-2-(tert-butoxycarbonylamino)-4-(3-(tert-butoxycarbonylamino)pyridin-4-yl)-6-methylcyclohexyl methanesulfonate (1.0 equiv.) in DMF (0.25 M) was added potassium thioacetate (6.0 equiv.). The mixture was stirred for 6 hours in a 60° C. bath under Ar. Upon cooling and the residue was partitioned between EtOAc and H₂O. The organic layer was washed with H₂O (3×), Na₂CO_{3(sat.)}, NaCl_{(sat.)}, dried over MgSO₄, filtered, concentrated and purified by ISCO SiO₂ chromatography to yield (+/−)S-((1 S,2R,4R,6S)-2-((tert-butoxycarbonyl)amino)-4-(3-((tert-butoxycarbonyl)amino)pyridin-4-yl)-6-methylcyclohexyl) ethanethioate in 87% yield. LC/MS (m/z)=480.3 (MH⁺), Rₜ=0.82 min.

### Synthesis of (+/−)tert-butyl ((1R,2S,3S,5R)-5-(3-aminopyridin-4-yl)-3-methyl-2-(methylsulfonyl)cyclohexyl)carbamate

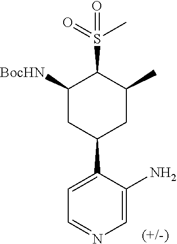 [SMILES: C[C@H]1C[C@@H](c2ccncc2N)C[C@@H](NC(=O)OC(C)(C)C)[C@H]1S(C)(=O)=O]

To a solution of (+/−)S-((1S,2R,4R,6S)-2-((tert-butoxycarbonyl)amino)-4-(3-((tert-butoxycarbonyl)amino)pyridin-4-yl)-6-methylcyclohexyl) ethanethioate (1.0 equiv.) in MeOH (0.09 M) was added potassium carbonate (3.0 equiv.). The mixture was stirred for 15 minutes at which time methyl iodide (1.1 eq.) was added and the solution was stirred at rt for 15 minutes. The volatiles were removed in vacuo and the residue was partitioned between EtOAc and H₂O. The organic layer was washed with H₂O, NaCl_{(sat.)}, dried over MgSO₄, filtered, concentrated and purified by ISCO SiO₂ chromatography to yield the methyl sulfide product in 99% yield. LC/MS (m/z)=452.3 (MH⁺), Rₜ=0.87 min. To a solution of methyl sulfide (1.0 eq.) in THF (0.05 M) at rt was added an aqueous solution of oxone (2.2 eq.) dropwise over 10 minutes. After stirring at rt for 1 hour the solution was partitioned between EtOAc and H₂O. The organic layer was washed with H₂O, NaCl_{(sat.)}, dried over MgSO₄, filtered, concentrated to yield the bis Boc protected methyl sulfone product in 95% yield. LC/MS (m/z)=484.2 (MH⁺), Rₜ=0.77 min. The bis boc procteced cyclohexyl sulfone (1.0 equiv) was treated with 4M HCl in dioxane for 3 hours to removed both Boc groups. Upon removal of the volatiles in vacuo, the residue was suspended in 1:1 dioxane/Na₂CO_{3(sat.)} and N-(tert-Butoxycarbonyloxy)succinimide (1.2 eq.) was added. After stirring for 1 hour, additional N-(tert-Butoxycarbonyloxy)succinimide (1.2 eq.) was added. After stirring for an additional 2 hours the solution was extracted with CH₂Cl₂, the combined organic layers were washed with H₂O, NaCl_{(sat.)}, dried over MgSO₄, filtered, concentrated and purified by ISCO SiO₂ chromatography to yield the (+/−)tert-butyl ((1R,2S,3S,5R)-5-(3-aminopyridin-4-yl)-3-methyl-2-(methylsulfonyl)cyclohexyl)carbamate in 56% yield. LC/MS (m/z)=384.3 (MH⁺), Rₜ=0.57 min. Chiral purification was completed via SFC (20% EtOH/80% n-heptanes, 20 mL/min, OJ column) to isolate the pure enantiomers. The second peak correlated with tert-butyl ((1R,2S,3S,5R)-5-(3-aminopyridin-4-yl)-3-methyl-2-(methylsulfonyl)cyclohexyl)carbamate.

### Synthesis of tert-butyl ((1R,2S,3S,5R)-5-(3-isothiocyanatopyridin-4-yl)-3-methyl-2-(methylsulfonyl)cyclohexyl)carbamate

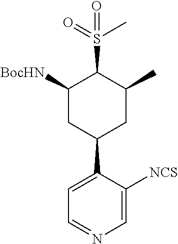

A solution of tert-butyl ((1R,2S,3S,5R)-5-(3-aminopyridin-4-yl)-3-methyl-2-(methylsulfonyl)cyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2S,3S,5R)-5-(3-isothiocyanatopyridin-4-yl)-3-methyl-2-(methylsulfonyl)cyclohexyl)carbamate is obtained.

### Synthesis of tert-butyl ((1R,2S,3S,5R)-5-(3-((tert-butoxycarbonyl)amino)pyridin-4-yl)-2-azido-3-methylcyclohexyl)carbamate and tert-butyl ((1S,2R,3R,5S)-5-(3-((tert-butoxycarbonyl)amino)pyridin-4-yl)-2-azido-3-methylcyclohexyl)carbamate

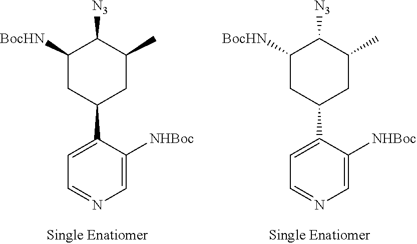 [SMILES: C[C@H]1C[C@@H](c2ccncc2NC(=O)OC(C)(C)C)C[C@@H](NC(=O)OC(C)(C)C)[C@H]1N=[N+]=[N-] ; C[C@@H]1C[C@H](c2ccncc2NC(=O)OC(C)(C)C)C[C@H](NC(=O)OC(C)(C)C)[C@@H]1N=[N+]=[N-]]

To a solution of (+/−)-(1R,2R,4R,6S)-2-(tert-butoxycarbonylamino)-4-(3-(tert-butoxycarbonylamino)pyridin-4-yl)-6-methylcyclohexyl methanesulfonate (1.0 equiv.) in DMF (0.13 M) was added NaN₃ (1.0 equiv.). The solution was submerged in an 80° C. oil bath and left stirring under Ar for 16 hrs. The solution was cooled to rt and left stirring under Ar overnight. The solution was partitioned between EtOAc and H₂O. The organic layer was washed with Na₂CO_{3(sat.)}, NaCl_{(sat.)}, dried over MgSO₄, filtered, concentrated and purified by ISCO SiO₂ chromatography. Purification was completed via SFC (15% IPA, 100 mL/min, IA column) to yield tert-butyl ((1R,2S,3S,5R)-5-(3-((tert-butoxycarbonyl)amino)pyridin-4-yl)-2-azido-3-methylcyclohexyl)carbamate (21% yield, 99% ee) and tert-butyl ((1S,2R,3R,5 S)-5-(3-((tert-butoxycarbonyl)amino)pyridin-4-yl)-2-azido-3-methylcyclohexyl)carbamate (22% yield, 99% ee). LC/MS (m/z)=447.3 (MH⁺), Rₜ=0.86 min.

### Synthesis of tert-butyl (1R,2S,3S,5R)-5-(3-aminopyridin-4-yl)-2-azido-3-methylcyclohexylcarbamate

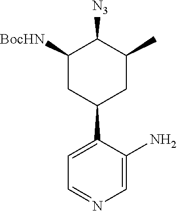 [SMILES: C[C@H]1C[C@@H](c2ccncc2N)C[C@@H](NC(=O)OC(C)(C)C)[C@H]1N=[N+]=[N-]]

A solution of 4 M HCl in dioxane (30.0 equiv.) was added to tert-butyl ((1R,2S,3S,5R)-5-(3-((tert-butoxycarbonyl)amino)pyridin-4-yl)-2-azido-3-methylcyclohexyl)carbamate (1.0 equiv.). The solution started to go homogeneous for a few minutes, but then a ppt formed and the solution went very thick. After sitting at rt for 1 hour, the volatiles were removed in vacuo and the solid was pumped on for 5 minutes on the high vac. To the residue was added CH₂Cl₂ (0.15 M), TEA (5.0 equiv.) and Boc₂O (1.0 equiv.). The solution was left stirring at rt for 1 hr. The volatiles were removed in vacuo and the residue was partitioned between EtOAc and H₂O. The organic layer was washed with Na₂CO_{3(sat.)}, NaCl_{(sat.)}, dried over MgSO₄, filtered, concentrated and purified by ISCO SiO₂ chromatography to yield tert-butyl (1R,2S,3S,5R)-5-(3-aminopyridin-4-yl)-2-azido-3-methylcyclohexylcarbamate in 57% yield. LC/MS (m/z)=347.3 (MH⁺), Rₜ=0.70 min. ¹H NMR (400 MHz, CHLOROFORM-d) δ ppm 1.13 (d, J=6.65 Hz, 3H), 1.43-1.58 (m, 11H), 1.79 (d, J=12.52 Hz, 1H), 1.95 (d, J=6.26 Hz, 1H), 2.60 (br. s., 1H), 3.61 (br. s., 2H), 3.77-3.91 (m, 2H), 4.78 (d, J=7.43 Hz, 1H), 6.96 (d, J=4.70 Hz, 1H), 7.97-8.07 (m, 2H).

### Synthesis of (+/−)-(1S,2R,65)-2-((tert-butoxycarbonyl)amino)-6-methyl-4-(3-nitropyridin-4-yl)cyclohex-3-en-1-yl methanesulfonate

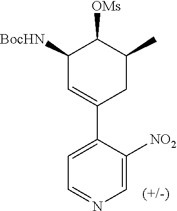 [SMILES: C[C@H]1CC(c2ccncc2[N+](=O)[O-])=C[C@@H](NC(=O)OC(C)(C)C)[C@H]1OS(C)(=O)=O]

To a solution of (+/−)-tert-butyl ((1R,5S,6S)-6-hydroxy-5-methyl-3-(3-nitropyridin-4-yl)cyclohex-2-en-1-yl)carbamate (1.0 equiv.) in pyridine (0.20 M) was added MsCl (5.0 equiv.). The capped solution was stirred for 5 minutes and then the homogeneous solution was left standing at rt for 16 hrs. The volatiles were removed in vacuo and the residue was partitioned between EtOAc and H₂O. The organic layer was washed with 10% CuSO₄, H₂O, Na₂CO_{3(sat.)}, NaCl_{(sat.)}, dried over MgSO₄, filtered, concentrated and purified by ISCO SiO₂ chromatography to yield (+/−)-(1S,2R,6S)-2-((tert-butoxycarbonyl)amino)-6-methyl-4-(3-nitropyridin-4-yl)cyclohex-3-en-1-yl methanesulfonate in 46% yield. LC/MS (m/z)=428.2 (MH⁺), Rₜ=0.89 min.

### Synthesis of (+/−)-(1S,2R,4R,6S)-4-(3-aminopyridin-4-yl)-2-((tert-butoxycarbonyl)amino)-6-methylcyclohexyl methanesulfonate

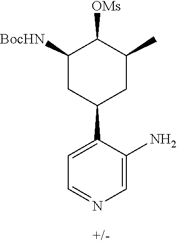 [SMILES: C[C@H]1C[C@@H](c2ccncc2N)C[C@@H](NC(=O)OC(C)(C)C)[C@H]1OS(C)(=O)=O]

Degass a solution of (+/−)-(1S,2R,6S)-2-((tert-butoxycarbonyl)amino)-6-methyl-4-(3-nitropyridin-4-yl)cyclohex-3-en-1-yl methanesulfonate (1.0 equiv.) in Ethanol (0.20 M). To this solution was added Pd/C (0.2 equiv.) and purge with Ar and H₂. The mixture was stirred under H₂ for 16 hrs. Filter the mixture over cetlite and wash the cake with MeOH. Concentrate the filtrate to yield (+/−)-(1S,2R,4R,6S)-4-(3-aminopyridin-4-yl)-2-((tert-butoxycarbonyl)amino)-6-methylcyclohexyl methanesulfonate in 49% yield). LC/MS (m/z)=400.3 (MH⁺), Rₜ=0.62 min.

### Synthesis of (+/−)-tert-butyl ((1R,2R,3S,5R)-5-(3-aminopyridin-4-yl)-2-azido-3-methylcyclohexyl)carbamate

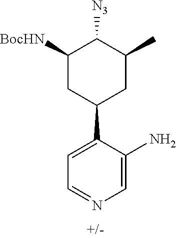 [SMILES: C[C@H]1C[C@@H](c2ccncc2N)C[C@@H](NC(=O)OC(C)(C)C)[C@@H]1N=[N+]=[N-]]

To a solution of (+/−)-(1S,2R,4R,6S)-4-(3-aminopyridin-4-yl)-2-((tert-butoxycarbonyl)amino)-6-methylcyclohexyl methanesulfonate (1.0 equiv.) in DMF (0.20 M) was added NaN₃ (7.0 equiv.). The solution was submerged in a 70° C. oil bath and left stirring under Ar for 4 hrs. The solution was cooled to rt and left stirring under Ar overnight. The solution was partitioned between EtOAc and H₂O. The organic layer was washed with Na₂CO_{3(sat.)}, NaCl_{(sat.)}, dried over MgSO₄, filtered, concentrated to yield (+/−)-tert-butyl ((1R,2R,3 S,5R)-5-(3-aminopyridin-4-yl)-2-azido-3-methyl cyclohexyl)carbamate in 87% yield. LC/MS (m/z)=347.3 (MH⁺), Rₜ=0.68 min.

### Synthesis of (+/−)-tert-butyl ((1R,5S,6R)-6-methoxy-5-methyl-3-(3-nitropyridin-4-yl)cyclohex-2-en-1-yl)carbamate

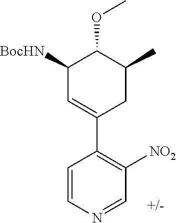 [SMILES: CO[C@@H]1[C@@H](C)CC(c2ccncc2[N+](=O)[O-])=C[C@H]1NC(=O)OC(C)(C)C]

(+/−)-Tert-butyl (1R,5S,6R)-6-hydroxy-5-methyl-3-(3-nitropyridin-4-yl)cyclohex-2-enylcarbamate (1.0 equiv.) was suspended in iodomethane (100.0 equiv.). Silver oxide (6.0 equiv.) was added to the mixture and the reaction vessel was wrapped in foil (kept dark) and allowed to stir 45° C. for 10 hrs. The reaction was diluted with THF and filtered through a pad of celite. The celite cake was further washed with MeOH. The organics were concentrated and the crude was taken up in DCM, washed with NaHCO_{3(aq.)}, dried over Na₂SO₄, filtered and concentrated. The crude was loaded onto silica gel and purified via ISCO to yield (+/−)-tert-butyl ((1R,5S,6R)-6-methoxy-5-methyl-3-(3-nitropyridin-4-yl)cyclohex-2-en-1-yl)carbamate in 35% yield. LC/MS (m/z)=364.1 (MH⁺), Rₜ=0.89 min.

### Method 1

### Synthesis of tert-butyl ((1S,2S,3R,5S)-5-(3-aminopyridin-4-yl)-2-methoxy-3-methylcyclohexyl)carbamate and tert-butyl ((1R,2R,3S,5R)-5-(3-aminopyridin-4-yl)-2-methoxy-3-methylcyclohexyl)carbamate

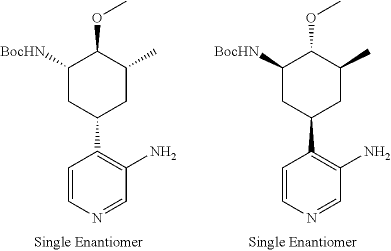 [SMILES: CO[C@H]1[C@H](C)C[C@H](c2ccncc2N)C[C@@H]1NC(=O)OC(C)(C)C ; CO[C@@H]1[C@@H](C)C[C@@H](c2ccncc2N)C[C@H]1NC(=O)OC(C)(C)C]

To a solution of (+/−)-tert-butyl ((1R,5S,6R)-6-methoxy-5-methyl-3-(3-nitropyridin-4-yl)cyclohex-2-en-1-yl)carbamate (1.0 equiv.) in degassed EtOH (0.10 M) was added Pd/C (0.1 equiv.). The mixture was purged with H₂, and allowed to to stir under an atm of H₂ overnight at RT. The reaction was filtered through a pad of celite and the cake was washed with MeOH. The organics were concentrated and purified by ISCO SiO₂ chromatography. Purification was completed via SFC (30% MeOH, 100 mL/min, AD column) to yield tert-butyl ((1S,2S,3R,5S)-5-(3-aminopyridin-4-yl)-2-methoxy-3-methylcyclohexyl)carbamate (15% yield, 99% ee) and tert-butyl ((1R,2R,3S,5R)-5-(3-aminopyridin-4-yl)-2-methoxy-3-methylcyclohexyl)carbamate (12% yield, 99% ee). LC/MS (m/z)=336.3 (MH⁺), Rₜ=0.58 min.

### Synthesis of tert-butyl ((1R,2R,3S,5R)-5-(3-isothiocyanatopyridin-4-yl)-2-methoxy-3-methylcyclohexyl)carbamate

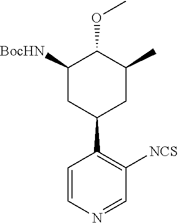

A solution of tert-butyl ((1R,2R,3S,5R)-5-(3-aminopyridin-4-yl)-2-methoxy-3-methylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2R,3S,5R)-5-(3-isothiocyanatopyridin-4-yl)-2-methoxy-3-methylcyclohexyl)carbamate is obtained.

### Synthesis of (+/−)-tert-butyl ((1R,5S,6S)-6-hydroxy-5-methyl-3-(3-nitropyridin-4-yl)cyclohex-2-en-1-yl)carbamate

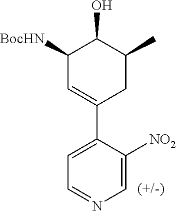 [SMILES: C[C@H]1CC(c2ccncc2[N+](=O)[O-])=C[C@@H](NC(=O)OC(C)(C)C)[C@H]1O]

To a solution of (+/−)-(3aR,7S,7aS)-tert-butyl 7-methyl-5-(3-nitropyridin-4-yl)-2-oxo-3a,6,7,7a-tetrahydrobenzo[d]oxazole-3(2H)-carboxylate (1.0 equiv.) in THF (0.20 M) was added 2M LiOH (3.0 equiv.) was added. The mixture was stirred overnight 20 hrs at 22° C. The mixture was diluted with EtOAc and NaHCO_{3(aq.)}. The layers were separated and the aqueous was extracted with EtOAc. The combined organics were washed with brine, dried over Na₂SO₄, filtered, and concentrated. The golden foam was purified by ISCO chromatography to afford (+/−)-tert-butyl ((1R,5S,6S)-6-hydroxy-5-methyl-3-(3-nitropyridin-4-yl)cyclohex-2-en-1-yl)carbamate in 83% yield. LC/MS (m/z)=350.2 (MH⁺), Rₜ=0.82 min.

### Synthesis of (+/−)-tert-butyl (1R,5S,6S)-6-(2-cyanoethoxy)-5-methyl-3-(3-nitropyridin-4-yl)cyclohex-2-enylcarbamate

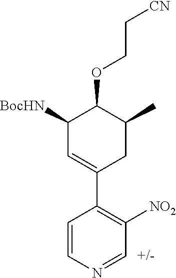 [SMILES: C[C@H]1CC(c2ccncc2[N+](=O)[O-])=C[C@@H](NC(=O)OC(C)(C)C)[C@H]1OCCC#N]

A mixture of (+/−)-Tert-butyl (1R,5S,6S)-6-hydroxy-5-methyl-3-(3-nitropyridin-4-yl)cyclohex-2-enylcarbamate (1.0 equiv.), acrylonitrile (30.0 equiv.) and CESIUM CARBONATE (1.2 equiv.) in t-BuOH (0.57 M) was stirred at 35° C. for 3 hrs. The reaction was cooled to room temperature, followed by the addition of NaHCO_{3(aq.)} and water. The mixture was extracted with EtOAc and the combined organics were dried over MgSO₄, filtered, and concentrated. The sample was purified by ISCO SiO₂ chromatography to yield (+/−)-tert-butyl (1R,5S,6S)-6-(2-cyanoethoxy)-5-methyl-3-(3-nitropyridin-4-yl)cyclohex-2-enylcarbamate in 94% yield. LC/MS (m/z)=403.3 (MH⁺), Rₜ=0.92 min. ¹H NMR (400 MHz, CHLOROFORM-d) δ ppm 1.13 (d, J=6.46 Hz, 3H), 1.46 (s, 9H), 1.99-2.18 (m, 2H), 2.20-2.36 (m, 1H), 2.65 (t, J=6.06 Hz, 2H), 3.68 (br. s., 1H), 3.88 (t, J=5.99 Hz, 2H), 4.51 (br. s., 1H), 4.99 (d, J=9.15 Hz, 1H), 5.39 (br. s., 1H), 7.25 (d, J=4.99 Hz, 1H), 8.73 (d, J=4.94 Hz, 1H), 9.10 (s, 1H).

### Synthesis of tert-butyl ((1S,2R,3R,5S)-5-(3-aminopyridin-4-yl)-2-(2-cyanoethoxy)-3-methylcyclohexyl)carbamate and tert-butyl ((1R,2S,3S,5R)-5-(3-aminopyridin-4-yl)-2-(2-cyanoethoxy)-3-methylcyclohexyl)carbamate

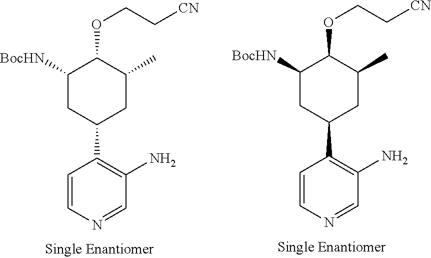 [SMILES: C[C@@H]1C[C@H](c2ccncc2N)C[C@H](NC(=O)OC(C)(C)C)[C@@H]1OCCC#N ; C[C@H]1C[C@@H](c2ccncc2N)C[C@@H](NC(=O)OC(C)(C)C)[C@H]1OCCC#N]

Method 1 was followed using (+/−)-tert-butyl (1R,5S,6S)-6-(2-cyanoethoxy)-5-methyl-3-(3-nitropyridin-4-yl)cyclohex-2-enylcarbamate with SFC (15% EtOH, 100 mL/min, OJ column) to yield tert-butyl ((1S,2R,3R,5S)-5-(3-aminopyridin-4-yl)-2-(2-cyanoethoxy)-3-methylcyclohexyl)carbamate (31% yield, 99% ee) and tert-butyl ((1R,2S,3S,5R)-5-(3-aminopyridin-4-yl)-2-(2-cyanoethoxy)-3-methylcyclohexyl) carbamate (26% yield, 99% ee). LC/MS (m/z)=375.3 (WO, Rₜ=0.65 min.

### Synthesis of tert-butyl ((1R,2S,3S,5R)-2-(2-cyanoethoxy)-5-(3-isothiocyanatopyridin-4-yl)-3-methylcyclohexyl)carbamate

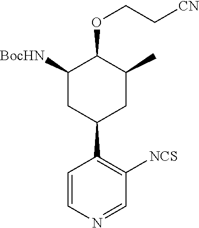

A solution of tert-butyl ((1R,2S,3S,5R)-5-(3-aminopyridin-4-yl)-2-(2-cyanoethoxy)-3-methylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2S,3S,5R)-2-(2-cyanoethoxy)-5-(3-isothiocyanatopyridin-4-yl)-3-methylcyclohexyl)carbamate is obtained.

### Synthesis of (+/−)-tert-butyl ((1R,5S,6S)-6-methoxy-5-methyl-3-(3-nitropyridin-4-yl)cyclohex-2-en-1-yl)carbamate

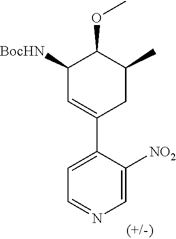 [SMILES: CO[C@H]1[C@@H](C)CC(c2ccncc2[N+](=O)[O-])=C[C@H]1NC(=O)OC(C)(C)C]

To a solution of (+/−)-tert-butyl ((1R,5 S,6S)-6-hydroxy-5-methyl-3-(3-nitropyridin-4-yl)cyclohex-2-en-1-yl)carbamate(1.0 equiv.) in MeI (100.0 equiv.) was added Ag₂O (5.5 equiv.). A reflux condenser was attached and the heterogeneous solution under Ar was submerged in a 50° C. bath and the reaction was gently refluxing for 6 hrs. The solids were filtered, rinsed with CH₂Cl₂. The volatiles were removed in vacuo, the residue was partitioned between CH₂Cl₂ and NaHCO₃(sat.). The organic layer was dried over MgSO₄, filtered, concentrated and purified by ISCO SiO₂ chromatography to yield (+/−)-tert-butyl ((1R,5 S,6S)-6-hydroxy-5-methyl-3-(3-nitropyridin-4-yl)cyclohex-2-en-1-yl)carbamate in 59% yield. LC/MS (m/z)=364.5 (MH⁺), Rt=1.02 min. ¹H NMR (400 MHz, <cdcl3>) δ ppm 1.11 (d, J=6.65 Hz, 3H), 1.46 (s, 9H), 1.95-2.13 (m, 2H), 2.18-2.28 (m, 1H), 3.47 (d, J=3.52 Hz, 1H), 3.57 (s, 3H), 4.45 (d, J=7.83 Hz, 1H), 5.01 (d, J=9.39 Hz, 1H), 5.44 (br. s., 1H), 7.24 (s, 1H), 8.71 (d, J=5.09 Hz, 1H), 9.08 (s, 1H).

### Synthesis of tert-butyl ((1 S,2R,3R,5 S)-5-(3-aminopyridin-4-yl)-2-methoxy-3-methylcyclohexyl)carbamate and tert-butyl ((1R,2S,3 S,5R)-5-(3-aminopyridin-4-yl)-2-methoxy-3-methylcyclohexyl)carbamate

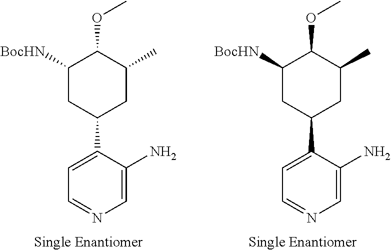 [SMILES: CO[C@@H]1[C@H](C)C[C@H](c2ccncc2N)C[C@@H]1NC(=O)OC(C)(C)C ; CO[C@H]1[C@@H](C)C[C@@H](c2ccncc2N)C[C@H]1NC(=O)OC(C)(C)C]

To a degassed solution of (+/−)-tert-butyl ((1R,5S,6S)-6-hydroxy-5-methyl-3-(3-nitropyridin-4-yl)cyclohex-2-en-1-yl)carbamate (1.0 equiv.) in i-PrOH (0.07 M) was added Pd/C (0.1 equiv.). The solution was degassed and purged to H₂ and left stirring under a balloon of H₂ at rt for 16 hrs. The solution was degassed and purged to Ar, diluted with CH₂Cl₂, filtered through a pad of celite, concentrated and purified by ISCO SiO₂ chromatography. Purification was completed via SFC (20% MeOH, 100 mL/min, AD column) to yield tert-butyl ((1S,2R,3R,5S)-5-(3-aminopyridin-4-yl)-2-methoxy-3-methylcyclohexyl)carbamate (42% yield, 99% ee) and tert-butyl ((1R,2S,3S,5R)-5-(3-aminopyridin-4-yl)-2-methoxy-3-methylcyclohexyl)carbamate (39% yield, 99% ee). LC/MS (m/z)=336.2 (MH⁺), Rₜ=0.67 min. ¹H NMR (400 MHz, <cdcl3>) δ ppm 1.08 (d, J=7.04 Hz, 3H), 1.43-1.49 (m, 10H), 1.52-1.64 (m, 2H), 1.70-1.81 (m, 2H), 2.52-2.64 (m, 1H), 3.39 (br. s., 1H), 3.52-3.57 (m, 3H), 3.62 (br. s., 2H), 3.66-3.75 (m, 1H), 4.75-4.87 (m, 1H), 6.98 (d, J=5.09 Hz, 1H), 7.95-8.05 (m, 2H).

### Synthesis of tert-butyl ((1R,2S,3S,5R)-5-(3-isothiocyanatopyridin-4-yl)-2-methoxy-3-methylcyclohexyl)carbamate

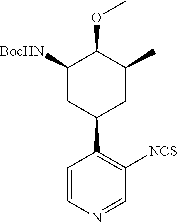

A solution of tert-butyl ((1R,2S,3S,5R)-5-(3-aminopyridin-4-yl)-2-methoxy-3-methylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2S,3S,5R)-5-(3-isothiocyanatopyridin-4-yl)-2-methoxy-3-methylcyclohexyl)carbamate is obtained.

### Synthesis of (+/−)-tert-butyl (1R,5S,6S)-6-ethoxy-5-methyl-3-(3-nitropyridin-4-yl)cyclohex-2-enylcarbamate

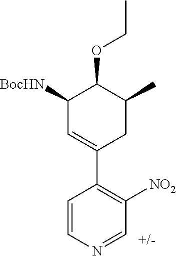 [SMILES: CCO[C@H]1[C@@H](C)CC(c2ccncc2[N+](=O)[O-])=C[C@H]1NC(=O)OC(C)(C)C]

(+/−)-Tert-butyl (1R,5S,6S)-6-hydroxy-5-methyl-3-(3-nitropyridin-4-yl)cyclohex-2-enylcarbamate (1.0 equiv.) was suspended in iodoethane (100.0 equiv.). Silver oxide (6.0 equiv.) was added to the mixture and the reaction vessel was wrapped in foil (kept dark) and allowed to stir 55° C. for 10 hrs. The reaction was diluted with THF and filtered through a pad of celite. The celite cake was further washed with MeOH. The organics were concentrated and the crude was taken up in DCM, washed with NaHCO₃ (aq), dried over Na₂SO₄, filtered and concentrated. The crude was loaded onto silica gel and purified via ISCO to yield (+/−)-tert-butyl (1R,5S,6S)-6-ethoxy-5-methyl-3-(3-nitropyridin-4-yl)cyclohex-2-enylcarbamate in 31% yield. LC/MS (m/z)=378.1 (MH⁺), Rₜ=0.99 min.

### Synthesis of tert-butyl ((1S,2R,3R,5S)-5-(3-aminopyridin-4-yl)-2-ethoxy-3-methylcyclohexyl)carbamate and tert-butyl ((1R,2S,3S,5R)-5-(3-aminopyridin-4-yl)-2-ethoxy-3-methylcyclohexyl)carbamate

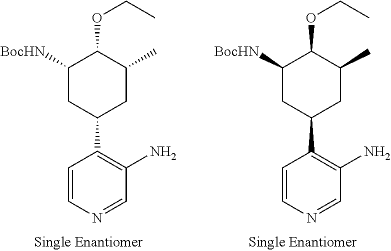 [SMILES: CCO[C@@H]1[C@H](C)C[C@H](c2ccncc2N)C[C@@H]1NC(=O)OC(C)(C)C ; CCO[C@H]1[C@@H](C)C[C@@H](c2ccncc2N)C[C@H]1NC(=O)OC(C)(C)C]

Method 1 was followed using (+/−)-tert-butyl (1R,5S,6S)-6-ethoxy-5-methyl-3-(3-nitropyridin-4-yl)cyclohex-2-enylcarbamate with Chiral HPLC (Heptane/EtOH=90/10, 20 mL/min, IC column) to yield tert-butyl ((1S,2R,3R,5S)-5-(3-aminopyridin-4-yl)-2-ethoxy-3-methylcyclohexyl)carbamate (33% yield, 99% ee) and tert-butyl ((1R,2S,3S,5R)-5-(3-aminopyridin-4-yl)-2-ethoxy-3-methylcyclohexyl)carbamate (28% yield, 99% ee). LC/MS (m/z)=350.2 (MH⁺), Rₜ=0.72 min.

### Synthesis of tert-butyl ((1R,2S,3S,5R)-5-(3-isothiocyanatopyridin-4-yl)-2-ethoxy-3-methylcyclohexyl)carbamate

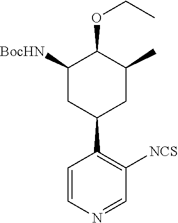

A solution of tert-butyl ((1R,2S,3S,5R)-5-(3-aminopyridin-4-yl)-2-ethoxy-3-methylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2S,3S,5R)-5-(3-isothiocyanatopyridin-4-yl)-2-ethoxy-3-methylcyclohexyl)carbamate is obtained.

### Synthesis of (+/−)-tert-butyl (1R,5S,6S)-5-methyl-6-(2-(methylsulfonyl)ethoxy)-3-(3-nitropyridin-4-yl)cyclohex-2-enylcarbamate

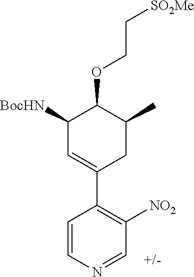 [SMILES: C[C@H]1CC(c2ccncc2[N+](=O)[O-])=C[C@@H](NC(=O)OC(C)(C)C)[C@H]1OCCS(C)(=O)=O]

A mixture of (+/−)-Tert-butyl (1R,5S,6S)-6-hydroxy-5-methyl-3-(3-nitropyridin-4-yl)cyclohex-2-enylcarbamate (1.0 equiv.), methylsulfonylethene (30.0 equiv.) and CESIUM CARBONATE (1.2 equiv.) in t-BuOH (0.22 M) was stirred at 22° C. for 5 hrs. The reaction was cooled to room temperature, followed by the addition of NaHCO₃(aq.) and water. The mixture was extracted with EtOAc and the combined organics were dried over MgSO₄, filtered, and concentrated. The sample was purified by ISCO chromatography to yield (+/−)-tert-butyl (1R,5S,6S)-5-methyl-6-(2-(methylsulfonyl)ethoxy)-3-(3-nitropyridin-4-yl)cyclohex-2-enylcarbamate in 84% yield. LC/MS (m/z)=456.2 (MH⁺), Rₜ=0.87 min. ¹H NMR (400 MHz, CHLOROFORM-d) δ ppm 1.10 (d, J=6.60 Hz, 3H), 1.47 (s, 9H), 1.92-2.16 (m, 2H), 2.17-2.32 (m, 1H), 2.93-3.00 (m, 1H), 3.09 (s, 3H), 3.18 (d, J=14.87 Hz, 1H), 3.38-3.52 (m, 1H), 3.63 (d, J=2.40 Hz, 1H), 3.95-4.06 (m, 1H), 4.21 (td, J=9.84, 2.42 Hz, 1H), 4.56 (d, J=7.58 Hz, 1H), 5.58 (br. s., 1H), 5.66 (d, J=9.44 Hz, 1H), 7.20 (d, J=4.99 Hz, 1H), 8.73 (d, J=4.99 Hz, 1H), 9.05 (s, 1H).

### Synthesis of (+/−)-tert-butyl (1R,2S,3S,5R)-5-(3-aminopyridin-4-yl)-3-methyl-2-(2-(methylsulfonyl)ethoxy)cyclohexylcarbamate

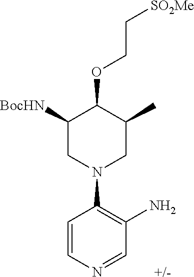

To a solution of (+/−)-tert-butyl (1R,5S,6S)-5-methyl-6-(2-(methylsulfonyl)ethoxy)-3-(3-nitropyridin-4-yl)cyclohex-2-enylcarbamate (1.0 equiv.) in degassed EtOH (0.17 M) was added Pd/C (0.3 equiv.). The mixture was purged with H₂, and allowed to stir under H₂ overnight at RT. The reaction was filtered through a pad of celite and the cake was washed with MeOH. The organics were concentrated and purified by ISCO SiO₂ chromatography to yield (+/−)-tert-butyl (1R,2S,3S,5R)-5-(3-aminopyridin-4-yl)-3-methyl-2-(2-(methylsulfonyl)ethoxy)cyclohexylcarbamate in 55% yield. LC/MS (m/z)=428.2 (MH⁺), Rₜ=0.59 min. ¹H NMR (400 MHz, CHLOROFORM-d) δ ppm 1.07 (d, J=6.85 Hz, 3H), 1.46 (s, 9H), 1.72-1.88 (m, 2H), 2.62 (tt, J=12.23, 3.30 Hz, 1H), 3.08 (s, 3H), 3.21 (d, J=14.62 Hz, 1H), 3.35-3.47 (m, 1H), 3.58 (br. s., 1H), 3.64 (br. s., 2H), 3.70-3.86 (m, 1H), 3.94-4.10 (m, 1H), 4.10-4.22 (m, 1H), 5.43 (d, J=9.00 Hz, 1H), 6.89 (d, J=5.04 Hz, 1H), 7.98 (d, J=4.99 Hz, 1H), 8.03 (s, 1H).

### Synthesis of tert-butyl ((1R,2S,3S,5R)-5-(3-isothiocyanatopyridin-4-yl)-3-methyl-2-(2-(methylsulfonyl)ethoxy)cyclohexyl)carbamate

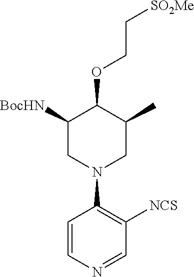

A solution of tert-butyl ((1R,2S,3S,5R)-5-(3-aminopyridin-4-yl)-3-methyl-2-(2-(methylsulfonyl)ethoxy)cyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2S,3S,5R)-5-(3-isothiocyanatopyridin-4-yl)-3-methyl-2-(2-(methylsulfonyl)ethoxy)cyclohexyl)carbamate is obtained.

### Synthesis of (+/−)-tert-butyl ((1R,2S,3S,5R)-5-(3-((tert-butoxycarbonyl)amino)pyridin-4-yl)-2-amino-3-methylcyclohexyl)carbamate

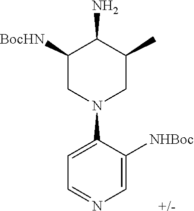

To a degassed a solution of (+/−)-tert-butyl ((1R,2S,3S,5R)-5-(3-((tert-butoxycarbonyl)amino)pyridin-4-yl)-2-azido-3-methylcyclohexyl)carbamate (1.0 equiv.) in ethanol (0.10 M) was added Pd/C (0.2 equiv.). The mixture was stirred under H₂ for 4 hrs. Filter the mixture over cetlite and wash the cake with MeOH. Concentrate the filtrate to yield (+/−)-tert-butyl ((1R,2S,3S,5R)-5-(3-((tert-butoxycarbonyl)amino)pyridin-4-yl)-2-amino-3-methylcyclohexyl)carbamate in 88% yield. LC/MS (m/z)=421.3 (MH⁺), Rₜ=0.58 min.

### Synthesis of (+/−)-tert-butyl ((1R,2S,3S,5R)-5-(3-((tert-butoxycarbonyl)amino)pyridin-4-yl)-3-methyl-2-(methylamino)cyclohexyl)carbamate

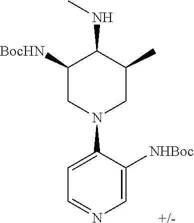

To a solution of (+/−)-tert-butyl ((1R,2S,3S,5R)-5-(3-((tert-butoxycarbonyl)amino)pyridin-4-yl)-2-amino-3-methylcyclohexyl)carbamate (1.0 equiv.) in MeOH (0.10 M) was added benzaldehyde (1.3 equiv.). After 3 hrs, sodium cyanotrihydroborate (2.5 equiv.) was added and the mixture was stirred at rt for 16 hrs. The reaction mixture was quenched by the addition of water, and volatiles were removed in vacuo. The mixture was extracted with ethyl acetate. The combined organic phases were dried with sodium sulfate, filtered and concentrated. The residue was dissolved in MeOH (0.10 M) and paraformaldehyde (5.0 equiv.) was added. After 16 hrs, sodium cyanotrihydroborate (5.0 equiv.) was added and the mixture was left stirred at rt for 16 hrs. The reaction was quenched by the addition of water, and volatiles were removed in vacuo. The mixture was extracted with DCM. The combined organic phases were dried with sodium sulfate, filtered, concentrated and purified by ISCO Chromatography. The product was dissolved in MeOH (0.10 M) and treated with Pd(OH)₂ (0.50 equiv.) under H₂ for 5 hrs at RT. The reaction was filtered through a pad of celite and the cake was washed with MeOH. The organics were concentrated to yield (+/−)-tert-butyl ((1R,2S,3S,5R)-5-(3-((tert-butoxycarbonyl)amino)pyridin-4-yl)-3-methyl-2-(methylamino)cyclohexyl)carbamate in 75% yield. LC/MS (m/z)=435.2 (MH⁺), Rₜ=0.64 min.

### Synthesis of (+/−)-methyl ((1S,2R,4R,6S)-4-(3-aminopyridin-4-yl)-2-((tert-butoxycarbonyl)amino)-6-methylcyclohexyl)(methyl)carbamate

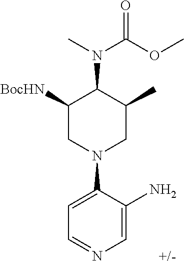

To a solution of (+/−)-tert-butyl ((1R,2S,3S,5R)-5-(3-((tert-butoxycarbonyl)amino)pyridin-4-yl)-3-methyl-2-(methylamino)cyclohexyl)carbamate (1.0 equiv.) in DCM (0.05 M) at 0° C. was added DIEA (3.0 equiv.) and then methyl chloroformate (1.5 equiv.). The homogeneous solution was left standing at 0° C. at for 4 hrs. The reaction was quenched partitioned between NaHCO₃ solution and EtOAc. The organic layer was washed with Brine, dried over Na₂SO₄, concentrated and purified by ISCO chromatography. The product was treated with 4 M HCl in dioxane (30.0 equiv.) at rt for 1 hour. The volatiles were removed in vacuo and the solid was pumped on for 5 minutes on the high vac. To the residue was added CH₂Cl₂ (0.15 M), DIEA (5.0 equiv.) and tert-butyl 2,5-dioxopyrrolidin-1-yl carbonate (1.6 equiv.). The solution was left stirring at rt for 1 hr. The volatiles were removed in vacuo and the residue was partitioned between EtOAc and H₂O. The organic layer was washed with Na₂CO_{3(sat.)}, NaCl_{(sat.)}, dried over MgSO₄, filtered, concentrated and purified by ISCO SiO₂ chromatography to yield (+/−)-methyl ((1 S,2R,4R,6S)-4-(3-aminopyridin-4-yl)-2-((tert-butoxycarbonyl)amino)-6-methylcyclohexyl)(methyl)carbamate in 20% yield. LC/MS (m/z)=393.2 (MH⁺), Rₜ=0.60 min.

### Synthesis of methyl ((1S,2R,4R,6S)-2-((tert-butoxycarbonyl)amino)-4-(3-isothiocyanatopyridin-4-yl)-6-methylcyclohexyl)(methyl)carbamate

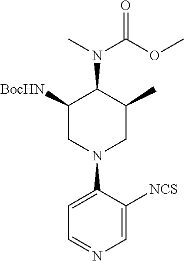

A solution of methyl ((1S,2R,4R,6S)-4-(3-aminopyridin-4-yl)-2-((tert-butoxycarbonyl)amino)-6-methylcyclohexyl)(methyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, methyl ((1S,2R,4R,6S)-2-((tert-butoxycarbonyl)amino)-4-(3-isothiocyanatopyridin-4-yl)-6-methylcyclohexyl)(methyl)carbamate is obtained.

### Synthesis of (+/−)-4-(5-methyl-3-(trimethylsilyloxy)cyclohexa-1,3-dienyl)-3-nitropyridine

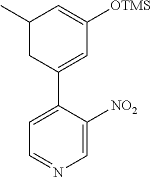

A solution of (+/−)-5-methyl-3-(3-nitropyridin-4-yl)cyclohex-2-enone (1.0 equiv.) and TMSCl (1.1 equiv.) in THF was added LiHMDS (1.0M in THF, 1.05 equiv.) at 0° C. slowly over 1 hour. The reaction mixture was warmed up to room temperature and stirred for 2 h. The reaction mixture was quenched with NaHCO₃ aqueous solution and removed THF in vacuo. The residue was extracted with EtOAc 3 times. The organic layer was washed with water and brine, dried over anhydrous K₂CO₃ and filtered, concentrated in vacuo to yield crude (+/−)-4-(5-methyl-3-(trimethylsilyloxy) cyclohexa-1,3-dienyl)-3-nitropyridine in 99% yield. ¹H NMR (400 MHz, CHLOROFORM-d) δ ppm 9.14-9.00 (m, 1H), 8.80-8.64 (m, 1H), 7.42-7.25 (m, 1H), 6.00-5.88 (m, 1H), 4.98 (br. s., 1H), 2.86-2.53 (m, 1H), 2.51-2.29 (m, 1H), 2.27-2.03 (m, 1H), 1.21-1.03 (m, 3H), 0.36-0.15 (m, 9H).

### Synthesis of (+/−)-6-((dimethylamino)methyl)-5-methyl-3-(3-nitropyridin-4-yl)cyclohex-2-enone

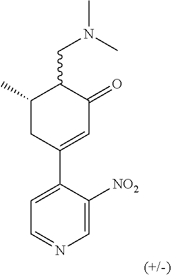 [SMILES: C[C@H]1CC(c2ccncc2[N+](=O)[O-])=CC(=O)C1CN(C)C]

To a solution of Eschenmoser's salt (1.1 equiv.) in DCM (0.3 M) was added (+/−)-4-(5-methyl-3-(trimethylsilyloxy)cyclohexa-1,3-dienyl)-3-nitropyridine in DCM (0.2 M) at 0° C. slowly over 60 min. The reaction mixture was allowed to warm up to room temperature and stirred for 18 h. After the reaction mixture was transferred to larger vessel and diluted with DCM (100 mL), 1 M HCl (60 mL) was added to the reaction mixture, which was stirred for 20 min in 0° C. 2 N NaOH (80 mL) was slowly added to aqueous phase at 0° C. The reaction mixture was stirred for 1 h, and then adjusted pH to 12 by 3N NaOH. After the organic layer was separated, aqueous phase was extracted with CH₂Cl₂ 3 times. The combined organic layer was dried over anhydrous Na₂SO₄ and concentrated in vacuo to yield crude (+/−)-6-((dimethylamino)methyl)-5-methyl-3-(3-nitropyridin-4-yl)cyclohex-2-enone in 99% yield. LCMS (m/z): 290.0 (MH⁺), Rₜ=0.40 min.

### Synthesis of (+/−)-5-methyl-6-methylene-3-(3-nitropyridin-4-yl)cyclohex-2-enone

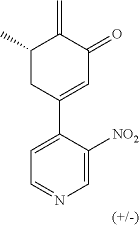 [SMILES: C=C1C(=O)C=C(c2ccncc2[N+](=O)[O-])C[C@@H]1C]

To a solution of (+/−)-6-((dimethylamino)methyl)-5-methyl-3-(3-nitropyridin-4-yl) cyclohex-2-enone (1.0 equiv.) in THF (0.3 M) was added iodomethane (1.3 equiv.) slowly at 0° C. The reaction mixture was allowed to warm up to room temperature and stirred at room temperature for 18 h. After saturated NaHCO₃ solution was added, the reaction mixture was stirred at room temperature for 5 h, diluted with EtOAc and stirred at room temperature for another 6 hr. After the organic layer was separated, the aqueous phase was extracted with EtOAc 3 times, the combined organic layer was washed with water and brine, dried over anhydrous Na₂SO₄, concentrated in vacuo to give crude (+/−)-5-methyl-6-methylene-3-(3-nitropyridin-4-yl)cyclohex-2-enone in 99% yield. LCMS (m/z): 245 (MH⁺), Rₜ=0.40 min. ¹H NMR (400 M Hz, CHLOROFORM-d) δ ppm 9.33 (s, 1H), 8.88 (d, J=5.1 Hz, 1H), 7.32-7.26 (m, 1H), 6.22-6.09 (m, 2H), 5.42 (s, 1H), 3.15 (dt, J=4.6, 2.2 Hz, 1H), 2.59 (dd, J=17.4, 5.3 Hz, 1H), 2.43 (ddd, J=7.3, 9.5, 2.2 Hz, 1H), 1.31 (d, J=6.7 Hz, 3H).

### Synthesis of (+/−)-(1R,5S)-5-methyl-6-methylene-3-(3-nitropyridin-4-yl)cyclohex-2-enol

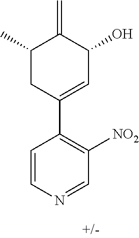 [SMILES: C=C1[C@H](O)C=C(c2ccncc2[N+](=O)[O-])C[C@@H]1C]

To a solution of (+/−)-5-methyl-6-methylene-3-(3-nitropyridin-4-yl)cyclohex-2-enone (1.0 equiv.) in methanol (0.3 M) was added CERIUM(III) CHLORIDE HEPTAHYDRATE (1.1 equiv.). The reaction mixture was stirred at room temperature for 1 h. After cooled down to at 0° C., NaBH₄ (1.0 equiv) was added slowly and stirred for 30 min. After quenched with water, the volatile materials were removed in vacuo and sat. NaHCO₃ was added into mixture with vigorous stirring. The reaction mixture was extracted with EtOAc and the organic layer was washed with brine, and dried over anhydrous sodium sulfate, filtered and concentrated in vacuo. The crude product was purified by silica chromatography (Heptane:EtOAc, 80:20 to 20:80) to give (+/−)-(1R,5S)-5-methyl-6-methylene-3-(3-nitropyridin-4-yl)cyclohex-2-enol as yellow solid in 50% yield. LCMS (m/z): 247 (MH⁺), Rₜ=0.70 min. ¹H NMR (400 M Hz, CHLOROFORM-d) δ ppm 9.13 (s, 1H), 8.75 (d, J=4.7 Hz, 1H), 7.26 (s, 1H), 5.73 (br. s., 1H), 5.25 (s, 1H), 5.03 (br. s., 1H), 4.86 (br. s., 1H), 2.67 (d, J=4.7 Hz, 1H), 2.39 (dd, J=16.6, 4.9 Hz, 1H), 2.11 (br. s., 1H), 1.79 (d, J=8.6 Hz, 1H), 1.23 (d, J=6.7 Hz, 3H).

### Synthesis of (+/−)-(1R,2R,6S)-1-(bromomethyl)-6-methyl-4-(3-nitropyridin-4-yl)cyclohex-3-ene-1,2-diol

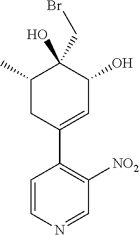 [SMILES: C[C@H]1CC(c2ccncc2[N+](=O)[O-])=C[C@@H](O)[C@@]1(O)CBr]

To a solution of (+/−)-(1R,5S)-5-methyl-6-methylene-3-(3-nitropyridin-4-yl)cyclohex-2-enol (1.0 equiv.) in THF:H₂O (1:1, 0.3 M) was added NBS (1.5 equiv.) at room temperature. The reaction mixture was stirred at room temperature for 5 min. After quenched with sodium thiosulfite, the reaction mixture was then extracted by EtOAc and washed with NaHCO₃ solution, water and brine, dried over anhydrous sodium sulfate, filtered and concentrated in vacuo. The crude product was used in next step reaction. LCMS (m/z): 342.9/344.9 (MH⁺), Rₜ=0.62 min. ¹H NMR (400 M Hz, CDCl₃) δ ppm 9.13 (s, 1H), 8.77 (d, J=5.1 Hz, 1H), 7.29 (d, J=5.1 Hz, 1H), 5.75-5.71 (m, 1H), 4.27 (br. s., 1H), 4.06 (d, J=10.6 Hz, 1H), 3.77 (d, J=11.0 Hz, 1H), 2.76-2.69 (m, 1H), 2.34 (br. s., 1H), 2.31-2.23 (m, 1H), 2.14 (dd, J=17.8, 5.7 Hz, 1H), 1.20 (d, J=7.4 Hz, 3H).

### Synthesis of 4-((+/−)-6-(bromomethyl)-5-methyl-7-oxabicyclo[4.1.0]hept-2-en-3-yl)-3-nitropyridine

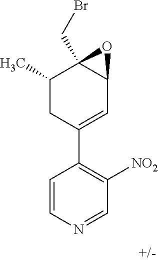 [SMILES: C[C@H]1CC(c2ccncc2[N+](=O)[O-])=C[C@@H]2O[C@@]21CBr]

To a 0.15 M solution of (+/−)-1-(bromomethyl)-6-methyl-4-(3-nitropyridin-4-yl)cyclohex-3-ene-1,2-diol (1.0 equiv) in DCM was added TEA (2.0 equiv) at 0° C. MsCl (1.4 equiv) was added dropwise over 10 minutes. The reaction mixture was stirred at 0° C. for 1 hour. The reaction mixture was quenched with saturated aqueous sodium bicarbonate and stirred for 20 minutes. The reaction mixture was extracted with DCM. The combined organic layers were washed sequentially with water and brine, dried over anhydrous sodium sulfate, filtered and concentrated under reduced pressure to give crude 4-((+/−)-6-(bromomethyl)-5-methyl-7-oxabicyclo[4.1.0]hept-2-en-3-yl)-3-nitropyridine in quantitative yield. LC/MS (m/z): 325/327 (MH⁺), Rₜ=0.84 min.

### Synthesis of 4-((+/−)-4-azido-8-methyl-1-oxaspiro[2.5]oct-5-en-6-yl)-3-nitropyridine

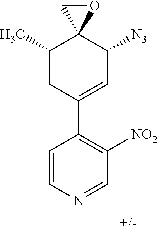 [SMILES: C[C@H]1CC(c2ccncc2[N+](=O)[O-])=C[C@@H](N=[N+]=[N-])[C@@]12CO2]

To a 0.25 M solution of 4-((+/−)-6-(bromomethyl)-5-methyl-7-oxabicyclo[4.1.0]hept-2-en-3-yl)-3-nitropyridine (1.0 equiv.) in 3:1 ethanol:water was added ammonium chloride (1.5 equiv.) and sodium azide (1.5 equiv.). The reaction mixture was stirred at room temperature for 18 hours. The reaction mixture was treated with an equal volume of saturated aqueous sodium bicarbonate and acetonitrile and stirred for 2 hours. Volatiles were removed under reduced pressure. The mixture was extracted with ethyl acetate. The combined organic layers were washed with brine, dried over anhydrous sodium sulfate, filtered, and concentrated. The crude material was purified by flash chromatography over silica gel (heptanes with 20% to 80% ethyl acetate gradient) to give 4-((+/−)-4-azido-8-methyl-1-oxaspiro[2.5]oct-5-en-6-yl)-3-nitropyridine in 57% yield as a yellow oil. LC/MS (m/z): 288.0 (MH⁺), Rₜ=0.80 min.

### Synthesis of tert-butyl (+/−)-5-(3-aminopyridin-4-yl)-2-hydroxy-2,3-dimethylcyclohexylcarbamate

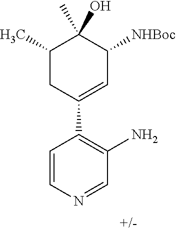

A 0.05 M solution of 4-((+/−)-4-azido-8-methyl-1-oxaspiro[2.5]oct-5-en-6-yl)-3-nitropyridine (1.0 equiv.) in ethanol was degassed for 10 minutes. Pyridine (10 equiv.) and 10% palladium on carbon (0.3 equiv) were added. The reaction vessel was purged and flushed three times with hydrogen. The reaction was stirred under a hydrogen atmosphere for 4 days. The reaction mixture was purged of hydrogen, diluted with DCM/MeOH, and filtered. The filter cake was rinsed with additional DCM/MeOH. The filtrate was concentrated. The residue was dissolved in ethanol to make a 0.1 M solution and treated with di-tert-butyl dicarbonate (1.2 equiv.). The mixture was stirred for 1 hr at ambient temperature and then concentrated under reduced pressure. The residue was purified by flash chromatography over silica gel (95:5 DCM:MeOH+0.5% NH₄OH to 90:10 DCM:MeOH+1% NH₄OH) to give racemic tert-butyl (+/−)-5-(3-aminopyridin-4-yl)-2-hydroxy-2,3-dimethylcyclohexylcarbamate in 42% yield. The enantiomers could be separated using an AD column eluting with heptanes/IPA. LC/MS (m/z): 336.1 (MH⁺), Rₜ=0.50 min. ¹H-NMR (400 MHz, methanol-d4): δ ppm 7.94 (s, 1H) 7.78 (d, J=5.09 Hz, 1H) 7.08 (d, J=5.09 Hz, 1H) 3.67 (m, 1H) 2.84-3.04 (m, 1H) 1.69-1.95 (m, 2H) 1.69-1.79 (m, 1H) 1.41-1.57 (m, 10H) 1.29-1.41 (m, 1H) 1.08 (s, 3H) 1.03 (d, J=6.65 Hz, 3H).

### Synthesis of tert-butyl ((1R,2R,3S,5R)-2-hydroxy-5-(3-isothiocyanatopyridin-4-yl)-2,3-dimethylcyclohexyl)carbamate

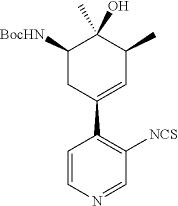

A solution of tert-butyl ((1R,2R,3S,5R)-5-(3-aminopyridin-4-yl)-2-hydroxy-2,3-dimethylcyclohexyl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,2R,3S,5R)-2-hydroxy-5-(3-isothiocyanatopyridin-4-yl)-2,3-dimethylcyclohexyl)carbamate is obtained.

### Synthesis of (+/−)-tert-butyl ((1R,5S,6S)-6-hydroxy-5-methyl-3-(3-nitropyridin-4-yl)cyclohex-2-en-1-yl)carbamate

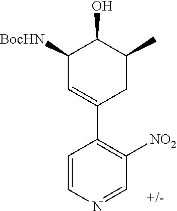 [SMILES: C[C@H]1CC(c2ccncc2[N+](=O)[O-])=C[C@@H](NC(=O)OC(C)(C)C)[C@H]1O]

To a solution of (+/−)-tert-butyl 7-methyl-5-(3-nitropyridin-4-yl)-2-oxo-3a,6,7,7a-tetrahydrobenzo[d]oxazole-3(2H)-carboxylate (1.0 equiv.) in tetrahydrofuran (0.2 M) was added 2M LiOH_{(aq.)} (3.0 equiv). After stirring at rt for 20 hours the solution was diluted with EtOAc, washed with NaHCO_{3(sat.)}, the aqueous layer was further extracted with EtOAc (3×), the combined organics were washed with NaCl_{(sat.)}, dried over MgSO₄, filtered, concentrated, and purified by ISCO SiO₂ chromatography to yield (+/−)-tert-butyl ((1R,5S,6S)-6-hydroxy-5-methyl-3-(3-nitropyridin-4-yl)cyclohex-2-en-1-yl)carbamate as a white foam in 84% yield. LC/MS=350.2 (M+H), Rₜ=0.82 min.

### Synthesis of (+/−)-(1S,2R,65)-2-((tert-butoxycarbonyl)amino)-6-methyl-4-(3-nitropyridin-4-yl)cyclohex-3-en-1-yl methanesulfonate

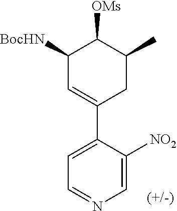 [SMILES: C[C@H]1CC(c2ccncc2[N+](=O)[O-])=C[C@@H](NC(=O)OC(C)(C)C)[C@H]1OS(C)(=O)=O]

To a solution of (+/−)-tert-butyl ((1R,5S,6S)-6-hydroxy-5-methyl-3-(3-nitropyridin-4-yl)cyclohex-2-en-1-yl)carbamate (1.0 equiv.) in pyridine (0.20 M) was added MsCl (5.0 equiv.). The capped solution was stirred for 5 minutes and then the homogeneous solution was left standing at rt for 5 hrs. The volatiles were removed in vacuo and the residue was partitioned between EtOAc and H₂O. The organic layer was washed with 10% CuSO₄, H₂O, Na₂CO_{3(sat.)}, NaCl_{(sat.)}, dried over MgSO₄, filtered, concentrated and purified by ISCO SiO₂ chromatography to yield (+/−)-(1S,2R,6S)-2-((tert-butoxycarbonyl)amino)-6-methyl-4-(3-nitropyridin-4-yl)cyclohex-3-en-1-yl methanesulfonate in 97% yield. LC/MS (m/z)=428.1 (MH⁺), Rₜ=0.86 min.

### Synthesis of (+/−)-(1S,2R,4R,6S)-4-(3-aminopyridin-4-yl)-2-((tert-butoxycarbonyl)amino)-6-methylcyclohexyl methanesulfonate

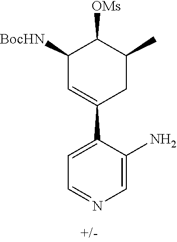

To a solution of (+/−)-(1S,2R,65)-2-((tert-butoxycarbonyl)amino)-6-methyl-4-(3-nitropyridin-4-yl)cyclohex-3-en-1-yl methanesulfonate (1.0 equiv.) in isopropanol (0.20 M). To this solution was added Pd/C (0.2 equiv.) and after pulling vacuum and purging to H₂ 3× the solution was left stirring under a balloon of H₂ for 16 hrs. The reaction was degassed, purged to Ar. filtered over celite, rinsed with CH₂Cl₂/i-PrOH, concentrated and purified by ISCO SiO₂ chromatography to yield (+/−)-(1S,2R,4R,6S)-4-(3-aminopyridin-4-yl)-2-((tert-butoxycarbonyl)amino)-6-methylcyclohexyl methanesulfonate in 72% yield). LC/MS (m/z)=400.2 (MH⁺), Rₜ=0.60 min.

### Synthesis of tert-butyl ((1S,5S)-5-(3-aminopyridin-4-yl)-3-methylcyclohex-2-en-1-yl)carbamate and tert-butyl ((1R,5R)-5-(3-aminopyridin-4-yl)-3-methylcyclohex-2-en-1-yl)carbamate

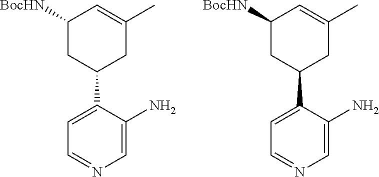 [SMILES: CC1=C[C@@H](NC(=O)OC(C)(C)C)C[C@@H](c2ccncc2N)C1 ; CC1=C[C@H](NC(=O)OC(C)(C)C)C[C@H](c2ccncc2N)C1]

A solution of (+/−)-(1S,2R,4R,6S)-4-(3-aminopyridin-4-yl)-2-((tert-butoxycarbonyl)amino)-6-methylcyclohexyl methanesulfonate (1.0 equiv.) and DBU (3.0 equiv) in DMF (0.39 M) was heated at 100° C. for three hours. Upon cooling, the solution was diluted with EtOAc, washed with H₂O (4×), Na₂CO_{3(sat.)}, NaCl_{(sat.)}, dried over MgSO₄, filtered, concentrated and purified by ISCO SiO₂ chromatography to yield (+/−)-tert-butyl ((1R,5R)-5-(3-aminopyridin-4-yl)-3-methylcyclohex-2-en-1-yl)carbamate in 69% yield. LC/MS (m/z)=304.1 (MH⁺), Rₜ=0.63 min. ¹H-NMR (400 MHz, CDCl₃) 6 ppm: 8.06 (s, 1H), 8.02 (d, J=4.0, 1H), 6.97 (d, J=4.0, 1H), 5.37 (br. s, 1H), 4.35-4.52 (m, 2H), 3.62-3.68 (m, 2H), 2.70-2.90 (m, 1H), 2.16-2.32 (m, 2H), 2.02-2.12 (m, 1H), 1.45 (s, 3H), 1.45-1.48 (m, 1H). The racemic material was resolved using an AD-H chiral column (15% EtOH/n-heptanes, 20 mL/min flow rate) to yield tert-butyl (1S,5S)-5-(3-aminopyridin-4-yl)-3-methylcyclohex-2-en-1-yl)carbamate (peak#1, Rₜ=6.13 min on AD-H chiral analytical column, 15% EtOH/85% n-heptanes, 1 mL/min) and tert-butyl ((1R,5R)-5-(3-aminopyridin-4-yl)-3-methylcyclohex-2-en-1-yl)carbamate (peak#2, Rₜ=9.18 min on AD-H chiral analytical column, 15% EtOH/85% n-heptanes, 1 mL/min).

### Synthesis of tert-butyl ((1R,5R)-5-(3-isothiocyanatopyridin-4-yl)-3-methylcyclohex-2-en-1-yl)carbamate

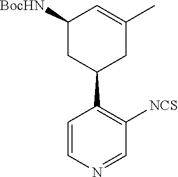

A solution of tert-butyl ((1R,5R)-5-(3-aminopyridin-4-yl)-3-methylcyclohex-2-en-1-yl)carbamate (1.0 equiv.) in tetrahydrofuran (0.1 M concentration) is treated with 1,1′-thiocarbonyldiimidazole (2.0 equiv.) and heated at 60° C. for 2 hours. Upon concentration and purification by SiO₂ chromatography, tert-butyl ((1R,5R)-5-(3-isothiocyanatopyridin-4-yl)-3-methylcyclohex-2-en-1-yl)carbamate is obtained.

### Method 2

### Synthesis of 2-(2,6-difluoro-4-methylphenyl)-4,4,5,5-tetramethyl-1,3,2-dioxaborolane

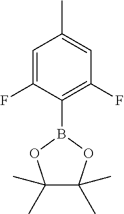 [SMILES: Cc1cc(F)c(B2OC(C)(C)C(C)(C)O2)c(F)c1]

To a solution of 1,3-difluoro-5-methylbenzene (1.0 eq) in dry THF (0.2M) under an atmosphere of N₂ at −78° C. was added n-butyllithium (l eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield 2-(2,6-difluoro-4-methylphenyl)-4,4,5,5-tetramethyl-1,3,2-dioxaboroane as a white solid in 92%. 1H NMR (400 MHz, <cdcl3>) δ ppm 6.67 (dd, J=9.39, 0.78 Hz, 2H), 2.34 (s, 3H), 1.38 (s, 12H).

### Synthesis of (2-(3,5-difluorophenyl)propan-2-yloxy)triisopropylsilane

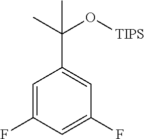

To a solution of 1-(3,5-difluorophenyl)ethanone (1.0 equiv) in THF (0.2 M) at 0° C. was added methylmagnesium bromide (1.0 M in THF, 1.15 equiv). After stirring for 4 hours the reaction was quenched by addition of NH₄Cl_{(sat.)}, diluted with EtOAc, washed with NaCl_{(sat.)}, dried over MgSO₄, filtered, concentrated and purified by ISCO SiO₂ chromatography to yield 2-(3,5-difluorophenyl)propan-2-ol. To a solution of 2-(3,5-difluorophenyl)propan-2-ol in CH₂Cl₂ (0.1 M) at 0° C. was added 2,6 lutidine (6 equiv.) and than triisopropylsilyl trifluoromethanesulfonate (3.0 equiv.). After stirring for 3 hours at 0° C. and six hours at rt the solution was partitioned between EtOAc and NaHCO_{3(sat.)}, separated, washed with NaCl_{(sat.)}, dried over MgSO₄, filtered, concentrated and purified by ISCO SiO₂ chromatography to yield (2-(3,5-difluorophenyl)propan-2-yloxy)triisopropylsilane. (400 MHz, <cdcl3>) δ ppm 1.05-1.08 (m, 21H) 1.57 (s, 6H) 6.63 (s, 1H) 7.00 (dd, J=9.39, 2.35 Hz, 2H).

### Synthesis of (2-(3,5-difluoro-4-(4,4,5,5-tetramethyl-1,3,2-dioxaborolan-2-yl)phenyl)propan-2-yloxy)triisopropylsilane

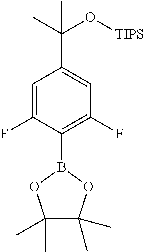

To a solution of (2-(3,5-difluorophenyl)propan-2-yloxy)triisopropylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield (2-(3,5-difluoro-4-(4,4,5,5-tetramethyl-1,3,2-dioxaborolan-2-yl)phenyl)propan-2-yloxy)triisopropylsilane in 99%. ¹H NMR (400 MHz, <cdcl3>) 6 ppm 1.03-1.08 (m, 21H) 1.24 (s, 12H) 1.38 (s, 3H) 1.57 (s, 3H) 6.92-7.03 (m, 2H).

### Synthesis of tert-butyl(3,5-difluorophenoxy)dimethylsilane

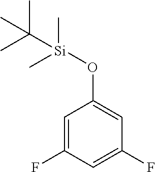 [SMILES: CC(C)(C)[Si](C)(C)Oc1cc(F)cc(F)c1]

To a solution of 3,5-difluorophenol (1.0 equiv.) and imidazole (2.2 equiv.) in

DMF (0.8 M) at 0° C. was added TBDMSCl (1.1 equiv.). The ice bath was removed and after stirring for 3 hours the solution was diluted with EtOAc, washed with water, brine, dried over MgSO₄, filtered, concentrated and purified by SiO₂ chromatography to yield tert-butyl(3,5-difluorophenoxy)dimethylsilane in 73% yield. ¹H NMR (400 MHz, <cdcl3>) δ ppm 0.23 (s, 6H) 0.99 (s, 9H) 6.33-6.40 (m, 2H) 6.44 (tt 1H).

### Synthesis of tert-butyl(3,5-difluoro-4-(4,4,5,5-tetramethyl-1,3,2-dioxaborolan-2-yl)phenoxy)dimethylsilane

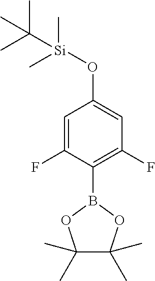 [SMILES: CC1(C)OB(c2c(F)cc(O[Si](C)(C)C(C)(C)C)cc2F)OC1(C)C]

To a solution of tert-butyl(3,5-difluorophenoxy)dimethylsilane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield tert-butyl(3,5-difluoro-4-(4,4,5,5-tetramethyl-1,3,2-dioxaborolan-2-yl)phenoxy)dimethylsilane in 91% yield. ¹H NMR (400 MHz, <cdcl3>) δ ppm 0.21 (s, 6H) 0.97 (s, 9H) 1.37 (s, 12H) 6.33 (d, J=9.39 Hz, 2H).

### Synthesis of 1,3-difluoro-5-(2-methoxyethoxyl)benzene

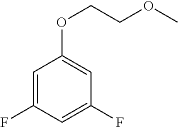

To a solution of 3,5-difluorophenol (1.0 equiv.), 2-methoxyethanol (3.0 equiv.) and triphenylphosphine (3.0 equiv) in THF (0.1 M) was added DIAD (3.0 equiv.). After stirring at rt for 18 hours, the volatiles were removed in vacuo and the residue was purified by SiO₂ chromatography to yield 1,3-difluoro-5-(2-methoxyethoxyl)benzene in 95% yield. ¹H NMR (400 MHz, <cdcl3>) δ ppm 6.41-6.47 m (3H), 4.08 (t, 2H), 3.74 (t, 2H), 3.45 (s, 3H).

### Synthesis of 2-(2,6-difluoro-4-(2-methoxyethoxyl)phenyl)-4,4,5,5-tetramethyl-1,3,2-dioxaborolane

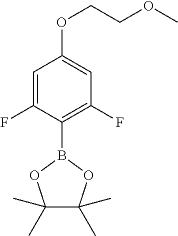 [SMILES: COCCOc1cc(F)c(B2OC(C)(C)C(C)(C)O2)c(F)c1]

To a solution of 1,3-difluoro-5-(2-methoxyethoxyl)benzene (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 1 hr at −78° C., followed by the addition of 2-isopropoxy-4,4,5,5-tetramethyl-1,3,2-dioxaborolane (2.1 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield 2-(2,6-difluoro-4-(2-methoxyethoxyl)phenyl)-4,4,5,5-tetramethyl-1,3,2-dioxaborolane. ¹H NMR (400 MHz, <cdcl3>) δ ppm 6.42 (d, 2H), 4.10 (m, 2H), 3.74 (m, 2H), 3.44 (s, 3H), 1.37 (s, 12H).

### Synthesis of 2-(2,6-difluoro-4-(methylthio)phenyl)-4,4,5,5-tetramethyl-1,3,2-dioxaborolane

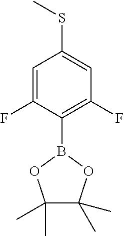 [SMILES: CSc1cc(F)c(B2OC(C)(C)C(C)(C)O2)c(F)c1]

To a solution of (3,5-difluorophenyl)(methyl)sulfane (1.0 eq) in dry THF (0.2M) under an atmosphere of N₂ at −78° C. was added n-butyllithium (1 eq, 1.6M in hexanes) slowly keeping the internal temperature below −65° C. The reaction was stirred for 2 hrs at −78° C., followed by the addition of 2-isopropoxy-4,4,5,5-tetramethyl-1,3,2-dioxaborolane (1.15 eq). The reaction was allowed to warm to room temperature. Upon completion, the reaction was quenched with NaHCO_{3(sat.)} and extracted with EtOAc. The organics were washed with brine, dried over Na₂SO₄, filtered and concentrated to yield a 2-(2,6-difluoro-4-(methylthio)phenyl)-4,4,5,5-tetramethyl-1,3,2-dioxaborolane in 91%. 1H NMR (400 MHz, <cdcl3>) δ ppm 6.71 (dd, 2H), 2.48 (s, 3H), 1.37 (s, 12H).

### Synthesis of 3-(3,5-difluorophenyl)oxetan-3-ol

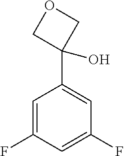 [SMILES: OC1(c2cc(F)cc(F)c2)COC1]

To a solution of 1-bromo-3,5-difluorobenzene in THF (0.27 M) under Ar was added Mg turnings (1.6 M). A reflux condenser was attached and the solution was submerged in a 90° C. oil bath and refluxed for two hours. The oxetan-3-one (1.0 equiv.) was added in THF via syringe. The solution was left stirring at rt under Ar overnight. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 3-(3,5-difluorophenyl)oxetan-3-ol in 56% yield. ¹H NMR (400 MHz, CHLOROFORM-d) δ ppm 4.82 (d, J=7.63 Hz, 2H), 4.91 (d, J=7.63 Hz, 2H), 7.16-7.23 (m, 2H).

### Synthesis of 3-(3,5-difluoro-4-(4,4,5,5-tetramethyl-1,3,2-dioxaborolan-2-yl)phenyl)oxetan-3-ol

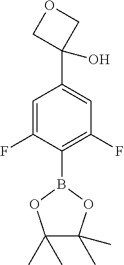 [SMILES: CC1(C)OB(c2c(F)cc(C3(O)COC3)cc2F)OC1(C)C]

Method 2 was followed using 2-isopropoxy-4,4,5,5-tetramethyl-1,3,2-dioxaborolane (2.5 equiv.), butyllithium (2.4 equiv.) and 3-(3,5-difluorophenyl)oxetan-3-ol (1.0 equiv.) to give 3-(3,5-difluoro-4-(4,4,5,5-tetramethyl-1,3,2-dioxaborolan-2-yl)phenyl)oxetan-3-ol in 79% yield. 1H NMR (400 MHz, <cdcl3>) δ ppm 1.34-1.42 (m, 12H), 4.79 (d, J=7.63 Hz, 2H), 4.90 (d, J=7.34 Hz, 2H), 7.17 (d, J=8.22 Hz, 2H).

### Synthesis of 3-(3,5-difluorophenyl)-3-methoxyoxetane

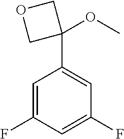

A solution of 3-(3,5-difluorophenyl)oxetan-3-ol (1.0 equiv.) in DMF (0.23 M) was cooled in an ice water bath. NaH, 60% dispersion in mineral oil (1.1 equiv.) was added. The mixture was stirred for 1 hr. iodomethane (1.1 equiv.) was added in a dropwise fashion. The ice bath was removed, and the mixture was stirred for 2 hr at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (2:1 pentane:ether) to give 3-(3,5-difluorophenyl)-3-methoxyoxetane in 83% yield. ¹H NMR (400 MHz, CHLOROFORM-d) 5 ppm 3.18 (s, 3H), 4.70 (d, J=7.04 Hz, 2H), 4.92 (d, J=7.43 Hz, 2H), 6.80 (tt, J=8.66, 2.30 Hz, 1H), 6.99-7.08 (m, 2H).

### Synthesis of 2-(2,6-difluoro-4-(3-methoxyoxetan-3-yl)phenyl)-4,4,5,5-tetramethyl-1,3,2-dioxaborolane

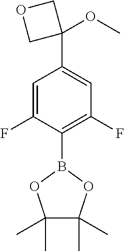 [SMILES: COC1(c2cc(F)c(B3OC(C)(C)C(C)(C)O3)c(F)c2)COC1]

Method 2 was followed using 2-isopropoxy-4,4,5,5-tetramethyl-1,3,2-dioxaborolane (1.3 equiv.), butyllithium (1.3 equiv.) and 3-(3,5-difluorophenyl)-3-methoxyoxetane (1.0 equiv.) to give 2-(2,6-difluoro-4-(3-methoxyoxetan-3-yl)phenyl)-4,4,5,5-tetramethyl-1,3,2-dioxaborolane in 100% yield. 1H NMR (400 MHz, CHLOROFORM-d) δ ppm 1.22-1.26 (m, 12H), 3.16 (s, 3H), 4.67-4.73 (m, 2H), 4.89-4.94 (m, 2H), 7.00 (d, J=8.22 Hz, 2H).

### Synthesis of 1,3-difluoro-5-(isopropoxymethyl)benzene

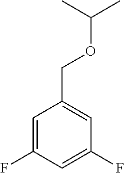

2-propanol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (4:1 pentane:ether) to give 1,3-difluoro-5-(isopropoxymethyl)benzene in 54% yield. 1H NMR (400 MHz, CHLOROFORM-d) δ ppm 1.22 (d, J=5.87 Hz, 6H), 3.68 (spt, J=6.13 Hz, 1H), 4.48 (s, 2H), 6.69 (tt, J=9.00, 2.35 Hz, 1H), 6.83-6.92 (m, 2H).

### Synthesis of 2-(2,6-difluoro-4-(isopropoxymethyl)phenyl)-4,4,5,5-tetramethyl-1,3,2-dioxaborolane

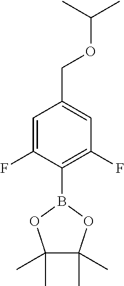 [SMILES: CC(C)OCc1cc(F)c(B2OC(C)(C)C(C)(C)O2)c(F)c1]

Method 2 was followed using 2-isopropoxy-4,4,5,5-tetramethyl-1,3,2-dioxaborolane (1.5 equiv.), butyllithium (1.5 equiv.) and 1,3-difluoro-5-(isopropoxymethyl)benzene (1.0 equiv.) to give 2-(2,6-difluoro-4-(isopropoxymethyl)phenyl)-4,4,5,5-tetramethyl-1,3,2-dioxaborolane in 95% yield.

### Synthesis of 4((3,5-difluorobenzyl)oxy)tetrahydro-2H-pyran

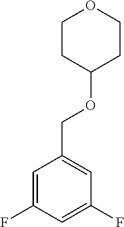 [SMILES: Fc1cc(F)cc(COC2CCOCC2)c1]

Tetrahydro-2H-pyran-4-ol (1.0 equiv.) was dissolved in DMF (0.20 M). Sodium hydride, 60% in mineral oil (1.1 equiv.) was added. The reaction mixture was stirred at ambient temperature for 1 hr. 3,5-difluorobenzyl bromide (1.1 equiv.) was added in a dropwise fashion. The mixture was stirred overnight at ambient temperature. The reaction mixture was quenched by the addition of water. The mixture was extracted with ether. The combined extracts were washed sequentially with water and brine, dried over sodium sulfate, filtered, and concentrated. The crude material was purified by flash chromatography over silica gel (5:2 pentane:ether) to give 4-((3,5-difluorobenzyl)oxy)tetrahydro-2H-pyran in 49% yield. ¹H NMR (400 MHz, CHLOROFORM-d) δ ppm 1.61-1.72 (m, 2H), 1.89-1.98 (m, 2H), 3.46 (ddd, J=11.64, 9.49, 2.74 Hz, 2H), 3.59 (tt, J=8.66, 4.26 Hz, 1H), 3.97 (dt, J=11.74, 4.50 Hz, 2H), 4.54 (s, 2H), 6.71 (tt, J=8.95, 2.20 Hz, 1H), 6.83-6.92 (m, 2H).

### Synthesis of 2-(2,6-difluoro-4-(((tetrahydro-2H-pyran-4-yl)oxy)methyl)phenyl)-4,4,5,5-tetramethyl-1,3,2-dioxaborolane

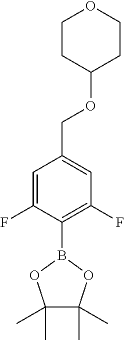 [SMILES: CC1(C)OB(c2c(F)cc(COC3CCOCC3)cc2F)OC1(C)C]

Method 2 was followed using 2-isopropoxy-4,4,5,5-tetramethyl-1,3,2-dioxaborolane (1.6 equiv.), butyllithium (1.6 equiv.) and 4-((3,5-difluorobenzyl)oxy)tetrahydro-2H-pyran (1.0 equiv.) to give 2-(2,6-difluoro-4-(((tetrahydro-2H-pyran-4-yl)oxy)methyl)phenyl)-4,4,5,5-tetramethyl-1,3,2-dioxaborolane in 97% yield.

### Synthesis of 1-(3,5-difluorophenyl)cyclopentanol

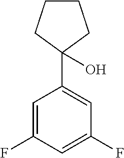 [SMILES: OC1(c2cc(F)cc(F)c2)CCCC1]

To a solution of Mg (6.7 equiv.) in THF (0.14 M) under nitrogen at 0° C. was added 1,4-dibromo butane (3.5 equiv.) dropwise. The reaction was allowed to warm to rt. After stirring for 1 hr at rt, the reaction was cooled to 0° C. and methyl 3,5-difluorobenzoate (1.0 equiv.) in THF (0.14 M) was added dropwise. The cloudy solution became clear and allowed to warm to rt. After 1 hr, the reaction was quenched by the addition of NH4Cl (sat.) and extracted with ethyl acetate. The organic phase was dried with sodium sulfate, filtered and concentrated. The crude material was purified via ISCO SiO2 chromatography (ethyl acetate and heptanes 0-20% ethyl acetate). The pure fractions were concentrated to give 1-(3,5-difluorophenyl)cyclopentanolin 100% yield. 1H NMR (400 MHz, <cdcl3>) δ ppm 1.77-2.11 (m, 8H), 6.67 (tt, J=8.80, 2.35 Hz, 1H), 6.92-7.08 (m, 2H).

### Synthesis of 1-(3,5-difluoro-4-(4,4,5,5-tetramethyl-1,3,2-dioxaborolan-2-yl)phenyl)cyclopentanol

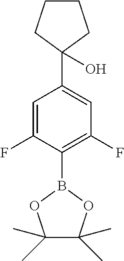 [SMILES: CC1(C)OB(c2c(F)cc(C3(O)CCCC3)cc2F)OC1(C)C]

Method 2 was followed using 2-isopropoxy-4,4,5,5-tetramethyl-1,3,2-dioxaborolane (2.5 equiv.), butyllithium (2.4 equiv.) and 1-(3,5-difluorophenyl)cyclopentanol (1.0 equiv.) to give 1-(3,5-difluoro-4-(4,4,5,5-tetramethyl-1,3,2-dioxaborolan-2-yl)phenyl)cyclopentanol in 100% yield. 1H NMR (400 MHz, <cdcl3>) δ ppm 1.24 (s, 12H), 1.80-2.04 (m, 8H), 6.97 (d, J=9.00 Hz, 2H).

### Synthesis of 4-(3,5-difluorophenyl)tetrahydro-2H-pyran-4-ol

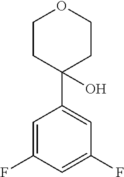 [SMILES: OC1(c2cc(F)cc(F)c2)CCOCC1]

To a solution of 1-bromo-3,5-difluorobenzene (1.6 equiv.) in THF (0.26 M) under Ar was added Mg turnings (1.6 equiv.). A reflux condenser was attached and the solution was submerged in a 90° C. oil bath and refluxed for two hours. The dihydro-2H-pyran-4(3H)-one (1.0 equiv.) was added in THF via syringe. The solution was left stirring at rt under Ar for 5 hrs. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 4-(3,5-difluorophenyl)tetrahydro-2H-pyran-4-ol in 71% yield. ¹H NMR (400 MHz, CHLOROFORM-d) δ ppm 1.59-1.68 (m, 3H), 2.07-2.19 (m, 2H), 3.87-3.93 (m, 4H), 6.72 (tt, J=8.75, 2.20 Hz, 1H), 6.97-7.06 (m, 2H).

### Synthesis of 4-(3,5-difluorophenyl)-3,6-dihydro-2H-pyran

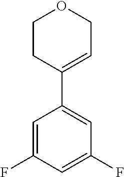 [SMILES: Fc1cc(F)cc(C2=CCOCC2)c1]

4-(3,5-difluorophenyl)tetrahydro-2H-pyran-4-ol (1.0 equiv.) was dissolved in DCM (0.2 M) and cooled to 0° C. TEA (2.8 equiv.) was added to the solution, followed by MsCl (1.3 equiv.). The reaction was stirred at rt for 2 hrs. The solution was cooled to 0° C. and DBU (3.0 equiv.) was added. The reaction was stirred at rt for 18 hrs. The solution was concentrated and the residue was purified by SiO₂ chromatography (0-100% EtOAc in Heptanes) to afford 4-(3,5-difluorophenyl)-3,6-dihydro-2H-pyran in 38% yield. ¹H NMR (400 MHz, <cdcl3>) δ ppm 2.42-2.49 (m, 2H), 3.93 (t, J=5.48 Hz, 2H), 4.32 (q, J=2.74 Hz, 2H), 6.16-6.22 (m, 1H), 6.70 (tt, J=8.80, 2.35 Hz, 1H), 6.85-6.94 (m, 2H).

### Synthesis of 4-(3,5-difluorophenyl)tetrahydro-2H-pyran

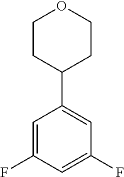 [SMILES: Fc1cc(F)cc(C2CCOCC2)c1]

To a solution of 4-(3,5-difluorophenyl)-3,6-dihydro-2H-pyran (1.0 equiv.) in methanol (0.2 M) was added 10% Pd/C (0.05 equiv.). The reaction was placed under an atmosphere of hydrogen and stirred for 18 hours. Upon completion, the solution was filtered over a pad of Celite, the pad was washed with DCM, the filtrate was concentrated in vacuo to give 4-(3,5-difluorophenyl)tetrahydro-2H-pyran in 71% yield. ¹H NMR (400 MHz, <cdcl3>) δ ppm 1.76 (br. s., 4H), 2.75 (br. s., 1H), 3.50 (br. s., 2H), 4.08 (d, J=9.78 Hz, 2H), 6.56-6.94 (m, 3H).

### Synthesis of 2-(2,6-difluoro-4-(tetrahydro-2H-pyran-4-yl)phenyl)-4,4,5,5-tetramethyl-1,3,2-dioxaborolane

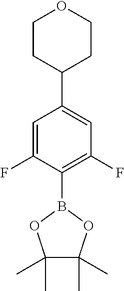 [SMILES: CC1(C)OB(c2c(F)cc(C3CCOCC3)cc2F)OC1(C)C]

Method 2 was followed using 2-isopropoxy-4,4,5,5-tetramethyl-1,3,2-dioxaborolane (2.2 equiv.), butyllithium (1.1 equiv.) and 4-(3,5-difluorophenyl)tetrahydro-2H-pyran (1.0 equiv.) to give 2-(2,6-difluoro-4-(tetrahydro-2H-pyran-4-yl)phenyl)-4,4,5,5-tetramethyl-1,3,2-dioxaborolane in 100% yield. ¹H NMR (400 MHz, <cdcl3>) δ ppm 1.16-1.19 (m, 12H), 1.65-1.74 (m, 4H), 2.60-2.75 (m, 1H), 3.37-3.51 (m, 2H), 4.01 (dt, J=11.54, 3.42 Hz, 2H), 6.67 (d, J=8.22 Hz, 2H).

### Synthesis of 4-(3,5-difluoro-4-(4,4,5,5-tetramethyl-1,3,2-dioxaborolan-2-yl)phenyl)tetrahydro-2H-pyran-4-ol

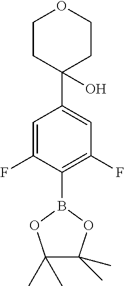 [SMILES: CC1(C)OB(c2c(F)cc(C3(O)CCOCC3)cc2F)OC1(C)C]

Method 2 was followed using 2-isopropoxy-4,4,5,5-tetramethyl-1,3,2-dioxaborolane (2.5 equiv.), butyllithium (2.4 equiv.) and 4-(3,5-difluorophenyl)tetrahydro-2H-pyran-4-ol (1.0 equiv.) to give 4-(3,5-difluoro-4-(4,4,5,5-tetramethyl-1,3,2-dioxaborolan-2-yl)phenyl)tetrahydro-2H-pyran-4-ol in 97% yield. ¹H NMR (400 MHz, <cdcl3>) δ ppm 1.32-1.42 (m, 12H), 1.56-1.65 (m, 2H), 2.11 (d, J=3.13 Hz, 2H), 3.86-3.92 (m, 4H), 6.99 (d, J=9.00 Hz, 2H).

### Synthesis of 1-(3,5-difluorophenyl)cyclobutanol

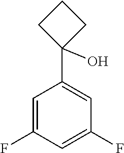 [SMILES: OC1(c2cc(F)cc(F)c2)CCC1]

To a solution of 1-bromo-3,5-difluorobenzene (1.0 equiv.) in THF (0.26 M) under Ar was added Mg turnings (1.6 equiv.). A reflux condenser was attached and the solution was submerged in a 90° C. oil bath and refluxed for two hours. The cyclobutanone (1.0 equiv.) was added in THF via syringe. The solution was left stirring at rt under Ar for 5 hrs. The reaction solution was quenched by addition of NH₄Cl₍ₛₐₜ₎ and the solution was extracted with EtOAc, washed with NaCl_{(sat.)}, dried over MgSO₄, filtered, concentrated and purified by ISCO SiO₂ chromatography (0-100% EtOAc/n-heptanes gradient) to yield 1-(3,5-difluorophenyl)cyclobutanol in 54% yield. ¹H NMR (400 MHz, CHLOROFORM-d) δ ppm 1.69-1.83 (m, 1H), 2.03-2.13 (m, 1H), 2.31-2.43 (m, 2H), 2.45-2.56 (m, 2H), 6.71 (tt, J=8.80, 2.35 Hz, 1H), 6.98-7.07 (m, 2H).

### Synthesis of 1-(3,5-difluoro-4-(4,4,5,5-tetramethyl-1,3,2-dioxaborolan-2-yl)phenyl)cyclobutanol

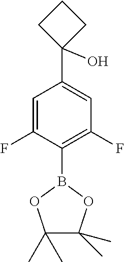 [SMILES: CC1(C)OB(c2c(F)cc(C3(O)CCC3)cc2F)OC1(C)C]

Method 2 was followed using 2-isopropoxy-4,4,5,5-tetramethyl-1,3,2-dioxaborolane (2.5 equiv.), butyllithium (2.4 equiv.) and 1-(3,5-difluorophenyl)cyclobutanol (1.0 equiv.) to give 1-(3,5-difluoro-4-(4,4,5,5-tetramethyl-1,3,2-dioxaborolan-2-yl)phenyl)cyclobutanol in 100% yield. ¹H NMR (400 MHz, <cdcl3>) δ ppm 1.23-1.25 (m, 12H), 1.69-1.82 (m, 1H), 2.05-2.12 (m, 1H), 2.37 (br. s., 2H), 2.47 (br. s., 2H), 7.00 (d, J=8.80 Hz, 2H).

### Synthesis of 4-(3,5-difluorophenoxyl)tetrahydro-2H-pyran

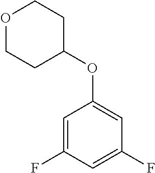 [SMILES: Fc1cc(F)cc(OC2CCOCC2)c1]

To a solution of 3,5-difluorophenol (1.0 equiv.), tetrahydro-2H-pyran-4-ol (1.2 equiv.), and triphenylphosphine (2.0 equiv.) in THF (0.33 M) at 0° C. was added DIAD (2.0 equiv.) dropwise. The reaction mixture was stirred at rt overnight. The mixture was concentrated and purified by flash chromatography over silica gel (heptanes:ethyl acetate gradient) to give 4-(3,5-difluorophenoxyl)tetrahydro-2H-pyran in 90% yield. 1H NMR (400 MHz, <cdcl3>) δ ppm 1.72-1.84 (m, 2H), 1.96-2.09 (m, 2H), 3.59 (ddd, J=11.64, 8.31, 3.52 Hz, 2H), 3.90-4.04 (m, 2H), 4.44 (tt, J=7.78, 3.77 Hz, 1H), 6.32-6.53 (m, 3H).

### Synthesis of 2-(2,6-difluoro-4-((tetrahydro-2H-pyran-4-yl)oxy)phenyl)-4,4,5,5-tetramethyl-1,3,2-dioxaborolane

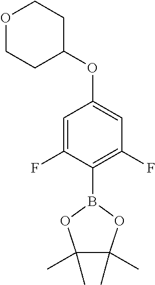 [SMILES: CC1(C)OB(c2c(F)cc(OC3CCOCC3)cc2F)OC1(C)C]

Method 2 was followed using 2-isopropoxy-4,4,5,5-tetramethyl-1,3,2-dioxaborolane (1.5 equiv.), butyllithium (1.3 equiv.) and 4-(3,5-difluorophenoxyl)tetrahydro-2H-pyran (1.0 equiv.) to give 2-(2,6-difluoro-4-((tetrahydro-2H-pyran-4-yl)oxy)phenyl)-4,4,5,5-tetramethyl-1,3,2-dioxaborolane in 33% yield. 1H NMR (400 MHz, <cdcl3>) δ ppm 1.21-1.34 (m, 12H), 1.78 (dtd, J=12.72, 8.31, 8.31, 3.91 Hz, 2H), 1.93-2.09 (m, 2H), 3.59 (ddd, J=11.64, 8.31, 3.13 Hz, 2H), 3.89-4.01 (m, 2H), 4.48 (tt, J=7.78, 3.77 Hz, 1H), 6.40 (d, J=9.39 Hz, 2H).

### Synthesis of 1,3-difluoro-5-isopropoxybenzene

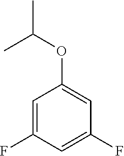

To a solution of 3,5-difluorophenol (1.0 equiv.) in DMF (0.26 M) was added potassium carbonate (2.2 equiv.) followed by 2-iodopropane (1.1 equiv.) and the reaction was stirred overnight at room temperature. The reaction was poured into a separatory funnel and diluted with a 3:1 (v/v) solution of EtOAc:heptanes. The organic phase was washed with water, then sat′d NaHCO₃. The remaining organic phase was dried over MgSO₄, filtered and concentrated in vacuo to provide 1,3-difluoro-5-isopropoxybenzene in 88% yield. 1H NMR (400 MHz, <cdcl3>) δ ppm 1.33 (d, J=6.26 Hz, 6H), 4.48 (dt, J=11.93, 6.16 Hz, 1H), 6.31-6.47 (m, 3H).

### Synthesis of 2-(2,6-difluoro-4-isopropoxyphenyl)-4,4,5,5-tetramethyl-1,3,2-dioxaborolane

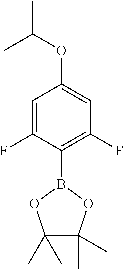 [SMILES: CC(C)Oc1cc(F)c(B2OC(C)(C)C(C)(C)O2)c(F)c1]

Method 2 was followed using 2-isopropoxy-4,4,5,5-tetramethyl-1,3,2-dioxaborolane (2.2 equiv.), butyllithium (1.2 equiv.) and 1,3-difluoro-5-isopropoxybenzene (1.0 equiv.) to give 2-(2,6-difluoro-4-isopropoxyphenyl)-4,4,5,5-tetramethyl-1,3,2-dioxaborolane in 99% yield. 1H NMR (400 MHz, <cdcl3>) δ ppm 1.24 (s, 12H), 1.31-1.33 (m, 6H), 4.43-4.56 (m, 1H), 6.31-6.44 (m, 2H).

### Synthesis of 3-(3,5-difluorophenyl)oxetane

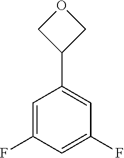 [SMILES: Fc1cc(F)cc(C2COC2)c1]

3,5-difluorophenylboronic acid (2.0 equiv.), (1R,2R)-2-aminocyclohexanol (0.06 equiv.), NaHMDS (2.0 equiv.), and nickel(II) iodide (0.06 equiv.) were dissolved in 2-propanol (0.35 M). The mixture was degassed with N₂, stirred at rt for 10 min and then a solution of 3-iodooxetane (1.0 equiv.) in 2-Propanol (0.70 M) was added. The mixture was sealed and heated at 80° C. in the microwave for 20 minutes. The mixture was filtered through celite, eluting with EtOH and concentrated. The crude residue was purified by ISCO SiO₂ chromatography eluting with 0-100% EtOAc in Heptanes to afford 3-(3,5-difluorophenyl)oxetane in 63% yield. 1H NMR (400 MHz, <cdcl3>) δ 6.88-6.96 (m, 2H), 6.72 (tt, J=2.20, 8.95 Hz, 1H), 5.08 (dd, J=6.26, 8.22 Hz, 2H), 4.71 (t, J=6.26 Hz, 2H), 4.14-4.24 (m, 1H).

### Synthesis of 2-(2,6-difluoro-4-(oxetan-3-yl)phenyl)-4,4,5,5-tetramethyl-1,3,2-dioxaborolane

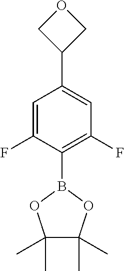 [SMILES: CC1(C)OB(c2c(F)cc(C3COC3)cc2F)OC1(C)C]

Method 2 was followed using 2-isopropoxy-4,4,5,5-tetramethyl-1,3,2-dioxaborolane (1.3 equiv.), butyllithium (1.1 equiv.) and 3-(3,5-difluorophenyl)oxetane (1.0 equiv.) to give 2-(2,6-difluoro-4-(oxetan-3-yl)phenyl)-4,4,5,5-tetramethyl-1,3,2-dioxaborolane in 8% yield. 1H NMR (400 MHz, <cdcl3>) δ ppm 6.90 (d, J=8.22 Hz, 2H), 5.07 (dd, J=6.06, 8.41 Hz, 2H), 4.70 (t, J=6.26 Hz, 2H), 4.13-4.23 (m, 1H), 1.39 (s, 12H).

### Synthesis of tert-butyl(3,5-difluorophenethoxy)dimethylsilane

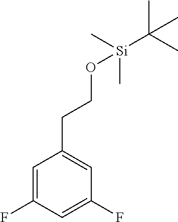 [SMILES: CC(C)(C)[Si](C)(C)OCCc1cc(F)cc(F)c1]

To a solution of 2-(3,5-difluorophenyl)ethanol (1.0 equiv.) in DMF (0.8 M) was added imidazole (2.2 equiv.), followed by TBDMSCl (1.1 equiv.). The reaction was stirred at rt for 3 days. The clear solution was diluted with EtOAc and washed with water, brine, dried over sodium sulfate, filtered and concentrated to tert-butyl(3,5-difluorophenethoxy)dimethylsilane in 88% yield. 1H NMR (400 MHz, <cdcl3>) δ 6.75 (dd, J=2.35, 8.61 Hz, 2H), 6.65 (tt, J=2.35, 9.00 Hz, 1H), 3.81 (t, J=6.65 Hz, 2H), 2.79 (t, J=6.65 Hz, 2H), 0.87 (s, 9H), −0.03-−0.01 (m, 6H).

### Synthesis of tert-butyl(3,5-difluoro-4-(4,4,5,5-tetramethyl-1,3,2-dioxaborolan-2-yl)phenethoxy)dimethylsilane

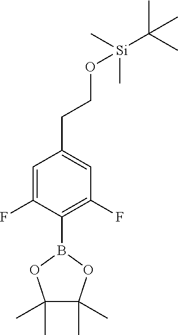 [SMILES: CC1(C)OB(c2c(F)cc(CCO[Si](C)(C)C(C)(C)C)cc2F)OC1(C)C]

Method 2 was followed using 2-isopropoxy-4,4,5,5-tetramethyl-1,3,2-dioxaborolane (1.05 equiv.), butyllithium (1.05 equiv.) and tert-butyl(3,5-difluorophenethoxy)dimethylsilane (1.0 equiv.) to give tert-butyl(3,5-difluoro-4-(4,4,5,5-tetramethyl-1,3,2-dioxaborolan-2-yl)phenethoxy)dimethylsilane in 34% yield. 1H NMR (400 MHz, <cdcl3>) δ ppm 0.00 (s, 6H), 0.91 (s, 9H), 1.40 (s, 12H), 2.80 (td, J=6.46, 3.52 Hz, 2H), 3.82 (td, J=6.46, 3.13 Hz, 2H), 6.71-6.81 (m, 2H).

### Method 3

### Synthesis of 3-(azidomethyl)-6-(2,6-difluoro-4-((tetrahydro-2H-pyran-4-yl)oxy)phenyl)pyridazine

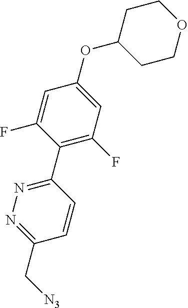 [SMILES: [N-]=[N+]=NCc1ccc(-c2c(F)cc(OC3CCOCC3)cc2F)nn1]

Step 1: To a solution of (6-chloropyridazin-3-yl)methanol (1.0 equiv.) in THF/H₂O (10:1, 0.1 M) is added 2-(2,6-difluoro-4-((tetrahydro-2H-pyran-4-yl)oxy)phenyl)-4,4,5,5-tetramethyl-1,3,2-dioxaborolane (2.5 equiv.) and KF (3.3 equiv.). The solution is degassed with Argon, Pd₂(dba)₃ (0.25 equiv.) and t-Bu₃P (0.5 equiv.) are added, and the solution is heated at 90° C. for 12 hours. The reaction is cooled to rt, the volatiles are removed in vacuo and the residue is purified by ISCO SiO₂ chromatography to yield (6-(2,6-difluoro-4-((tetrahydro-2H-pyran-4-yl)oxy)phenyl)pyridazin-3-yl)methanol.
Step 2: To a solution of (6-(2,6-difluoro-4-((tetrahydro-2H-pyran-4-yl)oxy)phenyl)pyridazin-3-yl)methanol (1.0 equiv.) in pyridine (0.3 M) is added methane sulfonylchloride (1.1 equiv). After stirring for 4 hours the volatiles are removed in vacuo, the residue is partitioned between EtOAc and H₂O, mixed, separated, washed further with H₂O, NaCl_{(sat.)}, dried over MgSO₄ and purified by ISCO SiO₂ chromatography to yield the (6-(2,6-difluoro-4-((tetrahydro-2H-pyran-4-yl)oxy)phenyl)pyridazin-3-yl)methyl methanesulfonate.
Step 3: A solution of (6-(2,6-difluoro-4-((tetrahydro-2H-pyran-4-yl)oxy)phenyl)pyridazin-3-yl)methyl methanesulfonate (1.0 equiv.) in DMF (0.25 M) is treated with sodium azide (5 equiv.) and stirred at RT for 15 hours. The reaction mixture is treated with H₂O, extracted with EtOAc and the organic layer is further washed with H₂O (2×), NaCl_{(sat.)}, dried over MgSO₄ and purified by ISCO SiO₂ chromatography to yield 3-(azidomethyl)-6-(2,6-difluoro-4-((tetrahydro-2H-pyran-4-yl)oxy)phenyl)pyridazine.

Method 3 can be utilized to prepare 3-(azidomethyl)-6-(substituted) pyridazines from boronate esters such as the ones described within. As described in Method 4,3-(azidomethyl)-6-(substituted) pyridazines can be utilized to prepare compounds of the invention.

### Method 4

### Synthesis of N-(4-((1R,3R,4S,5S)-3-amino-5-methyl-4-(2-(methylsulfonyl)ethoxy)cyclohexyl)pyridin-3-yl)-2-(2,6-difluorophenyl)imidazo[1,5-b]pyridazin-7-amine

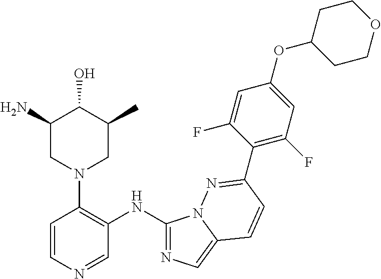 [SMILES: C[C@H]1CN(c2ccncc2Nc2ncc3ccc(-c4c(F)cc(OC5CCOCC5)cc4F)nn23)C[C@@H](N)[C@@H]1O]

PMe₃ (1.0 M solution in THF, 1.1 equiv.) is added dropwise to a solution of 3-(azidomethyl)-6-(2,6-difluoro-4-((tetrahydro-2H-pyran-4-yl)oxy)phenyl)pyridazine (1.0 equiv.) in THF (0.34 M concentration) at RT. The reaction is stirred for 15 min at RT. A solution of tert-butyl ((3R,4R,5S)-4-((tert-butyldimethylsilyl)oxy)-1-(3-isothiocyanatopyridin-4-yl)-5-methylpiperidin-3-yl)carbamate (1.24 equiv.) in THF (0.26 M) is added. After 15 min at RT, the mixture is diluted with EtOAc and is washed with H₂O. The aqueous phase is extracted with EtOAc, the combined organics are dried over MgSO₄, filtered and concentrated. The crude material is purified by ISCO SiO₂ chromatography or RP-HPLC chromatography to yield the O-TBDMS, N-Boc protected product. The O-TBDMS and N-Boc groups are removed by treating with 6N HCl, THF, methanol (1:2:1) at rt for 24 hours, at which time removal of the volatiles in vacuo and purification by RP-HPLC yields (3R,4R,5S)-3-amino-1-(3-((2-(2,6-difluoro-4-((tetrahydro-2H-pyran-4-yl)oxy)phenyl)imidazo[1,5-b]pyridazin-7-yl)amino)pyridin-4-yl)-5-methylpiperidin-4-ol as the TFA salt.

### Synthesis of N-(4-((1R,3R,4S,5S)-3-amino-5-methyl-4-(2-(methylsulfonyl)ethoxy)cyclohexyl)pyridin-3-yl)-2-(2,6-difluorophenyl)imidazo[1,5-b]pyridazin-7-amine

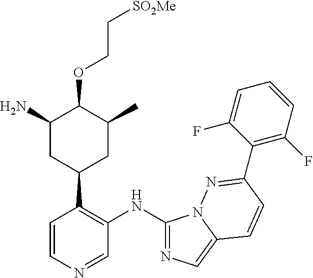 [SMILES: C[C@H]1C[C@@H](c2ccncc2Nc2ncc3ccc(-c4c(F)cccc4F)nn23)C[C@@H](N)[C@H]1OCCS(C)(=O)=O]

PMe₃ (1.0 M solution in THF, 1.1 equiv.) is added dropwise to a solution of 3-(azidomethyl)-6-(2,6-difluorophenyl)pyridazine (1.0 equiv., preparation described in WO2012/148775) in THF (0.34 M concentration) at RT. The reaction is stirred for 15 min at RT. A solution of tert-butyl ((1R,2S,3S,5R)-5-(3-isothio cyanatopyridin-4-yl)-3-methyl-2-(2-(methylsulfonyl)ethoxy)cyclohexyl)carbamate (1.24 equiv.) in THF (0.26 M) is added. After 15 min at RT, the mixture is diluted with EtOAc and is washed with H₂O. The aqueous phase is extracted with EtOAc, the combined organics are dried over MgSO₄, filtered and concentrated. The crude material is purified by ISCO SiO₂ chromatography or RP-HPLC chromatography to yield the Boc protected product. The Boc group is removed by treating with 25% TFA/CH₂Cl₂ for 1 hour or with 4M HCl in dioxane for 3 hours, at which time removal of the volatiles in vacuo and purification by RP-HPLC yields N-(4-((1R,3R,4S,5S)-3-amino-5-methyl-4-(2-(methylsulfonyl)ethoxy)cyclohexyl)pyridin-3-yl)-2-(2,6-difluorophenyl)imidazo[1,5-b]pyridazin-7-amine as the TFA salt.

### Synthesis of tert-butyl ((3R,4R,5 S)-4-((tert-butyldimethylsilyl)oxy)-1-(3-((2-chloroimidazo[1,5-b]pyridazin-7-yl)amino)pyridin-4-yl)-5-methylpiperidin-3-yl)carbamate

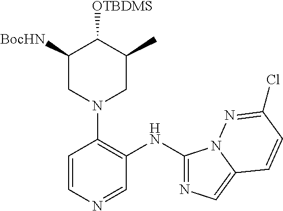 [SMILES: C[C@H]1CN(c2ccncc2Nc2ncc3ccc(Cl)nn23)C[C@@H](NC(=O)OC(C)(C)C)[C@@H]1O[Si](C)(C)C(C)(C)C]

PMe₃ (1.0 M solution in THF, 1.1 equiv.) is added dropwise to a solution of 3-(azidomethyl)-6-chloropyridazine (1.0 equiv., preparation described in WO2012/148775) in THF (0.34 M concentration) at RT. The reaction is stirred for 15 min at RT. A solution of tert-butyl ((3R,4R,5S)-4-((tert-butyldimethylsilyl)oxy)-1-(3-isothiocyanatopyridin-4-yl)-5-methylpiperidin-3-yl)carbamate (1.24 equiv.) in THF (0.26 M) is added. After 15 min at RT, the mixture is diluted with EtOAc and is washed with H₂O. The aqueous phase is extracted with EtOAc, the combined organics are dried over MgSO₄, filtered and concentrated. The crude material is purified by ISCO SiO₂ chromatography or RP-HPLC chromatography to yield tert-butyl ((3R,4R,5S)-4-((tert-butyldimethylsilyl)oxy)-1-(3-((2-chloroimidazo[1,5-b]pyridazin-7-yl)amino)pyridin-4-yl)-5-methylpiperidin-3-yl)carbamate.

### Synthesis of (1R,2R,4R,6S)-2-((tert-butoxycarbonyl)amino)-4-(3-((2-chloroimidazo[1,5-b]pyridazin-7-yl)amino)pyridin-4-yl)-6-methylcyclohexyl acetate

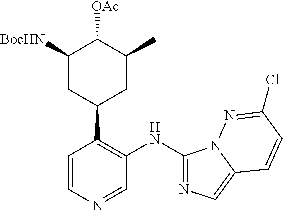 [SMILES: CC(=O)O[C@@H]1[C@@H](C)C[C@@H](c2ccncc2Nc2ncc3ccc(Cl)nn23)C[C@H]1NC(=O)OC(C)(C)C]

PMe₃ (1.0 M solution in THF, 1.1 equiv.) is added dropwise to a solution of 3-(azidomethyl)-6-chloropyridazine (1.0 equiv., preparation described in WO2012/148775) in THF (0.34 M concentration) at RT. The reaction is stirred for 15 min at RT. A solution of (1R,2R,4R,6S)-2-((tert-butoxycarbonyl)amino)-4-(3-isothiocyanatopyridin-4-yl)-6-methylcyclohexyl acetate (1.24 equiv.) in THF (0.26 M) is added. After 15 min at RT, the mixture is diluted with EtOAc and is washed with H₂O. The aqueous phase is extracted with EtOAc, the combined organics are dried over MgSO₄, filtered and concentrated. The crude material is purified by ISCO SiO₂ chromatography or RP-HPLC chromatography to yield (1R,2R,4R,6S)-2-((tert-butoxycarbonyl)amino)-4-(3-((2-chloroimidazo[1,5-b]pyridazin-7-yl)amino)pyridin-4-yl)-6-methylcyclohexyl acetate.

For compounds prepared utilizing Method 4, if an N-Boc protected amine is present, it is removed by treating with excess 4M HCl/dioxane for 14 hours or by treating with 25% TFA/CH₂Cl₂ for 2 hours. Upon removal of the volatiles in vacuo, the material is purified by RP HPLC yielding after lyophilization the amide product as the TFA salt. Alternatively, the HPLC fractions can be added to EtOAc and solid Na₂CO₃, separated and washed with NaCl_{(sat.)}. Upon drying over MgSO₄, filtering and removing the volatiles in vacuo the free base is obtained. Upon dissolving in MeCN/H₂O, adding 1 eq. of 1 N HCl and lyophilizing, the HCl salt of the amide product is obtained.

For compounds to be prepared utilizing Method 4, if a TBDMS ether is present, it is deprotected prior to Boc removal by treating with 6N HCl, THF, methanol (1:2:1) at room temperature for 12 h. After removal of volatiles in vacuo, the Boc amino group is deprotected as described above. Alternatively, the TBDMS ether and Boc group can be both deprotected with 6N HCl, THF, methanol (1:2:1) if left at rt for 24 hours, or heated at 60° C. for 3 hours.

For compounds prepared utilizing Method 4, if an N-Boc1,2 amino alcohol cyclic carbamate is present, prior to Boc deprotection the cyclic carbamate can be cleaved by treating with Cs₂CO₃ (0.5 eq) in ethanol at a concentration of 0.1 M for three hours. After removal of volatiles in vacuo, the Boc amino group is deprotected as described above.

For compounds prepared utilizing Method 4, if an N-Boc, OAc group were present, prior to Boc deprotection, the acetate group can be cleaved by treating with K₂CO₃ (2.0 equiv.) in ethanol at a concentration of 0.1 M for 24 hours.

For compounds prepared utilizing Method 4, if an N-phthalimide group is present, the amine is deprotected by treating with hydrazine in MeOH at 65° C. for three hours. Upon cooling and filtering off the white precipitate, the filtrate is concentrated and purified by RP HPLC to yield the deprotected product.

For compounds prepared utilizing Method 4, if an alkyl or aryl sulfide is present, it can be converted to the corresponding alkyl/aryl sulfone or sulfoxide by standard methods such as treating with oxone in THF/water mixtures or with meta-chloroperbenzoic acid in DCM.

### Method 5

### Synthesis of (3R,4R,5S)-3-amino-1-(3-((2-(2,6-difluoro-4-isopropoxyphenyl)imidazo[1,5-b]pyridazin-7-yl)amino)pyridin-4-yl)-5-methylpiperidin-4-ol

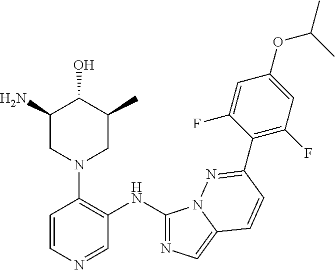 [SMILES: CC(C)Oc1cc(F)c(-c2ccc3cnc(Nc4cnccc4N4C[C@@H](N)[C@H](O)[C@@H](C)C4)n3n2)c(F)c1]

To a solution of tert-butyl ((3R,4R,5S)-4-((tert-butyldimethylsilyl)oxy)-1-(3-((2-chloroimidazo[1,5-b]pyridazin-7-yl)amino)pyridin-4-yl)-5-methylpiperidin-3-yl)carbamate (1.0 equiv.) in THF/H₂O (10:1, 0.1 M) is added 2-(2,6-difluoro-4-isopropoxyphenyl)-4,4,5,5-tetramethyl-1,3,2-dioxaborolane (2.5 equiv.) and KF (3.3 equiv.). The solution is degassed with Argon and Pd₂(dba)₃ (0.25 equiv.) and t-Bu₃P (0.5 equiv.) is added and the solution is heated at 90° C. for 12 hours. The reaction is cooled to rt, the volatiles are removed in vacuo and the residue is purified by ISCO SiO2 chromatography to yield the O-TBDMS, N-Boc protected product. The O-TBDMS and N-Boc groups are removed by treating with 6N HCl, THF, methanol (1:2:1) at rt for 24 hours, at which time removal of the volatiles in vacuo and purification by RP-HPLC yields (3R,4R,5 S)-3-amino-1-(3-((2-(2,6-difluoro-4-isopropoxyphenyl)imidazo[1,5-b]pyridazin-7-yl)amino)pyridin-4-yl)-5-methylpiperidin-4-ol as the TFA salt.

### Synthesis of (1R,2R,4R,6S)-2-amino-4-(3-((2-(2,6-difluoro-4-(2-methoxyethoxyl)phenyl)imidazo[1,5-b]pyridazin-7-yl)amino)pyridin-4-yl)-6-methylcyclohexanol

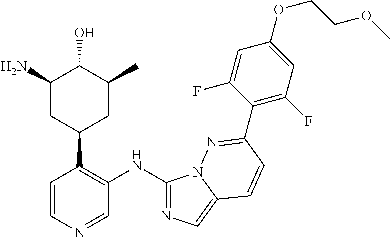 [SMILES: COCCOc1cc(F)c(-c2ccc3cnc(Nc4cnccc4[C@H]4C[C@@H](N)[C@H](O)[C@@H](C)C4)n3n2)c(F)c1]

To a solution of (1R,2R,4R,6S)-2-((tert-butoxycarbonyl)amino)-4-(3-((2-chloroimidazo[1,5-b]pyridazin-7-yl)amino)pyridin-4-yl)-6-methylcyclohexyl acetate (1.0 equiv.) in THF/H₂O (10:1, 0.1 M) is added 2-(2,6-difluoro-4-(2-methoxyethoxyl)phenyl)-4,4,5,5-tetramethyl-1,3,2-dioxaborolane (2.5 equiv.) and KF (3.3 equiv.). The solution is degassed with Argon, Pd₂(dba)₃ (0.25 equiv.) and t-Bu₃P (0.5 equiv.) is added and the solution is heated at 90° C. for 12 hours. The reaction is cooled to rt, the volatiles are removed in vacuo and the residue is purified by ISCO SiO₂ chromatography to yield the O-Acetyl, N-Boc protected product. The acetyl group is deprotected by treating with K₂CO₃ (2.0 equiv.) in ethanol (0.1 M concentration) for 24 hours at rt or for 3 hours at 65° C. Upon cooling to rt, filtering, and removal of the volatiles in vacuo, the Boc group is removed by treating with 25% TFA/CH₂Cl₂ for 1 hour. Upon removal of the volatiles in vacuo and purification by RP-HPLC yields (3R,4R,5S)-3-amino-1-(3-((2-(2,6-difluoro-4-isopropoxyphenyl)imidazo[1,5-b]pyridazin-7-yl)amino)pyridin-4-yl)-5-methylpiperidin-4-ol as the TFA salt.

For compounds prepared utilizing Method 5, in some instances it may be necessary to convert the 2-chloroimidazo[1,5-b]pyridazin-7-yl substrate to the corresponding bromide or iodide by standard methods (i.e. NaBr, acetonitrile, reflux) prior to the transition metal mediated carbon-carbon bond forming reactions. Additionally, in some instances it may be necessary to utilize aryl stannanes in place of the described aryl boronates. The aryl stannanes can be prepared utilizing standard methods from the aryl lithium described herein in the preparation of the aryl boronates.

For compounds prepared utilizing Method 5, if an N-Boc protected amine is present, it is removed by treating with excess 4M HCl/dioxane for 14 hours or by treating with 25% TFA/CH₂Cl₂ for 2 hours. Upon removal of the volatiles in vacuo, the material is purified by RP HPLC yielding after lyophilization the amide product as the TFA salt. Alternatively, the HPLC fractions can be added to EtOAc and solid Na₂CO₃, separated and washed with NaCl_{(sat.)}. Upon drying over MgSO₄, filtering and removing the volatiles in vacuo the free base is obtained. Upon dissolving in MeCN/H₂O, adding 1 eq. of 1 N HCl and lyophilizing, the HCl salt of the amide product is obtained.

For compounds prepared utilizing Method 5, if a TBDMS ether is present, it is deprotected prior to Boc removal by treating with 6N HCl, THF, methanol (1:2:1) at room temperature for 12 h. After removal of volatiles in vacuo, the Boc amino group is deprotected as described above. Alternatively, the TBDMS ether and Boc group can be both deprotected with 6N HCl, THF, methanol (1:2:1) if left at rt for 24 hours, or heated at 60° C. for 3 hours.

For compounds prepared utilizing Method 5, if an N-Boc1,2 amino alcohol cyclic carbamate is present, prior to Boc deprotection the cyclic carbamate can be cleaved by treating with Cs₂CO₃ (0.5 eq) in ethanol at a concentration of 0.1 M for three hours. After removal of volatiles in vacuo, the Boc amino group is deprotected as described above.

For compounds prepared utilizing Method 5, if an N-Boc, OAc group were present, prior to Boc deprotection, the acetate group can be cleaved by treating with K₂CO₃ (2.0 equiv.) in ethanol at a concentration of 0.1 M for 24 hours.

For compounds prepared utilizing Method 5, if an N-phthalimide group was present, the amine is s deprotected by treating with hydrazine in MeOH at 65° C. for three hours. Upon cooling and filtering off the white precipitate, the filtrate is concentrated and purified by RP HPLC to yield the deprotected product.

For compounds prepared utilizing Method 5, if a alkyl or aryl sulfide is present, it can be converted to the corresponding alkyl/aryl sulfone or sulfoxide by standard methods such as treating with oxone in THF/water mixtures or with meta-chloroperbenzoic acid in DCM.

Following the procedures described herein, various compounds of the invention can be prepared.

### Pim1, Pim2, Pim3 AlphaScreen Assays

Pim 1, Pim 2 & Pim 3 AlphaScreen assays using high ATP (11-125×ATP Km) are used to determine the biochemical activity of the inhibitors. The activity of Pim 1, Pim 2, & Pim 3 is measured using a homogeneous bead based system quantifying the amount of phosphorylated peptide substrate resulting from kinase-catalyzed phosphoryl transfer to a peptide substrate. Compounds to be tested are dissolved in 100% DMSO and directly distributed to a white 384-well plate at 0.25 μl per well. To start the reaction, 5 μl of 100 nM Bad peptide (Biotin-AGAGRSRHSSYPAGT-OH) and ATP (concentrations described below) in assay buffer (50 mM Hepes, pH=7.5, 5 mM MgCl₂, 0.05% BSA, 0.01% Tween-20, 1 mM DTT) is added to each well. This is followed by the addition of 5 μl/well of Pim 1, Pim 2 or Pim 3 kinase in assay buffer (concentrations described below). Final assay concentrations (described below) are in 2.5% DMSO. The reactions are performed for ˜2 hours, then stopped by the addition of 10 μl of 0.75 μg/ml anti-phospho Ser/Thr antibody (Cell Signaling), 10 μg/ml Protein A AlphaScreen beads (Perkin Elmer), and 10 μg/ml streptavidin coated AlphaScreen beads in stop/detection buffer (50 mM EDTA, 95 mM Tris, pH=7.5, 0.01% Tween-20). The stopped reactions are incubated overnight in the dark. The phosphorylated peptide is detected via an oxygen anion initiated chemiluminescence/fluorescence cascade using the Envision plate reader (Perkin Elmer).

AlphaScreen Assay Conditions

| | | b-BAD | | ATP Km |
|---|---|---|---|---|
| Enzyme | Enzyme conc. | peptide conc. | ATP conc. | (app) |
| source | (nM) | (nM) | (uM) | (uM) |
| Pim 1 (INV) | 0.0025 | 50 | 2800 | 246 |
| Pim 2 (INV) | 0.01 | 50 | 500 | 4 |
| Pim 3 (NVS) | 0.005 | 50 | 2500 | 50 |

Compounds of the foregoing examples are tested by the Pim 1, Pim 2 & Pim 3 AlphaScreen assays to determine the IC₅₀, the half maximal inhibitory concentration, which represents the concentration of test compound that is required for 50% inhibition of its target in vitro.

### Cell Proliferation Assay

KMS 11 (human myeloma cell line), are cultured in IMDM supplemented with 10% FBS, sodium pyruvate and antibiotics. Cells are plated in the same medium at a density of 2000 cells per well into 96 well tissue culture plates, with outside wells vacant, on the day of assay.

Test compounds supplied in DMSO are diluted into DMSO at 500 times the desired final concentrations before dilution into culture media to 2 times final concentrations. Equal volumes of 2× compounds are added to the cells in 96 well plates and incubated at 37° C. for 3 days.

After 3 days plates are equilibrated to room temperature and equal volume of CellTiter-Glow Reagent (Promega) is added to the culture wells. The plates are agitated briefly and luminescent signal was measured with luminometer. The percent inhibition of the signal seen in cells treated with DMSO alone vs. cells treated with control compound is calculated and used to determine EC₅₀ values (i.e., the concentration of a test compound that is required to obtain 50% of the maximum effect in the cells) for tested compounds.

Using the procedures of the Pim1, Pim2, Pim3 AlphaScreen Assays the IC₅₀ concentrations of compounds of the previous examples are determined.

Using the procedures of Cell Proliferation Assay, the EC₅₀ concentrations of compounds of the examples are determined in KMS 11 cells.

Compound structures in the tables marked as “Chiral” represent the optically active form of the compound, having the absolute stereochemistry as shown; other structures represent compounds in racemic form, and the depicted structure represents the relative stereochemistry at each chiral center. Where a single enantiomer of a racemic compound is shown, the isomer depicted is a preferred embodiment.

1. A compound of Formula (I): 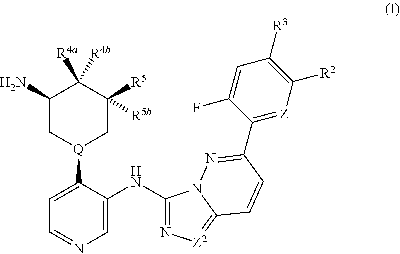
wherein:
Z is CF or N;
Z² is CH or N;
Q is CH or N;
R² is H or —C(O)NHR*;
R^{4a} is H, halo, Me, CF₃, OH, OR⁶, SO₂R⁶, NR′C(═O)R⁶ or NR′C(═O)OR⁶;
R^{4b} is H, halo, Me, CF₃, OH, OR⁶, SO₂R⁶, NR′C(═O)R⁶, or NR′C(═O)OR⁶, or R^{4b} can be taken together with R^{5b} to form a double bond;
provided that when R^{4a} is H and R^{4b} is H or OH, R² and R³ cannot both be H;
R⁵ is H or C₁₋₄ alkyl;
R^{5b} is H, or R^{4b} and R^{5b} taken together form a double bond between the carbon atoms to which they are attached;
R⁶ is C₁₋₄ alkyl optionally substituted with up to three groups selected from halo, CN, C₁₋₄ alkylsulfonyl, hydroxy, and C₁₋₄ alkoxy;
each R³ is independently selected from CN, hydroxy, C₁₋₄ haloalkyl, —S(O)ₚ—R*, C₁₋₄ haloalkoxy, —(CH₂)₀₋₃—OR*, —O—(CH₂)₁₋₃—OR*, —CONR*₂, —(CR′₂)₁₋₃—OR′ or —O—(CR′₂)₁₋₃—OR′, and an optionally substituted member selected from the group consisting of -L-C₁₋₆ alkyl, -L-C₁₋₆ alkylsulfonyl, -L-C₃₋₇ cycloalkyl, and -L-C₄₋₇ heterocycloalkyl, wherein each L is selected from a bond, —O—, —CH₂—, —CH₂—O— and —O—CH₂—, and each C₁₋₆ alkyl, C₁₋₆ alkylsulfonyl, C₃₋₇ cycloalkyl, and C₄₋₇ heterocycloalkyl is optionally substituted with up to two groups selected from halo, CN, hydroxy, C₁₋₄ alkoxy, and R*;
or R³ can be H when R² is —C(O)NHR*;
where each R′ is independently H or Me or Et,
and each R* is independently H or a 4-7 membered cyclic ether, 3-6 membered cycloalkyl, pyrrolidine, or C₁₋₆ alkyl, each of which is optionally substituted with up to three groups selected from halo, oxo, C₁₋₄ alkyl, C₁₋₄ alkoxy, OH, OMe, OEt, and CN; and
p is 0, 1 or 2;
or a pharmaceutically acceptable salt thereof.

2. The compound of claim 1, wherein R³ is selected from OH, OMe, OEt, —SO₂Me, -L-CH₂A, 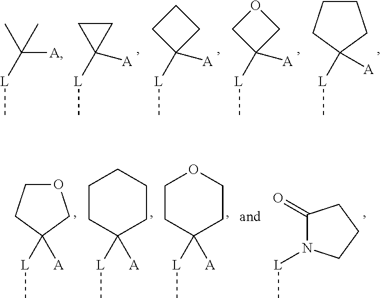
where each L is selected from a bond, —O—, —CH₂—, —OCH₂— and —CH₂O—,
each A is selected from H, OH, F, CN, —OMe and —OEt,
and the dashed bonds indicate where R³ is attached to the ring in Formula I.

3. The compound of claim 1 or 2, wherein R² is H.

4. The compound of any of claims 1-3, wherein Z is CF.

5. The compound of any of claims 1-3, wherein Z is N.

6. The compound of any of claims 1-5, wherein Z² is CH.

7. The compound of any of claims 1-6, wherein R^{4b} is H.

8. The compound of any of claims 1-7, wherein Q is CH.

9. The compound of any one of claims 1-7, wherein Q is N.

10. The compound of any of claims 1-9, wherein R^{4b} and R⁵ taken together form a double bond.

11. The compound of any of claims 1-10, wherein R^{4a} is not H.

12. The compound of any of claims 1-11, wherein R⁵ is Me.

13. The compound of any one of claims 1-12, wherein R³ is of the formula: 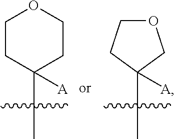
wherein A is H, CN, OH, OMe, or F.

14. The compound of any one of claims 1-12, wherein R³ is of the formula: 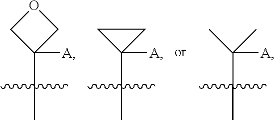
wherein A is H, CN, OH, OMe, or F.

15. The compound of any of claims 1-12, wherein the ring in Formula I that contains Q is selected from: 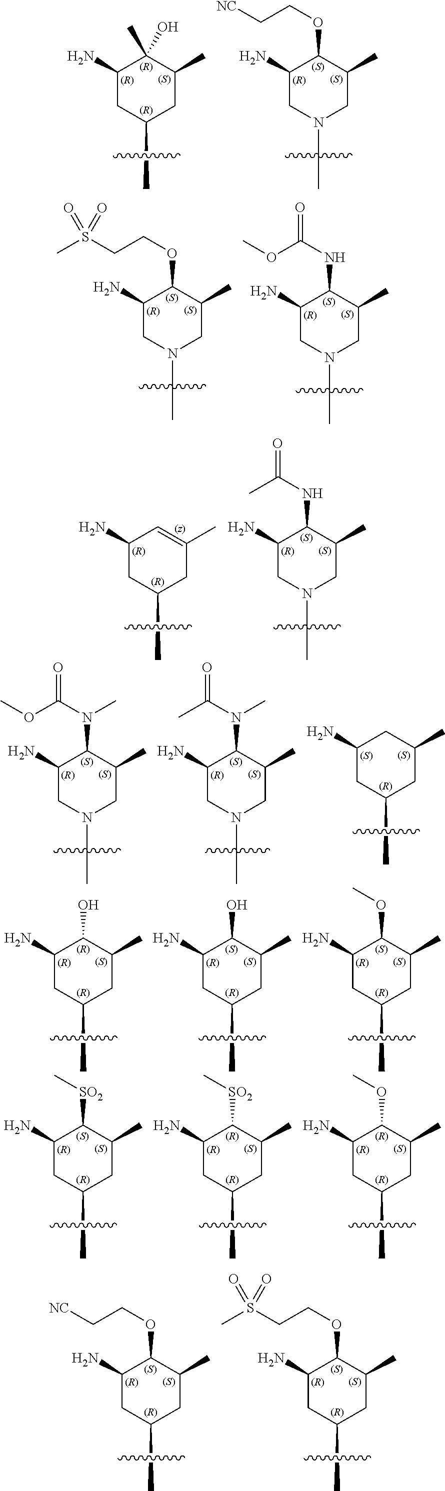 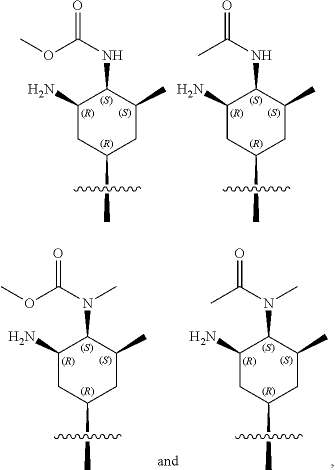
or, when R³ is not H, the ring containing Q can be

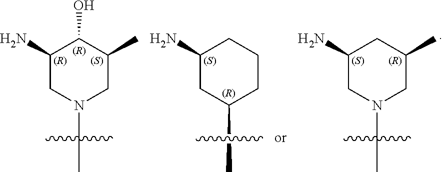

16. The compound of any of claims 1-15, wherein the ring in Formula I that contains Z is selected from:

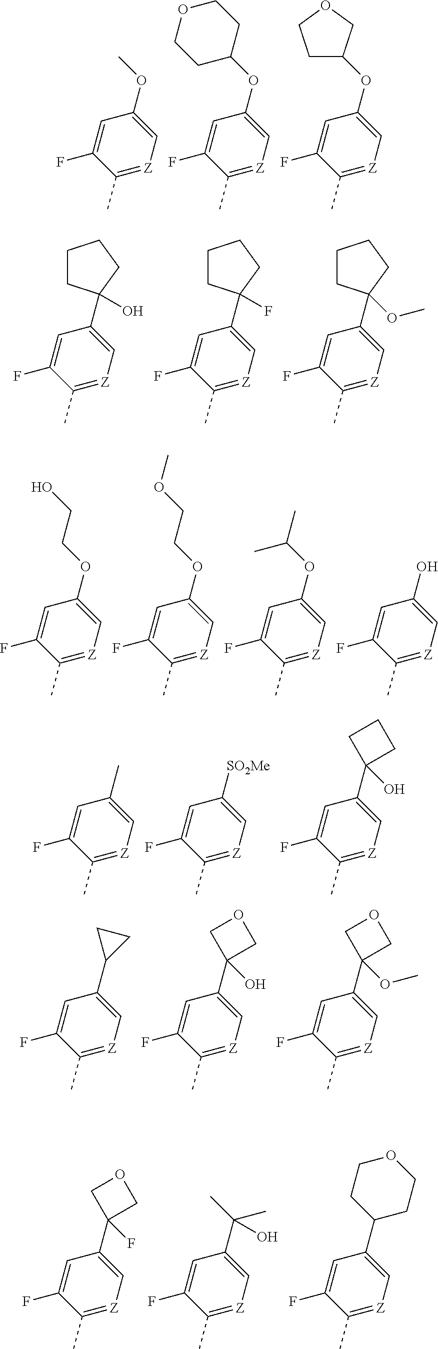

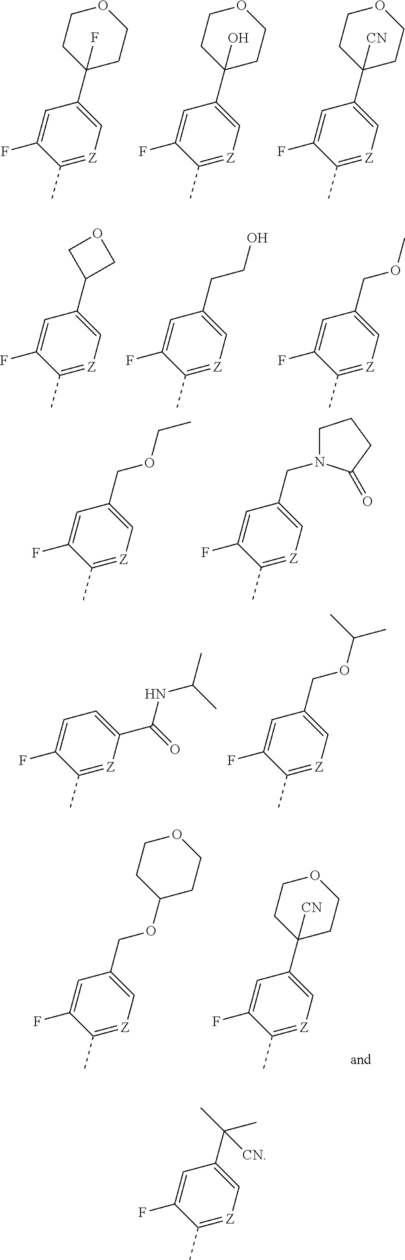

17. The compound of claim 1, which is selected from the compounds in Table I.

18. A pharmaceutical composition comprising a compound of any of the preceding claims and at least one pharmaceutically acceptable excipient.

19. The pharmaceutical composition of claim 18, further comprising a co-therapeutic agent.

20. The pharmaceutical composition of claim 19, wherein the co-therapeutic agent is selected from MEK inhibitors, Velcade, dexamethasone, clofarabine, Mylotarg, lenalidomide, bortezomib, irinotecan, topotecan, gemcitabine, 5-fluorouracil, cytarabine, daunorubicin, PI3 Kinase inhibitors, mTOR inhibitors, DNA synthesis inhibitors, leucovorin, carboplatin, cisplatin, taxanes, tezacitabine, cyclophosphamide, vinca alkaloids, imatinib, anthracyclines, rituximab, thalidomide, bortezomib, and trastuzumab.

21. A method to treat a condition caused or exacerbated by excessive Pim kinase activity, which comprises administering to a subject in need thereof an effective amount of a compound of any one of claims 1-17.

22. The method of claim 21, wherein the condition is a cancer.

23. The method of claim 21, wherein the cancer is selected from carcinoma of the lungs, pancreas, thyroid, ovary, bladder, breast, prostate, or colon, melanoma, myeloid leukemia, multiple myeloma, erythroleukemia, villous colon adenoma, gastric cancers, and osteosarcoma; or the autoimmune disorder is selected from Crohn's disease, inflammatory bowel disease, rheumatoid arthritis, and chronic inflammatory diseases.

24. A compound according to any of claims 1-17 for use in therapy.

25. Use of a compound according to any of claims 1-17 for the preparation of a medicament.
